SEQUENCE LISTING (19 sequences)

SEQ ID NO: 1 - protein, 16 aa, synthetic construct
KRPAATKKAGQAKKKK

SEQ ID NO: 2 - protein, 20 aa, synthetic construct
GRKKRRQRRRPPQPKKKRKV

SEQ ID NO: 3 - protein, 19 aa, synthetic construct
PLSSIFSRIGDPPKKKRKV

SEQ ID NO: 4 - protein, 24 aa, synthetic construct
GALFLGWLGAAGSTMGAPKKKRKV

SEQ ID NO: 5 - protein, 27 aa, synthetic construct
GALFLGFLGAAGSTMGAWSQPKKKRKV

SEQ ID NO: 6 - protein, 20 aa, synthetic construct
KETWWETWWTEWSQPKKKRK

SEQ ID NO: 7 - DNA, 18142 bp, synthetic construct
  source 1..18142: mol_type = other DNA
agtgtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccgaaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggaaaaatggatcattatcttgatattagacttagacctgatccagaatttccaccagctcaacttatgtctgttctttttggaaaacttcatcaagctcttgttgctcaaggaggagatagaattggagtttcttttcctgatcttgatgaatcaagatcaagacttggagaaagacttagaattcatgcttctgctgatgatcttagagctttgcttgctagaccttggcttgaaggacttagagatcatcttcaatttggagaaccagctgttgttccacatccaactccttatagacaagtttcaagagttcaagctaaatctaatccagaaagacttagaagaagacttatgagaagacatgatctttctgaagaagaagctagaaaaagaattcctgatactgttgctagagctttggatttgccttttgttacacttagatcacaatctactggacaacattttagactttttattagacatggaccacttcaagttactgctgaagaaggaggatttacttgttatggactttctaagggaggttttgttccttggtttggatctggagctactaatttttctcttcttaagcaagctggagatgttgaagaaaatcctggacccatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgacgtcgacgatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagccagagctctggtgacggacggcgcgctggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttcctgcaggttcactgccgtataggcagcattaacattaccattaacggttttagagctagaaatagcaagttaaaataaggctagtccgttatcaacttgaaaaagtggcaccgagtcggtgcgttcactgccgtataggcagagggacaccaatgtcctgctgttttagagctagaaatagcaagttaaaataaggctagtccgttatcaacttgaaaaagtggcaccgagtcggtgcgttcactgccgtataggcaggtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattagagctctaccggcgagctttgggtacgtcacgtggctcgagcgcgtagtcctcggtaggcaagcttatttaattcatacagaagcaatctttgtttcagatgttcactacaaaactcatcctcttcttcaatatttttggtttcggaatgatcgctatcttaactcttttccttacacatggccgcaaacgcgttgatgttcttggatggatttgcatgatctttgctttatgcgtgtttgttgcccccatgggtatcatggtgagaatgcgagtcgcaaatttcaacacttgcttctttctgtctctgacagttttttttttttcccctataattatattgattgatttttgttttctctcttctttactctattttccagagaaaagtgataaaaacgaagagtgtcgagttcatgccattttctttatcattcttcctcaccttgactgcggtgatgtggttcttctatggttttctaaagaaagacctttatgttgccgtaagttaactatcacgcatgcatcattatcacgtacatctttctttacattccaccaactttatctttcccattaatcatcaacccagcaactatttcttattcccttttgattaacttccacttacaatttcctttttcttgtcatgaacagattccaaacacattgggctttctttttgggattgtccagatggtgctttatttaatctacagaaaccccaagaaattacctgtagaggatcctaaacttcgcgaattgtccgagcacatcgtcgacgttgcaaagctgagtgcaaccctctgttccgagataaccacagtagtggttccacagcccatagacaatggaaatgatgttgaaggtcaaaaaattaaggaagaaaacgagcaggacattggtgtccctgcagacaaagttaagactaatctttttctctttctcatcttttcacttctccaatcattatcctcggccgaattcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcacttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaacatgatgagctttgataacattaacattaccattaacgtgatcttggttatgttttttctttttaattttgcatgtaatcgttcaaagtggtggtgccatgtctacttgtaaggctgcaatgcagccatgttgtctattatgtcaaatctagttccatttaatgtcaatctttattctcaacctaaaagaagaatatcaatctttatgtaatacgttttttcgagtaaataaaatgtccagtgaatttacagttaatgttaaatcagcattatattttaggaaaatagtattcaacttatagtttaatggttgaaattaaatattaatttttattttatgatgtaataattttaaatttaaattatagctcctggcaagagttattaataaaataatactgccaatatttttttctaaattttatttgaatttgttatttattttatggaaaatatttttaaaaaataattttcatatttttttatataagaagagctcaaaaaaattttaaatccatgttattttacactaaaaaacagaagtttaaataggggagaaatttttacattcgccaacaaaactatataaatttttgttttgaattataaaataataattatttttcctaaaaagaattcttcatgattgtgccaaataagtctcaatgcaattttaaaaaaaatccagacaaaatttgtcttatttctcactgtgctatttttctaataagcattttcattgtgcaattaaatctattggactctaatcaataataaagaaaagggatacctttaatcttttatcgaagatatcaactaattctagagcgcggtaatatcgcagaacaaaagtacctgatatcgagtgtacttcaagtcacaccggcg

SEQ ID NO: 8 - DNA, 22468 bp, synthetic construct
  source 1..22468: mol_type = other DNA
agtgtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccgaaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccaccctacttaaaaaccctttcgattaaatctattattattatttttcatattgttataattaaacaacgtagataaagtttaataaatttattttattaattaatttaattatataaaaaaagaaagaggtaaaaatgaaaaaggcaaaaaggtagttttcttgaacccaaaatttgtagagcatggtcctcttttttgaaaaaaaattaagacaaaacttgagagattttactttaataaatttagattgtaagattgaagaaggaatcatcaaagggggatttaatatttatattttatttttttataaaaaatttatttatttatattttataattaatttgatttataataaattgaggtctaggtaagtatttcacctgccgaatgttggcatatgggatcactatgacaaatcacaaagctgccaaatcaaatttgtctttgcctaaaccgctccatcctaataccacatcaaatcctgtcttcattcattctgagttaaagccttccacacaccataaatactccatccatgtactgcaaagctcccaccatctttatcttcacgaaaaaaaaatcccacttcctcgtactgaaatccagaggtcaccatggatcccattcgtccgcgcacgccaagtcctgcccacgaacttctggccggaccccagccggatagggttcagccgcagccgactgcagatcgtgggggggctccgcctgccggcagccccctggatggcttgcccgctcgacggacgatgtcccgaacccgtctcccgtctccccctgcacccttgcctgcgttctcagcgggcagtttcagcgatctgctccgtcagttcgatccgtcgcttcttgatacatcgctttttaattcgatgtctgccttcggcgctcctcatacagaggctgcctcaggagagggggatgaggtgcaatcgggtctgcgtgcagccgatgacccgcaagccaccgtgcaggtcgctgtgacggccgcgcgaccgccgcgcgccaagccggcgccgcgacggcgtgctgcgcacacctctgacgcttcgccggccgggcaggtcgatctatgcacgctcggctacagccagcagcagcaagagaagatcaaactgaaggctcgttcgacagtagcacagcaccacgaggcactgatcggccatgggtttacacgtgcgcacatcgttgcgctcagccaacacccggcagccttagggaccgtcgctgtcaagtaccaggccatgatcgcggcgttgccggaggcgacacacgaagacatcgttggcggcggcaaacagtggtccggcgcacgcgccctggaagcattgctcacggtgtcgggagagttgagaggtccaccgttacagttggacacaggtcaacttctcaagattgcaaaacgtggcggcgtgaccgcggtggaggcagtgcatgcatggcgcaatgcactgacgggcgctcccctgaacctgaccccggaccaggtggtggccatcgccagcaatattggcggcaagcaggcgctggagacggtgcagcggctgttgccggtgctgtgcgagcaacatggcctgaccctggaccaggtggtggccatcgccagcaatggcggcggcaagcaggcgctggagacggtgcagcggctgttgccggtgctgtgcgagcaacatggtctgaccccggaccaggtggtggctatcgccagcaatattggcggcaagcaggcgctggagacggtgcagcggctgttgccggtgctgtgcgagcaacatggtctgaccccggaccaggtggtggccatcgccagcaataacggcggcaagcaggcgctggagacggtgcagcggctgttgccggtgctgtgcgagcaacatggcctgaccccggaccaggtggtggctatcgccagcaatattggcggcaagcaggcgctggagacggtgcagcggctgttgccggtgctgcgccaggcccatggcctgaccccggcgcaggtggtggccatcgccagccacgatggcggcaagcaggcgctggagacggtgcagcagctgttgccggtgctgtgcgagcaacatggcctgaccccggcgcaggtggtggccatcgccagcaatagcggcggcaagcaggcgctggagacggtgcagcggctgttgccggtgctgcgccaggcccatggcctgaccccggaccaggtggtggccatcgccagcaatagcggcggcaagccggcgctggagacggtgcagcggctgttgccggtgctgtgcgagcaacatggtctgaccccggaccaggtggtggccatcgccagcaataacggcggcaagccggcgctggagacggtgcagcggctgttgccggtgctgtgcgagcaacatggcctgacccgggcgcaggtggtggccatcgccagcaatggcggcggcaagcaggcgctggagacggtgcagcggctgttgccggtgctgcgccaggcccatggcctgaccccggcgcaggtggtggccatcgccagccacgatggcggcaagcaggcgctggagacggtgcagcagctgttgccggtgctgtgcgagcaacatggcctgaccccggcgcaggtggtggccatcgccagcaatagcggcggcaagcaggcgctggagacggtgcagcggctgttgccggtgctgcgccaggcccatggcctgaccccggaccaggtggtggccatcgccagccacgatggcggcaagcaggcgctggagacggtgcagcggctgttgccggtgctgtgcgagcaacatggtctgaccccggaccaggtggtggccatcgccagcaataacggcggcaagcaggcgctggagacggtgcagcggctgttgccggtgctgtgcgagcaacatggcctgaccccggaccaggtggtggccatcgccagccacgatggcggcaagcaggcgctggagacggtgcagcggctgttgccggtgctgtgcgagcaacatggcctgaccccggaccaggtggtggccatcgccagccacgatggcggcaagcaggcgctggagacggtgcagcggctgttgccggtgctgcgccaggcccatggcctgaccccggcgcaggtggtggccatcgccagccacgatggcggcaagccggcgctggagacggtgcagcggctgttgccggtgctgtgcgagcaacatggcctgaccccggaccaggtggtggctatcgccagcaatattggcggcaagcaggcgctggagacggtgcagcggctgttgccggtgctgtgcgagcaacatggcctgaccccggaccaggtggtggccatcgccagcaatggcggcggcaagcaggcgctggagacggtgcagcggctgttgccggtgctgtgcgagcaacatggtctgaccccggcgcaggtggtggccatcgccagcaatggcggcggcaggccggcgctggagagcatttttgcccagttatctcgccctgatcaggcgttggccgcgttgaccaacgaccacctcgtcgccttggcctgcctcggcgggcgtcctgcgctggaggcagtgaaaaagggattgccgcacgcgccgaccttgatcaaaagaaccaatcgccgtcttcccgaacgcacgtcccatcgcgttgccgaccacgcgcaagtggctcgcgtgttgggttttttccagtgccactcccacccagcgcaagcatttgatgaagccatgacgcagttcgggatgagcaggcacgggttgttacagctatttcgcagagccggcgtcaccgaactcgaggcccacagtggaacgctccccccagcctcgcagcgttggcaccgtatcctccaggcatcagggatgaaaagggccgaaccgtccggtgcttccgctcaaacgccggaccaggcgtctttgcatgcattcgccgatgcgctggagcgtgagctggatgcgcccagcccaatagaccgggcgggccaggcgctggcaagcagcagccgtaaacggtcccgatcggagagttctgtcaccggctccttcgcacagcaagctgtcgaggtgcgcgttcccgaacagcgcgatgcgctgcatttcctccccctcagctggggtgtaaaacgcccgcgtaccaggatcgggggcggcctcccggatcctggtacgcccatggacgccgacctggcaccgtccagcaccgtgatgtgggaacaagatgctgaccccttcgcaggggcagcggatgattttccggcattcaacgaagaggagatggcatggttgatggagctatttcctcagtgaggggatcggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggaaaaatggatcattatcttgatattagacttagacctgatccagaatttccaccagctcaacttatgtctgttctttttggaaaacttcatcaagctcttgttgctcaaggaggagatagaattggagtttcttttcctgatcttgatgaatcaagatcaagacttggagaaagacttagaattcatgcttctgctgatgatcttagagctttgcttgctagaccttggcttgaaggacttagagatcatcttcaatttggagaaccagctgttgttccacatccaactccttatagacaagtttcaagagttcaagctaaatctaatccagaaagacttagaagaagacttatgagaagacatgatctttctgaagaagaagctagaaaaagaattcctgatactgttgctagagctttggatttgccttttgttacacttagatcacaatctactggacaacattttagactttttattagacatggaccacttcaagttactgctgaagaaggaggatttacttgttatggactttctaagggaggttttgttccttggtttggatctggagctactaatttttctcttcttaagcaagctggagatgttgaagaaaatcctggacccatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgacgtcgacgatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagccagagctctggtgacggacggcgcgctggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttcctgcaggttcactgccgtataggcagcattaacattaccattaacggttttagagctagaaatagcaagttaaaataaggctagtccgttatcaacttgaaaaagtggcaccgagtcggtgcgttcactgccgtataggcagagggacaccaatgtcctgctgttttagagctagaaatagcaagttaaaataaggctagtccgttatcaacttgaaaaagtggcaccgagtcggtgcgttcactgccgtataggcaggtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattagagctctaccggcgagctttgggtacgtcacgtggctcgagcgcgtagtcctcggtaggcaagcttatttaattcatacagaagcaatctttgtttcagatgttcactacaaaactcatcctcttcttcaatatttttggtttcggaatgatcgctatcttaactcttttccttacacatggccgcaaacgcgttgatgttcttggatggatttgcatgatctttgctttatgcgtgtttgttgcccccatgggtatcatggtgagaatgcgagtcgcaaatttcaacacttgcttctttctgtctctgacagttttttttttttcccctataattatattgattgatttttgttttctctcttctttactctattttccagagaaaagtgataaaaacgaagagtgtcgagttcatgccattttctttatcattcttcctcaccttgactgcggtgatgtggttcttctatggttttctaaagaaagacctttatgttgccgtaagttaactatcacgcatgcatcattatcacgtacatctttctttacattccaccaactttatctttcccattaatcatcaacccagcaactatttcttattcccttttgattaacttccacttacaatttcctttttcttgtcatgaacagattccaaacacattgggctttctttttgggattgtccagatggtgctttatttaatctacagaaaccccaagaaattacctgtagaggatcctaaacttcgcgaattgtccgagcacatcgtcgacgttgcaaagctgagtgcaaccctctgttccgagataaccacagtagtggttccacagcccatagacaatggaaatgatgttgaaggtcaaaaaattaaggaagaaaacgagcaggacattggtgtccctgcagacaaagttaagactaatctttttctctttctcatcttttcacttctccaatcattatcctcggccgaattcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcacttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaacatgatgagctttgataacattaacattaccattaacgtgatcttggttatgttttttctttttaattttgcatgtaatcgttcaaagtggtggtgccatgtctacttgtaaggctgcaatgcagccatgttgtctattatgtcaaatctagttccatttaatgtcaatctttattctcaacctaaaagaagaatatcaatctttatgtaatacgttttttcgagtaaataaaatgtccagtgaatttacagttaatgttaaatcagcattatattttaggaaaatagtattcaacttatagtttaatggttgaaattaaatattaatttttattttatgatgtaataattttaaatttaaattatagctcctggcaagagttattaataaaataatactgccaatatttttttctaaattttatttgaatttgttatttattttatggaaaatatttttaaaaaataattttcatatttttttatataagaagagctcaaaaaaattttaaatccatgttattttacactaaaaaacagaagtttaaataggggagaaatttttacattcgccaacaaaactatataaatttttgttttgaattataaaataataattatttttcctaaaaagaattcttcatgattgtgccaaataagtctcaatgcaattttaaaaaaaatccagacaaaatttgtcttatttctcactgtgctatttttctaataagcattttcattgtgcaattaaatctattggactctaatcaataataaagaaaagggatacctttaatcttttatcgaagatatcaactaattctagagcgcggtaatatcgcagaacaaaagtacctgatatcgagtgtacttcaagtcacaccggcg

SEQ ID NO: 9 - DNA, 22694 bp, synthetic construct
  source 1..22694: mol_type = other DNA
agtgtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccgaaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactatggatcccattcgtccgcgcacgccaagtcctgcccacgaacttctggccggaccccagccggatagggttcagccgcagccgactgcagatcgtgggggggctccgcctgccggcagccccctggatggcttgcccgctcgacggacgatgtcccgaacccgtctcccgtctccccctgcacccttgcctgcgttctcagcgggcagtttcagcgatctgctccgtcagttcgatccgtcgcttcttgatacatcgctttttaattcgatgtctgccttcggcgctcctcatacagaggctgcctcaggagagggggatgaggtgcaatcgggtctgcgtgcagccgatgacccgcaagccaccgtgcaggtcgctgtgacggccgcgcgaccgccgcgcgccaagccggcgccgcgacggcgtgctgcgcacacctctgacgcttcgccggccgggcaggtcgatctatgcacgctcggctacagccagcagcagcaagagaagatcaaactgaaggctcgttcgacagtagcacagcaccacgaggcactgatcggccatgggtttacacgtgcgcacatcgttgcgctcagccaacacccggcagccttagggaccgtcgctgtcaagtaccaggccatgatcgcggcgttgccggaggcgacacacgaagacatcgttggcggcggcaaacagtggtccggcgcacgcgccctggaagcattgctcacggtgtcgggagagttgagaggtccaccgttacagttggacacaggtcaacttctcaagattgcaaaacgtggcggcgtgaccgcggtggaggcagtgcatgcatggcgcaatgcactgacgggcgctcccctgaacctgaccccggaccaggtggtggccatcgccagcaatattggcggcaagcaggcgctggagacggtgcagcggctgttgccggtgctgtgcgagcaacatggcctgaccctggaccaggtggtggccatcgccagcaatggcggcggcaagcaggcgctggagacggtgcagcggctgttgccggtgctgtgcgagcaacatggtctgaccccggaccaggtggtggctatcgccagcaatattggcggcaagcaggcgctggagacggtgcagcggctgttgccggtgctgtgcgagcaacatggtctgaccccggaccaggtggtggccatcgccagcaataacggcggcaagcaggcgctggagacggtgcagcggctgttgccggtgctgtgcgagcaacatggcctgaccccggaccaggtggtggctatcgccagcaatattggcggcaagcaggcgctggagacggtgcagcggctgttgccggtgctgcgccaggcccatggcctgaccccggcgcaggtggtggccatcgccagccacgatggcggcaagcaggcgctggagacggtgcagcagctgttgccggtgctgtgcgagcaacatggcctgaccccggcgcaggtggtggccatcgccagcaatagcggcggcaagcaggcgctggagacggtgcagcggctgttgccggtgctgcgccaggcccatggcctgaccccggaccaggtggtggccatcgccagcaatagcggcggcaagccggcgctggagacggtgcagcggctgttgccggtgctgtgcgagcaacatggtctgaccccggaccaggtggtggccatcgccagcaataacggcggcaagccggcgctggagacggtgcagcggctgttgccggtgctgtgcgagcaacatggcctgacccgggcgcaggtggtggccatcgccagcaatggcggcggcaagcaggcgctggagacggtgcagcggctgttgccggtgctgcgccaggcccatggcctgaccccggcgcaggtggtggccatcgccagccacgatggcggcaagcaggcgctggagacggtgcagcagctgttgccggtgctgtgcgagcaacatggcctgaccccggcgcaggtggtggccatcgccagcaatagcggcggcaagcaggcgctggagacggtgcagcggctgttgccggtgctgcgccaggcccatggcctgaccccggaccaggtggtggccatcgccagccacgatggcggcaagcaggcgctggagacggtgcagcggctgttgccggtgctgtgcgagcaacatggtctgaccccggaccaggtggtggccatcgccagcaataacggcggcaagcaggcgctggagacggtgcagcggctgttgccggtgctgtgcgagcaacatggcctgaccccggaccaggtggtggccatcgccagccacgatggcggcaagcaggcgctggagacggtgcagcggctgttgccggtgctgtgcgagcaacatggcctgaccccggaccaggtggtggccatcgccagccacgatggcggcaagcaggcgctggagacggtgcagcggctgttgccggtgctgcgccaggcccatggcctgaccccggcgcaggtggtggccatcgccagccacgatggcggcaagccggcgctggagacggtgcagcggctgttgccggtgctgtgcgagcaacatggcctgaccccggaccaggtggtggctatcgccagcaatattggcggcaagcaggcgctggagacggtgcagcggctgttgccggtgctgtgcgagcaacatggcctgaccccggaccaggtggtggccatcgccagcaatggcggcggcaagcaggcgctggagacggtgcagcggctgttgccggtgctgtgcgagcaacatggtctgaccccggcgcaggtggtggccatcgccagcaatggcggcggcaggccggcgctggagagcatttttgcccagttatctcgccctgatcaggcgttggccgcgttgaccaacgaccacctcgtcgccttggcctgcctcggcgggcgtcctgcgctggaggcagtgaaaaagggattgccgcacgcgccgaccttgatcaaaagaaccaatcgccgtcttcccgaacgcacgtcccatcgcgttgccgaccacgcgcaagtggctcgcgtgttgggttttttccagtgccactcccacccagcgcaagcatttgatgaagccatgacgcagttcgggatgagcaggcacgggttgttacagctatttcgcagagccggcgtcaccgaactcgaggcccacagtggaacgctccccccagcctcgcagcgttggcaccgtatcctccaggcatcagggatgaaaagggccgaaccgtccggtgcttccgctcaaacgccggaccaggcgtctttgcatgcattcgccgatgcgctggagcgtgagctggatgcgcccagcccaatagaccgggcgggccaggcgctggcaagcagcagccgtaaacggtcccgatcggagagttctgtcaccggctccttcgcacagcaagctgtcgaggtgcgcgttcccgaacagcgcgatgcgctgcatttcctccccctcagctggggtgtaaaacgcccgcgtaccaggatcgggggcggcctcccggatcctggtacgcccatggacgccgacctggcaccgtccagcaccgtgatgtgggaacaagatgctgaccccttcgcaggggcagcggatgattttccggcattcaacgaagaggagatggcatggttgatggagctatttcctcagtgaggggatcggcgcgccagatttgccttttcaatttcagaaagaatgctaacccacagatggttagagaggcttacgcagcaggtatcatcaagacgatctacccgagcaataatctccaggaaatcaaatacttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggaaaaatggatcattatcttgatattagacttagacctgatccagaatttccaccagctcaacttatgtctgttctttttggaaaacttcatcaagctcttgttgctcaaggaggagatagaattggagtttcttttcctgatcttgatgaatcaagatcaagacttggagaaagacttagaattcatgcttctgctgatgatcttagagctttgcttgctagaccttggcttgaaggacttagagatcatcttcaatttggagaaccagctgttgttccacatccaactccttatagacaagtttcaagagttcaagctaaatctaatccagaaagacttagaagaagacttatgagaagacatgatctttctgaagaagaagctagaaaaagaattcctgatactgttgctagagctttggatttgccttttgttacacttagatcacaatctactggacaacattttagactttttattagacatggaccacttcaagttactgctgaagaaggaggatttacttgttatggactttctaagggaggttttgttccttggtttggatctggagctactaatttttctcttcttaagcaagctggagatgttgaagaaaatcctggacccatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgacgtcgacgatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagccagagctctggtgacggacggcgcgctggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttcctgcaggttcactgccgtataggcagcattaacattaccattaacggttttagagctagaaatagcaagttaaaataaggctagtccgttatcaacttgaaaaagtggcaccgagtcggtgcgttcactgccgtataggcagagggacaccaatgtcctgctgttttagagctagaaatagcaagttaaaataaggctagtccgttatcaacttgaaaaagtggcaccgagtcggtgcgttcactgccgtataggcaggtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattagagctctaccggcgagctttgggtacgtcacgtggctcgagcgcgtagtcctcggtaggcaagcttatttaattcatacagaagcaatctttgtttcagatgttcactacaaaactcatcctcttcttcaatatttttggtttcggaatgatcgctatcttaactcttttccttacacatggccgcaaacgcgttgatgttcttggatggatttgcatgatctttgctttatgcgtgtttgttgcccccatgggtatcatggtgagaatgcgagtcgcaaatttcaacacttgcttctttctgtctctgacagttttttttttttcccctataattatattgattgatttttgttttctctcttctttactctattttccagagaaaagtgataaaaacgaagagtgtcgagttcatgccattttctttatcattcttcctcaccttgactgcggtgatgtggttcttctatggttttctaaagaaagacctttatgttgccgtaagttaactatcacgcatgcatcattatcacgtacatctttctttacattccaccaactttatctttcccattaatcatcaacccagcaactatttcttattcccttttgattaacttccacttacaatttcctttttcttgtcatgaacagattccaaacacattgggctttctttttgggattgtccagatggtgctttatttaatctacagaaaccccaagaaattacctgtagaggatcctaaacttcgcgaattgtccgagcacatcgtcgacgttgcaaagctgagtgcaaccctctgttccgagataaccacagtagtggttccacagcccatagacaatggaaatgatgttgaaggtcaaaaaattaaggaagaaaacgagcaggacattggtgtccctgcagacaaagttaagactaatctttttctctttctcatcttttcacttctccaatcattatcctcggccgaattcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcacttatggtgttcaatgcttttcaagatacccagatcatatgaagcggcacgacttcttcaagagcgccatgcctgagggatacgtgcaggagaggaccatctctttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcacggcagacaaacaaaagaatggaatcaaagctaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaacatgatgagctttgataacattaacattaccattaacgtgatcttggttatgttttttctttttaattttgcatgtaatcgttcaaagtggtggtgccatgtctacttgtaaggctgcaatgcagccatgttgtctattatgtcaaatctagttccatttaatgtcaatctttattctcaacctaaaagaagaatatcaatctttatgtaatacgttttttcgagtaaataaaatgtccagtgaatttacagttaatgttaaatcagcattatattttaggaaaatagtattcaacttatagtttaatggttgaaattaaatattaatttttattttatgatgtaataattttaaatttaaattatagctcctggcaagagttattaataaaataatactgccaatatttttttctaaattttatttgaatttgttatttattttatggaaaatatttttaaaaaataattttcatatttttttatataagaagagctcaaaaaaattttaaatccatgttattttacactaaaaaacagaagtttaaataggggagaaatttttacattcgccaacaaaactatataaatttttgttttgaattataaaataataattatttttcctaaaaagaattcttcatgattgtgccaaataagtctcaatgcaattttaaaaaaaatccagacaaaatttgtcttatttctcactgtgctatttttctaataagcattttcattgtgcaattaaatctattggactctaatcaataataaagaaaagggatacctttaatcttttatcgaagatatcaactaattctagagcgcggtaatatcgcagaacaaaagtacctgatatcgagtgtacttcaagtcacaccggcg

SEQ ID NO: 10 - DNA, 1097 bp, Oryza sativa
  source 1..1097: mol_type = genomic DNA
ccaggcttcatcctaaccattacaggcaagatgttgtatgaagaagggcgaacatgcagattgttaaactgacacgtgatggacaagaatgaccgattggtgaccggtctgacaatggtcatgtcgtcagcagacagccatctcccacgtcgcgcctgcttccggtgaaagtggaggtaggtatgggccgtcccgtcagaaggtgattcggatggcagcgatacaaatctccgtccattaatgaagagaagtcaagttgaaagaaagggagggagagatggtgcatgtgggatccccttgggatataaaaggaggaccttgcccacttagaaaggagaggagaaagcaatcccagaagaatcgggggctgactggcactttgtagcttcttcatacgcgaatccaccaaaacacaggagtagggtattacgcttctcagcggcccgaacctgtatacatcgcccgtgtcttgtgtgtttccgctcttgcgaaccttccacagattgggagcttagaacctcacccagggcccccggccgaactggcaaaggggggcctgcgcggtctcccggtgaggagccccacgctccgtcagttctaaattacccgatgagaaagggaggggggggggaaaatctgccttgtttatttacgatccaacggatttggtcgacaccgatgaggtgtcttaccagttaccacgagctagattatagtactaattacttgaggattcggttcctaattttttacccgatcgacttcgccatggaaaattttttattcgggggagaatatccaccctgtttcgctcctaattaagataggaattgttacgattagcaacctaattcagatcagaattgttagttagcggcgttggatccctcacctcatcccatcccaattcccaaacccaaactcctcttccagtcgccgacccaaacacgcatccgccgcctataaatcccacccgcatcgagcctatcaagcccaaaaaaccacaaacccaacgaagaaggaaaaaaaaaggaggaaaagaaaagaggaggaaagcgaagaggttggagagagacgctcgtctccacgtcgccgcc

SEQ ID NO: 11 - DNA, 1097 bp, synthetic construct
  source 1..1097: mol_type = other DNA
ccaggcttcatcctaaccattacaggcaagatgttgtatgaagaagggcgaacatgcagattgttaaactgacacgtgatggacaagaatgaccgattggtgaccggtctgacaatggtcatgtcgtcagcagacagccatctcccacgtcgcgcctgcttccggtgaaagtggaggtaggtatgggccgtcccgtcagaaggtgattcggatggcagcgatacaaatctccgtccattaatgaagagaagtcaagttgaaagaaagggagggagagatggtgcatgtgggatccccttgggatataaaaggaggaccttgcccacttagaaaggagaggagaaagcaatcccagaagaatcgggggctgactggcactttgtagcttcttcatacgcgaatccaccaaaacacaggagtagggtattacgcttctcagcggcccgaacctgtatacatcgcccgtgtcttgtgtgtttccgctcttgcgaaccttccacagattgggagcttagaacctcacccagggcccccggccgaactggcaaaggggggcctgcgcagtctcccggtgaggagccccacgctccgtcagttctaaattacccgatgagaaagggaggggcgggggaaaatctgccttgtttatttacgatccaacggatttggtcgacaccgatgaggtgtcttaccagttaccacgagctagattatagtactaattacttgaggattcggttcctaattttttacccgatcgacttcgccatggaaaattttttattcgggggagaatatccaccctgtttcgctcctaattaagataggaattgttacgattagcaacctaattcagatcagaattgttagttagcggcgttggatccctcacctcatcccatcccaattcccaaacccaaactcctcttccagtcgccgacccaaacacgcatccgccgcctataaatcccacccgcatcgagcctatcaagcccaaaaaaccacaaacccaacgaagaaggaaaaaaaaaggaggaaaagaaaagaggaggaaagcgaagaggttggagagagacactagtctccacgtcgccgcc

SEQ ID NO: 12 - DNA, 1097 bp, synthetic construct; identical to SEQ ID NO: 11
  source 1..1097: mol_type = other DNA

SEQ ID NO: 13 - DNA, 14252 bp, synthetic construct
  source 1..14252: mol_type = other DNA
atgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgcccttcaccatggattacaaggaccacgacggggattacaaggaccacgacattgattacaaggatgatgatgacaagatggctccgaagaagaagaggaaggttggcatccacggggtgccagctgctgacaagaagtactcgatcggcctcgacattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcacatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagaagggagacggctctggatcggggtcgggttctggctcagtcgacgatgctcttgacgattttgacctcgatatgctcgacgctcttgatgattttgatctcgacatgctcgatgcacttgatgactttgaccttgacatgctcgacgcactcgatgacttcgacctcgacatgcttgagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggaggcggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgttccggaactctagataagcttaccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctctggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttg

SEQ ID NO: 14 - DNA, 4687 bp, synthetic construct
  source 1..4687: mol_type = other DNA
ctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgaattaattctcatgtttgacagcttatcatcgattagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttacatgcaccattccttgcggcggcggtgctcaacggcctcaacctactactgggctgcttcctaatgcaggagtcgcataagggagagcgccgacccatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttgatcggggaagaacagtatgtcgagctattttttgacttactggggatcaagcctgattgggagaaaataaaatatcccctatagtgagtcgtattacatggtcatagctgtttcctggcagctctggcccgtgtctcaaaatctctgatgttacattgcacaagataaaaatatatcatcatgcctcctctagaggtctcgctatccaggcttcatcctaaccattacaggcaagatgttgtatgaagaagggcgaacatgcagattgttaaactgacacgtgatggacaagaatgaccgattggtgaccggtctgacaatggtcatgtcgtcagcagacagccatctcccacgtcgcgcctgcttccggtgaaagtggaggtaggtatgggccgtcccgtcagaaggtgattcggatggcagcgatacaaatctccgtccattaatgaagagaagtcaagttgaaagaaagggagggagagatggtgcatgtgggatccccttgggatataaaaggaggaccttgcccacttagaaaggagaggagaaagcaatcccagaagaatcgggggctgactggcactttgtagcttcttcatacgcgaatccaccaaaacacaggagtagggtattacgcttctcagcggcccgaacctgtatacatcgcccgtgtcttgtgtgtttccgctcttgcgaaccttccacagattgggagcttagaacctcacccagggcccccggccgaactggcaaaggggggcctgcgcagtctcccggtgaggagccccacgctccgtcagttctaaattacccgatgagaaagggaggggcgggggaaaatctgccttgtttatttacgatccaacggatttggtcgacaccgatgaggtgtcttaccagttaccacgagctagattatagtactaattacttgaggattcggttcctaattttttacccgatcgacttcgccatggaaaattttttattcgggggagaatatccaccctgtttcgctcctaattaagataggaattgttacgattagcaacctaattcagatcagaattgttagttagcggcgttggatccctcacctcatcccatcccaattcccaaacccaaactcctcttccagtcgccgacccaaacacgcatccgccgcctataaatcccacccgcatcgagcctatcaagcccaaaaaaccacaaacccaacgaagaaggaaaaaaaaaggaggaaaagaaaagaggaggaaagcgaagaggttggagagagacactagtctccacgtcgccgccaacaaagcaccagtggtctagtggtagaatagtaccctgccacggtacagacccgggttcgattcccggctggtgcaagagacgagatctagtctgagtcgaccgtctctgttttagagctaggccaacatgaggatcacccatgtctgcagggcctagcaagttaaaataaggctagtccgttatcaacttggccaacatgaggatcacccatgtctgcagggccaagtggcaccgagtcggtgcaacaaagcaccagtggtctagtggtagaatagtaccctgccacggtacagacccgggttcgattcccggctggtgcaatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagcatgggagaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgtt

SEQ ID NO: 15 - DNA, 4687 bp, synthetic construct
  source 1..4687: mol_type = other DNA
ctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgaattaattctcatgtttgacagcttatcatcgattagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttacatgcaccattccttgcggcggcggtgctcaacggcctcaacctactactgggctgcttcctaatgcaggagtcgcataagggagagcgccgacccatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttgatcggggaagaacagtatgtcgagctattttttgacttactggggatcaagcctgattgggagaaaataaaatatcccctatagtgagtcgtattacatggtcatagctgtttcctggcagctctggcccgtgtctcaaaatctctgatgttacattgcacaagataaaaatatatcatcatgcctcctctagaggtctcccatgccaggcttcatcctaaccattacaggcaagatgttgtatgaagaagggcgaacatgcagattgttaaactgacacgtgatggacaagaatgaccgattggtgaccggtctgacaatggtcatgtcgtcagcagacagccatctcccacgtcgcgcctgcttccggtgaaagtggaggtaggtatgggccgtcccgtcagaaggtgattcggatggcagcgatacaaatctccgtccattaatgaagagaagtcaagttgaaagaaagggagggagagatggtgcatgtgggatccccttgggatataaaaggaggaccttgcccacttagaaaggagaggagaaagcaatcccagaagaatcgggggctgactggcactttgtagcttcttcatacgcgaatccaccaaaacacaggagtagggtattacgcttctcagcggcccgaacctgtatacatcgcccgtgtcttgtgtgtttccgctcttgcgaaccttccacagattgggagcttagaacctcacccagggcccccggccgaactggcaaaggggggcctgcgcagtctcccggtgaggagccccacgctccgtcagttctaaattacccgatgagaaagggaggggcgggggaaaatctgccttgtttatttacgatccaacggatttggtcgacaccgatgaggtgtcttaccagttaccacgagctagattatagtactaattacttgaggattcggttcctaattttttacccgatcgacttcgccatggaaaattttttattcgggggagaatatccaccctgtttcgctcctaattaagataggaattgttacgattagcaacctaattcagatcagaattgttagttagcggcgttggatccctcacctcatcccatcccaattcccaaacccaaactcctcttccagtcgccgacccaaacacgcatccgccgcctataaatcccacccgcatcgagcctatcaagcccaaaaaaccacaaacccaacgaagaaggaaaaaaaaaggaggaaaagaaaagaggaggaaagcgaagaggttggagagagacactagtctccacgtcgccgccaacaaagcaccagtggtctagtggtagaatagtaccctgccacggtacagacccgggttcgattcccggctggtgcaagagacgagatctagtctgagtcgaccgtctctgttttagagctaggccaacatgaggatcacccatgtctgcagggcctagcaagttaaaataaggctagtccgttatcaacttggccaacatgaggatcacccatgtctgcagggccaagtggcaccgagtcggtgcaacaaagcaccagtggtctagtggtagaatagtaccctgccacggtacagacccgggttcgattcccggctggtgcaatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagggacggagaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgtt

SEQ ID NO: 16 - DNA, 4131 bp, synthetic construct
  source 1..4131: mol_type = other DNA
ctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgaattaattctcatgtttgacagcttatcatcgattagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttacatgcaccattccttgcggcggcggtgctcaacggcctcaacctactactgggctgcttcctaatgcaggagtcgcataagggagagcgccgacccatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttgatcggggaagaacagtatgtcgagctattttttgacttactggggatcaagcctgattgggagaaaataaaatatcccctatagtgagtcgtattacatggtcatagctgtttcctggcagctctggcccgtgtctcaaaatctctgatgttacattgcacaagataaaaatatatcatcatgcctcctctagaggtctcgctatgttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctagtctcctttcttaatatatctaacaaacactcctacctcttccaaaataacaaagcaccagtggtctagtggtagaatagtaccctgccacggtacagacccgggttcgattcccggctggtgcaagagacgagatctagtctgagtcgaccgtctctgttttagagctaggccaacatgaggatcacccatgtctgcagggcctagcaagttaaaataaggctagtccgttatcaacttggccaacatgaggatcacccatgtctgcagggccaagtggcaccgagtcggtgcaacaaagcaccagtggtctagtggtagaatagtaccctgccacggtacagacccgggttcgattcccggctggtgcaaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtaccatgttttttttcctctctttttgtaacttgcaagtgttgtgttgtactttatttggcttctttgtaagttggtaacggtggtctatatatggaaaaggtcttgttttgttaaacttatgttagttaactggattcgtctttaaccacaaaaagttttcaataagctacaaatttagacacgcaagccgatgcagtcattagtacatatatttattgcaagtgattacatggcaacccaaacttcaaaaacagtaggttgctccatttagtcatgggagaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgtt

SEQ ID NO: 17 - DNA, 4131 bp, synthetic construct
  source 1..4131: mol_type = other DNA
ctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgaattaattctcatgtttgacagcttatcatcgattagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttacatgcaccattccttgcggcggcggtgctcaacggcctcaacctactactgggctgcttcctaatgcaggagtcgcataagggagagcgccgacccatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttgatcggggaagaacagtatgtcgagctattttttgacttactggggatcaagcctgattgggagaaaataaaatatcccctatagtgagtcgtattacatggtcatagctgtttcctggcagctctggcccgtgtctcaaaatctctgatgttacattgcacaagataaaaatatatcatcatgcctcctctagaggtctcgcatggttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctagtctcctttcttaatatatctaacaaacactcctacctcttccaaaataacaaagcaccagtggtctagtggtagaatagtaccctgccacggtacagacccgggttcgattcccggctggtgcaagagacgagatctagtctgagtcgaccgtctctgttttagagctaggccaacatgaggatcacccatgtctgcagggcctagcaagttaaaataaggctagtccgttatcaacttggccaacatgaggatcacccatgtctgcagggccaagtggcaccgagtcggtgcaacaaagcaccagtggtctagtggtagaatagtaccctgccacggtacagacccgggttcgattcccggctggtgcaaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtaccatgttttttttcctctctttttgtaacttgcaagtgttgtgttgtactttatttggcttctttgtaagttggtaacggtggtctatatatggaaaaggtcttgttttgttaaacttatgttagttaactggattcgtctttaaccacaaaaagttttcaataagctacaaatttagacacgcaagccgatgcagtcattagtacatatatttattgcaagtgattacatggcaacccaaacttcaaaaacagtaggttgctccatttagtggacggagaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgtt

SEQ ID NO: 18 - DNA, 13501 bp, synthetic construct
  source 1..13501: mol_type = other DNA
gccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgaataaaagcatttgcgtttggtttatcattgcgtttatacaaggacagagatccactgagctggaatagcttaaaaccattatcagaacaaaataaaccattttttgttaagaatcagagcatagtaaacaacagaaacaacctaagagaggtaacttgtccaagaagatagctaattatatctattttataaaagttatcatagtttgtaagtcacaaaagatgcaaataacagagaaactaggagacttgagaatatacattcttgtatatttgtattcgagattgtgaaaatttgaccataagtttaaattcttaaaaagatatatctgatctaggtgatggttatagactgtaattttaccacatgtttaatgatggatagtgacacacatgacacatcgacaacactatagcatcttatttagattacaacatgaaatttttctgtaatacatgtctttgtacataatttaaaagtaattcctaagaaatatatttatacaaggagtttaaagaaaacatagcataaagttcaatgagtagtaaaaaccatatacagtatatagcataaagttcaatgagtttattacaaaagcattggttcactttctgtaacacgacgttaaaccttcgtctccaataggagcgctactgattcaacatgccaatatatactaaatacgtttctacagtcaaatgctttaacgtttcatgattaagtgactatttaccgtcaatcctttcccattcctcccactaatccaactttttaattactcttaaatcaccactaagctagtaacgcctatcatgaattagctctactaaatctagcaacctttcaaatttgcagtattgcaggtgtctctgtgtctttaaaatagttgccttatgatttcttcggtttcaagatgatcaaatagttatagatttcatgctcacacatgctcattagatgtgtacatactttacttacccaaatctattttctcgcaaagattttgatggtaaagctgatttggttctattgaactaaatcaaacgagtttcagactgagtgattctaatccggcccattagcccctaaacagacccactaattacgcagcttttaatagagtaattacacctagtttacccactaaaccactaagcactaattatctcacaatctaatgagcttccctcgtaattacttgggctttcactctaccatttatttgtaacagtcaagtctctactgtctctatataaactctctaaagttaacacacaattctcatcacaaacaaatcaaccaaagcaacttctactctttcttctttcgaccttatcaatctgttgagaacgcgccaagctatcaaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcaggcggggcgtaatctagaggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtcccccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttcgataattcttaattaactagttctagagcggccgccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccgggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagggaaggtgcgaacaagtccctgatatgagatcatgtttgtcatctggagccatagaacagggttcatcatgagtcatcaacttaccttcgccgacagtgaattcagcagtaagcgccgtcagaccagaaaagagattttcttgtcccgcatggagcagattctgccatggcaaaacatggtggaagtcatcgagccgttttaccccaaggctggtaatggccggcgaccttatccgctggaaaccatgctacgcattcactgcatgcagcattggtacaacctgagcgatggcgcgatggaagatgctctgtacgaaatcgcctccatgcgtctgtttgcccggttatccctggatagcgccttgccggaccgcaccaccatcatgaatttccgccacctgctggagcagcatcaactggcccgccaattgttcaagaccatcaatcgctggctggccgaagcaggcgtcatgatgactcaaggcaccttggtcgatgccaccatcattgaggcacccagctcgaccaagaacaaagagcagcaacgcgatccggagatgcatcagaccaagaaaggcaatcagtggcactttggcatgaaggcccacattggtgtcgatgccaagagtggcctgacccacagcctggtcaccaccgcggccaacgagcatgacctcaatcagctgggtaatctgctgcatggagaggagcaatttgtctcagccgatgccggctaccaaggggcgccacagcgcgaggagctggccgaggtggatgtggactggctgatcgccgagcgccccggcaaggtaagaaccttgaaacagcatccacgcaagaacaaaacggccatcaacatcgaatacatgaaagccagcatccgggccagggtggagcacccatttcgcatcatcaagcgacagttcggcttcgtgaaagccagatacaaggggttgctgaaaaacgataaccaactggcgatgttattcacgctggccaacctgtttcgggcggaccaaatgatacgtcagtgggagagatctcactaaaaactggggataacgccttaaatggcgaagaaacggtctaaataggctgattcaaggcatttacgggagaaaaaatcggctcaaacatgaagaaatgaaatgactgagtcagccgagaagaatttccccgcttattcgcaccttccttagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcac

SEQ ID NO: 19 - DNA, 12777 bp, synthetic construct
  source 1..12777: mol_type = other DNA
tttgcgaccgctcacgccgattcctcgggcttgggggttccagtgccattgcagggccggcaggcaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctttcgacaaagactggtcggtcggttttggtagacaattgaaattagatggatggtccggttcggtatactataagattaaaaacagttttaaattcagctaaaccgaactcatttgattttattaaaccggaatcatccgattcgagtttgtaaaaaataccgaaattgaaaacactaaacaaaaactgtattaaactgttactgaaataagagaatctcccaattcggtttacgtactactcttcagaaatcagaaccaaaaattcagaaatcggattgaaccaaacttaaattgacggtccggttagttttcggctctacaaattaaaggcccaagtttctgctttaaaagaacgaaatagttaatgggctcaaaccatagaccaggtaagtcatgggcttggttagtccgggtcaacccggtagacccgattcctgaagaaaacctagtggaaggtttaaagttgtaaactttccgaccaaataaacaaaatcgttttccagcttcttccgtcgccactaaaccctgaggctaaacctagacgagtcaaagtgtaaaatcgttaaaccctaagagggagtgagagagagaagaatctagataggagatcaacatgaggtacatggcgcgccaagctatcaaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcaggcggggcgtaatctagaggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtcccccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttcgataattcttaattaactagttctagagcggccgccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccgggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagggaaggtgcgaacaagtccctgatatgagatcatgtttgtcatctggagccatagaacagggttcatcatgagtcatcaacttaccttcgccgacagtgaattcagcagtaagcgccgtcagaccagaaaagagattttcttgtcccgcatggagcagattctgccatggcaaaacatggtggaagtcatcgagccgttttaccccaaggctggtaatggccggcgaccttatccgctggaaaccatgctacgcattcactgcatgcagcattggtacaacctgagcgatggcgcgatggaagatgctctgtacgaaatcgcctccatgcgtctgtttgcccggttatccctggatagcgccttgccggaccgcaccaccatcatgaatttccgccacctgctggagcagcatcaactggcccgccaattgttcaagaccatcaatcgctggctggccgaagcaggcgtcatgatgactcaaggcaccttggtcgatgccaccatcattgaggcacccagctcgaccaagaacaaagagcagcaacgcgatccggagatgcatcagaccaagaaaggcaatcagtggcactttggcatgaaggcccacattggtgtcgatgccaagagtggcctgacccacagcctggtcaccaccgcggccaacgagcatgacctcaatcagctgggtaatctgctgcatggagaggagcaatttgtctcagccgatgccggctaccaaggggcgccacagcgcgaggagctggccgaggtggatgtggactggctgatcgccgagcgccccggcaaggtaagaaccttgaaacagcatccacgcaagaacaaaacggccatcaacatcgaatacatgaaagccagcatccgggccagggtggagcacccatttcgcatcatcaagcgacagttcggcttcgtgaaagccagatacaaggggttgctgaaaaacgataaccaactggcgatgttattcacgctggccaacctgtttcgggcggaccaaatgatacgtcagtgggagagatctcactaaaaactggggataacgccttaaatggcgaagaaacggtctaaataggctgattcaaggcatttacgggagaaaaaatcggctcaaacatgaagaaatgaaatgactgagtcagccgagaagaatttccccgcttattcgcaccttccttagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatgg

(19) United States
(12) Patent Application Publication
(10) Pub. No.: US 2024/0132915 A1
(43) Pub. Date: Apr. 25, 2024

(54) HOMOLOGOUS RECOMBINATION VIA TRANSCRIPTIONAL ACTIVATION

(71) Applicant: Donald Danforth Plant Science Center, St. Louis, MO (US)

(72) Inventors: Blake Meyers, St. Louis, MO (US); Rebecca Bart, St. Louis, MO (US); Kira Veley, St. Louis, MO (US); Ihuoma Okwuonu, Umuahia (NG); Ryan DelPercio, St. Louis, MO (US)

(21) Appl. No.: 18/365,176

(22) Filed: Aug. 2, 2023

Related U.S. Application Data
(60) Provisional application No. 63/394,936, filed on Aug. 3, 2022.

(51) Int. Cl. C12N 15/90; C12N 9/22

(52) U.S. Cl. CPC C12N 15/902; C12N 9/22; C12N 2310/20

(57) ABSTRACT

Compositions and methods for efficiently generating and identifying accurate homologous recombination events are disclosed.

### CROSS-REFERENCE TO RELATED APPLICATIONS

This application claims priority from Provisional Application No. 63/394,936, filed Aug. 3, 2022, the entire contents of which are hereby incorporated by reference.

### SEQUENCE LISTING

This application contains a Sequence Listing that has been submitted in .XML format via PatentCenter and is hereby incorporated by reference in its entirety. The .XML is named 077875-768645 Sequence Listing, and is 140 kilobytes in size.

### FIELD OF THE INVENTION

The present disclosure provides compositions and methods of generating and identifying correct products of homologous recombination.

### BACKGROUND OF THE INVENTION

Genome editing is a revolutionary technology that promises the ability to improve or overcome current deficiencies in the genetic code as well as to introduce novel functionality. However, some applications of the technology do not always generate completely reliable results. For instance, in organisms where the frequency of homologous recombination (HR) is low, the technology as currently practiced is only able to create random ‘mistakes’ at a user-defined location in the genome. For instance, in plants, where the frequency of homologous recombination is less than 1%, editing applications that require replacing an endogenous sequence with a user-defined sequence is possible only in theory. This means, identifying nucleic acid modifications of interest requires laborious screening and has a poor likelihood of success. In fact, in a typical scenario, it simply isn't possible to obtain the optimal, desired change.

Therefore, there is a long-felt need for improved and effective means of genome editing, especially in organisms where the frequency of homologous recombination (HR) is low. More specifically, there is a need for methods of identifying and isolating successful products of homologous recombination in genome editing.

### SUMMARY OF THE INVENTION

One aspect of the present disclosure encompasses a homologous recombination composition. The composition comprises a homologous recombination system and a transcription activation system. The homologous recombination system comprises a programmable nucleic acid modification system, wherein the modification system targets a nucleic acid locus in a gene of interest. The programmable nucleic acid modification system comprises a donor polynucleotide encoding a reporter flanked by regions homologous to the nucleic acid locus. Expression of the reporter after homologous recombination and transcription activation of the gene of interest indicates an accurate homologous recombination event. The homologous recombination composition may generate an accurate homologous recombination event in a plant cell. The homologous recombination composition may be directed to one or more nucleic acid loci. The nucleic acid locus may be in a nuclear, organellar, or extrachromosomal gene of interest.

The programmable nucleic acid modification system may be an RNA-guided clustered regularly interspersed short palindromic repeats (CRISPR)/CRISPR-associated (Cas) (CRISPR/Cas) nuclease system, a CRISPR/Cpf1 nuclease system, a zinc finger nuclease (ZFN), a transcription activator-like effector nuclease (TALEN), a meganuclease, or a programmable DNA binding domain linked to a nuclease domain. The gene of interest may be a protein coding gene or an RNA coding gene, and the reporter may be a selectable or visual reporter. In some aspects, the programmable nucleic acid modification system is CRISPR/Cas system comprising a Cas9 nuclease comprising a transcriptional activator (Cas9-TA), a guide RNA (gRNA) comprising a sequence complementary to a target sequence, and one or more dead RNA (dRNAs) comprising a sequence complementary to a target sequence upstream of a gene of interest's (G01) transcription start-site (TSS).

The gene of interest may be a protein coding gene and the homologous recombination results in the reporter fused in frame with an open reading frame of the gene of interest, the reporter completely or partially replacing a coding sequence of the gene of interest, introduction of the reporter into an intron of the gene of interest, or in an untranslated region of a protein-producing gene of interest, or introduction of a stop codon such that expression of the gene of interest results in the expression of an unfused reporter, fusing the reporter at an N terminus, C terminus, or internally to a polypeptide fragment encoded by a partial open reading frame of the gene of interest. Alternatively, the gene of interest may be a protein-coding gene and the reporter may be a fluorescent RNA aptamer. Additionally, the gene of interest may be a RNA coding gene and the homologous recombination may further introduce a small RNA target site to knock out a lncRNA, one or more polymorphisms at 5′ or 3′ sequences of a miRNA precursor, or may further introduce in phase insertions or replacements of tasiRNAs or phasiRNAs in a tasi/phasiRNAs.

The transcription activation system may comprise a programmable endonuclease modified to lack all nuclease activity, a catalytically inactive Ago endonuclease, a catalytically inactive meganuclease, or a transcription activator-like effectors (TALEs) nucleic acid binding protein. The donor polynucleotide may further encodes sequence modifications in the gene of interest at or near a nucleic acid locus.

A promoter of the gene of interest may be replaced with a heterologous promoter. When a promoter is replaced, the donor polynucleotide may comprise a first nucleic acid sequence targeting a first nucleic acid locus for replacing endogenous promoter control sequences, and a second nucleic acid sequence at a second target nucleic acid locus for introducing the reporter in the gene of interest.

An intergenic nucleic acid sequence between two genes of interest may be modified. When an intergenic region is modified, the donor polynucleotide may encode a first replacement polynucleotide comprising a first reporter flanked by regions of homology to a first nucleic acid locus in a first gene of interest; a second replacement polynucleotide comprising a second reporter flanked by regions of homology to a second nucleic acid locus in a second gene of interest; and an intergenic construct flanked by the first replacement polynucleotide and the second replacement polynucleotide.

The transcription activation system and the homologous recombination system may be encoded on one or more expression constructs. In such an arrangement, expression of the transcription activation system may controlled by a tissue specific promoter. The tissue specific promoter may express the transcription activation system in screenable tissue.

Another aspect of the present disclosure encompasses a system of one or more nucleic acid constructs encoding one or more components of the homologous recombination compositions described above. The system may encode a programmable nucleic acid modification system, a donor polynucleotide encoding a reporter flanked by regions homologous to the nucleic acid locus, a transcription activation system specific for inducing expression of the gene of interest, and combinations thereof. Further, expression of the transcription activation system may be controlled by a tissue specific promoter.

Yet another aspect of the present disclosure encompasses a cell comprising the homologous recombination composition described above. The cell may be a eukaryotic cell, and the eukaryotic cell may be a plant cell. One or more components of the homologous recombination composition may be encoded by the one or more nucleic acid constructs described above.

Another aspect of the present disclosure encompasses a method of generating one or more accurate homologous recombination events in a cell. The method comprises providing one or more of the homologous recombination compositions described above; introducing into the cell the one or more homologous recombination compositions; and identifying an accurate homologous recombination event by identifying a cell expressing the reporter. The cell may be a eukaryotic cell, and the eukaryotic cell may be a plant cell. Additionally, the cell may be ex vivo.

An additional aspect of the present disclosure encompasses a library of homologous recombination compositions comprising two or more of the homologous recombination compositions described above. Each of the two or more homologous recombination compositions targets a distinct nucleic acid locus. The library may target all genes in a genome of a cell. Each of the two or more homologous recombination compositions may knock out a distinct gene of interest. The homologous recombination system may be a CRISPR nuclease system and the transcription activation system is based on a CRISPR nuclease system.

The library may comprise two or more homologous recombination constructs. Each construct comprises a nucleic acid cassette specific for a distinct nucleic acid locus comprising a nucleic acid expression construct encoding a gRNA of the CRISPR-based nucleic acid modification system specific for the nucleic acid locus, a nucleic acid expression construct encoding a gRNA of the CRISPR-based transcription activation system, and a donor polynucleotide encoding a reporter flanked by regions homologous to the nucleic acid locus; and a modular homologous recombination construct comprising a backbone encoding additional components of the CRISPR-based nucleic acid modification system and the CRISPR-based transcription activation system.

Another aspect of the present disclosure encompasses a kit comprising one or more of the homologous recombination compositions described above, wherein each of the homologous recombination compositions targets a distinct nucleic acid locus. Each of the one or more homologous recombination compositions may be encoded by a system of one or more of the nucleic acid constructs described above. The kit may comprise one or more cells comprising one or more of the homologous recombination compositions described above, a system of one or more nucleic acid constructs described above, or combinations thereof.

### Reference to Color Figures

The application file contains at least one figure executed in color. Copies of this patent application publication with color figure will be provided by the Office upon request and payment of the necessary fee.

### BRIEF DESCRIPTION OF THE FIGURES

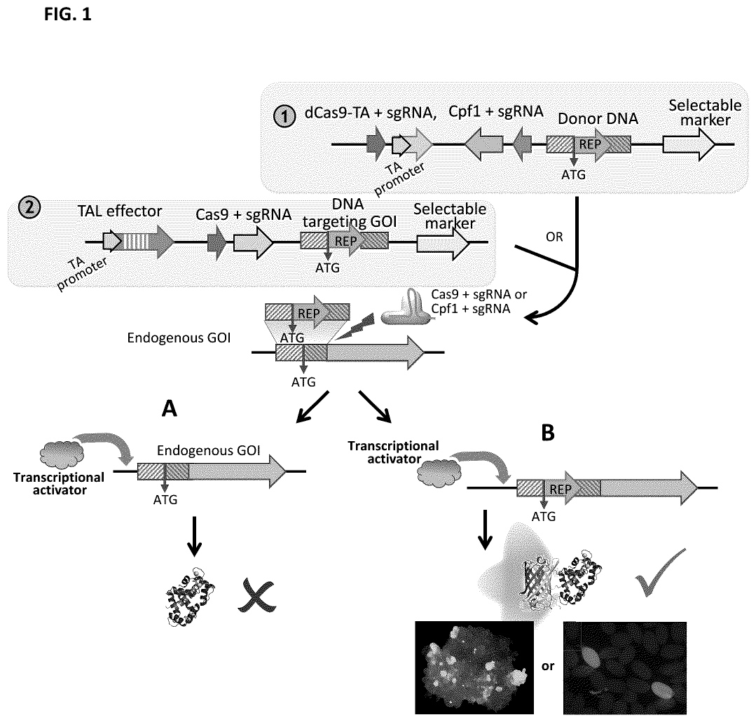

FIG. 1 depicts a schematic overview of the strategy of using a transcriptional activator to identify in-frame gene fusions, products of homologous recombination. Panel 1 depicts an aspect of a DNA construct encoding a donor polynucleotide (DNA targeting gene of interest (GOI)) comprising a reporter flanked by regions homologous to a nucleic acid locus, Cpf1 nickase and single guide RNA, and a CRISPR-based transcription activator with associated sgRNA. Panel 2 depicts an alternative aspect of a DNA construct of Panel 1. In Panel 2, a TAL effector is used for transcriptional activation instead of a CRISPR-based transcription activator. Panels A and B depict the two possible outcomes in the strategy. Panel A depicts an outcome wherein the reporter is not inserted. Panel B depicts an outcome wherein the reporter is inserted, and expressed by the transcription activator.

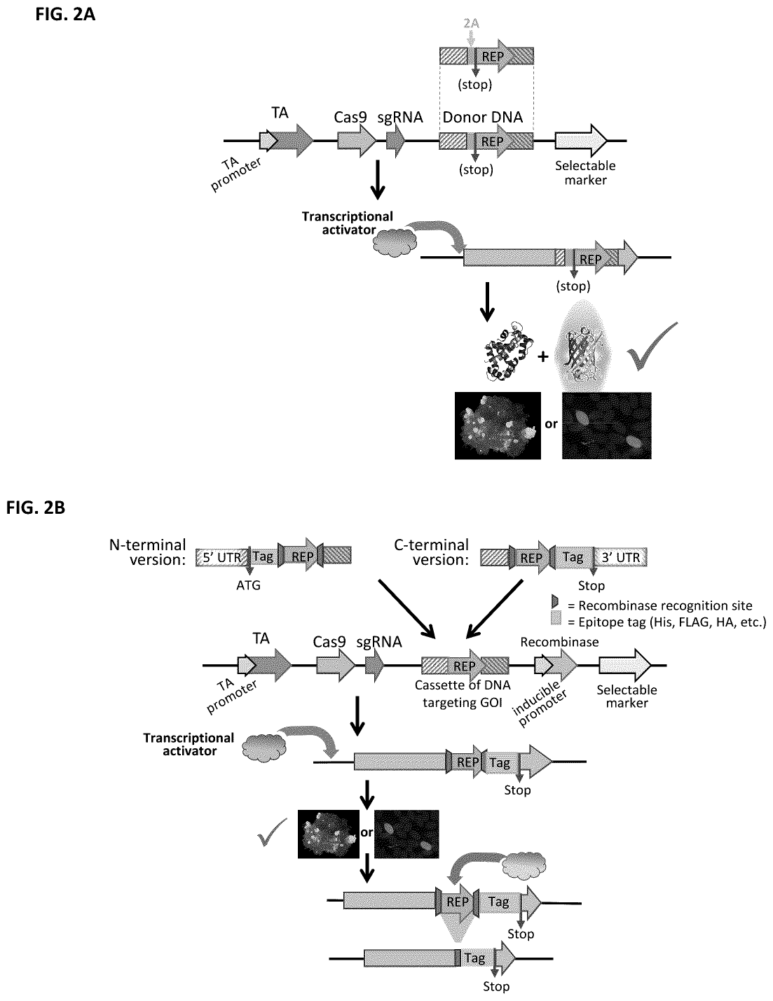

FIG. 2A schematically depict variations on the strategy depicted in FIG. 1, to identify products of homologous recombination without a permanent direct fusion of the reporter to a protein encoded by a gene of interest. In this aspect, the 2A self-cleaving peptide (2A) from the foot-and-mouth-disease virus (FMDV) is used. The 2A peptide is depicted fused at the N-terminus or the C terminus of the gene of interest, yielding a separate, unfused reporter protein. In the figure, an aspect is shown where epitope tags are added upstream of the 2A sequence.

FIG. 2B schematically depict variations on the strategy depicted in FIG. 1, to identify products of homologous recombination without a permanent direct fusion of the reporter to a protein encoded by a gene of interest. In this aspect, an alternative strategy is shown, wherein the reporter sequence may be flanked with sites for a site-specific recombinase (inducible, encoded on the T-DNA backbone), to remove the reporter from the genome after identification of the correct product of HR, leaving behind the adjacent epitope tag and a short (˜34 nt) recombinase site.

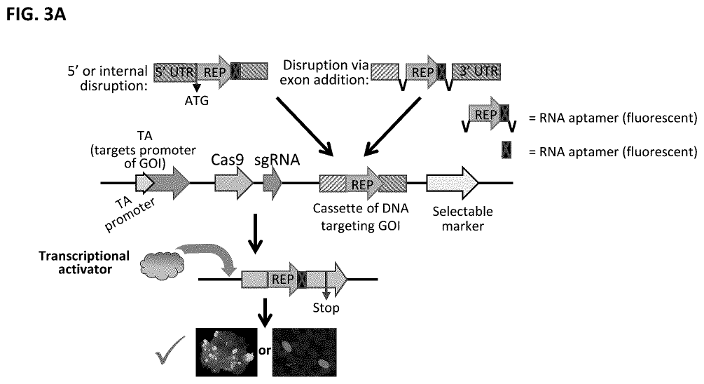

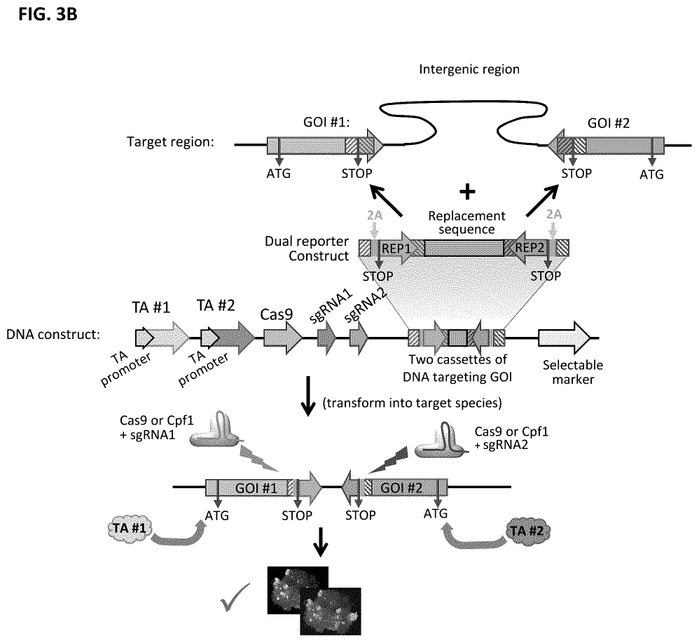

FIG. 3A schematically depict variations on the strategy depicted in FIG. 1 to accelerate targeted knockouts, without requiring large-scale genotyping. In this variation, a homologous recombination event introduces a reporter at the target nucleic acid locus, and disrupts the gene of interest by replacing the open reading frame, disrupting the start codon, and/or 5′, 3′, or internal coding exons, and providing a visual reporter that is induced by the TA in the tissue in which screening is performed (seed or callus).

FIG. 3B schematically depict variations on the strategy depicted in FIG. 1 to accelerate targeted knockouts to introduce deletions between two genes of interest (FIG. 3B). Deletions or sequence replacements between two genes of interest may be achieved by targeting a pair of genes that are located some distance from one another, using HR and a dual reporter deletion-spanning replacement construct to introduce two reporters (green and red) into these genes, while also replacing the intervening sequence with a different nucleic acid sequence. Expression of both reporters after HR indicates replacement of the original nucleic acid sequence between the two genes of interest.

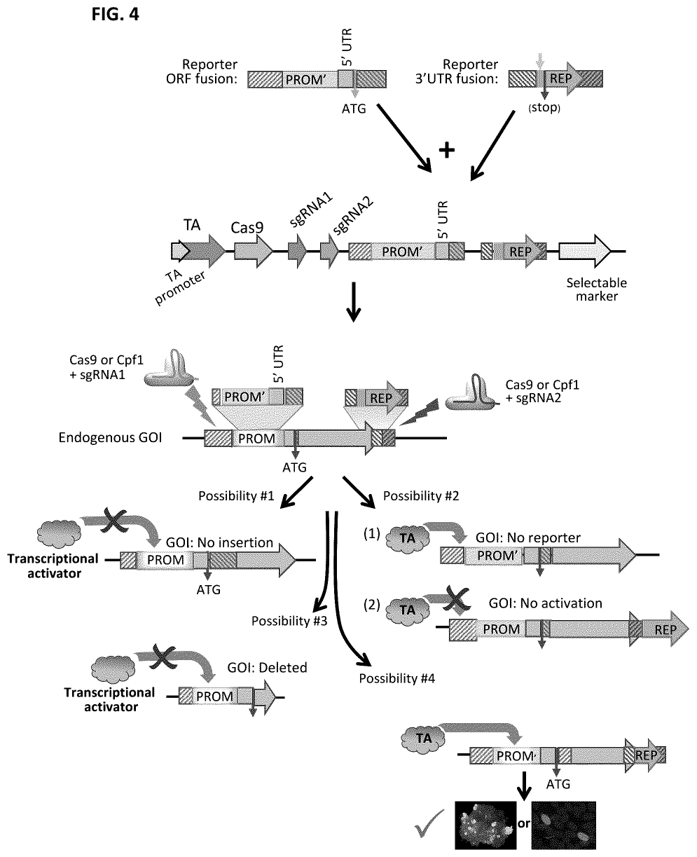

FIG. 4 schematically depicts variations on the strategy depicted in FIG. 1, to replace a promoter of an endogenous gene. The promoter (PROM) is replaced via homology at each end of the replacement cassette (Reporter ORF fusion) with the new promoter (PROM′) using sgRNA1. A second sgRNA (sgRNA2) will target the 3′ end of the target gene to trigger HR with a fragment to insert a 3′ reporter (reporter 3′ fusion), as shown in other figures. Four possible outcomes of the strategy are shown. Possibility #1: no HR, and no TA target site in the genome. Possibility #2: HR occurs as desired, but only at one of the two sites, yielding 2 possible fusions of reporter depicted as fusion (1) or fusion (2) but no activation in the presence of the TA. Possibility #3: The dual sgRNAs delete the GOI, and thus no HR has taken place. Possibility #4: The dual sgRNAs trigger two sites of HR, resulting in a TA-inducible GOI-reporter fusion with the promoter replaced as desired. Activation by a transcription activator specific for the new promoter expresses the reporter in the tissue in which screening is performed (for example, the seed or callus), and demonstrates that the new promoter plus the 3′ end reporter both successfully inserted, as no other gene would be activated by the TA to express the reporter.

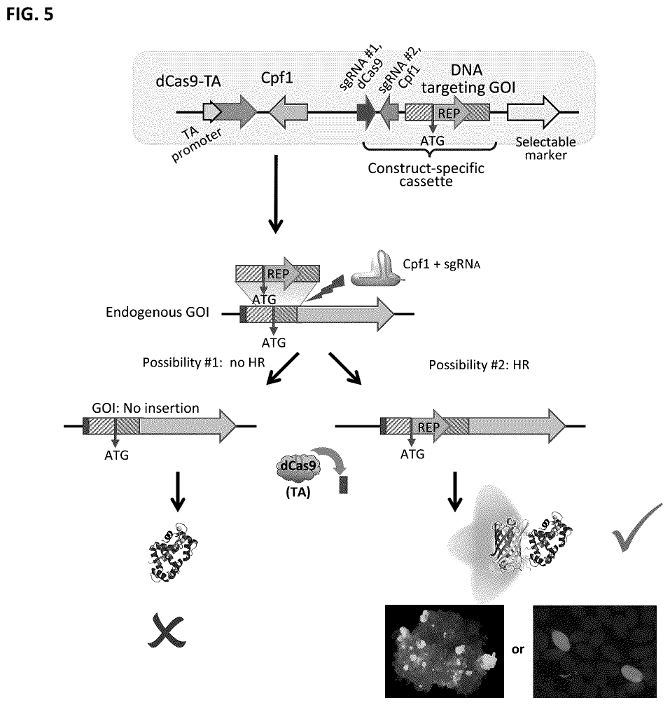

FIG. 5 schematically depicts a large-scale version of the strategy depicted in FIG. 1, but using a library of cassettes incorporating a transcriptional activator and insertion fragment specific to the gene of interest (GOD. Such cassettes may be generated in parallel (100 s to 1000 s of distinct cassettes) and incorporated into a construct encoding the additional components of the homologous recombination composition (T-DNA or other transformed DNA).

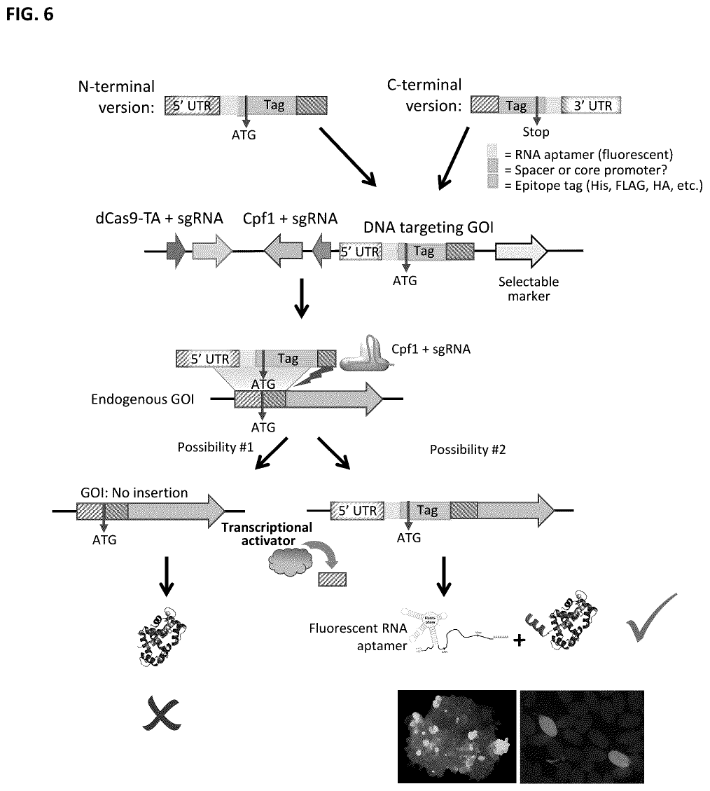

FIG. 6 schematically depicts a variant of the strategy depicted in FIG. 1, using a fluorescent RNA aptamer to detect in-frame fusions of non-visual epitope tags. In this variation, visual detection of insertion is detected using a short (40 to 100 nt) fluorescent RNA aptamer. In this aspect, these RNA aptamers are located in the 5′ UTR upstream of the start codon. A short epitope tag fused in-frame with the open reading frame of the gene of interest is also shown.

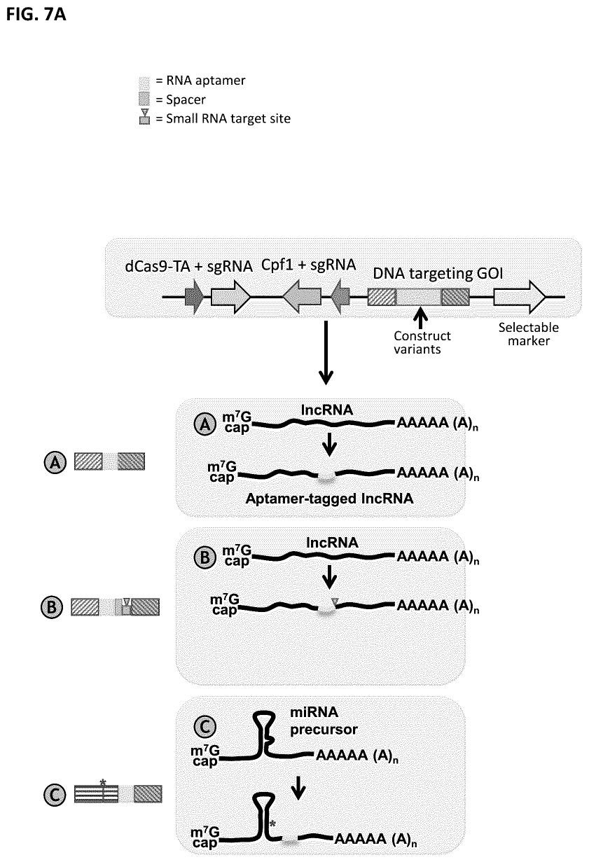

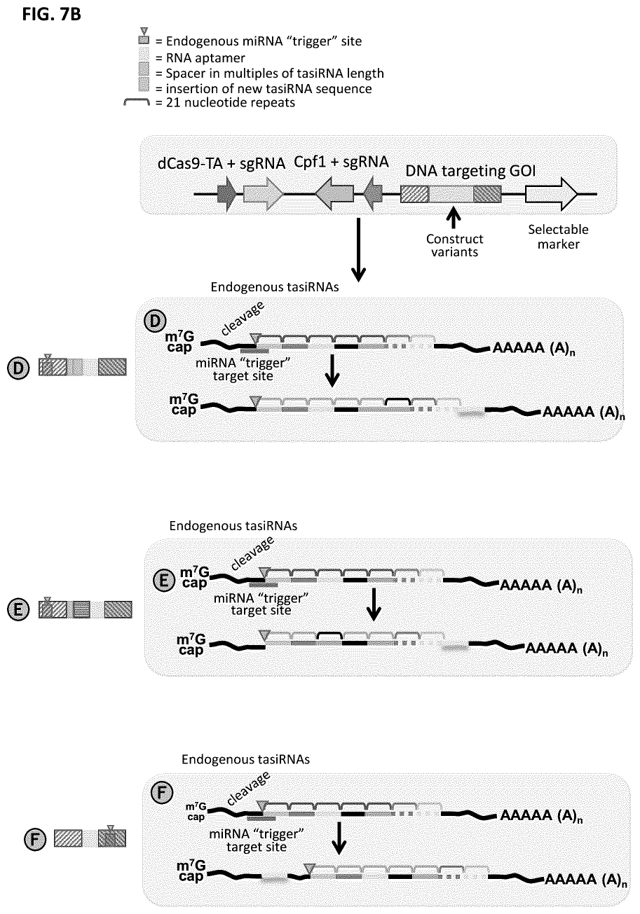

FIG. 7A schematically depicts a variant of the strategy depicted in FIG. 1, using a fluorescent RNA aptamer to create fusions with genes encoding lncRNA. Construct (A) and Panel (A) depicts an aspect wherein the gene encodes a long non-coding RNA. Construct (B) and Panel (B) depicts an aspect wherein a small RNA target site is introduced into the LNCRNA to “knock out” the lncRNA in addition to the fluorescent RNA aptamer. Construct (C) and Panel (C) depicts an aspect wherein the gene encodes a miRNA precursor, and a polymorphism is introduced (red asterisk) in addition to the fluorescent RNA aptamer.

FIG. 7B schematically depicts a variant of the strategy depicted in FIG. 1, using a fluorescent RNA aptamer to create fusions with genes encoding tasi/phasiRNAs. Construct (D) and Panel (D) depicts an aspect wherein a new tasiRNA (pink 21 nucleotide repeat) is added downstream of the primary, endogenous tasiRNAs, in-phase, with the RNA aptamer added further 3′. Construct (E) and Panel (E) depicts an aspect wherein a 3′ insertion or replacement of tasiRNAs (pink 21 nucleotide repeat) is performed in addition to adding an RNA aptamer. Construct (F) and Panel (F) depicts an aspect wherein an aptamer is added upstream of an miRNA target site.

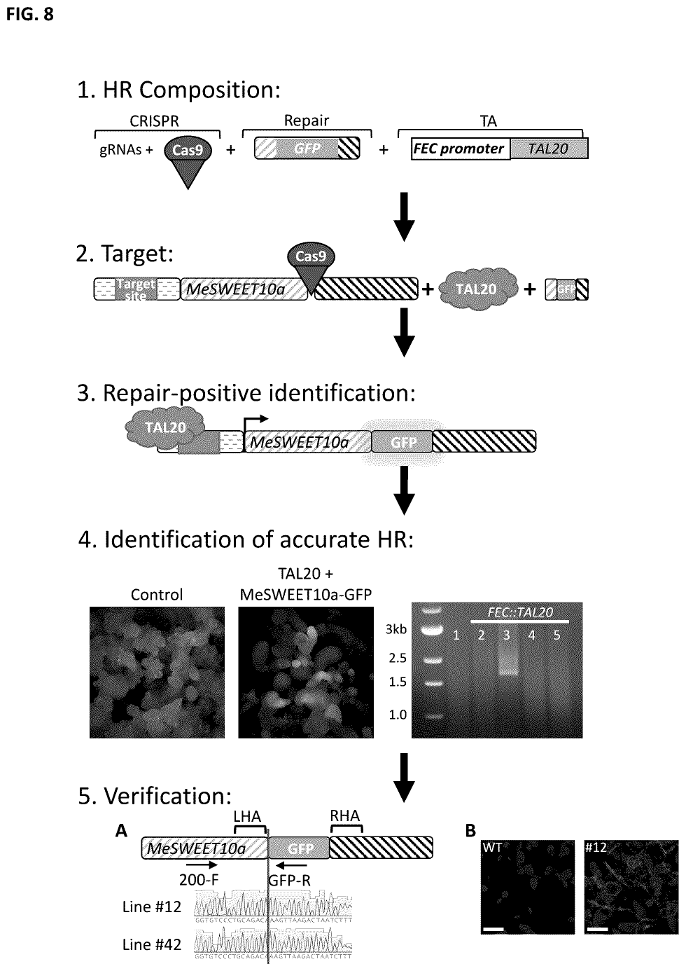

FIG. 8 depicts demonstrated use of a strategy used to fuse a GFP reporter at the C-terminus of the MeSWEET10a protein in cassava callus tissue. Panel 1: Schematic representation of the contents of DNA constructs used to introduce GFP at the endogenous MeSWEET10a locus of cassava. Represented are the CRISPR components (gRNAs and Cas9 nuclease), repair template (GFP plus left and right homology arms (LHA, RHA)), and tissue-specific (FEC) promoter driving expression of a MeSWEET10a-specific transcriptional activator (TAL20). Panel 2: Schematic representation of the repair process depicting digestion site of Cas9 nuclease at the cassava MeSWEET10a target, the TAL20 transcriptional activator, and the repair template. Panel 3: Accurate homologous recombination is visualized when the TAL20 transcription activator induces the expression of the MeSWEET10a/GFP fusion protein. Panel 4: Identification of accurate homologous recombination. Left: Fluorescence imaging photomicrographs of cassava callus tissue. A: negative control cassava callus transformed with a plasm id comprising all the components of homologous recombination composition except the transcription activator. B: positive control cassava callus transformed with all components of the homologous recombination composition, including the TAL20 transcription activator. Fluorescent cells, indicating an accurate homologous recombination event, are only seen in the positive control. Right: PCR screening for GFP integration at MeSWEET10a locus. Lane 1 control genomic DNA, lanes 2-5 GFP-positive lines with a FEC-specific promoter driving TAL20 expression. Panel 5: Depicts a schematic representation of sequence verification. (A) Top: Repair-positive lines identified through PCR were sequenced using a forward primer (200-F) outside of the left homology arm (LHA) and a GFP-specific reverse primer (GFP-R). Bottom: Sequence traces of two cassava cell lines identified as having homologous recombination. The red line indicates the predicted junction of MeSWEET10a and GFP, confirmed by sequencing depicted by the sequence traces. (B) Confocal images of WT and repair positive line #12 leaves depicting MeSWEET10a-GFP ER localization. Pseudocolors: red=chlorophyll, green=GFP. Scale bars=10 μm.

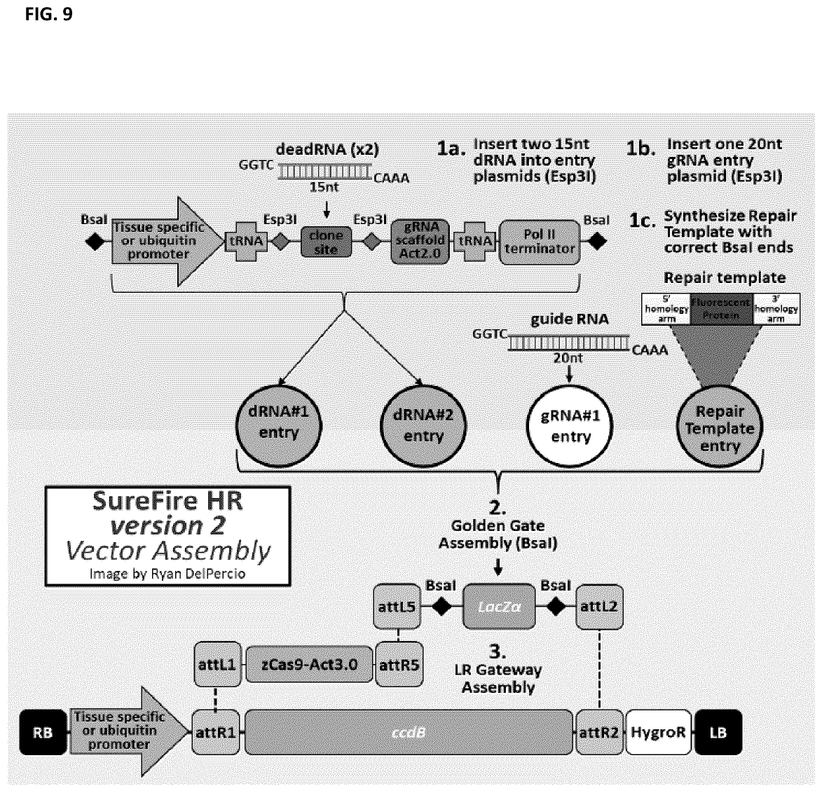

FIG. 9 depicts the 3-step SureFire HRv2 vector assembly strategy. All components colored in green were modifications made to CRISPR-Act2.0 and CRISPR-Act3.0, and unique to Surefire HRv2.

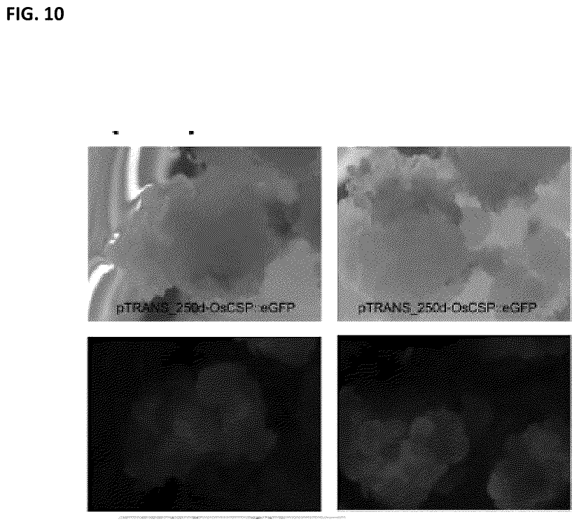

FIG. 10 shows images of rice calli expressing GFP.

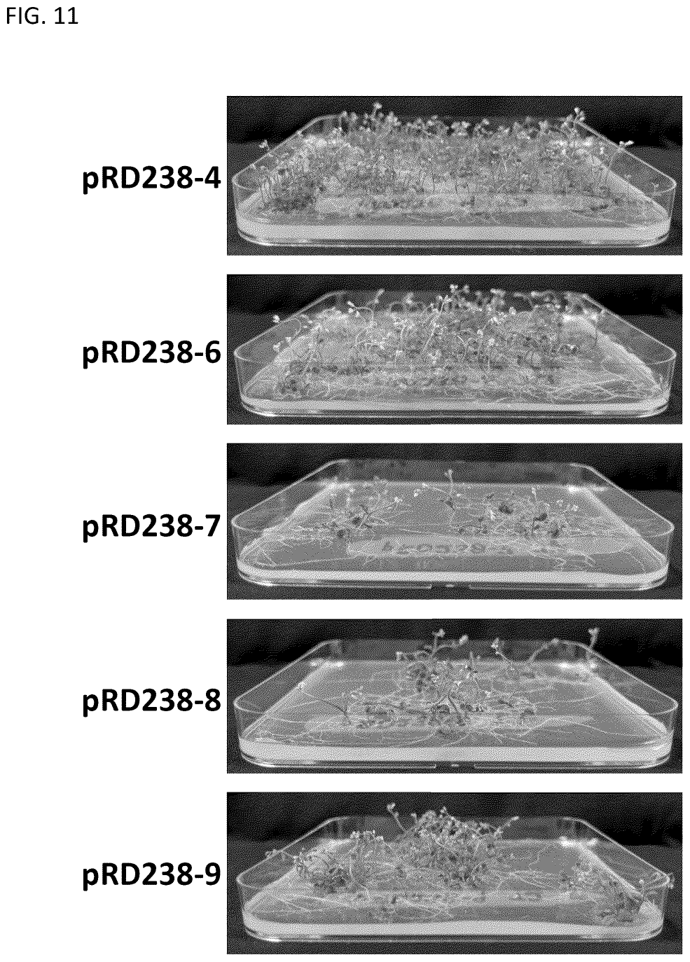

FIG. 11 are photographs of 21-day-old pRD238 Arabidopsis lines showing early flowering induced by dRNAs.

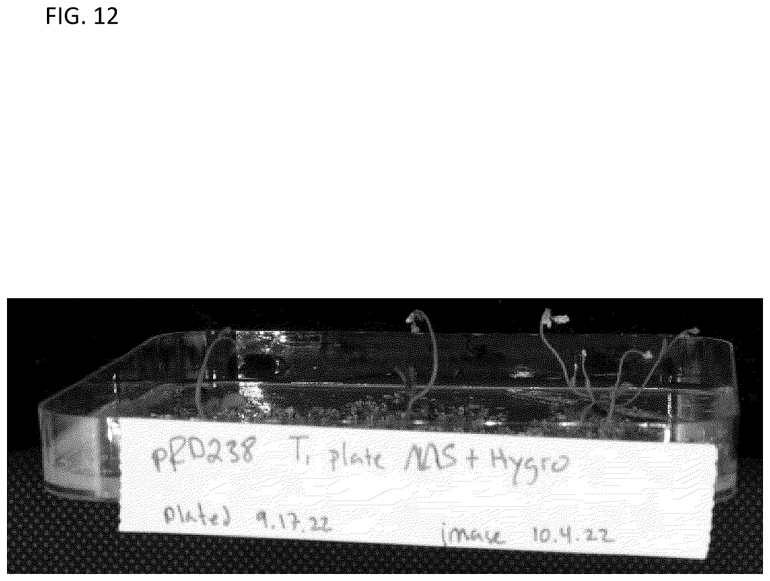

FIG. 12 is a photograph of 17-day-old pRD238 Arabidopsis plants showing early flowering induced by dRNAs.

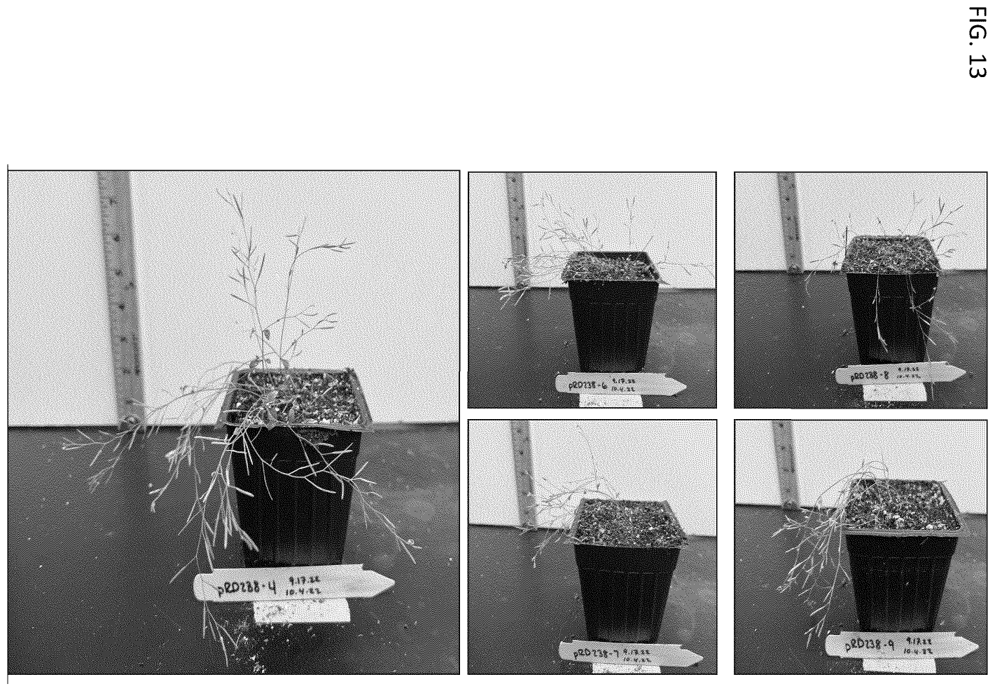

FIG. 13 shows photographs of 45-day-old pRD238 Arabidopsis T2 lines showing little to no presence of rosettes.

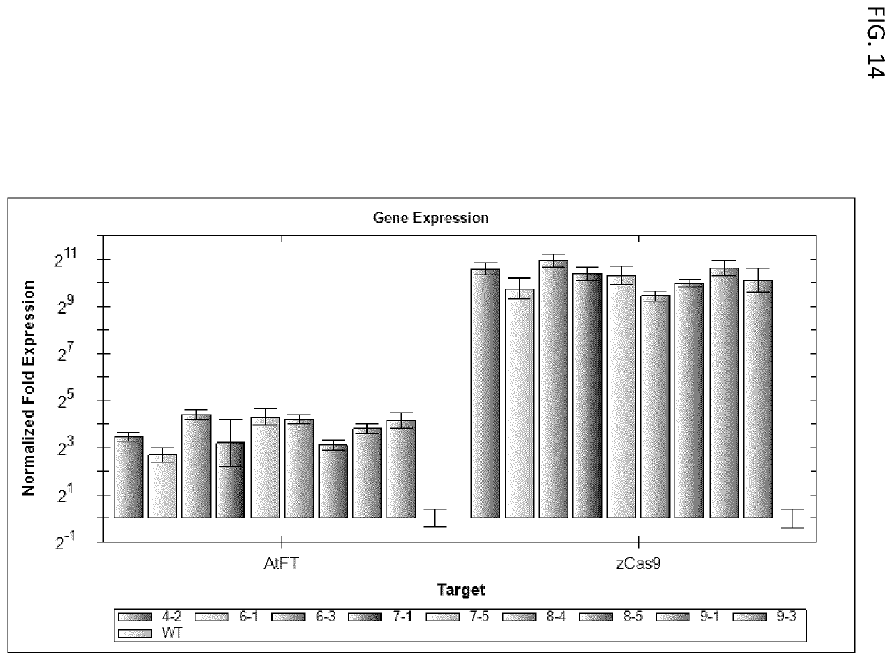

FIG. 14 is a plot of expression levels of the AtFT locus and zCas9-Act3.0 in pRD238 T3 lines indicated by At.FT expression in 45-day old Arabidopsis bud/floral tissue.

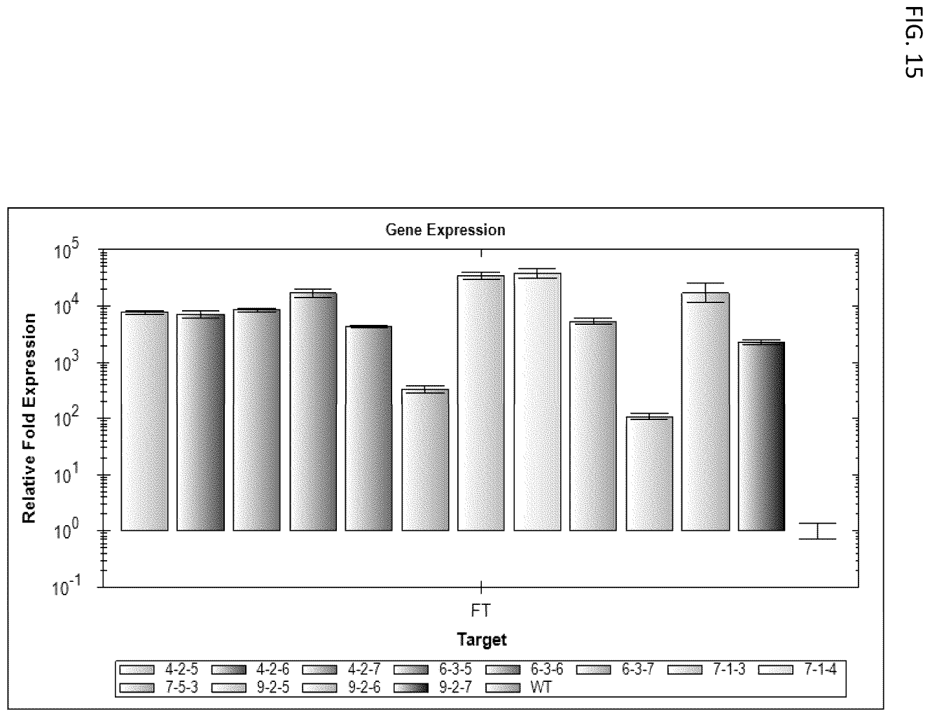

FIG. 15 is a plot of expression levels of the AtFT locus and zCas9-Act3.0 in pRD238 T3 lines indicated by At.FT expression in 18-day old Arabidopsis bud/floral tissue.

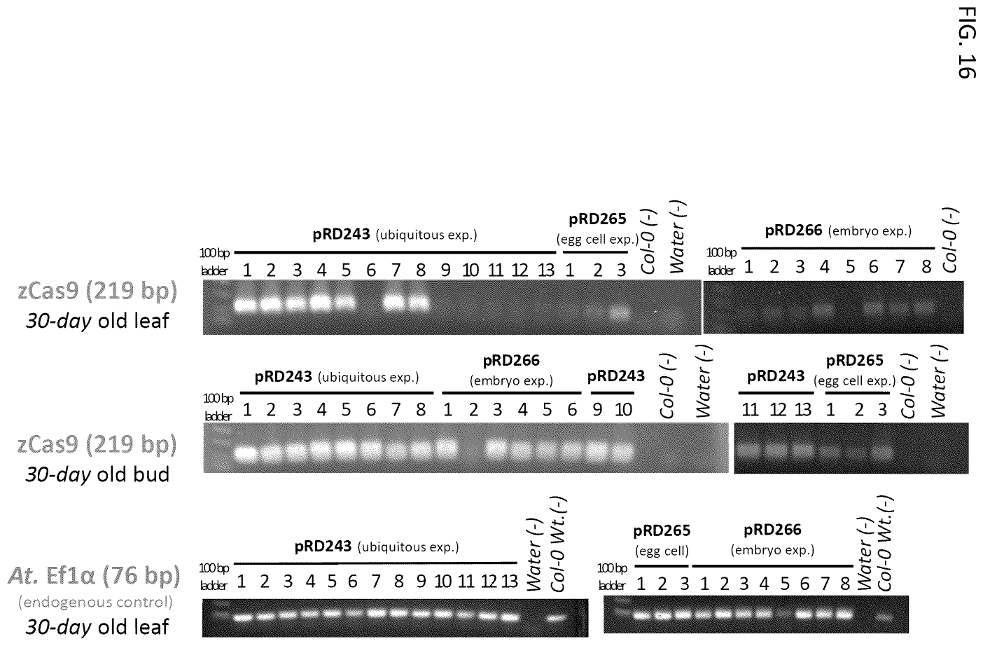

FIG. 16 are photographs of electrophoreses gels showing expression of zCas9 in 30-day old leaves (top panel) and in 30-day old bud (middle panel). Lower panel shows expression of At. Ef1α used as a control.

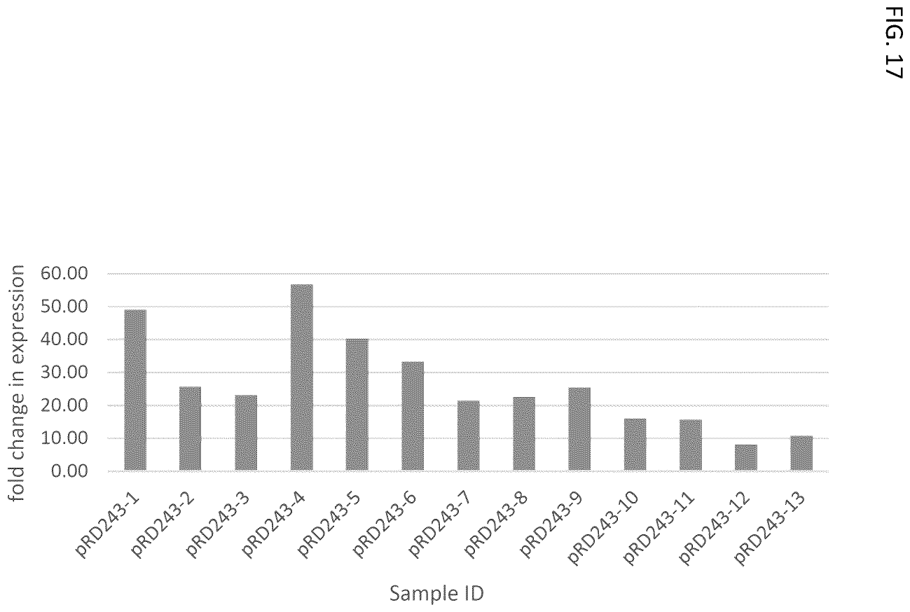

FIG. 17 is a plot showing fold expression of zCas9 in pRD243 lines relative to wild-type using the Cq method.

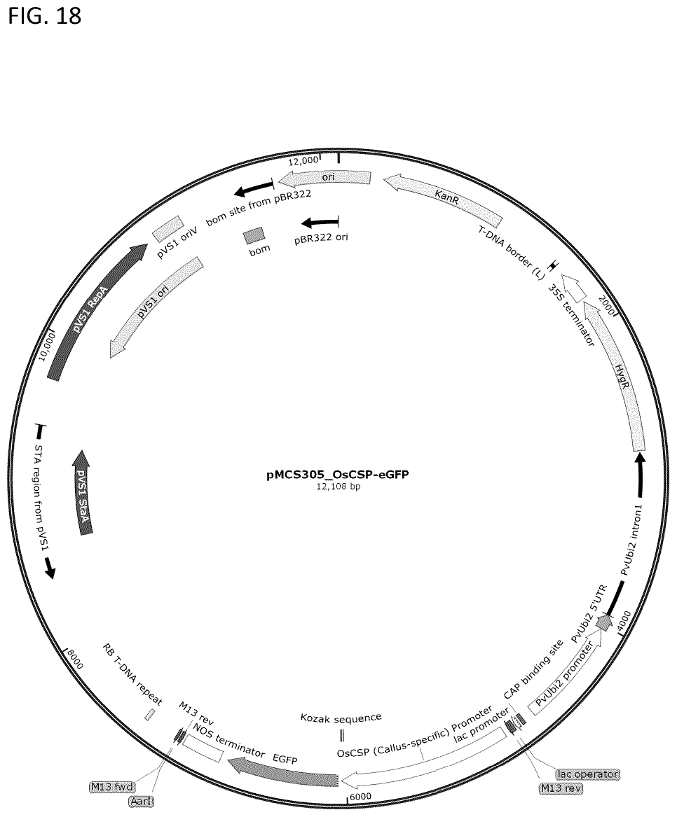

FIG. 18 depicts a map of plasm id pMCS305.

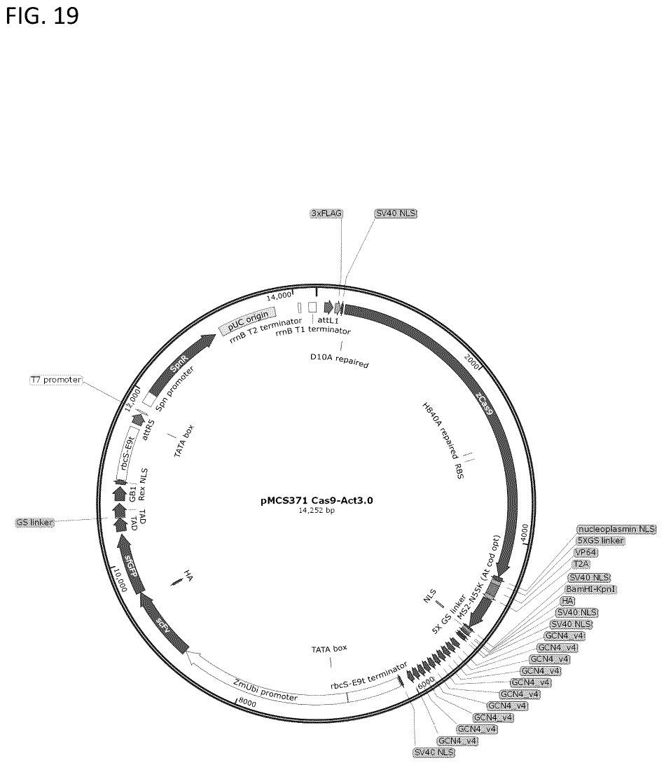

FIG. 19 depicts a map of plasm id pMCS371.

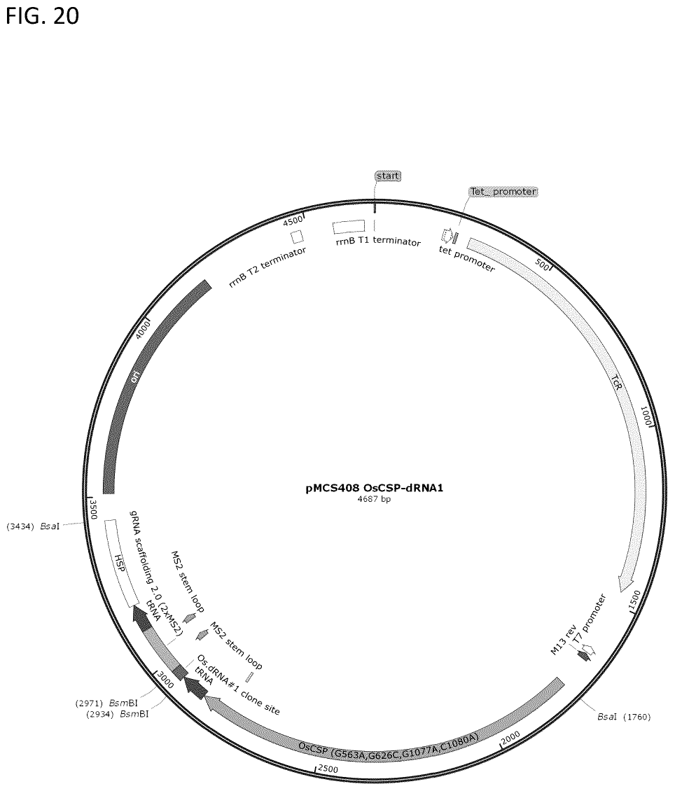

FIG. 20 depicts a map of plasm id pMCS408.

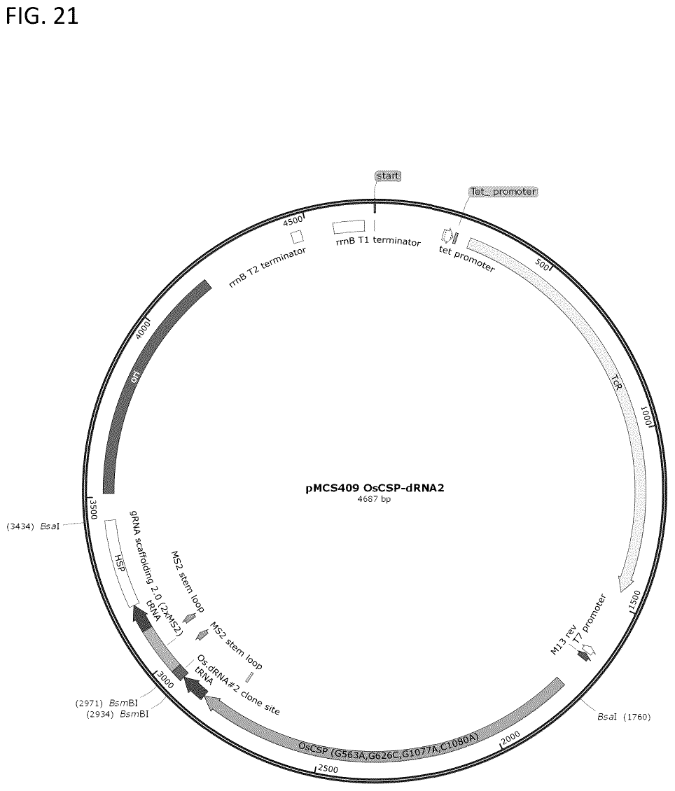

FIG. 21 depicts a map of plasmid pMCS409.

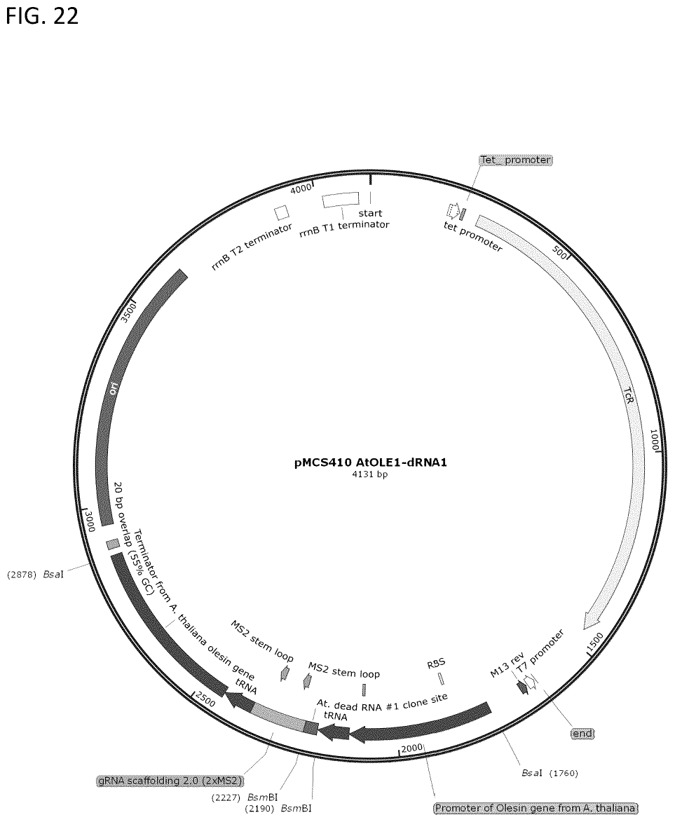

FIG. 22 depicts a map of plasm id pMCS410.

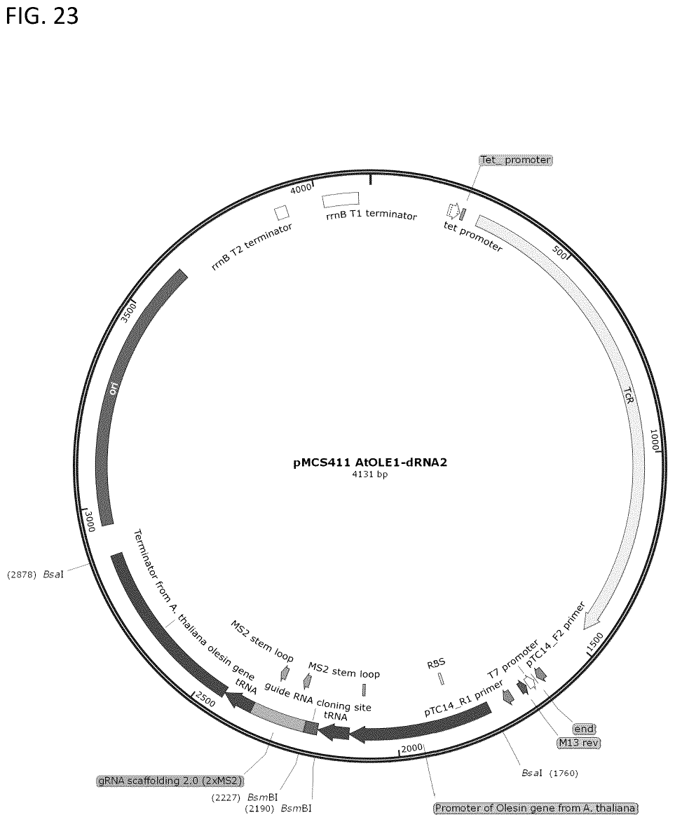

FIG. 23 depicts a map of plasm id pMCS411.

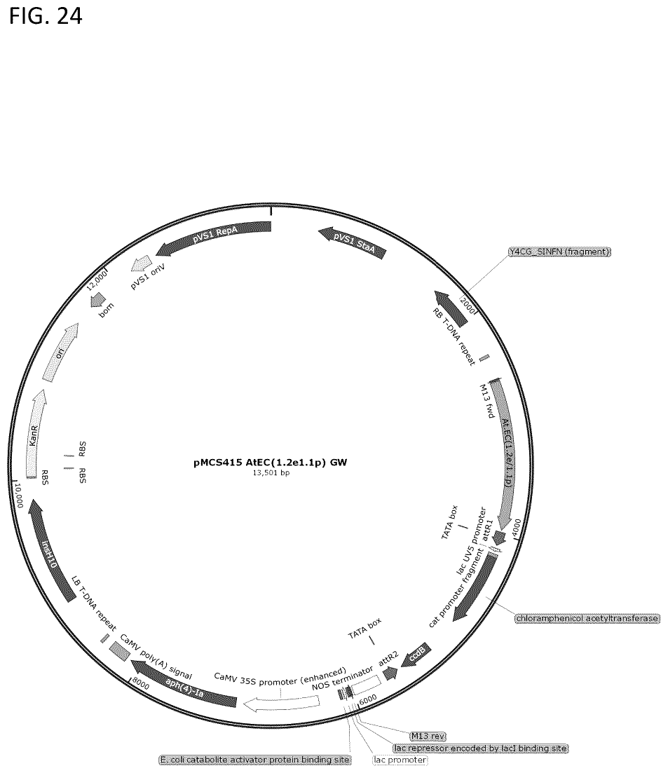

FIG. 24 depicts a map of plasm id pMCS415.

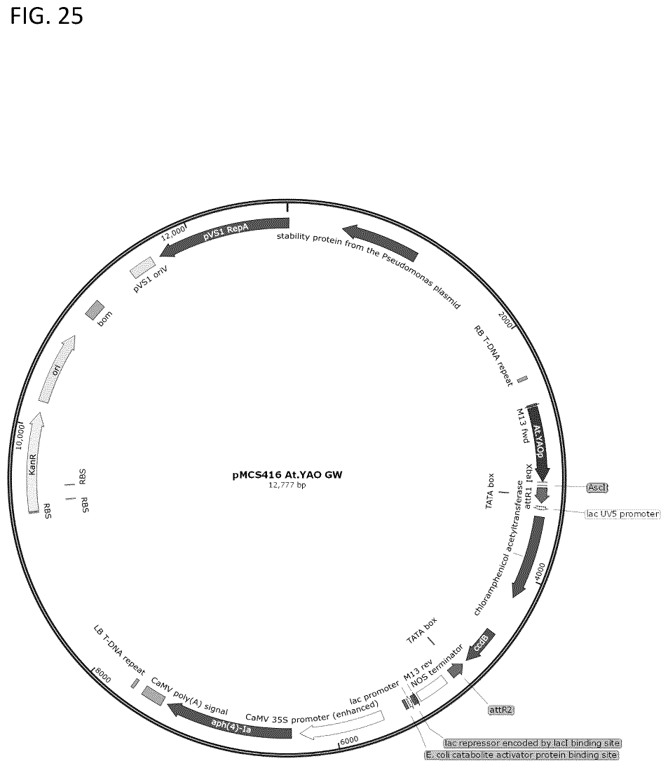

FIG. 25 depicts a map of plasm id pMCS416.

### DETAILED DESCRIPTION

The present disclosure is based in part on the surprising discovery that combining a homologous recombination system with a transcription activation system may be used to efficiently and consistently generate and identify an accurate homologous recombination event. Strategies of the discovery may be as depicted in FIGS. 1-7. Essentially, a homologous recombination system induces homologous recombination of a donor polynucleotide at a specific nucleic acid locus in a gene of interest (see, e.g., FIG. 1). A donor polynucleotide encodes a reporter flanked by regions homologous to the nucleic acid locus for introducing the reporter at the nucleic acid locus. A transcription activation system specifically induces expression of the gene of interest. In the event of an inaccurate homologous recombination event, the reporter is not introduced into the gene of interest at the target nucleic acid locus, and is not expressed when the expression of the gene of interest is activated by the transcription activation system (FIG. 1, Panel A). Conversely, expression of the reporter after homologous recombination indicates an accurate homologous recombination event, and identifies the accurate homologous recombination event (FIG. 1, Panel B).

I. Composition

In one aspect, the present disclosure provides a homologous recombination composition for inducing and identifying an accurate recombination event. The composition comprises a homologous recombination system and a transcription activation system. The homologous recombination system comprises a programmable nucleic acid modification system, wherein the modification system targets a nucleic acid locus in a gene of interest. The homologous recombination system further comprises a donor polynucleotide encoding a reporter flanked by regions homologous to the nucleic acid locus. The transcription activation system is specific for inducing expression of the gene of interest, wherein expression of the reporter after homologous recombination indicates an accurate homologous recombination event. A homologous recombination composition may be directed to one or more, or two or more nucleic acid loci.

(a) Homologous Recombination System

As used herein, the term “homologous recombination system” refers to any system capable of inducing and generating a homologous recombination event at a target nucleic acid locus, and that may lead to the replacement of nucleic acid sequences at or near the target nucleic acid locus with nucleic acid sequences of a donor polynucleotide. A homologous recombination system of the disclosure generally comprises a programmable nucleic acid modification system and a donor polynucleotide. The programmable nucleic acid modification system targets a nucleic acid locus in a gene of interest and induces homologous recombination at the target nucleic acid locus. The donor polynucleotide encodes a reporter flanked by regions homologous to the nucleic acid locus. In the presence of the donor polynucleotide, homologous recombination may lead to the replacement of nucleic acid sequences at or near the target nucleic acid locus with nucleic acid sequences of the donor polynucleotide.

A. Programmable Nucleic Acid Modification Systems

Programmable nucleic acid modification systems generally comprise a programmable, sequence-specific nucleic acid-binding domain, and a modification domain. The programmable nucleic acid-binding domain may be designed or engineered to recognize and bind different nucleic acid sequences. In some modification systems, the nucleic acid-binding domain is mediated by interaction between a protein and the target nucleic acid sequence. Thus, the nucleic acid-binding domain may be programmed to bind a nucleic acid sequence of interest by protein engineering. In other modification systems, the nucleic acid-binding domain is mediated by a guide nucleic acid that interacts with a protein of the modification system and the target nucleic acid sequence. In such instances, the programmable nucleic acid-binding domain may be targeted to a nucleic acid sequence of interest by designing the appropriate guide nucleic acid.

The programmable nucleic acid modification system further comprises a nuclease modification domain and, thus, has nuclease activity. Thus, a programmable nucleic acid modification protein of the modification system is a targeting endonuclease that cleaves a nucleic acid at a targeted site. The cleavage may be double-stranded or single-stranded. The cleavage may be repaired by homology directed repair (HDR) or non-homologous end-joining (NHEJ) repair processes. Non-limiting examples of programmable nucleic acid modification systems include, without limit, CRISPR/Cas nucleases, CRISPR/Cas nickases, DNA-guided Argonaute endonucleases, zinc finger nucleases, transcription activator-like effector nucleases, meganucleases, or chimeric proteins comprising a programmable nucleic acid-binding domain and a nuclease domain. Other suitable programmable nucleic acid modification systems will be recognized by individuals skilled in the art. Programmable nucleic acid modification systems may be as detailed below in Sections (I)(a)(A)(i)-(vii).

i. CRISPR Nuclease Systems

The programmable nucleic acid modification system may be a RNA-guided CRISPR nuclease system. The CRISPR system is guided by a guide RNA to a target sequence at which a protein of the system introduces a double-stranded break in a target nucleic acid sequence.

The CRISPR nuclease system may be derived from any type of CRISPR system, including a type I (i.e., IA, IB, IC, ID, IE, or IF), type II (i.e., IIA, IIB, or IIC), type III (i.e., IIIA or IIIB), or type V CRISPR system. The CRISPR/Cas system may be from Streptococcus sp. (e.g., Streptococcus pyogenes), Campylobacter sp. (e.g., Campylobacter jejuni), Francisella sp. (e.g., Francisella novicida), Acaryochloris sp., Acetohalobium sp., Acidaminococcus sp., Acidithiobacillus sp., Alicyclobacillus sp., Allochromatium sp., Ammonifex sp., Anabaena sp., Arthrospira sp., Bacillus sp., Burkholderiales sp., Caldicelulosiruptor sp., Candidatus sp., Clostridium sp., Crocosphaera sp., Cyanothece sp., Exiguobacterium sp., Finegoldia sp., Ktedonobacter sp., Lactobacillus sp., Lyngbya sp., Marinobacter sp., Methanohalobium sp., Microscilla sp., Microcoleus sp., Microcystis sp., Natranaerobius sp., Neisseria sp., Nitrosococcus sp., Nocardiopsis sp., Nodularia sp., Nostoc sp., Oscillatoria sp., Polaromonas sp., Pelotomaculum sp., Pseudoalteromonas sp., Petrotoga sp., Prevotella sp., Staphylococcus sp., Streptomyces sp., Streptosporangium sp., Synechococcus sp., or Thermosipho sp.

Non-limiting examples of suitable CRISPR systems include CRISPR/Cas systems, CRISPR/Cpf systems, CRISPR/Cmr systems, CRISPR/Csa systems, CRISPR/Csb systems, CRISPR/Csc systems, CRISPR/Cse systems, CRISPR/Csf systems, CRISPR/Csm systems, CRISPR/Csn systems, CRISPR/Csx systems, CRISPR/Csy systems, CRISPR/Csz systems, and derivatives or variants thereof. Preferably, the CRISPR system may be a type II Cas9 protein, a type V Cpf1 protein, or a derivative thereof. More preferably, the CRISPR/Cas nuclease may be Streptococcus pyogenes Cas9 (SpCas9), Streptococcus thermophilus Cas9 (StCas9), Campylobacter jejuni Cas9 (CjCas9), Francisella novicida Cas9 (FnCas9), or Francisella novicida Cpf1 (FnCpf1).

In general, a protein of the CRISPR system comprises a RNA recognition and/or RNA binding domain, which interacts with the guide RNA. A protein of the CRISPR system also comprises at least one nuclease domain having endonuclease activity. For example, a Cas9 protein may comprise a RuvC-like nuclease domain and a HNH-like nuclease domain, and a Cpf1 protein may comprise a RuvC-like domain. A protein of the CRISPR system may also comprise DNA binding domains, helicase domains, RNase domains, protein-protein interaction domains, dimerization domains, as well as other domains.

A protein of the CRISPR system may be associated with one or more guide RNAs (gRNA). The guide RNA may be a single guide RNA (i.e., sgRNA), or may comprise two RNA molecules (i.e., crRNA and tracrRNA). The guide RNA interacts with a protein of the CRISPR system to guide it to a target site in the DNA. The target site has no sequence limitation except that the sequence is bordered by a protospacer adjacent motif (PAM). For example, PAM sequences for Cas9 include 3′-NGG, 3′-NGGNG, 3′-NNAGAAW, and 3′-ACAY, and PAM sequences for Cpf1 include 5′-TTN (wherein N is defined as any nucleotide, W is defined as either A or T, and Y is defined as either C or T). Each gRNA comprises a sequence that is complementary to the target sequence (e.g., a Cas9 gRNA may comprise GN17-20GG). The gRNA may also comprise a scaffold sequence that forms a stem loop structure and a single-stranded region. The scaffold region may be the same in every gRNA. In some aspects, the gRNA may be a single molecule (i.e., sgRNA). In other aspects, the gRNA may be two separate molecules. Those skilled in the art are familiar with gRNA design and construction, e.g., gRNA design tools are available on the internet or from commercial sources.

A CRISPR system may comprise one or more nucleic acid binding domains associated with one or more, or two or more selected guide RNAs used to direct the CRISPR system to one or more, or two or more selected target nucleic acid loci. For instance, a nucleic acid binding domain may be associated with one or more, or two or more selected guide RNAs, each selected guide RNA, when complexed with a nucleic acid binding domain, causing the CRISPR system to localize to the target of the guide RNA.

ii. CRISPR Nickase Systems

The programmable nucleic acid modification system may also be a CRISPR nickase system. CRISPR nickase systems are similar to the CRISPR nuclease systems described above except that a CRISPR nuclease of the system is modified to cleave only one strand of a double-stranded nucleic acid sequence. Thus, a CRISPR nickase in combination with a guide RNA of the system may create a single-stranded break or nick in the target nucleic acid sequence. Alternatively, a CRISPR nickase in combination with a pair of offset gRNAs may create a double-stranded break in the nucleic acid sequence.

A CRISPR nuclease of the system may be converted to a nickase by one or more mutations and/or deletions. For example, a Cas9 nickase may comprise one or more mutations in one of the nuclease domains, wherein the one or more mutations may be D10A, E762A, and/or D986A in the RuvC-like domain, or the one or more mutations may be H840A (or H839A), N854A and/or N863A in the HNH-like domain.

iii. ssDNA-guided Argonaute systems

Alternatively, the programmable nucleic acid modification system may comprise a single-stranded DNA-guided Argonaute endonuclease. Argonautes (Agos) are a family of endonucleases that use 5′-phosphorylated short single-stranded nucleic acids as guides to cleave nucleic acid targets. Some prokaryotic Agos use single-stranded guide DNAs and create double-stranded breaks in nucleic acid sequences. The ssDNA-guided Ago endonuclease may be associated with a single-stranded guide DNA.

The Ago endonuclease may be derived from Alistipes sp., Aquifex sp., Archaeoglobus sp., Bacteroides sp., Bradyrhizobium sp., Burkholderia sp., Cellvibrio sp., Chlorobium sp., Geobacter sp., Mariprofundus sp., Natronobacterium sp., Parabacteriodes sp., Parvularcula sp., Planctomyces sp., Pseudomonas sp., Pyrococcus sp., Thermus sp., or Xanthomonas sp. For instance, the Ago endonuclease may be Natronobacterium gregoryi Ago (NgAgo). Alternatively, the Ago endonuclease may be Thermus thermophilus Ago (TtAgo). The Ago endonuclease may also be Pyrococcus furiosus (PfAgo).

The single-stranded guide DNA (gDNA) of a ssDNA-guided Argonaute system is complementary to the target site in the nucleic acid sequence. The target site has no sequence limitations and does not require a PAM. The gDNA generally ranges in length from about 15-30 nucleotides. The gDNA may comprise a 5′ phosphate group. Those skilled in the art are familiar with ssDNA oligonucleotide design and construction.

iv. Zinc Finger Nucleases

The programmable nucleic acid modification system may be a zinc finger nuclease (ZFN). A ZFN comprises a DNA-binding zinc finger region and a nuclease domain. The zinc finger region may comprise from about two to seven zinc fingers, for example, about four to six zinc fingers, wherein each zinc finger binds three nucleotides. The zinc finger region may be engineered to recognize and bind to any DNA sequence. Zinc finger design tools or algorithms are available on the internet or from commercial sources. The zinc fingers may be linked together using suitable linker sequences.

A ZFN also comprises a nuclease domain, which may be obtained from any endonuclease or exonuclease. Non-limiting examples of endonucleases from which a nuclease domain may be derived include, but are not limited to, restriction endonucleases and homing endonucleases. The nuclease domain may be derived from a type II-S restriction endonuclease. Type II-S endonucleases cleave DNA at sites that are typically several base pairs away from the recognition/binding site and, as such, have separable binding and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MboII, and SapI. The type II-S nuclease domain may be modified to facilitate dimerization of two different nuclease domains. For example, the cleavage domain of FokI may be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI nuclease domains are targets for modification. For example, one modified FokI domain may comprise Q486E, I499L, and/or N496D mutations, and the other modified FokI domain may comprise E490K, I538K, and/or H537R mutations.

v. Transcription Activator-Like Effector Nuclease Systems

The programmable nucleic acid modification system may also be a transcription activator-like effector nuclease (TALEN) or the like. TALENs comprise a DNA-binding domain composed of highly conserved repeats derived from transcription activator-like effectors (TALEs) that are linked to a nuclease domain. TALEs are proteins secreted by plant pathogen Xanthomonas to alter transcription of genes in host plant cells. TALE repeat arrays may be engineered via modular protein design to target any DNA sequence of interest. Other transcription activator-like effector nuclease systems may comprise, but are not limited to, the repetitive sequence, transcription activator like effector (RipTAL) system from the bacterial plant pathogenic Ralstonia solanacearum species complex (Rssc). The nuclease domain of TALEs may be any nuclease domain as described above in Section (I)(a)(A)(i).

vi. Meganucleases or Rare-Cutting Endonuclease Systems

The programmable nucleic acid modification system may also be a meganuclease or derivative thereof. Meganucleases are endodeoxyribonucleases characterized by long recognition sequences, i.e., the recognition sequence generally ranges from about 12 base pairs to about 45 base pairs. As a consequence of this requirement, the recognition sequence generally occurs only once in any given genome. Among meganucleases, the family of homing endonucleases named LAGLIDADG has become a valuable tool for the study of genomes and genome engineering. In some aspects, the meganuclease may be I-SceI or variants thereof. A meganuclease may be targeted to a specific nucleic acid sequence by modifying its recognition sequence using techniques well known to those skilled in the art.

The programmable DNA modification system having nuclease activity may be a rare-cutting endonuclease or derivative thereof. Rare-cutting endonucleases are site-specific endonucleases whose recognition sequence occurs rarely in a genome, preferably only once in a genome. The rare-cutting endonuclease may recognize a 7-nucleotide sequence, an 8-nucleotide sequence, or longer recognition sequence. Non-limiting examples of rare-cutting endonucleases include NotI, AscI, PacI, AsiSI, SbfI, and FseI.

vii. Optional Additional Domains

The programmable nucleic acid modification system may further comprise at least one nuclear localization signal (NLS), at least one cell-penetrating domain, at least one reporter domain, and/or at least one linker.

In general, an NLS comprises a stretch of basic amino acids. Nuclear localization signals are known in the art (see, e.g., Lange et al., J. Biol. Chem., 2007, 282:5101-5105). For example, in one aspect, the NLS may be a monopartite sequence, such as PKKKRKV (SEQ ID NO: 1) or PKKKRRV (SEQ ID NO: 2). Alternatively, the NLS may be a bipartite sequence. Further, the NLS may be KRPAATKKAGQAKKKK (SEQ ID NO: 1). The NLS may be located at the N-terminus, the C-terminal, or in an internal location of the fusion protein.

A cell-penetrating domain may be a cell-penetrating peptide sequence derived from the HIV-1 TAT protein. As an example, the TAT cell-penetrating sequence may be GRKKRRQRRRPPQPKKKRKV (SEQ ID NO: 2). Alternatively, the cell-penetrating domain may be TLM (PLSSIFSRIGDPPKKKRKV; SEQ ID NO: 3), a cell-penetrating peptide sequence derived from the human hepatitis B virus; MPG (GALFLGWLGAAGSTMGAPKKKRKV; SEQ ID NO: 4; or GALFLGFLGAAGSTMGAWSQPKKKRKV; SEQ ID NO: 5); or Pep-1 (KETVVWETVWVTEWSQPKKKRKV; SEQ ID NO: 6), VP22, a cell-penetrating peptide from Herpes simplex virus, or a polyarginine peptide sequence. The cell-penetrating domain may be located at the N-terminus, the C-terminal, or in an internal location of the fusion protein.

A programmable nucleic acid modification system may further comprise at least one reporter domain. Non-limiting examples of reporter domains include fluorescent proteins, purification tags, and epitope tags. In some aspects, the reporter domain may be a fluorescent protein. Non-limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalama1, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (e.g., mOrange, mKO, Kusabira-Orange, Monomeric Kusabira-Orange, mTangerine, tdTomato), or any other suitable fluorescent protein. In other aspects, the reporter domain may be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, HA, nus, Softag 1, Softag 3, Strep, SBP, Glu-Glu, HSV, KT3, S, 51, T7, V5, VSV-G, 6×His, biotin carboxyl carrier protein (BCCP), and calmodulin.

A programmable nucleic acid modification system may further comprise at least one linker. For example, the programmable nucleic acid modification system, the nuclease domain of a protein, and other optional domains may be linked via one or more linkers. The linker may be flexible (e.g., comprising small, non-polar (e.g., Gly) or polar (e.g., Ser, Thr) amino acids). Non-limiting examples of flexible linkers include GGSGGGSG, (GGGGS)1-4, and (Gly)6-8. Alternatively, the linker may be rigid, such as (EAAAK)1-4, A(EAAAK)2-5A, PAPAP (AP)6-8, and (XP)n, wherein X is any amino acid, but preferably Ala, Lys, or Glu. Examples of suitable linkers are well known in the art, and programs to design linkers are readily available (Crasto et al., Protein Eng., 2000, 13(5):3096-312). In alternate aspects, the programmable DNA modification protein, the cell cycle regulated protein, and other optional domains may be linked directly.

A programmable nucleic acid modification system may further comprise an organelle localization or targeting signal that directs a molecule to a specific organelle. A signal may be polynucleotide or polypeptide signal, or may be an organic or inorganic compound sufficient to direct an attached molecule to a desired organelle. Exemplary organelle localization signals may be as described in U.S. Patent Publication No. 20070196334, the disclosure of which is incorporated herein in its entirety.

B. Donor Polynucleotide

Programmable nucleic acid modification systems also comprise a donor polynucleotide. In the presence of the donor polynucleotide, homologous recombination may lead to the replacement of nucleic acid sequences at or near the target nucleic acid locus with nucleic acid sequences of the donor polynucleotide. The donor polynucleotide encodes a reporter flanked by regions homologous to the nucleic acid locus in a gene of interest.

A donor polynucleotide may be an RNA or DNA, single-stranded or double-stranded, linear or circular. The donor polynucleotide may be part of a vector, e.g., a plasm id or viral vector as described in Section II.

i. Reporter

The donor polynucleotide encodes a reporter. As used herein, the term “reporter” refers to any biomolecule that may be used as an indicator of transcription and/or translation through a promoter. A reporter may be a polypeptide. A reporter may also be a nucleic acid. Suitable polypeptide and nucleic acid reporters are known in the art, and may include visual reporters, selectable reporters, screenable reporters, and combinations thereof. Other types of reporters will be recognized by individuals of skill in the art.

Visual reporters typically result in a visual signal, such as a color change in the cell, or fluorescence or luminescence of the cell. Suitable visual reporters include fluorescent proteins, visible reporters, epitope tags, affinity tags, RNA aptamers, and the like. Non-limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalama1, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (e.g., mOrange, mKO, Kusabira-Orange, Monomeric Kusabira-Orange, mTangerine, tdTomato), or any other suitable fluorescent protein. Non-limiting examples of visual reporters include luciferase, alkaline phosphatase, beta-glucuronidase (GUS), beta-galactosidase, beta-lactamase, horseradish peroxidase, anthocyanin pigmentation, and variants thereof. Suitable epitope tags include, but are not limited to, myc, AcV5, AU1, AUS, E, ECS, E2, FLAG, HA, Maltose binding protein, nus, Softag 1, Softag 3, Strep, SBP, Glu-Glu, HSV, KT3, S, 51, T7, V5, VSV-G, 6×His, BCCP, and calmodulin. Non-limiting examples of affinity tags include chitin binding protein (CBP), thioredoxin (TRX), poly(NANP), tandem affinity purification (TAP) tag, and glutathione-S-transferase (GST). Non-limiting examples of RNA aptamers include fluorescent RNA aptamers that sequester small molecule dyes and activate their fluorescence, such as spinach, broccoli, mango, or biliverdin-binding variants thereof.

Other visual reporters may include fluorescent resonance energy transfer (FRET), lanthamide resonance energy transfer (LRET), fluorescence cross-correlation spectroscopy, fluorescence quenching, fluorescence polarization, scintillation proximity, chemiluminescence energy transfer, bioluminescence resonance energy transfer, excimer formation, phosphorescence, electrochemical changes, molecular beacons, and redox potential changes.

Selectable reporters typically confer a positively or negatively selectable trait to a cell, such as a drug resistance (e.g., antibiotic resistance) positive selection reporter. Examples of suitable selectable reporters include, without limit, herbicide resistance or tolerance such as resistance to glyphosate, glufosinate ammonium, bromoxynil, 2,4-dichlorophenoxyacetate (2,4-D), or sulfonylurea herbicides, antibiotic or chemical selectable reporters such as puromycin, zeomycin, streptomycin, chloramphenicol, gentamycin, neomycin, hydromycin, phleomycin, hygromycin, bleomycin, sulfonamide, bromoxynil, spectinomycin, methotrexate, and the like. Additional examples include dihydrofolate reductase, 5-eno/pyruvylshikimate-3-phosphate synthase, and acetolactate synthase, neomycin phosphotransferase I and II, cyanamide hydratase, aspartate kinase, dihydrodipicolinate synthase, bar gene, tryptophane decarboxylase, hygromycin phosphotransferase (HPT or HYG), dihydrofolate reductase (DHFR), phosphinothricin acetyltransferase, 2,2-dichloropropionic acid dehalogenase, acetohydroxyacid synthase, 5-enolpyruvyl-shikimate-phosphate synthase, haloarylnitrilase, acetyl-coenzyme A carboxylase, dihydropteroate synthase, and 32 kDa photosystem II polypeptide (psbA).

Additionally, selectable reporters may include environmental or artificial stress resistance or tolerance reporters including, but not limited to, high glucose tolerance, low phosphate tolerance, mannose tolerance, and/or drought tolerance, salt tolerance or cold tolerance. Reporters that confer environmental or artificial stress resistance or tolerance include, but are not limited to, trehalose phosphate synthase, phophomannose isomerase, Arabidopsis vacuolar H+-pyrophosphatase, AVPI, aldehyde resistance, and cyanamide resistance.

Other reporters may also be morphogenic reporters. A morphogenic reporter may be any reporter capable of inducing a morphogenic trait that may be used to identify and isolate successful products of homologous recombination. For instance, a morphogenic reporter may be used to activate proliferation of cells that have correct insertion in a desired target gene of interest, when transcriptional activation of the target in the callus occurs. Such a reporter causes cells with the successful event to out-proliferate any other cell. Alternatively, a morphogenic reporter may be used to induce organogenesis by cells that have an correct homologous recombination event in a desired target gene of interest, when transcriptional activation of the target in the callus occurs. Such a reporter causes cells with the successful event to produce a plant instead thereby identifying the successful event. Non-limiting examples of morphogenic reporters include promoters of cellular proliferation. For instance, a morphogenic reporter may be a transcription factor that promotes stem cell proliferation or organogenesis. Non-limiting examples of morphogenic promoters may include the maize (Zea mays) Baby boom (Bbm), the maize Wuschel2 (Wus2) genes, and combinations thereof.

It will be recognized that combinations of reporters may be used. For instance, a visual reporter fused to a protein expressed by the gene of interest may be used to identify an accurate homologous recombination event, but the visual reporter is not permanently fused to the protein (see, e.g. FIG. 2). A second reporter may be used in combination with the visual reporter, wherein the second reporter is permanently fused to the protein.

Additionally, irrespective of the reporter used in a donor polynucleotide, the reporter may be a split reporter system. Split reporter systems may be used to reduce the size of a reporter sequence introduced into a target nucleic acid locus. Non-limiting examples of suitable split reporter systems include split GFP systems, split 5-EnolpyruvylShikimate-3-Phosphate Synthase for glyphosate resistance, among others. Similarly, irrespective of the reporter used, a donor polynucleotide may encode an activator for activating a reporter encoded in a location other than the donor polynucleotide. For instance, a donor polynucleotide may encode an activator for activating a reporter encoded on nucleic acid sequences introduced into a cell with the donor polynucleotide, such as T-DNA nucleic acid sequences.

ii. Gene of Interest

As used herein, the term “gene” refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions. Therefore, a target nucleic acid locus may be within any sequence in the gene of interest, including, but not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

As used herein, the term “encode” refers to is understood to have its plain and ordinary meaning as used in the biological fields, i.e., specifying a biological sequence. The term “encode,” when used to describe the function of nucleic acid molecules, customarily means to identify one single amino acid sequence that makes up a unique polypeptide, or one nucleic acid sequence that makes up a unique RNA. That function is implemented by the particular nucleotide sequence of each nucleic acid molecule. In this aspect, the term “encode” refers to a reporter operably linked to the regions of homology such that the reporter is expressed upon accurate homologous recombination into the gene of interest and upon transcription activation of the gene of interest comprising the locus of interest. As used herein, the term “express” refers to the conversion of DNA sequence information into messenger RNA (mRNA) and/or protein. In this aspect, the term “express” refers to production of a detectable reporter signal as a result of an accurate homologous recombination event and transcription activation of the gene of interest.

The gene of interest may be a protein coding gene or an RNA coding gene. When the gene of interest is a protein coding gene, the reporter may be encoded in-frame with an open reading frame of the gene of interest such that expression of the gene of interest results in the expression of a fusion protein comprising the reporter polypeptide and the polypeptide encoded by the gene of interest (See, for example, FIGS. 1, 2, 4). In-frame reporters may be fused at the N terminus, C terminus, or internally to the polypeptide encoded by the gene of interest. In a variation, the reporter may completely or partially replace a coding sequence of the gene of interest, or introduce a stop codon such that expression of the gene of interest results in the expression of an unfused reporter, or a reporter fused at the N terminus, C terminus, or internally to a polypeptide fragment encoded by the partial open reading frame of the gene of interest (FIG. 3). Additionally, the reporter may be encoded in an intron of a gene of interest, or in an untranslated region of a protein-producing gene of interest, such that the reporter is expressed upon transcription of the gene of interest (FIG. 6).

The gene of interest may also be an RNA coding gene. Non-limiting examples of RNA coding genes may include genes encoding long non-translated RNAs (IntRNA), trans-acting siRNAs (tasiRNAs), antisense mRNAs, and the like (FIG. 7). When the gene of interest is an RNA coding gene, a reporter is preferably a fluorescent RNA aptamer, or other reporters that do not require translation to be expressed.

Additionally, irrespective of the reporter used in a donor polynucleotide or the gene of interest, a donor polynucleotide may further comprise elements for expressing a reporter without a permanent fusion of the reporter with products of the gene of interest. For instance, as depicted in FIG. 2A, a reporter sequence encoded in frame at the C-terminus of an open reading frame of the gene of interest may be preceded by a skipping sequence that replaces the endogenous STOP codon of the gene of interest. After an accurate homologous recombination event, the gene of interest is expressed, a peptide encoded by the gene of interest and a separate reporter polypeptide are generated as a result of ribosomal skipping mediated by the skipping sequence. Non-limiting examples of skipping sequences include the 2A self-cleaving peptide of picornaviruses or 2A-like sequences (also called CHYSEL (cis-acting hydrolase element)) such as 2A-like sequences of iflaviridae, tetraviridae, dicistroviridae, and reoviridae.

Alternatively, as depicted in FIG. 2B, a reporter sequence encoded in-frame with an open reading frame of the gene of interest may be flanked by recombinase recognition sites, and the homologous recombination composition may further comprise a recombinase. After an accurate homologous recombination event and expression of the gene of interest, a fusion protein comprising the reporter flanked by the recombinase recognition sites is expressed. The reporter may then be removed from the polypeptide of the gene of interest through the action of the recombinase. Non-limiting examples of a recombinase and recombinase recognition sites may include Cre recombinase and loxP recognition sites. Other strategies for expressing a reporter without a permanent fusion of the reporter with products of the gene of interest will be evident to an individual of skill in the art.

(iii) Homologous Regions

Typically, the reporter is flanked by upstream and downstream nucleic acid sequences homologous to the nucleic acid locus in a gene of interest. The upstream and downstream homologous sequences have substantial sequence identity to sequences located upstream and downstream, respectively, of the nucleic acid locus targeted by the targeting endonuclease. Because of these sequence similarities, the donor sequence may be integrated into (or exchanged with) a nucleic acid locus by homologous recombination. As used herein, the term “homologous” when used in reference to nucleic acid sequences, refers to sequences having at least about 75% sequence identity. Thus, the upstream and downstream sequences in the donor polynucleotide may have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with sequences upstream or downstream to the nucleic acid locus sequence. In specific aspects, the upstream and downstream sequences in the donor polynucleotide may have about 95% or 100% sequence identity with nucleic acid sequences upstream or downstream of the nucleic acid locus targeted by the targeting endonuclease.

As will be appreciated by those skilled in the art, the length of the donor polynucleotide may and does vary. For example, the construct sequence may vary in length from several base pairs to hundreds of base pairs to hundreds of thousands of base pairs. Each upstream or downstream sequence may range in length from about 20 base pairs to about 5000 base pairs. In some aspects, upstream and downstream sequences may comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2800, 3000, 3200, 3400, 3600, 3800, 4000, 4200, 4400, 4600, 4800, or 5000 base pairs. In specific aspects, upstream and downstream sequences may range in length from about 50 to about 1500 base pairs.

(iv) Other Nucleic Acid Modifications

In addition to encoding a reporter, a donor polynucleotide may further encode other sequence modifications throughout the gene of interest at or near a nucleic acid locus. Non-limiting examples of sequences or sequence modifications that may be encoded in the donor polynucleotide include point mutations, partial sequence deletions, replacements, or additions, ribosomal skipping sequences, antibody epitopes and tags such as AcV5, AU1, AUS, E, ECS, E2, FLAG, Glu-Glu, HSV, KT3, myc, S, S1, T7, V5, VSV-G, and 6×His and variants thereof, TAP tag, recombinase recognition sites, gene expression regulatory sequences, spacers, capture sequences, small RNA target sites, miRNA trigger sites, tasiRNA sequences. The following sections describe some aspects wherein a donor polynucleotide introduces a reporter, and further introduces other sequence modifications in the gene of interest. Other aspects will be readily apparent to individuals skilled in the art.

### Promoter Replacement

In some aspects, a donor polynucleotide may comprise more than one nucleic acid sequence to introduce more than one sequence or sequence modification at more than one locus in a gene of interest. For instance, when a donor polynucleotide further encodes a replacement of the endogenous promoter of a gene of interest, a reporter of the donor polynucleotide may be expressed, even when the homologous recombination is inaccurate or not at the intended target nucleic acid locus (FIG. 4). Therefore, to identify an accurate homologous recombination event wherein an endogenous promoter is replaced, a donor may comprise a first nucleic acid sequence targeting a first nucleic acid locus for replacing the endogenous promoter control sequences, and a second nucleic acid sequence at a second target nucleic acid locus for introducing a reporter. As shown in FIG. 4, the first nucleic acid sequence encodes the heterologous promoter flanked by regions of homology to the first locus, and the second nucleic acid sequence encodes a reporter flanked by regions homologous to a second nucleic acid locus in the gene of interest. Additionally, a programmable modification system of the composition may induce recombination at the first and second loci. For instance, the programmable nucleic acid modification system may encode two gRNAs, each specific for the first and second nucleic acid loci. In such an arrangement, a transcription activation system of the composition is specific to the heterologous promoter. Expression of the reporter after homologous recombination and transcription activation of the gene of interest indicates accurate homologous recombination events at the first and second loci in the gene of interest. Other strategies for using a donor polynucleotide comprising more than one nucleic acid sequence to introduce more than one sequence or sequence modification at more than one locus in a gene of interest may be envisioned by individuals skilled in the art.

### Further Modifications of RNA Coding Genes

When the RNA coding gene encodes lncRNAs that are not further processed, such as COOLAIR, a reporter may be integrated at non-essential regions anywhere in the transcript (FIG. 7, Panel A). Additionally, a small RNA target site may further be introduced to “knock out” the lncRNA by inducing post-transcriptional control of the lncRNA (FIG. 7, Panel B). When the RNA coding gene encodes a miRNA precursor, one nucleotide polymorphism to several polymorphisms may be introduced at the 5′ or 3′ sequences of the precursor in addition to the RNA aptamer (FIG. 7, Panel C). When the RNA coding gene encodes a transcript processed into tasi/phasiRNAs, “in phase” insertions or even replacements of existing (but non-targeting) tasiRNAs or phasiRNAs may be generated (FIG. 7, Panels D-F). For instance, a new tasiRNA may be added downstream of the primary, endogenous tasiRNAs, in-phase with a fluorescent RNA aptamer added further 3′ (FIG. 7, Panel D). Alternatively, a 3′ insertion or replacement of tasiRNAs is performed, also adding a fluorescent RNA aptamer, but wherein an endogenous set of tasiRNAs is used as a spacer (FIG. 7, Panel E). Additionally, a fluorescent RNA aptamer may be added upstream of an miRNA target site, wherein the target site may be replaced with a target mimic to prevent slicing. Other modifications of RNA coding genes may be envisioned by individuals of skill in the art.

### Modifying Intergenic Sequences Between Two Genes of Interest

In some aspects, an intergenic nucleic acid sequence between two genes may be modified. For instance, an intergenic sequence may be deleted and/or replaced with a different sequence (FIG. 3B). The size of the intergenic sequence may range from 0 base pairs to 100s of base pairs, 1000s of base pairs or longer. Further, the intergenic region may comprise coding sequences, regulatory sequences, or any other kind of sequence. As shown in FIG. 3B, a pair of genes are targeted using a donor polynucleotide encoding (1) a first replacement polynucleotide comprising a first reporter flanked by regions of homology to a first nucleic acid locus in a first gene of interest, and (2) a second replacement polynucleotide comprising a second reporter flanked by regions of homology to a second nucleic acid locus in a second gene of interest. The donor polynucleotide further comprises an intergenic construct flanked by the first replacement polynucleotide and the second replacement polynucleotide. The size of the intergenic construct may range from 0 base pairs to 100s of base pairs, 1000s of base pairs or longer. In such an arrangement, a programmable modification system of the composition may induce recombination at the first and second loci in the first and second genes of interest, respectively. For instance, a programmable modification system may encode two gRNAs, each specific for the first and second nucleic acid loci, thereby inducing homologous recombination at the two loci. Accurate recombination at the first and second loci results in the replacement of the intergenic sequence with the intergenic construct. Additionally, in such a system, a homologous recombination composition further comprises a first transcription activation system specific for inducing expression of the first gene of interest, and a second transcription activation system specific for inducing expression of the second gene of interest. Expression of the first and second reporters after homologous recombination and transcription activation of the genes of interest indicates accurate homologous recombination events at the first and second loci in the genes of interest and replacement of the intergenic region with the intergenic construct.

Individuals skilled in the art may envision various useful configurations of useful replacement intergenic constructs. For instance, an intergenic sequence may be deleted. Alternatively, an intergenic sequence may be replaced with a shorter or longer version of the intergenic sequence, or may introduce heterologous nucleic acid sequences.

(b) Transcription Activation System

The homologous recombination composition comprises a transcription activation system specific for inducing expression of a gene of interest. The transcription activation system comprises a transcription activator (or transcription complex recruiting domain). A transcription activator is a protein that increases transcription of a gene of interest by directly or indirectly interacting with the promoter of the gene of interest.

As a homologous recombination composition of the disclosure may further introduce sequences or sequence modifications throughout the gene of interest in addition to introducing a reporter at a target locus, it will be recognized that the transcription activator of the disclosure induces expression of the modified gene of interest resulting from any intended accurate recombination events. For instance, when the open reading frame of a gene of interest is completely replaced by the coding sequence of a reporter, a transcription activation system induces expression of the reporter coding sequence. Similarly, when the promoter of the gene of interest is replaced with a heterologous promoter, a transcription activation system specifically induces expression of the gene of interest by directly or indirectly interacting with the heterologous promoter.

A transcription activator may be a wild type transcription activator naturally specific for inducing transcription of a gene of interest, or modified versions of a wild type transcription activator naturally specific for inducing transcription of a gene of interest. For instance, the transcription activator may be a wild type TALE effector naturally specific for inducing transcription of a gene of interest. Alternatively, a transcription activator may be a synthetic or artificial programmable transcription activator. Programmable transcription activators are well known in the art. Programmable transcription activators generally comprise wild-type or naturally-occurring nucleic acid-binding and/or transcription activation domains, modified versions of naturally-occurring nucleic acid-binding and/or transcription activation domains, synthetic or artificial nucleic acid-binding and/or transcription activation domains, or combinations thereof. In general, engineered transcription activators comprise a programmable nucleic acid-binding domain and a transcription activation domain.

A transcriptional activation domain interacts with transcriptional control elements and/or transcriptional regulatory proteins (i.e., transcription factors, RNA polymerases, etc.) to increase and/or activate transcription of a gene. Suitable transcriptional activation domains include, without limit, herpes simplex virus VP16 domain, VP64 (which is a tetrameric derivative of VP16), VP160 (i.e., 10×VP16), p65 activation domain from NFκB, p53 activation domains 1 and 2, heat-shock factor 1 (HSF1) activation domain, MyoD1 activation domain, GCN4 peptide, 10×GCN4, viral R transactivator (Rta), VPR (a fusion of VP64-p65-Rta), p53 activation domains 1 and 2, CREB (cAMP response element binding protein) activation domains, E2A activation domains, activation domains from human heat-shock factor 1 (HSF1), NFAT (nuclear factor of activated T-cells) activation domains, a histone acetyltransferase, activation domains from the Arabidopsis thaliana MYB46, HAM1, HAM2, MYB112, WRKY11, ERF6, or a combination thereof. Engineered transcription activation systems may comprise one transcription activation domain, two transcription activation domains, three transcription activation domains, or more than three transcription activation domains.

Programmable nucleic acid-binding domains may be a programmable endonuclease (i.e., CRISPR/CAS nuclease, Ago nuclease, or meganuclease) modified to lack all nuclease activity. In some aspects, the programmable nucleic acid modification system is CRISPR/Cas system comprising a Cas9 nuclease comprising a transcriptional activator (Cas9-TA), a guide RNA (gRNA) comprising a sequence complementary to a target sequence, and one or more dead RNA (dRNAs) comprising a sequence complementary to a target sequence upstream of a gene of interest's (GOI) transcription start-site (TSS).

Alternatively, a programmable nucleic acid-binding domain may be a programmable nucleic acid-binding protein such as, e.g., a zinc finger protein or a TALE. For instance, a programmable nucleic acid-binding domain may be a catalytically inactive CRISPR/Cas nuclease in which the nuclease activity was eliminated by mutation and/or deletion. For example, the catalytically inactive CRISPR/Cas protein may be a catalytically inactive (dead) Cas9 (dCas9) in which the RuvC-like domain comprises a D10A, E762A, and/or D986A mutation and the HNH-like domain comprises a H840A (or H839A), N854A and/or N863A mutation. Alternatively, the catalytically inactive CRISPR/Cas protein may be a catalytically inactive (dead) Cpf1 protein comprising comparable mutations in the nuclease domain. A programmable nucleic acid-binding domain may also be a catalytically inactive Ago endonuclease in which nuclease activity was eliminated by mutation and/or deletion. Alternatively, a programmable nucleic acid-binding domain may be a catalytically inactive meganuclease in which nuclease activity was eliminated by mutation and/or deletion, e.g., the catalytically inactive meganuclease may comprise a C-terminal truncation. A programmable nucleic acid-binding domain may also be a transcription activator-like effectors (TALEs) nucleic acid-binding protein.

Transcriptional activation domains may be genetically fused to the nucleic acid binding protein or bound via noncovalent protein-protein, protein-RNA, or protein-DNA interactions. As described above in Section (I)(a)(A)(vii) for programmable nucleic acid modification systems, transcription activation systems may also comprise at least one nuclear localization signal, cell-penetrating domain, reporter domain, and/or detectable label.

(c) Optional Components

A composition may further comprise additional components to facilitate processes such as a nucleic acid modification. For instance, a composition may further comprise a programmable nucleic acid-modification protein. A programmable nucleic acid-modification protein may be a fusion protein comprising a non-nuclease domain and a programmable nucleic acid-binding domain. Suitable programmable nucleic acid-binding domains are described above in Section (I)(a)(A). Examples of suitable non-nuclease domains include epigenetic modification domains. In general, epigenetic modification domains alter gene expression by modifying the histone structure and/or nucleic acid structure. Suitable epigenetic modification domains include, without limit, histone acetyltransferase domains, histone deacetylase domains, histone methyltransferase domains, histone demethylase domains, DNA methyltransferase domains, DNA demethylase domains, transposase domains, integrase domains, recombinase domains, resolvase domains, invertase domains, protease domains, DNA methyltransferase domains, DNA hydroxylmethylase domains, DNA demethylase domains, histone acetylase domains, repressor domains, activator domains, cellular uptake activity associated domains, antibody presentation domains, recruiter of histone modifying enzymes, inhibitor of histone modifying enzymes, histone kinase domains, histone phosphatase domains, histone ribosylase domains, histone deribosylase domains, histone ubiquitinase domains, histone deubiquitinase domains, histone biotinase domains, and histone tail protease domains.

II. Nucleic Acids

A further aspect of the present disclosure provides a system of one or more nucleic acid constructs encoding one or more components of the homologous recombination composition described above in Section I. The system may comprise one or more nucleic acid expression constructs encoding a programmable nucleic acid modification system, one or more expression constructs encoding a transcription activation system specific for inducing expression of a gene of interest, and combinations thereof. A system further comprises a nucleic acid construct encoding a donor polynucleotide of the homologous recombination system.

Compositions may be expressed or encoded by single nucleic acid constructs or multiple nucleic acid constructs. The nucleic acid constructs may be DNA or RNA, linear or circular, single-stranded or double-stranded, or any combination thereof. The nucleic acid constructs may be codon optimized for efficient translation into protein in the cell of interest. Codon optimization programs are available as freeware or from commercial sources.

One or more of the nucleic acid constructs may be RNA. The RNA may be enzymatically synthesized in vitro. For this, DNA encoding the one or more nucleic acids may be operably linked to a promoter sequence that is recognized by a phage RNA polymerase for in vitro RNA synthesis. For example, the promoter sequence may be a T7, T3, or SP6 promoter sequence or a variation of a T7, T3, or SP6 promoter sequence. The DNA encoding the one or more nucleic acids may be part of a vector, as detailed below. In such aspects, the in vitro-transcribed RNA may be purified, capped, and/or polyadenylated. Alternatively, the RNA may be part of a self-replicating RNA (Yoshioka et al., Cell Stem Cell, 2013, 13:246-254). The self-replicating RNA may be derived from a noninfectious, self-replicating Venezuelan equine encephalitis (VEE) virus RNA replicon, which is a positive-sense, single-stranded RNA that is capable of self-replicating for a limited number of cell divisions, and which may be modified to code proteins of interest (Yoshioka et al., Cell Stem Cell, 2013, 13:246-254).

One or more nucleic acid constructs encoding the composition may also be DNA. When one or more of the nucleic acid constructs are DNA, each of the programmable nucleic acid modification system and the transcription activation system may be encoded by one or more nucleic acid expression constructs. The expression constructs comprise DNA coding sequences operably linked to at least one promoter control sequence for expression in a cell of interest. Preferably, promoter control sequences control expression in screenable tissue or cells.

Promoter control sequences may control expression of the programmable nucleic acid modification system and/or the transcription activation system in bacterial (e.g., E. coli) cells or eukaryotic (e.g., yeast, insect, mammalian, or plant) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters. Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-(3 promoter, Mb promoter, NphsI promoter, OG-2 promoter, SP-B promoter, SYN1 promoter, and WASP promoter.

Promoters may also be plant-specific promoters, or promoters that may be used in plants. A wide variety of plant promoters are known to those of ordinary skill in the art, as are other regulatory elements that may be used alone or in combination with promoters. Preferably, promoter control sequences control expression in cassava such as promoters disclosed in Wilson et al., 2017, The New Phytologoist, 213(4):1632-1641, the disclosure of which is incorporated herein in its entirety.

Promoters may be divided into two types, namely, constitutive promoters and non-constitutive promoters. Constitutive promoters are classified as providing for a range of constitutive expression. Thus, some are weak constitutive promoters, and others are strong constitutive promoters. Non-constitutive promoters include tissue-preferred promoters, tissue-specific promoters, cell-type specific promoters, and inducible-promoters. Suitable plant-specific constitutive promoter control sequences include, but are not limited to, a CaMV35S promoter, CaMV 19S, GOS2, Arabidopsis At6669 promoter, Rice cyclophilin, Maize H3 histone, Synthetic Super MAS, an opine promoter, a plant ubiquitin (Ubi) promoter, an actin 1 (Act-1) promoter, pEMU, Cestrum yellow leaf curling virus promoter (CYMLV promoter), and an alcohol dehydrogenase 1 (Adh-1) promoter. Other constitutive promoters include those in U.S. Pat. Nos. 5,659,026; 5,608,149; 5,608,144; 5,604,121; 5,569,597; 5,466,785; 5,399,680; 5,268,463; and 5,608,142.

Regulated plant promoters respond to various forms of environmental stresses, or other stimuli, including, for example, mechanical shock, heat, cold, flooding, drought, salt, anoxia, pathogens such as bacteria, fungi, and viruses, and nutritional deprivation, including deprivation during times of flowering and/or fruiting, and other forms of plant stress. For example, the promoter may be a promoter which is induced by one or more, but not limited to one of the following: abiotic stresses such as wounding, cold, desiccation, ultraviolet-B, heat shock or other heat stress, drought stress or water stress. The promoter may further be one induced by biotic stresses including pathogen stress, such as stress induced by a virus or fungi, stresses induced as part of the plant defense pathway or by other environmental signals, such as light, carbon dioxide, hormones or other signaling molecules such as auxin, hydrogen peroxide and salicylic acid, sugars and gibberellin or abscisic acid and ethylene. Suitable regulated plant promoter control sequences include, but are not limited to, salt-inducible promoters such as RD29A; drought-inducible promoters such as maize rab17 gene promoter, maize rab28 gene promoter, and maize Ivr2 gene promoter; heat-inducible promoters such as heat tomato hsp80-promoter from tomato.

Tissue-specific promoters may include, but are not limited to, fiber-specific, green tissue-specific, root-specific, stem-specific, flower-specific, callus-specific, pollen-specific, egg-specific, and seed coat-specific. Suitable tissue-specific plant promoter control sequences include, but are not limited to, leaf-specific promoters [such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993], seed-preferred promoters [e.g., from seed-specific genes (Simon et al., Plant Mol. Biol. 5. 191, 1985; Scofield et al., J. Biol. Chem. 262: 12202, 1987; Baszczynski et al., Plant Mol. Biol. 14: 633, 1990), Brazil Nut albumin (Pearson et al., Plant Mol. Biol. 18: 235-245, 1992), legumin (Ellis et al., Plant Mol. Biol. 10: 203-214, 1988), Glutelin (rice) (Takaiwa et al., Mol. Gen. Genet. 208: 15-22, 1986; Takaiwa et al., FEBS Letts. 221: 43-47, 1987), Zein (Matzke et al., Plant Mol Biol, 143: 323-32, 1990), napA (Stalberg et al., Planta 199: 515-519, 1996), Wheat SPA (Albanietal, Plant Cell, 9: 171-184, 1997), sunflower oleosin (Cummins et al., Plant Mol. Biol. 19: 873-876, 1992)], endosperm specific promoters [e.g., wheat LMW and HMW, glutenin-1 (Mol Gen Genet 216:81-90, 1989; NAR 17:461-2), wheat a, b and g gliadins (EMBO3:1409-15, 1984), Barley ltrl promoter, barley B1, C, D hordein (Theor Appl Gen 98:1253-62, 1999; Plant J 4:343-55, 1993; Mol Gen Genet 250:750-60, 1996), Barley DOF (Mena et al., The Plant Journal, 116(1): 53-62, 1998), Biz2 (EP99106056.7), Synthetic promoter (Vicente-Carbajosa et al., Plant J. 13: 629-640, 1998), rice prolamin NRP33, rice-globulin Glb-1 (Wu et al., Plant Cell Physiology 39(8) 885-889, 1998), rice alpha-globulin REB/OHP-1 (Nakase et al., Plant Mol. Biol. 33: 513-S22, 1997), rice ADP-glucose PP (Trans Res 6:157-68, 1997), maize ESR gene family (Plant J 12:235-46, 1997), sorgum gamma-kafirin (PMB 32:1029-35, 1996)], embryo-specific promoters [e.g., rice OSH1 (Sato et al., Proc. Natl. Acad. Sci. USA, 93: 8117-8122), KNOX (Postma-Haarsma et al., Plant Mol. Biol. 39:257-71, 1999), rice oleosin (Wu et al., J. Biochem., 123:386, 1998)], and flower-specific promoters [e.g., AtPRP4, chalene synthase (chsA) (Van der Meer et al., Plant Mol. Biol. 15, 95-109, 1990), LAT52 (Twell et al., Mol. Gen Genet. 217:240-245; 1989), apetala-3].

Promoter control sequences may also be promoter control sequences of the gene of interest, such that the expression pattern of the one or more nucleic acid constructs matches the expression pattern of the gene of interest. The promoter sequence may be wild type or it may be modified for more efficient or efficacious expression. The DNA coding sequence also may be linked to a polyadenylation signal (e.g., SV40 polyA signal, bovine growth hormone (BGH) polyA signal, etc.) and/or at least one transcriptional termination sequence. In some situations, the complex or fusion protein may be purified from the bacterial or eukaryotic cells.

Nucleic acids encoding one or more components of a homologous recombination system and/or transcription activation system may be present in a vector. Suitable vectors include plasmid vectors, viral vectors, and self-replicating RNA (Yoshioka et al., Cell Stem Cell, 2013, 13:246-254). For instance, the nucleic acid encoding one or more components of a homologous recombination system and/or transcription activation system may be present in a plasmid vector. Non-limiting examples of suitable plasmid vectors include pUC, pBR322, pET, pBluescript, and variants thereof. Alternatively, the nucleic acid encoding one or more components of a homologous recombination system and/or transcription activation system may be part of a viral vector (e.g., lentiviral vectors, adeno-associated viral vectors, adenoviral vectors, and so forth).

The plasm id or viral vector may comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The plasm id or viral vector may further comprise RNA processing elements such as glycine tRNAs, or Csy4 recognition sites. Such RNA processing elements may, for instance, intersperse polynucleotide sequences encoding multiple gRNAs under the control of a single promoter to produce the multiple gRNAs from a transcript encoding the multiple gRNAs. When a cys4 recognition cite is used, a vector may further comprise sequences for expression of Csy4 RNAse to process the gRNA transcript. Additional information about vectors and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

Below, nucleic acid constructs encoding each component of the homologous recombination system will be described. As explained above, the nucleic acid constructs may be encoded by a single nucleic acid construct or multiple nucleic acid constructs.

(a) Modification System Constructs

As described above, programmable nucleic acid modification systems generally comprise a programmable, sequence-specific nucleic acid-binding domain, and a modification domain. As such, the programmable, sequence-specific nucleic acid-binding domain and the modification domain may be encoded by one or more nucleic acid expression constructs. For instance, when the sequence-specific nucleic acid-binding domain and the modification domain are a single protein, a single nucleic acid construct may encode both functions of the modification system. Alternatively, the sequence-specific nucleic acid-binding domain may be encoded by a first construct, and the modification domain may be encoded by a second construct. Additionally, when nucleic acid binding is mediated by one or more guide RNAs, the guide RNAs may further be encoded by a third nucleic acid expression construct.

When the programmable nucleic acid modification system is encoded by more than one nucleic acid DNA construct, each construct may be operably linked to a promoter, wherein the promoter control sequences for expression in the cell of interest are the same. Alternatively, each expression construct may be operably linked to a different promoter control sequence for finer control of expression in a cell of interest.

When the programmable nucleic acid modification system is encoded by more than one nucleic acid DNA construct, the constructs may be part of one or more vectors. Not being bound by a theory, the ability to simultaneously deliver components of the programmable nucleic acid modification system through a single vector enables application to any cell type of interest, without the need to first generate cell lines that express various components of the programmable nucleic acid modification system.

(b) Donor Polynucleotide Constructs

A donor polynucleotide may be an RNA polynucleotide, an RNA polynucleotide encoded by a DNA construct, or a DNA polynucleotide. An RNA polynucleotide and RNA polynucleotide encoded by a DNA construct may be as described above. When a donor polynucleotide is a DNA polynucleotide, the donor polynucleotide may be a DNA plasmid, a bacterial artificial chromosome (BAC), a yeast artificial chromosome (YAC), a viral vector, a linear piece of DNA, or a PCR fragment. A donor polynucleotide may also be encoded on a nucleic acid construct expressing a programmable nucleic acid modification system and/or a transcription activation system.

(c) Transcription Activation System

As described above, the transcription activation system may comprise a wild type or a modified version of a transcription activator naturally specific for inducing transcription of a gene of interest. A transcription activator may also be a synthetic or artificial programmable transcription activator comprising a nucleic acid-binding and a transcription activation domain. As such, the programmable, sequence-specific nucleic acid-binding domain, and the transcription activation domain may be encoded by one or more nucleic acid constructs. For instance, when the sequence-specific nucleic acid-binding domain and the transcription activation domain are a single protein, a single nucleic acid construct may encode both functions of the transcription activation system. Alternatively, the sequence-specific nucleic acid-binding domain may be encoded by a first construct, and the transcription activation domain may be encoded by a second construct. Additionally, when nucleic acid binding is mediated by a guide RNA, the guide RNA may further be encoded by a third nucleic acid construct.

When the transcription activation system is encoded by more than one nucleic acid DNA construct, each construct may be operably linked to a promoter, wherein the promoter control sequence for expression in the cell of interest is the same. Alternatively, each construct may be operably linked to different promoter control sequences for finer control of expression in the cell of interest.

When the transcription activation system is encoded by more than one nucleic acid DNA construct, the constructs may be part of one or more vectors. Not being bound by a theory, the ability to simultaneously deliver components of the transcription activation system through a single vector enables application to any cell type of interest, without the need to first generate cell lines that express various components of the transcription activation system.

At least one of the constructs expressing a transcription activation system is preferably operably linked to a tissue-specific promoter, more preferably a promoter expressed in easily screenable tissue. For instance, if the homologous recombination is in a plant cell, the easily screenable tissue may include callus tissue or seed coat tissue.

III. Cells

In another aspect, the present disclosure provides a cell comprising a homologous recombination composition. A homologous recombination composition may be as described in Section I above. One or more components of the homologous recombination composition may be encoded by one or more nucleic acid constructs of a system of vectors. The system of vectors may be as described in Section II above.

A variety of cells are suitable for use in the methods disclosed herein. The cell may be a prokaryotic cell. Alternatively, the cell is a eukaryotic cell. For example, the cell may be a prokaryotic cell, a human mammalian cell, a non-human mammalian cell, a non-mammalian vertebrate cell, an invertebrate cell, an insect cell, a plant cell, a yeast cell, or a single cell eukaryotic organism. The cell may also be a one-cell embryo. For example, a non-human mammalian embryo including rat, hamster, rodent, rabbit, feline, canine, ovine, porcine, bovine, equine, plant, and primate embryos. The cell may also be a stem cell such as embryonic stem cells, ES-like stem cells, fetal stem cells, adult stem cells, and the like. The cell may be in vitro, ex vivo, or in vivo (i.e., within an organism or within a tissue of an organism).

Non-limiting examples of suitable mammalian cells or cell lines include human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells; Chinese hamster ovary (CHO) cells; baby hamster kidney (BHK) cells; mouse myeloma NSO cells; mouse embryonic fibroblast 3T3 cells (NIH3T3); mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells; mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; Afrimay green monkey kidney (VERO-76) cells. An extensive list of mammalian cell lines may be found in the Amerimay Type Culture Collection catalog (ATCC, Manassas, VA).

The cell may be a plant cell. Non-limiting examples of plant cells include parenchyma cells, sclerenchyma cells, collenchyma cells, xylem cells, and phloem cells. Preferably, the plant cell is a cell that allows for easy identification of an accurate homologous recombination event. Non-limiting examples of plant tissues that allow for easy identification of an accurate homologous recombination event include ptotoplast cells, cotyledon cells, callus cells, embryos, endosperm cells, and cells of the seed coat.

IV. Programmable RNA-Guided Nuclease Transcription Regulator System

Another aspect of the instant disclosure encompasses a homologous recombination system for detecting an accurate homologous recombination event in a gene of interest in a cell. The homologous recombination system comprises an expression construct for expressing a programmable RNA-guided nuclease transcription regulator; one or more expression constructs for expressing a guide RNA (gRNA) and a deadRNA (dRNA); and a donor polynucleotide comprising a nucleic acid sequence encoding a reporter flanked by regions homologous to nucleic acid sequences at the homologous recombination site. A programmable RNA-guided nuclease transcription regulator targeted by the gRNA induces a homologous recombination event at the homologous recombination site and a programmable RNA-guided nuclease transcription regulator targeted by the dRNA regulates expression of the gene of interest. The cell can be a plant or part thereof, plant cell, or seed.

The expression construct for expressing a programmable RNA-guided nuclease transcription regulator comprises a promoter operably linked to a nucleic acid sequence encoding the programmable RNA-guided nuclease transcription regulator. The one or more expression constructs comprising a promoter operably linked to a nucleic acid sequence comprising the gRNA, a promoter operably linked to a nucleic acid sequence comprising the dRNA an expression construct comprising a promoter operably linked to a dRNA, or a promoter operably linked to a nucleic acid sequence comprising the gRNA and a nucleic acid sequence comprising the dRNA, wherein the gRNA targets the programmable RNA-guided nuclease transcription regulator to a first nucleic acid sequence at a homologous recombination site in a gene of interest and the dRNA targets the programmable RNA-guided nuclease transcription regulator to a second nucleic acid sequence in a regulatory sequence of the gene of interest.

In some aspects, the programmable RNA-guided nuclease transcription regulator comprises an RNA-guided clustered regularly interspersed short palindromic repeats (CRISPR)/CRISPR-associated (Cas) (CRISPR/Cas) nuclease, a single guide RNA (sgRNA) scaffold comprising one or more aptamers, and one or more transcriptional activator linked to the one or more aptamers.

In some aspects, the expression constructs are under control of a ubiquitous promoter or a tissue-specific promoter. Non-limiting examples promoters include the ubiquitous promoter or tissue-specific promoter is At. UBQ10 ubiquitous promoter, Arabidopsis (oleosin1 Promoter—atOLE1) promoter, At U3 ubiquitous promoter, rice callus specific promoter (OsCSP; SEQ ID NO: 10), modified rice callus specific promoter (OsCSP; SEQ ID NO: 11), egg-cell specific promoter At.EC1.2e1.1p, or embryo specific promoter At.YAO.

In some aspects, the programmable RNA-guided nuclease transcription regulator is zCas9-Act3.0 transcriptional activator. In some aspects, the zCas9-Act3.0 transcriptional activator is encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with base 1 to base 6,250 of SEQ ID NO: 13. In some aspects, the zCas9-Act3.0 transcriptional activator is encoded by a nucleic acid sequence comprising at least about 75% or more, at least about 85% or more, at least about 95% or more, or 100% sequence identity with base 1 to base 6,250 of SEQ ID NO: 13.

The zCas9-Act3.0 transcriptional activator can be under control of the At. UBQ10 ubiquitous promoter, egg-cell specific promoter At.EC1.2e1.1p, or embryo specific promoter At.YAO. An expression construct for expressing a dRNA can comprise a modified OsCSP promoter operably linked to a nucleic acid sequence comprising a dRNA. In some aspects, the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with base 1,750 to base 3,340 of SEQ ID NO: 14. In some aspects, the expression construct comprises a nucleic acid sequence comprising at least about 75% or more, at least about 85% or more, at least about 95% or more, or 100% sequence identity with base 1,750 to base 3,340 of SEQ ID NO: 14.

In some aspects, the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with base 1,750 to base 3,340 of SEQ ID NO: 15. In some aspects, the expression construct comprises a nucleic acid sequence comprising at least about 75% or more, at least about 85% or more, at least about 95% or more, or 100% sequence identity with base 1,750 to base 3,340 of SEQ ID NO: 15.

In some aspects, an expression construct for expressing a dRNA comprises an AtOLE1 promoter operably linked to a nucleic acid sequence comprising a dRNA. In some aspects, the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with base 1,750 to base 3,340 of SEQ ID NO: 16. In some aspects, the expression construct comprises a nucleic acid sequence comprising at least about 75% or more, at least about 85% or more, at least about 95% or more, or 100% sequence identity with base 1,750 to base 3,340 of SEQ ID NO: 16. In some aspects, the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with base 1,750 to base 2,950 of SEQ ID NO: 17. In some aspects, the expression construct comprises a nucleic acid sequence comprising at least about 75% or more, at least about 85% or more, at least about 95% or more, or 100% sequence identity with base 1,750 to base 2,950 of SEQ ID NO: 17.

V. Genetically Modified Cells

An additional aspect of the instant disclosure encompasses a genetically modified cell for detecting an accurate homologous recombination event in a gene of interest of the cell. The cell comprises a stably integrated expression construct for expressing a programmable RNA-guided nuclease transcription regulator.

The expression construct can comprise a promoter operably linked to a nucleic acid sequence encoding the programmable RNA-guided nuclease transcription regulator. In some aspects, the programmable RNA-guided nuclease transcription regulator comprises an RNA-guided clustered regularly interspersed short palindromic repeats (CRISPR)/CRISPR-associated (Cas) (CRISPR/Cas) nuclease, a single guide RNA (sgRNA) scaffold comprising one or more aptamers, and one or more transcriptional activator linked to the one or more aptamers.

In some aspects, the programmable RNA-guided nuclease transcription regulator is zCas9-Act3.0 transcriptional activator. In some aspects, the zCas9-Act3.0 transcriptional activator is encoded by a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with base 1 to base 6,250 of SEQ ID NO: 13. In some aspects, the zCas9-Act3.0 transcriptional activator is encoded by a nucleic acid sequence comprising at least about 75% or more, at least about 85% or more, at least about 95% or more, or 100% sequence identity with base 1 to base 6,250 of SEQ ID NO: 13.

The zCas9-Act3.0 transcriptional activator can be under control of the At. UBQ10 ubiquitous promoter, egg-cell specific promoter At.EC1.2e1.1p, or embryo specific promoter At.YAO. An expression construct for expressing a dRNA can comprise a modified OsCSP promoter operably linked to a nucleic acid sequence comprising a dRNA. In some aspects, the expression construct comprises a nucleic acid sequence comprising at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with base 1,750 to base 3,340 of SEQ ID NO: 14. In some aspects, the expression construct comprises a nucleic acid sequence comprising at least about 75% or more, at least about 85% or more, at least about 95% or more, or 100% sequence identity with base 1,750 to base 3,340 of SEQ ID NO: 14.

VI. Methods

A further aspect of the present disclosure provides a method of generating one or more accurate homologous recombination events. The homologous recombination events may be generated in vitro (see, e.g., Liu et al., 2015, mBio vol/6, no. 6, e01714-15). Alternatively, the homologous recombination events may be generated in a cell at one or more nucleic acid loci in nucleic acid sequences of a cell. The cell may be ex vivo or in vivo. The nucleic acid sequences may be chromosomal sequences, organellar chromosomal sequences, or extrachromosomal sequences.

When homologous recombination is generated in a cell, the method comprises providing one or more homologous recombination compositions, and introducing into the cell the one or more homologous recombination compositions. The method further comprises identifying one or more accurate homologous recombination events by identifying one or more cells expressing a reporter. The one or more homologous recombination compositions may be as described in Section I; a system of nucleic acid constructs encoding one or more components of the homologous recombination compositions may be as described in Section II; and the cells may be as described in Section III.

The one or more accurate homologous recombination events may be achieved in a single gene of interest or more. For instance, the accurate homologous recombination event may be achieved in 100 or more unique genes of interest, in 1000 or more unique genes, or in 20,000 or more unique genes. The accurate homologous recombination events may also be achieved in the entire genome. Additionally, more than one accurate homologous recombination event may be generated in a single gene of interest.

(a) Introduction into the Cell

The method comprises introducing the one or more homologous recombination compositions into a cell of interest. The one or more homologous recombination compositions may be introduced into the cell as a purified isolated composition, purified isolated components of a composition, as one or more nucleic acids encoding the one or more homologous recombination compositions, or components of the homologous recombination composition, and combinations thereof.

Components of the one or more homologous recombination compositions may be separately introduced into a cell. For example, a programmable nucleic acid modification system, a donor nucleic acid construct, and a transcription activation system of a composition may be introduced into a cell sequentially. Alternatively, components of a composition may be introduced simultaneously. Similarly, the one or more homologous recombination compositions (or nucleic acids encoding the one or more homologous recombination compositions) may be introduced into a cell sequentially or simultaneously.

The one or more homologous recombination compositions described above may be introduced into the cell by a variety of means. Suitable delivery means include microinjection, electroporation, sonoporation, biolistics, calcium phosphate-mediated transfection, cationic transfection, liposomes and other lipids, dendrimer transfection, heat shock transfection, nucleofection transfection, gene gun delivery, dip transformation, supercharged proteins, cell-penetrating peptides, implantable devices, magnetofection, lipofection, impalefection, optical transfection, proprietary agent-enhanced uptake of nucleic acids, Agrobacterium tumefaciens mediated foreign gene transformation, proprietary agent-enhanced uptake of nucleic acids, and delivery via liposomes, immunoliposomes, virosomes, or artificial virions. In a specific aspect, the targeting endonuclease molecule(s) and polynucleotides(s) are introduced into the cell by nucleofection.

(b) Culturing a Cell

The method further comprises maintaining the cell under appropriate conditions such that the double-stranded break introduced by the targeting endonuclease may be repaired by (i) a non-homologous end-joining repair process such that a nucleic acid locus sequence is modified by a deletion, insertion and/or substitution of at least one base pair or, optionally, (ii) a homology-directed repair process such that the nucleic acid locus sequence is exchanged with the donor sequence of the donor polynucleotide such that the nucleic acid locus sequence is modified. In aspects in which nucleic acid(s) encoding the targeting endonuclease(s) are introduced into the cell, the method comprises maintaining the cell under appropriate conditions such that the cell expresses the targeting endonuclease(s). When the cell is in tissue ex vivo, or in vivo within an organism or within a tissue of an organism, the tissue and/or organism may also be maintained under appropriate conditions for homologous recombination.

In general, the cell is maintained under conditions appropriate for cell growth and/or maintenance. Suitable cell culture conditions are well known in the art and are described, for example, in Santiago et al. (2008) PNAS 105:5809-5814; Moehle et al. (2007) PNAS 104:3055-3060; Urnov et al. (2005) Nature 435:646-651; and Lombardo et al. (2007) Nat. Biotechnology 25:1298-1306; Taylor et al., (2012) Tropical Plant Biology 5: 127-139. Those of skill in the art appreciate that methods for culturing cells are known in the art and may and will vary depending on the cell type. Routine optimization may be used, in all cases, to determine the best techniques for a particular cell type.

During this step of the process, the targeting endonuclease(s) recognizes, binds, and creates a double-stranded break(s) at the targeted cleavage site(s) in the nucleic acid locus sequence. In some aspects, repair of the double-stranded break(s) by NHEJ leads to a deletion, insertion, and/or substitution of at least one base pair in targeted nucleic acid locus sequence(s) such that the targeted nucleic acid locus sequence is inactivated and the cell produces less of the protein of interest. In aspects in which a donor polynucleotide is present, repair of the double-stranded break by a homology-directed process leads to integration of the donor sequence in the donor polynucleotide into the targeted nucleic acid locus such that the cell produces an exogenous protein or more of the protein of interest.

(c) Identification of an Accurate Homologous Recombination Event

The method further comprises identifying an accurate homologous recombination event. The accurate homologous recombination event may be identified by identifying a cell expressing the reporter. Methods of identifying a cell expressing a reporter may and will vary depending on the reporter, the cell, the tissue or the organism comprising the cell, among others. For instance, if a reporter is a visual reporter, a cell expressing a reporter may be identified by observing a visual signal in the cell. If a reporter is a selectable reporter such as antibiotic resistance, a cell expressing a reporter may be identified by selecting an antibiotic resistant cell.

Upon confirmation that an accurate homologous recombination event has occurred, single cell clones may be isolated. Additionally, cells comprising one accurate homologous recombination event may undergo one or more additional rounds of targeted modification to modify additional nucleic acid loci sequences.

VII. Library of Compositions

A further aspect of the present disclosure provides a library of homologous recombination compositions comprising two or more homologous recombination compositions. As homologous recombination compositions and systems described herein may be used to efficiently and cost-effectively target numerous nucleic acid loci, the homologous recombination compositions may comprise a genome wide library of compositions. Such a library may provide for determining the function of genes, cellular pathways genes are involved in, and how any alteration in gene expression may result in a particular biological process. Using the library of homologous recombination compositions would accelerate the identification of accurately targeted homologous recombination events, without requiring large-scale genotyping. Homologous recombination compositions may be as described in Section I. Preferably, each homologous recombination composition is encoded by a system of one or more nucleic acid constructs encoding the homologous recombination composition. Systems of nucleic acid constructs encoding one or more components of the homologous recombination composition may be as described in Section II.

Preferably, each homologous recombination composition comprises a programmable nucleic acid modification system and a programmable transcription activator having a nucleic acid targeting domain that may be provided independently of their respective nuclease, nickase or transcription activation domains. For instance, if the homologous recombination system is a CRISPR nuclease system, each homologous recombination composition may comprise a guide RNA which may be provided independently from the other components of the CRISPR nuclease homologous recombination system. Similarly, if the transcription activation system is based on a CRISPR nuclease system modified to lack all nuclease activity, each homologous recombination composition may comprise a guide RNA which may be provided independently from the other components of the transcription activation system. This arrangement would enable libraries of nucleic acid constructs comprising a cassette of a donor polynucleotide, a gRNA of the CRISPR-based nucleic acid modification system, and a gRNA of the CRISPR-based transcription activation system, all specific for generating a homologous recombination event at a specific nucleic acid locus. Such cassettes may be generated in parallel (100s to 1000s of distinct cassettes) and incorporated into a construct for introducing into cells. Additional components of the CRISPR-based nucleic acid modification system and the CRISPR-based transcription activation system may then be provided independently. Preferably, all the components of the homologous recombination system are encoded on a modular homologous recombination construct comprising a backbone encoding the additional components of the CRISPR-based nucleic acid modification system and the CRISPR-based transcription activation system, and further comprising a cassette comprising the donor polynucleotide, a nucleic acid sequence encoding the gRNA of the CRISPR-based nucleic acid modification system, and a nucleic acid sequence encoding the gRNA of the CRISPR-based transcription activation system. Generating libraries of nucleic acid constructs using such modular constructs would only require inserting into the backbone cassettes comprising the donor polynucleotides and nucleic acid sequences encoding the gRNAs, wherein each cassette is specific for a target nucleic acid locus. One aspect of such a strategy may be as schematically depicted in FIG. 5.

Other strategies for generating libraries of constructs may be envisioned by individuals skilled in the art.

VIII. Kits

A further aspect of the present disclosure provides kits comprising one or more recombination compositions detailed above in Section I, wherein each of the homologous recombination compositions targets a distinct nucleic acid locus. The one or more homologous recombination compositions may be encoded by a system of one or more nucleic acid constructs described above in Section III. Alternatively, the kit may comprise one or more cells comprising one or more homologous recombination compositions, a system of one or more nucleic acid constructs, or combinations thereof.

The kits may further comprise transfection reagents, cell growth media, selection media, in-vitro transcription reagents, nucleic acid purification reagents, protein purification reagents, buffers, and the like. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

NUCLEIC ACID SEQUENCES

| SEQ. | | |
|---|---|---|
| ID. NO. | Sequence | Description. |
| 1 | KRPAATKKAGQAKKKK | Amino acid |
| | | Artificial |
| | | sequence |
| 2 | GRKKRRQRRRPPQPKKKRKV | Amino acid |
| | | Artificial |
| | | sequence |
| 3 | PLSSIFSRIGDPPKKKRKV | Amino acid |
| | | Artificial |
| | | sequence |
| 4 | GALFLGWLGAAGSTMGAPKKKRKV | Amino acid |
| | | Artificial |
| | | sequence |
| 5 | GALFLGFLGAAGSTMGAWSQPKKKRKV | Amino acid |
| | | Artificial |
| | | sequence |
| 6 | KETWWETWWTEWSQPKKKRK | Amino acid |
| | | Artificial |
| | | sequence |
| 7 | AGTGTGGCACTGGCCGTCGTTTTACAACGTCGTGACTGGGAAAACCCTGGCGTTACCCAACTTAATC | Comprises a |
| | GCCTTGCAGCACATCCCCCTTTCGCCAGCTGGCGTAATAGCGAAGAGGCCCGCACCGATCGCCCTTC | construct for |
| | CCAACAGTTGCGCAGCCTGAATGGCGAATGCTAGAGCAGCTTGAGCTTGGATCAGATTGTCGTTTCC | expressing |
| | CGCCTTCAGTTTAAACTATCAGTGTTTGACAGGATATATTGGCGGGTAAACCTAAGAGAAAAGAGCG | the AtCas9 |
| | TTTATTAGAATAATCGGATATTTAAAAGGGCGTGAAAAGGTTTATCCGTTCGTCCATTTGTATGTGC | protein in |
| | ATGCCAACCACAGGGTTCCCCTCGGGATCAAAGTACTTTGATCCAACCCCTCCGCTGCTATAGTGCA | combination |
| | GTCGGCTTCTGACGTTCAGTGCAGCCGTCTTCTGAAAACGACATGTCGCACAAGTCCTAAGTTACGC | with the cys4 |
| | GACAGGCTGCCGCCCTGCCCTTTTCCTGGCGTTTTCTTGTCGCGTGTTTTAGTCGCATAAAGTAGAA | CRISPR RNA |
| | TACTTGCGACTAGAACCGGAGACATTACGCCATGAACAAGAGCGCCGCCGCTGGCCTGCTGGGCTAT | processing |
| | GCCCGCGTCAGCACCGACGACCAGGACTTGACCAACCAACGGGCCGAACTGCACGCGGCCGGCTGCA | protein from |
| | CCAAGCTGTTTTCCGAGAAGATCACCGGCACCAGGCGCGACCGCCCGGAGCTGGCCAGGATGCTTGA | Pseudomonas |
| | CCACCTACGCCCTGGCGACGTTGTGACAGTGACCAGGCTAGACCGCCTGGCCCGCAGCACCCGCGAC | aeruginosa |
| | CTACTGGACATTGCCGAGCGCATCCAGGAGGCCGGCGCGGGCCTGCGTAGCCTGGCAGAGCCGTGGG | under the |
| | CCGACACCACCACGCCGGCCGGCCGCATGGTGTTGACCGTGTTCGCCGGCATTGCCGAGTTCGAGCG | control of |
| | TTCCCTAATCATCGACCGCACCCGGAGCGGGCGCGAGGCCGCCAAGGCCCGAGGCGTGAAGTTTGGC | the 35S |
| | CCCCGCCCTACCCTCACCCCGGCACAGATCGCGCACGCCCGCGAGCTGATCGACCAGGAAGGCCGCA | promoter |
| | CCGTGAAAGAGGCGGCTGCACTGCTTGGCGTGCATCGCTCGACCCTGTACCGCGCACTTGAGCGCAG | |
| | CGAGGAAGTGACGCCCACCGAGGCCAGGCGGCGCGGTGCCTTCCGTGAGGACGCATTGACCGAGGCC | |
| | GACGCCCTGGCGGCCGCCGAGAATGAACGCCAAGAGGAACAAGCATGAAACCGCACCAGGACGGCCA | |
| | GGACGAACCGTTTTTCATTACCGAAGAGATCGAGGCGGAGATGATCGCGGCCGGGTACGTGTTCGAG | |
| | CCGCCCGCGCACGGCTCAACCGTGCGGCTGCATGAAATCCTGGCCGGTTTGTCTGATGCCAAGCTGG | |
| | CGGCCTGGCCGGCCAGCTTGGCCGCTGAAGAAACCGAGCGCCGCCGTCTAAAAAGGTGATGTGTATT | |
| | TGAGTAAAACAGCTTGCGTCATGCGGTCGCTGCGTATATGATGCGATGAGTAAATAAACAAATACGC | |
| | AAGGGGAACGCATGAAGGTTATCGCTGTACTTAACCAGAAAGGCGGGTCAGGCAAGACGACCATCGC | |
| | AACCCATCTAGCCCGCGCCCTGCAACTCGCCGGGGCCGATGTTCTGTTAGTCGATTCCGATCCCCAG | |
| | GGCAGTGCCCGCGATTGGGCGGCCGTGCGGGAAGATCAACCGCTAACCGTTGTCGGCATCGACCGCC | |
| | CGACGATTGACCGCGACGTGAAGGCCATCGGCCGGCGCGACTTCGTAGTGATCGACGGAGCGCCCCA | |
| | GGCGGCGGACTTGGCTGTGTCCGCGATCAAGGCAGCCGACTTCGTGCTGATTCCGGTGCAGCCAAGC | |
| | CCTTACGACATATGGGCCACCGCCGACCTGGTGGAGCTGGTTAAGCAGCGCATTGAGGTCACGGATG | |
| | GAAGGCTACAAGCGGCCTTTGTCGTGTCGCGGGCGATCAAAGGCACGCGCATCGGCGGTGAGGTTGC | |
| | CGAGGCGCTGGCCGGGTACGAGCTGCCCATTCTTGAGTCCCGTATCACGCAGCGCGTGAGCTACCCA | |
| | GGCACTGCCGCCGCCGGCACAACCGTTCTTGAATCAGAACCCGAGGGCGACGCTGCCCGCGAGGTCC | |
| | AGGCGCTGGCCGCTGAAATTAAATCAAAACTCATTTGAGTTAATGAGGTAAAGAGAAAATGAGCAAA | |
| | AGCACAAACACGCTAAGTGCCGGCCGTCCGAGCGCACGCAGCAGCAAGGCTGCAACGTTGGCCAGCC | |
| | TGGCAGACACGCCAGCCATGAAGCGGGTCAACTTTCAGTTGCCGGCGGAGGATCACACCAAGCTGAA | |
| | GATGTACGCGGTACGCCAAGGCAAGACCATTACCGAGCTGCTATCTGAATACATCGCGCAGCTACCA | |
| | GAGTAAATGAGCAAATGAATAAATGAGTAGATGAATTTTAGCGGCTAAAGGAGGCGGCATGGAAAAT | |
| | CAAGAACAACCAGGCACCGACGCCGTGGAATGCCCCATGTGTGGAGGAACGGGCGGTTGGCCAGGCG | |
| | TAAGCGGCTGGGTTGTCTGCCGGCCCTGCAATGGCACTGGAACCCCCAAGCCCGAGGAATCGGCGTG | |
| | ACGGTCGCAAACCATCCGGCCCGGTACAAATCGGCGCGGCGCTGGGTGATGACCTGGTGGAGAAGTT | |
| | GAAGGCCGCGCAGGCCGCCCAGCGGCAACGCATCGAGGCAGAAGCACGCCCCGGTGAATCGTGGCAA | |
| | GCGGCCGCTGATCGAATCCGCAAAGAATCCCGGCAACCGCCGGCAGCCGGTGCGCCGTCGATTAGGA | |
| | AGCCGCCCAAGGGCGACGAGCAACCAGATTTTTTCGTTCCGATGCTCTATGACGTGGGCACCCGCGA | |
| | TAGTCGCAGCATCATGGACGTGGCCGTTTTCCGTCTGTCGAAGCGTGACCGACGAGCTGGCGAGGTG | |
| | ATCCGCTACGAGCTTCCAGACGGGCACGTAGAGGTTTCCGCAGGGCCGGCCGGCATGGCCAGTGTGT | |
| | GGGATTACGACCTGGTACTGATGGCGGTTTCCCATCTAACCGAATCCATGAACCGATACCGGGAAGG | |
| | GAAGGGAGACAAGCCCGGCCGCGTGTTCCGTCCACACGTTGCGGACGTACTCAAGTTCTGCCGGCGA | |
| | GCCGATGGCGGAAAGCAGAAAGACGACCTGGTAGAAACCTGCATTCGGTTAAACACCACGCACGTTG | |
| | CCATGCAGCGTACGAAGAAGGCCAAGAACGGCCGCCTGGTGACGGTATCCGAGGGTGAAGCCTTGAT | |
| | TAGCCGCTACAAGATCGTAAAGAGCGAAACCGGGCGGCCGGAGTACATCGAGATCGAGCTAGCTGAT | |
| | TGGATGTACCGCGAGATCACAGAAGGCAAGAACCCGGACGTGCTGACGGTTCACCCCGATTACTTTT | |
| | TGATCGATCCCGGCATCGGCCGTTTTCTCTACCGCCTGGCACGCCGCGCCGCAGGCAAGGCAGAAGC | |
| | CAGATGGTTGTTCAAGACGATCTACGAACGCAGTGGCAGCGCCGGAGAGTTCAAGAAGTTCTGTTTC | |
| | ACCGTGCGCAAGCTGATCGGGTCAAATGACCTGCCGGAGTACGATTTGAAGGAGGAGGCGGGGCAGG | |
| | CTGGCCCGATCCTAGTCATGCGCTACCGCAACCTGATCGAGGGCGAAGCATCCGCCGGTTCCTAATG | |
| | TACGGAGCAGATGCTAGGGCAAATTGCCCTAGCAGGGGAAAAAGGTCGAAAAGGCCTCTTTCCTGTG | |
| | GATAGCACGTACATTGGGAACCCAAAGCCGTACATTGGGAACCGGAACCCGTACATTGGGAACCCAA | |
| | AGCCGTACATTGGGAACCGGTCACACATGTAAGTGACTGATATAAAAGAGAAAAAAGGCGATTTTTC | |
| | CGCCTAAAACTCTTTAAAACTTATTAAAACTCTTAAAACCCGCCTGGCCTGTGCATAACTGTCTGGC | |
| | CAGCGCACAGCCCAAGAGCTGCAAAAAGCGCCTACCCTTCGGTCGCTGCGCTCCCTACGCCCCGCCG | |
| | CTTCGCGTCGGCCTATCGCGGCCGCTGGCCGCTCAAAAATGGCTGGCCTACGGCCAGGCAATCTACC | |
| | AGGGCGCGGACAAGCCGCGCCGTCGCCACTCGACCGCCGGCGCCCACATCAAGGCACCCTGCCTCGC | |
| | GCGTTTCGGTGATGACGGTGAAAACCTCTGACACATGCAGCTCCCGGAAACGGTCACAGCTTGTCTG | |
| | TAAGCGGATGCCGGGAGCAGACAAGCCCGTCAGGGCGCGTCAGCGGGTGTTGGCGGGTGTCGGGGCG | |
| | CAGCCATGACCCAGTCACGTAGCGATAGCGGAGTGTATACTGGCTTAACTATGCGGCATCAGAGCAG | |
| | ATTGTACTGAGAGTGCACCATATGCGGTGTGAAATACCGCACAGATGCGTAAGGAGAAAATACCGCA | |
| | TCAGGCCCTCTTCCGCTTCCTCGCTCACTGACTCGCTGCGCTCGGTCGTTCGGCTGCGGCGAGCGGT | |
| | ATCAGCTCACTCAAAGGCGGTAATACGGTTATCCACAGAATCAGGGGATAACGCAGGAAAGAACATG | |
| | TGAGCAAAAGGCCAGCAAAAGGCCAGGAACCGTAAAAAGGCCGCGTTGCTGGCGTTTTTCCATAGGC | |
| | TCCGCCCCCCTGACGAGCATCACAAAAATCGACGCTCAAGTCAGAGGTGGCGAAACCCGACAGGACT | |
| | ATAAAGATACCAGGCGTTTCCCCCTGGAAGCTCCCTCGTGCGCTCTCCTGTTCCGACCCTGCCGCTT | |
| | ACCGGATACCTGTCCGCCTTTCTCCCTTCGGGAAGCGTGGCGCTTTCTCATAGCTCACGCTGTAGGT | |
| | ATCTCAGTTCGGTGTAGGTCGTTCGCTCCAAGCTGGGCTGTGTGCACGAACCCCCCGTTCAGCCCGA | |
| | CCGCTGCGCCTTATCCGGTAACTATCGTCTTGAGTCCAACCCGGTAAGACACGACTTATCGCCACTG | |
| | GCAGCAGCCACTGGTAACAGGATTAGCAGAGCGAGGTATGTAGGCGGTGCTACAGAGTTCTTGAAGT | |
| | GGTGGCCTAACTACGGCTACACTAGAAGGACAGTATTTGGTATCTGCGCTCTGCTGAAGCCAGTTAC | |
| | CTTCGGAAAAAGAGTTGGTAGCTCTTGATCCGGCAAACAAACCACCGCTGGTAGCGGTGGTTTTTTT | |
| | GTTTGCAAGCAGCAGATTACGCGCAGAAAAAAAGGATCTCAAGAAGATCCTTTGATCTTTTCTACGG | |
| | GGTCTGACGCTCAGTGGAACGAAAACTCACGTTAAGGGATTTTGGTCATGCATTCTAGGTACTAAAA | |
| | CAATTCATCCAGTAAAATATAATATTTTATTTTCTCCCAATCAGGCTTGATCCCCAGTAAGTCAAAA | |
| | AATAGCTCGACATACTGTTCTTCCCCGATATCCTCCCTGATCGACCGGACGCAGAAGGCAATGTCAT | |
| | ACCACTTGTCCGCCCTGCCGCTTCTCCCAAGATCAATAAAGCCACTTACTTTGCCATCTTTCACAAA | |
| | GATGTTGCTGTCTCCCAGGTCGCCGTGGGAAAAGACAAGTTCCTCTTCGGGCTTTTCCGTCTTTAAA | |
| | AAATCATACAGCTCGCGCGGATCTTTAAATGGAGTGTCTTCTTCCCAGTTTTCGCAATCCACATCGG | |
| | CCAGATCGTTATTCAGTAAGTAATCCAATTCGGCTAAGCGGCTGTCTAAGCTATTCGTATAGGGACA | |
| | ATCCGATATGTCGATGGAGTGAAAGAGCCTGATGCACTCCGCATACAGCTCGATAATCTTTTCAGGG | |
| | CTTTGTTCATCTTCATACTCTTCCGAGCAAAGGACGCCATCGGCCTCACTCATGAGCAGATTGCTCC | |
| | AGCCATCATGCCGTTCAAAGTGCAGGACCTTTGGAACAGGCAGCTTTCCTTCCAGCCATAGCATCAT | |
| | GTCCTTTTCCCGTTCCACATCATAGGTGGTCCCTTTATACCGGCTGTCCGTCATTTTTAAATATAGG | |
| | TTTTCATTTTCTCCCACCAGCTTATATACCTTAGCAGGAGACATTCCTTCCGTATCTTTTACGCAGC | |
| | GGTATTTTTCGATCAGTTTTTTCAATTCCGGTGATATTCTCATTTTAGCCATTTATTATTTCCTTCC | |
| | TCTTTTCTACAGTATTTAAAGATACCCCAAGAAGCTAATTATAACAAGACGAACTCCAATTCACTGT | |
| | TCCTTGCATTCTAAAACCTTAAATACCAGAAAACAGCTTTTTCAAAGTTGTTTTCAAAGTTGGCGTA | |
| | TAACATAGTATCGACGGAGCCGATTTTGAAACCGCGGTGATCACAGGCAGCAACGCTCTGTCATCGT | |
| | TACAATCAACATGCTACCCTCCGCGAGATCATCCGTGTTTCAAACCCGGCAGCTTAGTTGCCGTTCT | |
| | TCCGAATAGCATCGGTAACATGAGCAAAGTCTGCCGCCTTACAACGGCTCTCCCGCTGACGCCGTCC | |
| | CGGACTGATGGGCTGCCTGTATCGAGTGGTGATTTTGTGCCGAGCTGCCGGTCGGGGAGCTGTTGGC | |
| | TGGCTGGTGGCAGGATATATTGTGGTGTAAACAAATTGACGCTTAGACAACTTAATAACACATTGCG | |
| | GACGTTTTTAATGTACTGAATTAACGCCGAATTAATTCGGGGGATCTGGATTTTAGTACTGGATTTT | |
| | GGTTTTAGGAATTAGAAATTTTATTGATAGAAGTATTTTACAAATACAAATACATACTAAGGGTTTC | |
| | TTATATGCTCAACACATGAGCGAAACCCTATAGGAACCCTAATTCCCTTATCTGGGAACTACTCACA | |
| | CATTATTATGGAGAAACTCGAGCTTGTCGATCGACTCTAGCTAGAGGATCGATCCGAACCCCAGAGT | |
| | CCCGCTCAGAAGAACTCGTCAAGAAGGCGATAGAAGGCGATGCGCTGCGAATCGGGAGCGGCGATAC | |
| | CGTAAAGCACGAGGAAGCGGTCAGCCCATTCGCCGCCAAGTTCTTCAGCAATATCACGGGTAGCCAA | |
| | CGCTATGTCCTGATAGCGGTCCGCCACACCCAGCCGGCCACAGTCGATGAATCCAGAAAAGCGGCCA | |
| | TTTTCCACCATGATATTCGGCAAGCAGGCATCGCCATGTGTCACGACGAGATCCTCGCCGTCGGGCA | |
| | TGCGCGCCTTGAGCCTGGCGAACAGTTCGGCTGGCGCGAGCCCCTGATGTTCTTCGTCCAGATCATC | |
| | CTGATCGACAAGACCGGCTTCCATCCGAGTACGTGCTCGCTCGATGCGATGTTTCGCTTGGTGGTCG | |
| | AATGGGCAGGTAGCCGGATCAAGCGTATGCAGCCGCCGCATTGCATCAGCCATGATGGATACTTTCT | |
| | CGGCAGGAGCAAGGTGAGATGACAGGAGATCCTGCCCCGGCACTTCGCCCAATAGCAGCCAGTCCCT | |
| | TCCCGCTTCAGTGACAACGTCGAGCACAGCTGCGCAAGGAACGCCCGTCGTGGCCAGCCACGATAGC | |
| | CGCGCTGCCTCGTCCTGGAGTTCATTCAGGGCACCGGACAGGTCGGTCTTGACAAAAAGAACCGGGC | |
| | GCCCCTGCGCTGACAGCCGAAACACGGCGGCATCAGAGCAGCCGATTGTCTGTTGTGCCCAGTCATA | |
| | GCCGAATAGCCTCTCCACCCAAGCGGCCGGAGAACCTGCGTGCAATCCATCTTGTTCAATCCCCATG | |
| | GTCGATCGACAGATCTGCGAAAGCTCGAGAGAGATAGATTTGTAGAGAGAGACTGGTGATTTCAGCG | |
| | TGTCCTCTCCAAATGAAATGAACTTCCTTATATAGAGGAAGGGTCTTGCGAAGGATAGTGGGATTGT | |
| | GCGTCATCCCTTACGTCAGTGGAGATATCACATCAATCCACTTGCTTTGAAGACGTGGTTGGAACGT | |
| | CTTCTTTTTCCACGATGCTCCTCGTGGGTGGGGGTCCATCTTTGGGACCACTGTCGGCAGAGGCATC | |
| | TTGAACGATAGCCTTTCCTTTATCGCAATGATGGCATTTGTAGGTGCCACCTTCCTTTTCTACTGTC | |
| | CTTTTGATGAAGTGACAGATAGCTGGGCAATGGAATCCGAGGAGGTTTCCCGATATTACCCTTTGTT | |
| | GAAAAGTCTCAATAGCCCTTTGGTCTTCTGAGACTGTATCTTTGATATTCTTGGAGTAGACGAGAGT | |
| | GTCGTGCTCCACCATGTTCACATCAATCCACTTGCTTTGAAGACGTGGTTGGAACGTCTTCTTTTTC | |
| | CACGATGCTCCTCGTGGGTGGGGGTCCATCTTTGGGACCACTGTCGGCAGAGGCATCTTGAACGATA | |
| | GCCTTTCCTTTATCGCAATGATGGCATTTGTAGGTGCCACCTTCCTTTTCTACTGTCCTTTTGATGA | |
| | AGTGACAGATAGCTGGGCAATGGAATCCGAGGAGGTTTCCCGATATTACCCTTTGTTGAAAAGTCTC | |
| | AATAGCCCTTTGGTCTTCTGAGACTGTATCTTTGATATTCTTGGAGTAGACGAGAGTGTCGTGCTCC | |
| | ACCATGTTGGCAAGCTGCTCTAGCCAATACGCAAACCGCCTCTCCCCGCGCGTTGGCCGATTCATTA | |
| | ATGCAGCTGGCACGACAGGTTTCCCGACTGGAAAGCGGGCAGTGAGCGCAACGCAATTAATGTGAGT | |
| | TAGCTCACTCATTAGGCACCCCAGGCTTTACACTTTATGCTTCCGGCTCGTATGTTGTGTGGAATTG | |
| | TGAGCGGATAACAATTTCACACAGGAAACAGCTATGACATGATTACGAATTCGAGCTCGGTACCCGG | |
| | GGATCGGCGCGCCAGATTTGCCTTTTCAATTTCAGAAAGAATGCTAACCCACAGATGGTTAGAGAGG | |
| | CTTACGCAGCAGGTATCATCAAGACGATCTACCCGAGCAATAATCTCCAGGAAATCAAATACCTTCC | |
| | CAAGAAGGTTAAAGATGCAGTCAAAAGATTCAGGACTAACTGCATCAAGAACACAGAGAAAGATATA | |
| | TTTCTCAAGATCAGAAGTACTATTCCAGTATGGACGATTCAAGGCTTGCTTCACAAACCAAGGCAAG | |
| | TAATAGAGATTGGAGTCTCTAAAAAGGTAGTTCCCACTGAATCAAAGGCCATGGAGTCAAAGATTCA | |
| | AATAGAGGACCTAACAGAACTCGCCGTAAAGACTGGCGAACAGTTCATACAGAGTCTCTTACGACTC | |
| | AATGACAAGAAGAAAATCTTCGTCAACATGGTGGAGCACGACACACTTGTCTACTCCAAAAATATCA | |
| | AAGATACAGTCTCAGAAGACCAAAGGGCAATTGAGACTTTTCAACAAAGGGTAATATCCGGAAACCT | |
| | CCTCGGATTCCATTGCCCAGCTATCTGTCACTTTATTGTGAAGATAGTGGAAAAGGAAGGTGGCTCC | |
| | TACAAATGCCATCATTGCGATAAAGGAAAGGCCATCGTTGAAGATGCCTCTGCCGACAGTGGTCCCA | |
| | AAGATGGACCCCCACCCACGAGGAGCATCGTGGAAAAAGAAGACGTTCCAACCACGTCTTCAAAGCA | |
| | AGTGGATTGATGTGATATCTCCACTGACGTAAGGGATGACGCACAATCCCACTATCCTTCGCAAGAC | |
| | CCTTCCTCTATATAAGGAAGTTCATTTCATTTGGAGAGAACACGGGGGACTCCTGCAGGAAAAATGG | |
| | ATCATTATCTTGATATTAGACTTAGACCTGATCCAGAATTTCCACCAGCTCAACTTATGTCTGTTCT | |
| | TTTTGGAAAACTTCATCAAGCTCTTGTTGCTCAAGGAGGAGATAGAATTGGAGTTTCTTTTCCTGAT | |
| | CTTGATGAATCAAGATCAAGACTTGGAGAAAGACTTAGAATTCATGCTTCTGCTGATGATCTTAGAG | |
| | CTTTGCTTGCTAGACCTTGGCTTGAAGGACTTAGAGATCATCTTCAATTTGGAGAACCAGCTGTTGT | |
| | TCCACATCCAACTCCTTATAGACAAGTTTCAAGAGTTCAAGCTAAATCTAATCCAGAAAGACTTAGA | |
| | AGAAGACTTATGAGAAGACATGATCTTTCTGAAGAAGAAGCTAGAAAAAGAATTCCTGATACTGTTG | |
| | CTAGAGCTTTGGATTTGCCTTTTGTTACACTTAGATCACAATCTACTGGACAACATTTTAGACTTTT | |
| | TATTAGACATGGACCACTTCAAGTTACTGCTGAAGAAGGAGGATTTACTTGTTATGGACTTTCTAAG | |
| | GGAGGTTTTGTTCCTTGGTTTGGATCTGGAGCTACTAATTTTTCTCTTCTTAAGCAAGCTGGAGATG | |
| | TTGAAGAAAATCCTGGACCCATGGATAAGAAGTACTCTATCGGACTCGATATCGGAACTAACTCTGT | |
| | GGGATGGGCTGTGATCACCGATGAGTACAAGGTGCCATCTAAGAAGTTCAAGGTTCTCGGAAACACC | |
| | GATAGGCACTCTATCAAGAAAAACCTTATCGGTGCTCTCCTCTTCGATTCTGGTGAAACTGCTGAGG | |
| | CTACCAGACTCAAGAGAACCGCTAGAAGAAGGTACACCAGAAGAAAGAACAGGATCTGCTACCTCCA | |
| | AGAGATCTTCTCTAACGAGATGGCTAAAGTGGATGATTCATTCTTCCACAGGCTCGAAGAGTCATTC | |
| | CTCGTGGAAGAAGATAAGAAGCACGAGAGGCACCCTATCTTCGGAAACATCGTTGATGAGGTGGCAT | |
| | ACCACGAGAAGTACCCTACTATCTACCACCTCAGAAAGAAGCTCGTTGATTCTACTGATAAGGCTGA | |
| | TCTCAGGCTCATCTACCTCGCTCTCGCTCACATGATCAAGTTCAGAGGACACTTCCTCATCGAGGGT | |
| | GATCTCAACCCTGATAACTCTGATGTGGATAAGTTGTTCATCCAGCTCGTGCAGACCTACAACCAGC | |
| | TTTTCGAAGAGAACCCTATCAACGCTTCAGGTGTGGATGCTAAGGCTATCCTCTCTGCTAGGCTCTC | |
| | TAAGTCAAGAAGGCTTGAGAACCTCATTGCTCAGCTCCCTGGTGAGAAGAAGAACGGACTTTTCGGA | |
| | AACTTGATCGCTCTCTCTCTCGGACTCACCCCTAACTTCAAGTCTAACTTCGATCTCGCTGAGGATG | |
| | CAAAGCTCCAGCTCTCAAAGGATACCTACGATGATGATCTCGATAACCTCCTCGCTCAGATCGGAGA | |
| | TCAGTACGCTGATTTGTTCCTCGCTGCTAAGAACCTCTCTGATGCTATCCTCCTCAGTGATATCCTC | |
| | AGAGTGAACACCGAGATCACCAAGGCTCCACTCTCAGCTTCTATGATCAAGAGATACGATGAGCACC | |
| | ACCAGGATCTCACACTTCTCAAGGCTCTTGTTAGACAGCAGCTCCCAGAGAAGTACAAAGAGATTTT | |
| | CTTCGATCAGTCTAAGAACGGATACGCTGGTTACATCGATGGTGGTGCATCTCAAGAAGAGTTCTAC | |
| | AAGTTCATCAAGCCTATCCTCGAGAAGATGGATGGAACCGAGGAACTCCTCGTGAAGCTCAATAGAG | |
| | AGGATCTTCTCAGAAAGCAGAGGACCTTCGATAACGGATCTATCCCTCATCAGATCCACCTCGGAGA | |
| | GTTGCACGCTATCCTTAGAAGGCAAGAGGATTTCTACCCATTCCTCAAGGATAACAGGGAAAAGATT | |
| | GAGAAGATTCTCACCTTCAGAATCCCTTACTACGTGGGACCTCTCGCTAGAGGAAACTCAAGATTCG | |
| | CTTGGATGACCAGAAAGTCTGAGGAAACCATCACCCCTTGGAACTTCGAAGAGGTGGTGGATAAGGG | |
| | TGCTAGTGCTCAGTCTTTCATCGAGAGGATGACCAACTTCGATAAGAACCTTCCAAACGAGAAGGTG | |
| | CTCCCTAAGCACTCTTTGCTCTACGAGTACTTCACCGTGTACAACGAGTTGACCAAGGTTAAGTACG | |
| | TGACCGAGGGAATGAGGAAGCCTGCTTTTTTGTCAGGTGAGCAAAAGAAGGCTATCGTTGATCTCTT | |
| | GTTCAAGACCAACAGAAAGGTGACCGTGAAGCAGCTCAAAGAGGATTACTTCAAGAAAATCGAGTGC | |
| | TTCGATTCAGTTGAGATTTCTGGTGTTGAGGATAGGTTCAACGCATCTCTCGGAACCTACCACGATC | |
| | TCCTCAAGATCATTAAGGATAAGGATTTCTTGGATAACGAGGAAAACGAGGATATCTTGGAGGATAT | |
| | CGTTCTTACCCTCACCCTCTTTGAAGATAGAGAGATGATTGAAGAAAGGCTCAAGACCTACGCTCAT | |
| | CTCTTCGATGATAAGGTGATGAAGCAGTTGAAGAGAAGAAGATACACTGGTTGGGGAAGGCTCTCAA | |
| | GAAAGCTCATTAACGGAATCAGGGATAAGCAGTCTGGAAAGACAATCCTTGATTTCCTCAAGTCTGA | |
| | TGGATTCGCTAACAGAAACTTCATGCAGCTCATCCACGATGATTCTCTCACCTTTAAAGAGGATATC | |
| | CAGAAGGCTCAGGTTTCAGGACAGGGTGATAGTCTCCATGAGCATATCGCTAACCTCGCTGGATCTC | |
| | CTGCAATCAAGAAGGGAATCCTCCAGACTGTGAAGGTTGTGGATGAGTTGGTGAAGGTGATGGGAAG | |
| | GCATAAGCCTGAGAACATCGTGATCGAAATGGCTAGAGAGAACCAGACCACTCAGAAGGGACAGAAG | |
| | AACTCTAGGGAAAGGATGAAGAGGATCGAGGAAGGTATCAAAGAGCTTGGATCTCAGATCCTCAAAG | |
| | AGCACCCTGTTGAGAACACTCAGCTCCAGAATGAGAAGCTCTACCTCTACTACCTCCAGAACGGAAG | |
| | GGATATGTATGTGGATCAAGAGTTGGATATCAACAGGCTCTCTGATTACGATGTTGATCATATCGTG | |
| | CCACAGTCATTCTTGAAGGATGATTCTATCGATAACAAGGTGCTCACCAGGTCTGATAAGAACAGGG | |
| | GTAAGAGTGATAACGTGCCAAGTGAAGAGGTTGTGAAGAAAATGAAGAACTATTGGAGGCAGCTCCT | |
| | CAACGCTAAGCTCATCACTCAGAGAAAGTTCGATAACTTGACTAAGGCTGAGAGGGGAGGACTCTCT | |
| | GAATTGGATAAGGCAGGATTCATCAAGAGGCAGCTTGTGGAAACCAGGCAGATCACTAAGCACGTTG | |
| | CACAGATCCTCGATTCTAGGATGAACACCAAGTACGATGAGAACGATAAGTTGATCAGGGAAGTGAA | |
| | GGTTATCACCCTCAAGTCAAAGCTCGTGTCTGATTTCAGAAAGGATTTCCAATTCTACAAGGTGAGG | |
| | GAAATCAACAACTACCACCACGCTCACGATGCTTACCTTAACGCTGTTGTTGGAACCGCTCTCATCA | |
| | AGAAGTATCCTAAGCTCGAGTCAGAGTTCGTGTACGGTGATTACAAGGTGTACGATGTGAGGAAGAT | |
| | GATCGCTAAGTCTGAGCAAGAGATCGGAAAGGCTACCGCTAAGTATTTCTTCTACTCTAACATCATG | |
| | AATTTCTTCAAGACCGAGATTACCCTCGCTAACGGTGAGATCAGAAAGAGGCCACTCATCGAGACAA | |
| | ACGGTGAAACAGGTGAGATCGTGTGGGATAAGGGAAGGGATTTCGCTACCGTTAGAAAGGTGCTCTC | |
| | TATGCCACAGGTGAACATCGTTAAGAAAACCGAGGTGCAGACCGGTGGATTCTCTAAAGAGTCTATC | |
| | CTCCCTAAGAGGAACTCTGATAAGCTCATTGCTAGGAAGAAGGATTGGGACCCTAAGAAATACGGTG | |
| | GTTTCGATTCTCCTACCGTGGCTTACTCTGTTCTCGTTGTGGCTAAGGTTGAGAAGGGAAAGAGTAA | |
| | GAAGCTCAAGTCTGTTAAGGAACTTCTCGGAATCACTATCATGGAAAGGTCATCTTTCGAGAAGAAC | |
| | CCAATCGATTTCCTCGAGGCTAAGGGATACAAAGAGGTTAAGAAGGATCTCATCATCAAGCTCCCAA | |
| | AGTACTCACTCTTCGAACTCGAGAACGGTAGAAAGAGGATGCTCGCTTCTGCTGGTGAGCTTCAAAA | |
| | GGGAAACGAGCTTGCTCTCCCATCTAAGTACGTTAACTTTCTTTACCTCGCTTCTCACTACGAGAAG | |
| | TTGAAGGGATCTCCAGAAGATAACGAGCAGAAGCAACTTTTCGTTGAGCAGCACAAGCACTACTTGG | |
| | ATGAGATCATCGAGCAGATCTCTGAGTTCTCTAAAAGGGTGATCCTCGCTGATGCAAACCTCGATAA | |
| | GGTGTTGTCTGCTTACAACAAGCACAGAGATAAGCCTATCAGGGAACAGGCAGAGAACATCATCCAT | |
| | CTCTTCACCCTTACCAACCTCGGTGCTCCTGCTGCTTTCAAGTACTTCGATACAACCATCGATAGGA | |
| | AGAGATACACCTCTACCAAAGAAGTGCTCGATGCTACCCTCATCCATCAGTCTATCACTGGACTCTA | |
| | CGAGACTAGGATCGATCTCTCACAGCTCGGTGGTGATTCAAGGGCTGATCCTAAGAAGAAGAGGAAG | |
| | GTTTGACGTCGACGATATGAAGATGAAGATGAAATATTTGGTGTGTCAAATAAAAAGCTTGTGTGCT | |
| | TAAGTTTGTGTTTTTTTCTTGGCTTGTTGTGTTATGAATTTGTGGCTTTTTCTAATATTAAATGAAT | |
| | GTAAGATCACATTATAATGAATAAACAAATGTTTCTATAATCCATTGTGAATGTTTTGTTGGATCTC | |
| | TTCTGCAGCATATAACTACTGTATGTGCTATGGTATGGACTATGGAATATGATTAAAGATAAGCCAG | |
| | AGCTCTGGTGACGGACGGCGCGCTGGCAGACATACTGTCCCACAAATGAAGATGGAATCTGTAAAAG | |
| | AAAACGCGTGAAATAATGCGTCTGACAAAGGTTAGGTCGGCTGCCTTTAATCAATACCAAAGTGGTC | |
| | CCTACCACGATGGAAAAACTGTGCAGTCGGTTTGGCTTTTTCTGACGAACAAATAAGATTCGTGGCC | |
| | GACAGGTGGGGGTCCACCATGTGAAGGCATCTTCAGACTCCAATAATGGAGCAATGACGTAAGGGCT | |
| | TACGAAATAAGTAAGGGTAGTTTGGGAAATGTCCACTCACCCGTCAGTCTATAAATACTTAGCCCCT | |
| | CCCTCATTGTTAAGGGAGCAAAATCTCAGAGAGATAGTCCTAGAGAGAGAAAGAGAGCAAGTAGCCT | |
| | AGAAGTAGTCAAGGCGGCGAAGTATTCAGGCACGTGGCCAGGAAGAAGAAAAGCCAAGACGACGAAA | |
| | ACAGGTAAGAGCTAAGCTTCCTGCAGGTTCACTGCCGTATAGGCAGCATTAACATTACCATTAACGG | |
| | TTTTAGAGCTAGAAATAGCAAGTTAAAATAAGGCTAGTCCGTTATCAACTTGAAAAAGTGGCACCGA | |
| | GTCGGTGCGTTCACTGCCGTATAGGCAGAGGGACACCAATGTCCTGCTGTTTTAGAGCTAGAAATAG | |
| | CAAGTTAAAATAAGGCTAGTCCGTTATCAACTTGAAAAAGTGGCACCGAGTCGGTGCGTTCACTGCC | |
| | GTATAGGCAGGTCGATCGACAAGCTCGAGTTTCTCCATAATAATGTGTGAGTAGTTCCCAGATAAGG | |
| | GAATTAGGGTTCCTATAGGGTTTCGCTCATGTGTTGAGCATATAAGAAACCCTTAGTATGTATTTGT | |
| | ATTTGTAAAATACTTCTATCAATAAAATTTCTAATTCCTAAAACCAAAATCCAGTACTAAAATCCAG | |
| | ATCCCCCGAATTAGAGCTCTACCGGCGAGCTTTGGGTACGTCACGTGGCTCGAGCGCGTAGTCCTCG | |
| | GTAGGCAAGCTTATTTAATTCATACAGAAGCAATCTTTGTTTCAGATGTTCACTACAAAACTCATCC | |
| | TCTTCTTCAATATTTTTGGTTTCGGAATGATCGCTATCTTAACTCTTTTCCTTACACATGGCCGCAA | |
| | ACGCGTTGATGTTCTTGGATGGATTTGCATGATCTTTGCTTTATGCGTGTTTGTTGCCCCCATGGGT | |
| | ATCATGGTGAGAATGCGAGTCGCAAATTTCAACACTTGCTTCTTTCTGTCTCTGACAGTTTTTTTTT | |
| | TTTCCCCTATAATTATATTGATTGATTTTTGTTTTCTCTCTTCTTTACTCTATTTTCCAGAGAAAAG | |
| | TGATAAAAACGAAGAGTGTCGAGTTCATGCCATTTTCTTTATCATTCTTCCTCACCTTGACTGCGGT | |
| | GATGTGGTTCTTCTATGGTTTTCTAAAGAAAGACCTTTATGTTGCCGTAAGTTAACTATCACGCATG | |
| | CATCATTATCACGTACATCTTTCTTTACATTCCACCAACTTTATCTTTCCCATTAATCATCAACCCA | |
| | GCAACTATTTCTTATTCCCTTTTGATTAACTTCCACTTACAATTTCCTTTTTCTTGTCATGAACAGA | |
| | TTCCAAACACATTGGGCTTTCTTTTTGGGATTGTCCAGATGGTGCTTTATTTAATCTACAGAAACCC | |
| | CAAGAAATTACCTGTAGAGGATCCTAAACTTCGCGAATTGTCCGAGCACATCGTCGACGTTGCAAAG | |
| | CTGAGTGCAACCCTCTGTTCCGAGATAACCACAGTAGTGGTTCCACAGCCCATAGACAATGGAAATG | |
| | ATGTTGAAGGTCAAAAAATTAAGGAAGAAAACGAGCAGGACATTGGTGTCCCTGCAGACAAAGTTAA | |
| | GACTAATCTTTTTCTCTTTCTCATCTTTTCACTTCTCCAATCATTATCCTCGGCCGAATTCAGTAAA | |
| | GGAGAAGAACTTTTCACTGGAGTTGTCCCAATTCTTGTTGAATTAGATGGTGATGTTAATGGGCACA | |
| | AATTTTCTGTCAGTGGAGAGGGTGAAGGTGATGCAACATACGGAAAACTTACCCTTAAATTTATTTG | |
| | CACTACTGGAAAACTACCTGTTCCATGGCCAACACTTGTCACTACTTTCACTTATGGTGTTCAATGC | |
| | TTTTCAAGATACCCAGATCATATGAAGCGGCACGACTTCTTCAAGAGCGCCATGCCTGAGGGATACG | |
| | TGCAGGAGAGGACCATCTCTTTCAAGGACGACGGGAACTACAAGACACGTGCTGAAGTCAAGTTTGA | |
| | GGGAGACACCCTCGTCAACAGGATCGAGCTTAAGGGAATCGATTTCAAGGAGGACGGAAACATCCTC | |
| | GGCCACAAGTTGGAATACAACTACAACTCCCACAACGTATACATCACGGCAGACAAACAAAAGAATG | |
| | GAATCAAAGCTAACTTCAAAATTAGACACAACATTGAAGATGGAAGCGTTCAACTAGCAGACCATTA | |
| | TCAACAAAATACTCCAATTGGCGATGGCCCTGTCCTTTTACCAGACAACCATTACCTGTCCACACAA | |
| | TCTGCCCTTTCGAAAGATCCCAACGAAAAGAGAGACCACATGGTCCTTCTTGAGTTTGTAACAGCTG | |
| | CTGGGATTACACATGGCATGGATGAACTATACAAACATGATGAGCTTTGATAACATTAACATTACCA | |
| | TTAACGTGATCTTGGTTATGTTTTTTCTTTTTAATTTTGCATGTAATCGTTCAAAGTGGTGGTGCCA | |
| | TGTCTACTTGTAAGGCTGCAATGCAGCCATGTTGTCTATTATGTCAAATCTAGTTCCATTTAATGTC | |
| | AATCTTTATTCTCAACCTAAAAGAAGAATATCAATCTTTATGTAATACGTTTTTTCGAGTAAATAAA | |
| | ATGTCCAGTGAATTTACAGTTAATGTTAAATCAGCATTATATTTTAGGAAAATAGTATTCAACTTAT | |
| | AGTTTAATGGTTGAAATTAAATATTAATTTTTATTTTATGATGTAATAATTTTAAATTTAAATTATA | |
| | GCTCCTGGCAAGAGTTATTAATAAAATAATACTGCCAATATTTTTTTCTAAATTTTATTTGAATTTG | |
| | TTATTTATTTTATGGAAAATATTTTTAAAAAATAATTTTCATATTTTTTTATATAAGAAGAGCTCAA | |
| | AAAAATTTTAAATCCATGTTATTTTACACTAAAAAACAGAAGTTTAAATAGGGGAGAAATTTTTACA | |
| | TTCGCCAACAAAACTATATAAATTTTTGTTTTGAATTATAAAATAATAATTATTTTTCCTAAAAAGA | |
| | ATTCTTCATGATTGTGCCAAATAAGTCTCAATGCAATTTTAAAAAAAATCCAGACAAAATTTGTCTT | |
| | ATTTCTCACTGTGCTATTTTTCTAATAAGCATTTTCATTGTGCAATTAAATCTATTGGACTCTAATC | |
| | AATAATAAAGAAAAGGGATACCTTTAATCTTTTATCGAAGATATCAACTAATTCTAGAGCGCGGTAA | |
| | TATCGCAGAACAAAAGTACCTGATATCGAGTGTACTTCAAGTCACACCGGCG | |
| 8 | AGTGTGGCACTGGCCGTCGTTTTACAACGTCGTGACTGGGAAAACCCTGGCGTTACCCAACTTAATC | Comprises a |
| | GCCTTGCAGCACATCCCCCTTTCGCCAGCTGGCGTAATAGCGAAGAGGCCCGCACCGATCGCCCTTC | construct for |
| | CCAACAGTTGCGCAGCCTGAATGGCGAATGCTAGAGCAGCTTGAGCTTGGATCAGATTGTCGTTTCC | expressing |
| | CGCCTTCAGTTTAAACTATCAGTGTTTGACAGGATATATTGGCGGGTAAACCTAAGAGAAAAGAGCG | the AtCas9 |
| | TTTATTAGAATAATCGGATATTTAAAAGGGCGTGAAAAGGTTTATCCGTTCGTCCATTTGTATGTGC | protein in |
| | ATGCCAACCACAGGGTTCCCCTCGGGATCAAAGTACTTTGATCCAACCCCTCCGCTGCTATAGTGCA | combination |
| | GTCGGCTTCTGACGTTCAGTGCAGCCGTCTTCTGAAAACGACATGTCGCACAAGTCCTAAGTTACGC | with the cys4 |
| | GACAGGCTGCCGCCCTGCCCTTTTCCTGGCGTTTTCTTGTCGCGTGTTTTAGTCGCATAAAGTAGAA | CRISPR RNA |
| | TACTTGCGACTAGAACCGGAGACATTACGCCATGAACAAGAGCGCCGCCGCTGGCCTGCTGGGCTAT | processing |
| | GCCCGCGTCAGCACCGACGACCAGGACTTGACCAACCAACGGGCCGAACTGCACGCGGCCGGCTGCA | protein from |
| | CCAAGCTGTTTTCCGAGAAGATCACCGGCACCAGGCGCGACCGCCCGGAGCTGGCCAGGATGCTTGA | Pseudomonas |
| | CCACCTACGCCCTGGCGACGTTGTGACAGTGACCAGGCTAGACCGCCTGGCCCGCAGCACCCGCGAC | aeruginosa |
| | CTACTGGACATTGCCGAGCGCATCCAGGAGGCCGGCGCGGGCCTGCGTAGCCTGGCAGAGCCGTGGG | under the |
| | CCGACACCACCACGCCGGCCGGCCGCATGGTGTTGACCGTGTTCGCCGGCATTGCCGAGTTCGAGCG | control of |
| | TTCCCTAATCATCGACCGCACCCGGAGCGGGCGCGAGGCCGCCAAGGCCCGAGGCGTGAAGTTTGGC | the 35S |
| | CCCCGCCCTACCCTCACCCCGGCACAGATCGCGCACGCCCGCGAGCTGATCGACCAGGAAGGCCGCA | promoter |
| | CCGTGAAAGAGGCGGCTGCACTGCTTGGCGTGCATCGCTCGACCCTGTACCGCGCACTTGAGCGCAG | And |
| | CGAGGAAGTGACGCCCACCGAGGCCAGGCGGCGCGGTGCCTTCCGTGAGGACGCATTGACCGAGGCC | construct for |
| | GACGCCCTGGCGGCCGCCGAGAATGAACGCCAAGAGGAACAAGCATGAAACCGCACCAGGACGGCCA | the TAL20 |
| | GGACGAACCGTTTTTCATTACCGAAGAGATCGAGGCGGAGATGATCGCGGCCGGGTACGTGTTCGAG | transcription |
| | CCGCCCGCGCACGGCTCAACCGTGCGGCTGCATGAAATCCTGGCCGGTTTGTCTGATGCCAAGCTGG | activator |
| | CGGCCTGGCCGGCCAGCTTGGCCGCTGAAGAAACCGAGCGCCGCCGTCTAAAAAGGTGATGTGTATT | under the |
| | TGAGTAAAACAGCTTGCGTCATGCGGTCGCTGCGTATATGATGCGATGAGTAAATAAACAAATACGC | control of |
| | AAGGGGAACGCATGAAGGTTATCGCTGTACTTAACCAGAAAGGCGGGTCAGGCAAGACGACCATCGC | the tissue- |
| | AACCCATCTAGCCCGCGCCCTGCAACTCGCCGGGGCCGATGTTCTGTTAGTCGATTCCGATCCCCAG | specific |
| | GGCAGTGCCCGCGATTGGGCGGCCGTGCGGGAAGATCAACCGCTAACCGTTGTCGGCATCGACCGCC | Manes.17G095200 |
| | CGACGATTGACCGCGACGTGAAGGCCATCGGCCGGCGCGACTTCGTAGTGATCGACGGAGCGCCCCA | |
| | GGCGGCGGACTTGGCTGTGTCCGCGATCAAGGCAGCCGACTTCGTGCTGATTCCGGTGCAGCCAAGC | |
| | CCTTACGACATATGGGCCACCGCCGACCTGGTGGAGCTGGTTAAGCAGCGCATTGAGGTCACGGATG | |
| | GAAGGCTACAAGCGGCCTTTGTCGTGTCGCGGGCGATCAAAGGCACGCGCATCGGCGGTGAGGTTGC | |
| | CGAGGCGCTGGCCGGGTACGAGCTGCCCATTCTTGAGTCCCGTATCACGCAGCGCGTGAGCTACCCA | |
| | GGCACTGCCGCCGCCGGCACAACCGTTCTTGAATCAGAACCCGAGGGCGACGCTGCCCGCGAGGTCC | |
| | AGGCGCTGGCCGCTGAAATTAAATCAAAACTCATTTGAGTTAATGAGGTAAAGAGAAAATGAGCAAA | |
| | AGCACAAACACGCTAAGTGCCGGCCGTCCGAGCGCACGCAGCAGCAAGGCTGCAACGTTGGCCAGCC | |
| | TGGCAGACACGCCAGCCATGAAGCGGGTCAACTTTCAGTTGCCGGCGGAGGATCACACCAAGCTGAA | |
| | GATGTACGCGGTACGCCAAGGCAAGACCATTACCGAGCTGCTATCTGAATACATCGCGCAGCTACCA | |
| | GAGTAAATGAGCAAATGAATAAATGAGTAGATGAATTTTAGCGGCTAAAGGAGGCGGCATGGAAAAT | |
| | CAAGAACAACCAGGCACCGACGCCGTGGAATGCCCCATGTGTGGAGGAACGGGCGGTTGGCCAGGCG | |
| | TAAGCGGCTGGGTTGTCTGCCGGCCCTGCAATGGCACTGGAACCCCCAAGCCCGAGGAATCGGCGTG | |
| | ACGGTCGCAAACCATCCGGCCCGGTACAAATCGGCGCGGCGCTGGGTGATGACCTGGTGGAGAAGTT | |
| | GAAGGCCGCGCAGGCCGCCCAGCGGCAACGCATCGAGGCAGAAGCACGCCCCGGTGAATCGTGGCAA | |
| | GCGGCCGCTGATCGAATCCGCAAAGAATCCCGGCAACCGCCGGCAGCCGGTGCGCCGTCGATTAGGA | |
| | AGCCGCCCAAGGGCGACGAGCAACCAGATTTTTTCGTTCCGATGCTCTATGACGTGGGCACCCGCGA | |
| | TAGTCGCAGCATCATGGACGTGGCCGTTTTCCGTCTGTCGAAGCGTGACCGACGAGCTGGCGAGGTG | |
| | ATCCGCTACGAGCTTCCAGACGGGCACGTAGAGGTTTCCGCAGGGCCGGCCGGCATGGCCAGTGTGT | |
| | GGGATTACGACCTGGTACTGATGGCGGTTTCCCATCTAACCGAATCCATGAACCGATACCGGGAAGG | |
| | GAAGGGAGACAAGCCCGGCCGCGTGTTCCGTCCACACGTTGCGGACGTACTCAAGTTCTGCCGGCGA | |
| | GCCGATGGCGGAAAGCAGAAAGACGACCTGGTAGAAACCTGCATTCGGTTAAACACCACGCACGTTG | |
| | CCATGCAGCGTACGAAGAAGGCCAAGAACGGCCGCCTGGTGACGGTATCCGAGGGTGAAGCCTTGAT | |
| | TAGCCGCTACAAGATCGTAAAGAGCGAAACCGGGCGGCCGGAGTACATCGAGATCGAGCTAGCTGAT | |
| | TGGATGTACCGCGAGATCACAGAAGGCAAGAACCCGGACGTGCTGACGGTTCACCCCGATTACTTTT | |
| | TGATCGATCCCGGCATCGGCCGTTTTCTCTACCGCCTGGCACGCCGCGCCGCAGGCAAGGCAGAAGC | |
| | CAGATGGTTGTTCAAGACGATCTACGAACGCAGTGGCAGCGCCGGAGAGTTCAAGAAGTTCTGTTTC | |
| | ACCGTGCGCAAGCTGATCGGGTCAAATGACCTGCCGGAGTACGATTTGAAGGAGGAGGCGGGGCAGG | |
| | CTGGCCCGATCCTAGTCATGCGCTACCGCAACCTGATCGAGGGCGAAGCATCCGCCGGTTCCTAATG | |
| | TACGGAGCAGATGCTAGGGCAAATTGCCCTAGCAGGGGAAAAAGGTCGAAAAGGCCTCTTTCCTGTG | |
| | GATAGCACGTACATTGGGAACCCAAAGCCGTACATTGGGAACCGGAACCCGTACATTGGGAACCCAA | |
| | AGCCGTACATTGGGAACCGGTCACACATGTAAGTGACTGATATAAAAGAGAAAAAAGGCGATTTTTC | |
| | CGCCTAAAACTCTTTAAAACTTATTAAAACTCTTAAAACCCGCCTGGCCTGTGCATAACTGTCTGGC | |
| | CAGCGCACAGCCCAAGAGCTGCAAAAAGCGCCTACCCTTCGGTCGCTGCGCTCCCTACGCCCCGCCG | |
| | CTTCGCGTCGGCCTATCGCGGCCGCTGGCCGCTCAAAAATGGCTGGCCTACGGCCAGGCAATCTACC | |
| | AGGGCGCGGACAAGCCGCGCCGTCGCCACTCGACCGCCGGCGCCCACATCAAGGCACCCTGCCTCGC | |
| | GCGTTTCGGTGATGACGGTGAAAACCTCTGACACATGCAGCTCCCGGAAACGGTCACAGCTTGTCTG | |
| | TAAGCGGATGCCGGGAGCAGACAAGCCCGTCAGGGCGCGTCAGCGGGTGTTGGCGGGTGTCGGGGCG | |
| | CAGCCATGACCCAGTCACGTAGCGATAGCGGAGTGTATACTGGCTTAACTATGCGGCATCAGAGCAG | |
| | ATTGTACTGAGAGTGCACCATATGCGGTGTGAAATACCGCACAGATGCGTAAGGAGAAAATACCGCA | |
| | TCAGGCCCTCTTCCGCTTCCTCGCTCACTGACTCGCTGCGCTCGGTCGTTCGGCTGCGGCGAGCGGT | |
| | ATCAGCTCACTCAAAGGCGGTAATACGGTTATCCACAGAATCAGGGGATAACGCAGGAAAGAACATG | |
| | TGAGCAAAAGGCCAGCAAAAGGCCAGGAACCGTAAAAAGGCCGCGTTGCTGGCGTTTTTCCATAGGC | |
| | TCCGCCCCCCTGACGAGCATCACAAAAATCGACGCTCAAGTCAGAGGTGGCGAAACCCGACAGGACT | |
| | ATAAAGATACCAGGCGTTTCCCCCTGGAAGCTCCCTCGTGCGCTCTCCTGTTCCGACCCTGCCGCTT | |
| | ACCGGATACCTGTCCGCCTTTCTCCCTTCGGGAAGCGTGGCGCTTTCTCATAGCTCACGCTGTAGGT | |
| | ATCTCAGTTCGGTGTAGGTCGTTCGCTCCAAGCTGGGCTGTGTGCACGAACCCCCCGTTCAGCCCGA | |
| | CCGCTGCGCCTTATCCGGTAACTATCGTCTTGAGTCCAACCCGGTAAGACACGACTTATCGCCACTG | |
| | GCAGCAGCCACTGGTAACAGGATTAGCAGAGCGAGGTATGTAGGCGGTGCTACAGAGTTCTTGAAGT | |
| | GGTGGCCTAACTACGGCTACACTAGAAGGACAGTATTTGGTATCTGCGCTCTGCTGAAGCCAGTTAC | |
| | CTTCGGAAAAAGAGTTGGTAGCTCTTGATCCGGCAAACAAACCACCGCTGGTAGCGGTGGTTTTTTT | |
| | GTTTGCAAGCAGCAGATTACGCGCAGAAAAAAAGGATCTCAAGAAGATCCTTTGATCTTTTCTACGG | |
| | GGTCTGACGCTCAGTGGAACGAAAACTCACGTTAAGGGATTTTGGTCATGCATTCTAGGTACTAAAA | |
| | CAATTCATCCAGTAAAATATAATATTTTATTTTCTCCCAATCAGGCTTGATCCCCAGTAAGTCAAAA | |
| | AATAGCTCGACATACTGTTCTTCCCCGATATCCTCCCTGATCGACCGGACGCAGAAGGCAATGTCAT | |
| | ACCACTTGTCCGCCCTGCCGCTTCTCCCAAGATCAATAAAGCCACTTACTTTGCCATCTTTCACAAA | |
| | GATGTTGCTGTCTCCCAGGTCGCCGTGGGAAAAGACAAGTTCCTCTTCGGGCTTTTCCGTCTTTAAA | |
| | AAATCATACAGCTCGCGCGGATCTTTAAATGGAGTGTCTTCTTCCCAGTTTTCGCAATCCACATCGG | |
| | CCAGATCGTTATTCAGTAAGTAATCCAATTCGGCTAAGCGGCTGTCTAAGCTATTCGTATAGGGACA | |
| | ATCCGATATGTCGATGGAGTGAAAGAGCCTGATGCACTCCGCATACAGCTCGATAATCTTTTCAGGG | |
| | CTTTGTTCATCTTCATACTCTTCCGAGCAAAGGACGCCATCGGCCTCACTCATGAGCAGATTGCTCC | |
| | AGCCATCATGCCGTTCAAAGTGCAGGACCTTTGGAACAGGCAGCTTTCCTTCCAGCCATAGCATCAT | |
| | GTCCTTTTCCCGTTCCACATCATAGGTGGTCCCTTTATACCGGCTGTCCGTCATTTTTAAATATAGG | |
| | TTTTCATTTTCTCCCACCAGCTTATATACCTTAGCAGGAGACATTCCTTCCGTATCTTTTACGCAGC | |
| | GGTATTTTTCGATCAGTTTTTTCAATTCCGGTGATATTCTCATTTTAGCCATTTATTATTTCCTTCC | |
| | TCTTTTCTACAGTATTTAAAGATACCCCAAGAAGCTAATTATAACAAGACGAACTCCAATTCACTGT | |
| | TCCTTGCATTCTAAAACCTTAAATACCAGAAAACAGCTTTTTCAAAGTTGTTTTCAAAGTTGGCGTA | |
| | TAACATAGTATCGACGGAGCCGATTTTGAAACCGCGGTGATCACAGGCAGCAACGCTCTGTCATCGT | |
| | TACAATCAACATGCTACCCTCCGCGAGATCATCCGTGTTTCAAACCCGGCAGCTTAGTTGCCGTTCT | |
| | TCCGAATAGCATCGGTAACATGAGCAAAGTCTGCCGCCTTACAACGGCTCTCCCGCTGACGCCGTCC | |
| | CGGACTGATGGGCTGCCTGTATCGAGTGGTGATTTTGTGCCGAGCTGCCGGTCGGGGAGCTGTTGGC | |
| | TGGCTGGTGGCAGGATATATTGTGGTGTAAACAAATTGACGCTTAGACAACTTAATAACACATTGCG | |
| | GACGTTTTTAATGTACTGAATTAACGCCGAATTAATTCGGGGGATCTGGATTTTAGTACTGGATTTT | |
| | GGTTTTAGGAATTAGAAATTTTATTGATAGAAGTATTTTACAAATACAAATACATACTAAGGGTTTC | |
| | TTATATGCTCAACACATGAGCGAAACCCTATAGGAACCCTAATTCCCTTATCTGGGAACTACTCACA | |
| | CATTATTATGGAGAAACTCGAGCTTGTCGATCGACTCTAGCTAGAGGATCGATCCGAACCCCAGAGT | |
| | CCCGCTCAGAAGAACTCGTCAAGAAGGCGATAGAAGGCGATGCGCTGCGAATCGGGAGCGGCGATAC | |
| | CGTAAAGCACGAGGAAGCGGTCAGCCCATTCGCCGCCAAGTTCTTCAGCAATATCACGGGTAGCCAA | |
| | CGCTATGTCCTGATAGCGGTCCGCCACACCCAGCCGGCCACAGTCGATGAATCCAGAAAAGCGGCCA | |
| | TTTTCCACCATGATATTCGGCAAGCAGGCATCGCCATGTGTCACGACGAGATCCTCGCCGTCGGGCA | |
| | TGCGCGCCTTGAGCCTGGCGAACAGTTCGGCTGGCGCGAGCCCCTGATGTTCTTCGTCCAGATCATC | |
| | CTGATCGACAAGACCGGCTTCCATCCGAGTACGTGCTCGCTCGATGCGATGTTTCGCTTGGTGGTCG | |
| | AATGGGCAGGTAGCCGGATCAAGCGTATGCAGCCGCCGCATTGCATCAGCCATGATGGATACTTTCT | |
| | CGGCAGGAGCAAGGTGAGATGACAGGAGATCCTGCCCCGGCACTTCGCCCAATAGCAGCCAGTCCCT | |
| | TCCCGCTTCAGTGACAACGTCGAGCACAGCTGCGCAAGGAACGCCCGTCGTGGCCAGCCACGATAGC | |
| | CGCGCTGCCTCGTCCTGGAGTTCATTCAGGGCACCGGACAGGTCGGTCTTGACAAAAAGAACCGGGC | |
| | GCCCCTGCGCTGACAGCCGAAACACGGCGGCATCAGAGCAGCCGATTGTCTGTTGTGCCCAGTCATA | |
| | GCCGAATAGCCTCTCCACCCAAGCGGCCGGAGAACCTGCGTGCAATCCATCTTGTTCAATCCCCATG | |
| | GTCGATCGACAGATCTGCGAAAGCTCGAGAGAGATAGATTTGTAGAGAGAGACTGGTGATTTCAGCG | |
| | TGTCCTCTCCAAATGAAATGAACTTCCTTATATAGAGGAAGGGTCTTGCGAAGGATAGTGGGATTGT | |
| | GCGTCATCCCTTACGTCAGTGGAGATATCACATCAATCCACTTGCTTTGAAGACGTGGTTGGAACGT | |
| | CTTCTTTTTCCACGATGCTCCTCGTGGGTGGGGGTCCATCTTTGGGACCACTGTCGGCAGAGGCATC | |
| | TTGAACGATAGCCTTTCCTTTATCGCAATGATGGCATTTGTAGGTGCCACCTTCCTTTTCTACTGTC | |
| | CTTTTGATGAAGTGACAGATAGCTGGGCAATGGAATCCGAGGAGGTTTCCCGATATTACCCTTTGTT | |
| | GAAAAGTCTCAATAGCCCTTTGGTCTTCTGAGACTGTATCTTTGATATTCTTGGAGTAGACGAGAGT | |
| | GTCGTGCTCCACCATGTTCACATCAATCCACTTGCTTTGAAGACGTGGTTGGAACGTCTTCTTTTTC | |
| | CACGATGCTCCTCGTGGGTGGGGGTCCATCTTTGGGACCACTGTCGGCAGAGGCATCTTGAACGATA | |
| | GCCTTTCCTTTATCGCAATGATGGCATTTGTAGGTGCCACCTTCCTTTTCTACTGTCCTTTTGATGA | |
| | AGTGACAGATAGCTGGGCAATGGAATCCGAGGAGGTTTCCCGATATTACCCTTTGTTGAAAAGTCTC | |
| | AATAGCCCTTTGGTCTTCTGAGACTGTATCTTTGATATTCTTGGAGTAGACGAGAGTGTCGTGCTCC | |
| | ACCATGTTGGCAAGCTGCTCTAGCCAATACGCAAACCGCCTCTCCCCGCGCGTTGGCCGATTCATTA | |
| | ATGCAGCTGGCACGACAGGTTTCCCGACTGGAAAGCGGGCAGTGAGCGCAACGCAATTAATGTGAGT | |
| | TAGCTCACTCATTAGGCACCCCAGGCTTTACACTTTATGCTTCCGGCTCGTATGTTGTGTGGAATTG | |
| | TGAGCGGATAACAATTTCACACAGGAAACAGCTATGACATGATTACGAATTCGAGCTCGGTACCCAC | |
| | CCTACTTAAAAACCCTTTCGATTAAATCTATTATTATTATTTTTCATATTGTTATAATTAAACAACG | |
| | TAGATAAAGTTTAATAAATTTATTTTATTAATTAATTTAATTATATAAAAAAAGAAAGAGGTAAAAA | |
| | TGAAAAAGGCAAAAAGGTAGTTTTCTTGAACCCAAAATTTGTAGAGCATGGTCCTCTTTTTTGAAAA | |
| | AAAATTAAGACAAAACTTGAGAGATTTTACTTTAATAAATTTAGATTGTAAGATTGAAGAAGGAATC | |
| | ATCAAAGGGGGATTTAATATTTATATTTTATTTTTTTATAAAAAATTTATTTATTTATATTTTATAA | |
| | TTAATTTGATTTATAATAAATTGAGGTCTAGGTAAGTATTTCACCTGCCGAATGTTGGCATATGGGA | |
| | TCACTATGACAAATCACAAAGCTGCCAAATCAAATTTGTCTTTGCCTAAACCGCTCCATCCTAATAC | |
| | CACATCAAATCCTGTCTTCATTCATTCTGAGTTAAAGCCTTCCACACACCATAAATACTCCATCCAT | |
| | GTACTGCAAAGCTCCCACCATCTTTATCTTCACGAAAAAAAAATCCCACTTCCTCGTACTGAAATCC | |
| | AGAGGTCACCATGGATCCCATTCGTCCGCGCACGCCAAGTCCTGCCCACGAACTTCTGGCCGGACCC | |
| | CAGCCGGATAGGGTTCAGCCGCAGCCGACTGCAGATCGTGGGGGGGCTCCGCCTGCCGGCAGCCCCC | |
| | TGGATGGCTTGCCCGCTCGACGGACGATGTCCCGAACCCGTCTCCCGTCTCCCCCTGCACCCTTGCC | |
| | TGCGTTCTCAGCGGGCAGTTTCAGCGATCTGCTCCGTCAGTTCGATCCGTCGCTTCTTGATACATCG | |
| | CTTTTTAATTCGATGTCTGCCTTCGGCGCTCCTCATACAGAGGCTGCCTCAGGAGAGGGGGATGAGG | |
| | TGCAATCGGGTCTGCGTGCAGCCGATGACCCGCAAGCCACCGTGCAGGTCGCTGTGACGGCCGCGCG | |
| | ACCGCCGCGCGCCAAGCCGGCGCCGCGACGGCGTGCTGCGCACACCTCTGACGCTTCGCCGGCCGGG | |
| | CAGGTCGATCTATGCACGCTCGGCTACAGCCAGCAGCAGCAAGAGAAGATCAAACTGAAGGCTCGTT | |
| | CGACAGTAGCACAGCACCACGAGGCACTGATCGGCCATGGGTTTACACGTGCGCACATCGTTGCGCT | |
| | CAGCCAACACCCGGCAGCCTTAGGGACCGTCGCTGTCAAGTACCAGGCCATGATCGCGGCGTTGCCG | |
| | GAGGCGACACACGAAGACATCGTTGGCGGCGGCAAACAGTGGTCCGGCGCACGCGCCCTGGAAGCAT | |
| | TGCTCACGGTGTCGGGAGAGTTGAGAGGTCCACCGTTACAGTTGGACACAGGTCAACTTCTCAAGAT | |
| | TGCAAAACGTGGCGGCGTGACCGCGGTGGAGGCAGTGCATGCATGGCGCAATGCACTGACGGGCGCT | |
| | CCCCTGAACCTGACCCCGGACCAGGTGGTGGCCATCGCCAGCAATATTGGCGGCAAGCAGGCGCTGG | |
| | AGACGGTGCAGCGGCTGTTGCCGGTGCTGTGCGAGCAACATGGCCTGACCCTGGACCAGGTGGTGGC | |
| | CATCGCCAGCAATGGCGGCGGCAAGCAGGCGCTGGAGACGGTGCAGCGGCTGTTGCCGGTGCTGTGC | |
| | GAGCAACATGGTCTGACCCCGGACCAGGTGGTGGCTATCGCCAGCAATATTGGCGGCAAGCAGGCGC | |
| | TGGAGACGGTGCAGCGGCTGTTGCCGGTGCTGTGCGAGCAACATGGTCTGACCCCGGACCAGGTGGT | |
| | GGCCATCGCCAGCAATAACGGCGGCAAGCAGGCGCTGGAGACGGTGCAGCGGCTGTTGCCGGTGCTG | |
| | TGCGAGCAACATGGCCTGACCCCGGACCAGGTGGTGGCTATCGCCAGCAATATTGGCGGCAAGCAGG | |
| | CGCTGGAGACGGTGCAGCGGCTGTTGCCGGTGCTGCGCCAGGCCCATGGCCTGACCCCGGCGCAGGT | |
| | GGTGGCCATCGCCAGCCACGATGGCGGCAAGCAGGCGCTGGAGACGGTGCAGCAGCTGTTGCCGGTG | |
| | CTGTGCGAGCAACATGGCCTGACCCCGGCGCAGGTGGTGGCCATCGCCAGCAATAGCGGCGGCAAGC | |
| | AGGCGCTGGAGACGGTGCAGCGGCTGTTGCCGGTGCTGCGCCAGGCCCATGGCCTGACCCCGGACCA | |
| | GGTGGTGGCCATCGCCAGCAATAGCGGCGGCAAGCCGGCGCTGGAGACGGTGCAGCGGCTGTTGCCG | |
| | GTGCTGTGCGAGCAACATGGTCTGACCCCGGACCAGGTGGTGGCCATCGCCAGCAATAACGGCGGCA | |
| | AGCCGGCGCTGGAGACGGTGCAGCGGCTGTTGCCGGTGCTGTGCGAGCAACATGGCCTGACCCGGGC | |
| | GCAGGTGGTGGCCATCGCCAGCAATGGCGGCGGCAAGCAGGCGCTGGAGACGGTGCAGCGGCTGTTG | |
| | CCGGTGCTGCGCCAGGCCCATGGCCTGACCCCGGCGCAGGTGGTGGCCATCGCCAGCCACGATGGCG | |
| | GCAAGCAGGCGCTGGAGACGGTGCAGCAGCTGTTGCCGGTGCTGTGCGAGCAACATGGCCTGACCCC | |
| | GGCGCAGGTGGTGGCCATCGCCAGCAATAGCGGCGGCAAGCAGGCGCTGGAGACGGTGCAGCGGCTG | |
| | TTGCCGGTGCTGCGCCAGGCCCATGGCCTGACCCCGGACCAGGTGGTGGCCATCGCCAGCCACGATG | |
| | GCGGCAAGCAGGCGCTGGAGACGGTGCAGCGGCTGTTGCCGGTGCTGTGCGAGCAACATGGTCTGAC | |
| | CCCGGACCAGGTGGTGGCCATCGCCAGCAATAACGGCGGCAAGCAGGCGCTGGAGACGGTGCAGCGG | |
| | CTGTTGCCGGTGCTGTGCGAGCAACATGGCCTGACCCCGGACCAGGTGGTGGCCATCGCCAGCCACG | |
| | ATGGCGGCAAGCAGGCGCTGGAGACGGTGCAGCGGCTGTTGCCGGTGCTGTGCGAGCAACATGGCCT | |
| | GACCCCGGACCAGGTGGTGGCCATCGCCAGCCACGATGGCGGCAAGCAGGCGCTGGAGACGGTGCAG | |
| | CGGCTGTTGCCGGTGCTGCGCCAGGCCCATGGCCTGACCCCGGCGCAGGTGGTGGCCATCGCCAGCC | |
| | ACGATGGCGGCAAGCCGGCGCTGGAGACGGTGCAGCGGCTGTTGCCGGTGCTGTGCGAGCAACATGG | |
| | CCTGACCCCGGACCAGGTGGTGGCTATCGCCAGCAATATTGGCGGCAAGCAGGCGCTGGAGACGGTG | |
| | CAGCGGCTGTTGCCGGTGCTGTGCGAGCAACATGGCCTGACCCCGGACCAGGTGGTGGCCATCGCCA | |
| | GCAATGGCGGCGGCAAGCAGGCGCTGGAGACGGTGCAGCGGCTGTTGCCGGTGCTGTGCGAGCAACA | |
| | TGGTCTGACCCCGGCGCAGGTGGTGGCCATCGCCAGCAATGGCGGCGGCAGGCCGGCGCTGGAGAGC | |
| | ATTTTTGCCCAGTTATCTCGCCCTGATCAGGCGTTGGCCGCGTTGACCAACGACCACCTCGTCGCCT | |
| | TGGCCTGCCTCGGCGGGCGTCCTGCGCTGGAGGCAGTGAAAAAGGGATTGCCGCACGCGCCGACCTT | |
| | GATCAAAAGAACCAATCGCCGTCTTCCCGAACGCACGTCCCATCGCGTTGCCGACCACGCGCAAGTG | |
| | GCTCGCGTGTTGGGTTTTTTCCAGTGCCACTCCCACCCAGCGCAAGCATTTGATGAAGCCATGACGC | |
| | AGTTCGGGATGAGCAGGCACGGGTTGTTACAGCTATTTCGCAGAGCCGGCGTCACCGAACTCGAGGC | |
| | CCACAGTGGAACGCTCCCCCCAGCCTCGCAGCGTTGGCACCGTATCCTCCAGGCATCAGGGATGAAA | |
| | AGGGCCGAACCGTCCGGTGCTTCCGCTCAAACGCCGGACCAGGCGTCTTTGCATGCATTCGCCGATG | |
| | CGCTGGAGCGTGAGCTGGATGCGCCCAGCCCAATAGACCGGGCGGGCCAGGCGCTGGCAAGCAGCAG | |
| | CCGTAAACGGTCCCGATCGGAGAGTTCTGTCACCGGCTCCTTCGCACAGCAAGCTGTCGAGGTGCGC | |
| | GTTCCCGAACAGCGCGATGCGCTGCATTTCCTCCCCCTCAGCTGGGGTGTAAAACGCCCGCGTACCA | |
| | GGATCGGGGGCGGCCTCCCGGATCCTGGTACGCCCATGGACGCCGACCTGGCACCGTCCAGCACCGT | |
| | GATGTGGGAACAAGATGCTGACCCCTTCGCAGGGGCAGCGGATGATTTTCCGGCATTCAACGAAGAG | |
| | GAGATGGCATGGTTGATGGAGCTATTTCCTCAGTGAGGGGATCGGCGCGCCAGATTTGCCTTTTCAA | |
| | TTTCAGAAAGAATGCTAACCCACAGATGGTTAGAGAGGCTTACGCAGCAGGTATCATCAAGACGATC | |
| | TACCCGAGCAATAATCTCCAGGAAATCAAATACCTTCCCAAGAAGGTTAAAGATGCAGTCAAAAGAT | |
| | TCAGGACTAACTGCATCAAGAACACAGAGAAAGATATATTTCTCAAGATCAGAAGTACTATTCCAGT | |
| | ATGGACGATTCAAGGCTTGCTTCACAAACCAAGGCAAGTAATAGAGATTGGAGTCTCTAAAAAGGTA | |
| | GTTCCCACTGAATCAAAGGCCATGGAGTCAAAGATTCAAATAGAGGACCTAACAGAACTCGCCGTAA | |
| | AGACTGGCGAACAGTTCATACAGAGTCTCTTACGACTCAATGACAAGAAGAAAATCTTCGTCAACAT | |
| | GGTGGAGCACGACACACTTGTCTACTCCAAAAATATCAAAGATACAGTCTCAGAAGACCAAAGGGCA | |
| | ATTGAGACTTTTCAACAAAGGGTAATATCCGGAAACCTCCTCGGATTCCATTGCCCAGCTATCTGTC | |
| | ACTTTATTGTGAAGATAGTGGAAAAGGAAGGTGGCTCCTACAAATGCCATCATTGCGATAAAGGAAA | |
| | GGCCATCGTTGAAGATGCCTCTGCCGACAGTGGTCCCAAAGATGGACCCCCACCCACGAGGAGCATC | |
| | GTGGAAAAAGAAGACGTTCCAACCACGTCTTCAAAGCAAGTGGATTGATGTGATATCTCCACTGACG | |
| | TAAGGGATGACGCACAATCCCACTATCCTTCGCAAGACCCTTCCTCTATATAAGGAAGTTCATTTCA | |
| | TTTGGAGAGAACACGGGGGACTCCTGCAGGAAAAATGGATCATTATCTTGATATTAGACTTAGACCT | |
| | GATCCAGAATTTCCACCAGCTCAACTTATGTCTGTTCTTTTTGGAAAACTTCATCAAGCTCTTGTTG | |
| | CTCAAGGAGGAGATAGAATTGGAGTTTCTTTTCCTGATCTTGATGAATCAAGATCAAGACTTGGAGA | |
| | AAGACTTAGAATTCATGCTTCTGCTGATGATCTTAGAGCTTTGCTTGCTAGACCTTGGCTTGAAGGA | |
| | CTTAGAGATCATCTTCAATTTGGAGAACCAGCTGTTGTTCCACATCCAACTCCTTATAGACAAGTTT | |
| | CAAGAGTTCAAGCTAAATCTAATCCAGAAAGACTTAGAAGAAGACTTATGAGAAGACATGATCTTTC | |
| | TGAAGAAGAAGCTAGAAAAAGAATTCCTGATACTGTTGCTAGAGCTTTGGATTTGCCTTTTGTTACA | |
| | CTTAGATCACAATCTACTGGACAACATTTTAGACTTTTTATTAGACATGGACCACTTCAAGTTACTG | |
| | CTGAAGAAGGAGGATTTACTTGTTATGGACTTTCTAAGGGAGGTTTTGTTCCTTGGTTTGGATCTGG | |
| | AGCTACTAATTTTTCTCTTCTTAAGCAAGCTGGAGATGTTGAAGAAAATCCTGGACCCATGGATAAG | |
| | AAGTACTCTATCGGACTCGATATCGGAACTAACTCTGTGGGATGGGCTGTGATCACCGATGAGTACA | |
| | AGGTGCCATCTAAGAAGTTCAAGGTTCTCGGAAACACCGATAGGCACTCTATCAAGAAAAACCTTAT | |
| | CGGTGCTCTCCTCTTCGATTCTGGTGAAACTGCTGAGGCTACCAGACTCAAGAGAACCGCTAGAAGA | |
| | AGGTACACCAGAAGAAAGAACAGGATCTGCTACCTCCAAGAGATCTTCTCTAACGAGATGGCTAAAG | |
| | TGGATGATTCATTCTTCCACAGGCTCGAAGAGTCATTCCTCGTGGAAGAAGATAAGAAGCACGAGAG | |
| | GCACCCTATCTTCGGAAACATCGTTGATGAGGTGGCATACCACGAGAAGTACCCTACTATCTACCAC | |
| | CTCAGAAAGAAGCTCGTTGATTCTACTGATAAGGCTGATCTCAGGCTCATCTACCTCGCTCTCGCTC | |
| | ACATGATCAAGTTCAGAGGACACTTCCTCATCGAGGGTGATCTCAACCCTGATAACTCTGATGTGGA | |
| | TAAGTTGTTCATCCAGCTCGTGCAGACCTACAACCAGCTTTTCGAAGAGAACCCTATCAACGCTTCA | |
| | GGTGTGGATGCTAAGGCTATCCTCTCTGCTAGGCTCTCTAAGTCAAGAAGGCTTGAGAACCTCATTG | |
| | CTCAGCTCCCTGGTGAGAAGAAGAACGGACTTTTCGGAAACTTGATCGCTCTCTCTCTCGGACTCAC | |
| | CCCTAACTTCAAGTCTAACTTCGATCTCGCTGAGGATGCAAAGCTCCAGCTCTCAAAGGATACCTAC | |
| | GATGATGATCTCGATAACCTCCTCGCTCAGATCGGAGATCAGTACGCTGATTTGTTCCTCGCTGCTA | |
| | AGAACCTCTCTGATGCTATCCTCCTCAGTGATATCCTCAGAGTGAACACCGAGATCACCAAGGCTCC | |
| | ACTCTCAGCTTCTATGATCAAGAGATACGATGAGCACCACCAGGATCTCACACTTCTCAAGGCTCTT | |
| | GTTAGACAGCAGCTCCCAGAGAAGTACAAAGAGATTTTCTTCGATCAGTCTAAGAACGGATACGCTG | |
| | GTTACATCGATGGTGGTGCATCTCAAGAAGAGTTCTACAAGTTCATCAAGCCTATCCTCGAGAAGAT | |
| | GGATGGAACCGAGGAACTCCTCGTGAAGCTCAATAGAGAGGATCTTCTCAGAAAGCAGAGGACCTTC | |
| | GATAACGGATCTATCCCTCATCAGATCCACCTCGGAGAGTTGCACGCTATCCTTAGAAGGCAAGAGG | |
| | ATTTCTACCCATTCCTCAAGGATAACAGGGAAAAGATTGAGAAGATTCTCACCTTCAGAATCCCTTA | |
| | CTACGTGGGACCTCTCGCTAGAGGAAACTCAAGATTCGCTTGGATGACCAGAAAGTCTGAGGAAACC | |
| | ATCACCCCTTGGAACTTCGAAGAGGTGGTGGATAAGGGTGCTAGTGCTCAGTCTTTCATCGAGAGGA | |
| | TGACCAACTTCGATAAGAACCTTCCAAACGAGAAGGTGCTCCCTAAGCACTCTTTGCTCTACGAGTA | |
| | CTTCACCGTGTACAACGAGTTGACCAAGGTTAAGTACGTGACCGAGGGAATGAGGAAGCCTGCTTTT | |
| | TTGTCAGGTGAGCAAAAGAAGGCTATCGTTGATCTCTTGTTCAAGACCAACAGAAAGGTGACCGTGA | |
| | AGCAGCTCAAAGAGGATTACTTCAAGAAAATCGAGTGCTTCGATTCAGTTGAGATTTCTGGTGTTGA | |
| | GGATAGGTTCAACGCATCTCTCGGAACCTACCACGATCTCCTCAAGATCATTAAGGATAAGGATTTC | |
| | TTGGATAACGAGGAAAACGAGGATATCTTGGAGGATATCGTTCTTACCCTCACCCTCTTTGAAGATA | |
| | GAGAGATGATTGAAGAAAGGCTCAAGACCTACGCTCATCTCTTCGATGATAAGGTGATGAAGCAGTT | |
| | GAAGAGAAGAAGATACACTGGTTGGGGAAGGCTCTCAAGAAAGCTCATTAACGGAATCAGGGATAAG | |
| | CAGTCTGGAAAGACAATCCTTGATTTCCTCAAGTCTGATGGATTCGCTAACAGAAACTTCATGCAGC | |
| | TCATCCACGATGATTCTCTCACCTTTAAAGAGGATATCCAGAAGGCTCAGGTTTCAGGACAGGGTGA | |
| | TAGTCTCCATGAGCATATCGCTAACCTCGCTGGATCTCCTGCAATCAAGAAGGGAATCCTCCAGACT | |
| | GTGAAGGTTGTGGATGAGTTGGTGAAGGTGATGGGAAGGCATAAGCCTGAGAACATCGTGATCGAAA | |
| | TGGCTAGAGAGAACCAGACCACTCAGAAGGGACAGAAGAACTCTAGGGAAAGGATGAAGAGGATCGA | |
| | GGAAGGTATCAAAGAGCTTGGATCTCAGATCCTCAAAGAGCACCCTGTTGAGAACACTCAGCTCCAG | |
| | AATGAGAAGCTCTACCTCTACTACCTCCAGAACGGAAGGGATATGTATGTGGATCAAGAGTTGGATA | |
| | TCAACAGGCTCTCTGATTACGATGTTGATCATATCGTGCCACAGTCATTCTTGAAGGATGATTCTAT | |
| | CGATAACAAGGTGCTCACCAGGTCTGATAAGAACAGGGGTAAGAGTGATAACGTGCCAAGTGAAGAG | |
| | GTTGTGAAGAAAATGAAGAACTATTGGAGGCAGCTCCTCAACGCTAAGCTCATCACTCAGAGAAAGT | |
| | TCGATAACTTGACTAAGGCTGAGAGGGGAGGACTCTCTGAATTGGATAAGGCAGGATTCATCAAGAG | |
| | GCAGCTTGTGGAAACCAGGCAGATCACTAAGCACGTTGCACAGATCCTCGATTCTAGGATGAACACC | |
| | AAGTACGATGAGAACGATAAGTTGATCAGGGAAGTGAAGGTTATCACCCTCAAGTCAAAGCTCGTGT | |
| | CTGATTTCAGAAAGGATTTCCAATTCTACAAGGTGAGGGAAATCAACAACTACCACCACGCTCACGA | |
| | TGCTTACCTTAACGCTGTTGTTGGAACCGCTCTCATCAAGAAGTATCCTAAGCTCGAGTCAGAGTTC | |
| | GTGTACGGTGATTACAAGGTGTACGATGTGAGGAAGATGATCGCTAAGTCTGAGCAAGAGATCGGAA | |
| | AGGCTACCGCTAAGTATTTCTTCTACTCTAACATCATGAATTTCTTCAAGACCGAGATTACCCTCGC | |
| | TAACGGTGAGATCAGAAAGAGGCCACTCATCGAGACAAACGGTGAAACAGGTGAGATCGTGTGGGAT | |
| | AAGGGAAGGGATTTCGCTACCGTTAGAAAGGTGCTCTCTATGCCACAGGTGAACATCGTTAAGAAAA | |
| | CCGAGGTGCAGACCGGTGGATTCTCTAAAGAGTCTATCCTCCCTAAGAGGAACTCTGATAAGCTCAT | |
| | TGCTAGGAAGAAGGATTGGGACCCTAAGAAATACGGTGGTTTCGATTCTCCTACCGTGGCTTACTCT | |
| | GTTCTCGTTGTGGCTAAGGTTGAGAAGGGAAAGAGTAAGAAGCTCAAGTCTGTTAAGGAACTTCTCG | |
| | GAATCACTATCATGGAAAGGTCATCTTTCGAGAAGAACCCAATCGATTTCCTCGAGGCTAAGGGATA | |
| | CAAAGAGGTTAAGAAGGATCTCATCATCAAGCTCCCAAAGTACTCACTCTTCGAACTCGAGAACGGT | |
| | AGAAAGAGGATGCTCGCTTCTGCTGGTGAGCTTCAAAAGGGAAACGAGCTTGCTCTCCCATCTAAGT | |
| | ACGTTAACTTTCTTTACCTCGCTTCTCACTACGAGAAGTTGAAGGGATCTCCAGAAGATAACGAGCA | |
| | GAAGCAACTTTTCGTTGAGCAGCACAAGCACTACTTGGATGAGATCATCGAGCAGATCTCTGAGTTC | |
| | TCTAAAAGGGTGATCCTCGCTGATGCAAACCTCGATAAGGTGTTGTCTGCTTACAACAAGCACAGAG | |
| | ATAAGCCTATCAGGGAACAGGCAGAGAACATCATCCATCTCTTCACCCTTACCAACCTCGGTGCTCC | |
| | TGCTGCTTTCAAGTACTTCGATACAACCATCGATAGGAAGAGATACACCTCTACCAAAGAAGTGCTC | |
| | GATGCTACCCTCATCCATCAGTCTATCACTGGACTCTACGAGACTAGGATCGATCTCTCACAGCTCG | |
| | GTGGTGATTCAAGGGCTGATCCTAAGAAGAAGAGGAAGGTTTGACGTCGACGATATGAAGATGAAGA | |
| | TGAAATATTTGGTGTGTCAAATAAAAAGCTTGTGTGCTTAAGTTTGTGTTTTTTTCTTGGCTTGTTG | |
| | TGTTATGAATTTGTGGCTTTTTCTAATATTAAATGAATGTAAGATCACATTATAATGAATAAACAAA | |
| | TGTTTCTATAATCCATTGTGAATGTTTTGTTGGATCTCTTCTGCAGCATATAACTACTGTATGTGCT | |
| | ATGGTATGGACTATGGAATATGATTAAAGATAAGCCAGAGCTCTGGTGACGGACGGCGCGCTGGCAG | |
| | ACATACTGTCCCACAAATGAAGATGGAATCTGTAAAAGAAAACGCGTGAAATAATGCGTCTGACAAA | |
| | GGTTAGGTCGGCTGCCTTTAATCAATACCAAAGTGGTCCCTACCACGATGGAAAAACTGTGCAGTCG | |
| | GTTTGGCTTTTTCTGACGAACAAATAAGATTCGTGGCCGACAGGTGGGGGTCCACCATGTGAAGGCA | |
| | TCTTCAGACTCCAATAATGGAGCAATGACGTAAGGGCTTACGAAATAAGTAAGGGTAGTTTGGGAAA | |
| | TGTCCACTCACCCGTCAGTCTATAAATACTTAGCCCCTCCCTCATTGTTAAGGGAGCAAAATCTCAG | |
| | AGAGATAGTCCTAGAGAGAGAAAGAGAGCAAGTAGCCTAGAAGTAGTCAAGGCGGCGAAGTATTCAG | |
| | GCACGTGGCCAGGAAGAAGAAAAGCCAAGACGACGAAAACAGGTAAGAGCTAAGCTTCCTGCAGGTT | |
| | CACTGCCGTATAGGCAGCATTAACATTACCATTAACGGTTTTAGAGCTAGAAATAGCAAGTTAAAAT | |
| | AAGGCTAGTCCGTTATCAACTTGAAAAAGTGGCACCGAGTCGGTGCGTTCACTGCCGTATAGGCAGA | |
| | GGGACACCAATGTCCTGCTGTTTTAGAGCTAGAAATAGCAAGTTAAAATAAGGCTAGTCCGTTATCA | |
| | ACTTGAAAAAGTGGCACCGAGTCGGTGCGTTCACTGCCGTATAGGCAGGTCGATCGACAAGCTCGAG | |
| | TTTCTCCATAATAATGTGTGAGTAGTTCCCAGATAAGGGAATTAGGGTTCCTATAGGGTTTCGCTCA | |
| | TGTGTTGAGCATATAAGAAACCCTTAGTATGTATTTGTATTTGTAAAATACTTCTATCAATAAAATT | |
| | TCTAATTCCTAAAACCAAAATCCAGTACTAAAATCCAGATCCCCCGAATTAGAGCTCTACCGGCGAG | |
| | CTTTGGGTACGTCACGTGGCTCGAGCGCGTAGTCCTCGGTAGGCAAGCTTATTTAATTCATACAGAA | |
| | GCAATCTTTGTTTCAGATGTTCACTACAAAACTCATCCTCTTCTTCAATATTTTTGGTTTCGGAATG | |
| | ATCGCTATCTTAACTCTTTTCCTTACACATGGCCGCAAACGCGTTGATGTTCTTGGATGGATTTGCA | |
| | TGATCTTTGCTTTATGCGTGTTTGTTGCCCCCATGGGTATCATGGTGAGAATGCGAGTCGCAAATTT | |
| | CAACACTTGCTTCTTTCTGTCTCTGACAGTTTTTTTTTTTTCCCCTATAATTATATTGATTGATTTT | |
| | TGTTTTCTCTCTTCTTTACTCTATTTTCCAGAGAAAAGTGATAAAAACGAAGAGTGTCGAGTTCATG | |
| | CCATTTTCTTTATCATTCTTCCTCACCTTGACTGCGGTGATGTGGTTCTTCTATGGTTTTCTAAAGA | |
| | AAGACCTTTATGTTGCCGTAAGTTAACTATCACGCATGCATCATTATCACGTACATCTTTCTTTACA | |
| | TTCCACCAACTTTATCTTTCCCATTAATCATCAACCCAGCAACTATTTCTTATTCCCTTTTGATTAA | |
| | CTTCCACTTACAATTTCCTTTTTCTTGTCATGAACAGATTCCAAACACATTGGGCTTTCTTTTTGGG | |
| | ATTGTCCAGATGGTGCTTTATTTAATCTACAGAAACCCCAAGAAATTACCTGTAGAGGATCCTAAAC | |
| | TTCGCGAATTGTCCGAGCACATCGTCGACGTTGCAAAGCTGAGTGCAACCCTCTGTTCCGAGATAAC | |
| | CACAGTAGTGGTTCCACAGCCCATAGACAATGGAAATGATGTTGAAGGTCAAAAAATTAAGGAAGAA | |
| | AACGAGCAGGACATTGGTGTCCCTGCAGACAAAGTTAAGACTAATCTTTTTCTCTTTCTCATCTTTT | |
| | CACTTCTCCAATCATTATCCTCGGCCGAATTCAGTAAAGGAGAAGAACTTTTCACTGGAGTTGTCCC | |
| | AATTCTTGTTGAATTAGATGGTGATGTTAATGGGCACAAATTTTCTGTCAGTGGAGAGGGTGAAGGT | |
| | GATGCAACATACGGAAAACTTACCCTTAAATTTATTTGCACTACTGGAAAACTACCTGTTCCATGGC | |
| | CAACACTTGTCACTACTTTCACTTATGGTGTTCAATGCTTTTCAAGATACCCAGATCATATGAAGCG | |
| | GCACGACTTCTTCAAGAGCGCCATGCCTGAGGGATACGTGCAGGAGAGGACCATCTCTTTCAAGGAC | |
| | GACGGGAACTACAAGACACGTGCTGAAGTCAAGTTTGAGGGAGACACCCTCGTCAACAGGATCGAGC | |
| | TTAAGGGAATCGATTTCAAGGAGGACGGAAACATCCTCGGCCACAAGTTGGAATACAACTACAACTC | |
| | CCACAACGTATACATCACGGCAGACAAACAAAAGAATGGAATCAAAGCTAACTTCAAAATTAGACAC | |
| | AACATTGAAGATGGAAGCGTTCAACTAGCAGACCATTATCAACAAAATACTCCAATTGGCGATGGCC | |
| | CTGTCCTTTTACCAGACAACCATTACCTGTCCACACAATCTGCCCTTTCGAAAGATCCCAACGAAAA | |
| | GAGAGACCACATGGTCCTTCTTGAGTTTGTAACAGCTGCTGGGATTACACATGGCATGGATGAACTA | |
| | TACAAACATGATGAGCTTTGATAACATTAACATTACCATTAACGTGATCTTGGTTATGTTTTTTCTT | |
| | TTTAATTTTGCATGTAATCGTTCAAAGTGGTGGTGCCATGTCTACTTGTAAGGCTGCAATGCAGCCA | |
| | TGTTGTCTATTATGTCAAATCTAGTTCCATTTAATGTCAATCTTTATTCTCAACCTAAAAGAAGAAT | |
| | ATCAATCTTTATGTAATACGTTTTTTCGAGTAAATAAAATGTCCAGTGAATTTACAGTTAATGTTAA | |
| | ATCAGCATTATATTTTAGGAAAATAGTATTCAACTTATAGTTTAATGGTTGAAATTAAATATTAATT | |
| | TTTATTTTATGATGTAATAATTTTAAATTTAAATTATAGCTCCTGGCAAGAGTTATTAATAAAATAA | |
| | TACTGCCAATATTTTTTTCTAAATTTTATTTGAATTTGTTATTTATTTTATGGAAAATATTTTTAAA | |
| | AAATAATTTTCATATTTTTTTATATAAGAAGAGCTCAAAAAAATTTTAAATCCATGTTATTTTACAC | |
| | TAAAAAACAGAAGTTTAAATAGGGGAGAAATTTTTACATTCGCCAACAAAACTATATAAATTTTTGT | |
| | TTTGAATTATAAAATAATAATTATTTTTCCTAAAAAGAATTCTTCATGATTGTGCCAAATAAGTCTC | |
| | AATGCAATTTTAAAAAAAATCCAGACAAAATTTGTCTTATTTCTCACTGTGCTATTTTTCTAATAAG | |
| | CATTTTCATTGTGCAATTAAATCTATTGGACTCTAATCAATAATAAAGAAAAGGGATACCTTTAATC | |
| | TTTTATCGAAGATATCAACTAATTCTAGAGCGCGGTAATATCGCAGAACAAAAGTACCTGATATCGA | |
| | GTGTACTTCAAGTCACACCGGCG | |
| 9 | AGTGTGGCACTGGCCGTCGTTTTACAACGTCGTGACTGGGAAAACCCTGGCGTTACCCAACTTAATC | Comprises a |
| | GCCTTGCAGCACATCCCCCTTTCGCCAGCTGGCGTAATAGCGAAGAGGCCCGCACCGATCGCCCTTC | construct for |
| | CCAACAGTTGCGCAGCCTGAATGGCGAATGCTAGAGCAGCTTGAGCTTGGATCAGATTGTCGTTTCC | expressing |
| | CGCCTTCAGTTTAAACTATCAGTGTTTGACAGGATATATTGGCGGGTAAACCTAAGAGAAAAGAGCG | the AtCas9 |
| | TTTATTAGAATAATCGGATATTTAAAAGGGCGTGAAAAGGTTTATCCGTTCGTCCATTTGTATGTGC | protein in |
| | ATGCCAACCACAGGGTTCCCCTCGGGATCAAAGTACTTTGATCCAACCCCTCCGCTGCTATAGTGCA | combination |
| | GTCGGCTTCTGACGTTCAGTGCAGCCGTCTTCTGAAAACGACATGTCGCACAAGTCCTAAGTTACGC | with the cys4 |
| | GACAGGCTGCCGCCCTGCCCTTTTCCTGGCGTTTTCTTGTCGCGTGTTTTAGTCGCATAAAGTAGAA | CRISPR RNA |
| | TACTTGCGACTAGAACCGGAGACATTACGCCATGAACAAGAGCGCCGCCGCTGGCCTGCTGGGCTAT | processing |
| | GCCCGCGTCAGCACCGACGACCAGGACTTGACCAACCAACGGGCCGAACTGCACGCGGCCGGCTGCA | protein from |
| | CCAAGCTGTTTTCCGAGAAGATCACCGGCACCAGGCGCGACCGCCCGGAGCTGGCCAGGATGCTTGA | Pseudomonas |
| | CCACCTACGCCCTGGCGACGTTGTGACAGTGACCAGGCTAGACCGCCTGGCCCGCAGCACCCGCGAC | aeruginosa |
| | CTACTGGACATTGCCGAGCGCATCCAGGAGGCCGGCGCGGGCCTGCGTAGCCTGGCAGAGCCGTGGG | under the |
| | CCGACACCACCACGCCGGCCGGCCGCATGGTGTTGACCGTGTTCGCCGGCATTGCCGAGTTCGAGCG | control of |
| | TTCCCTAATCATCGACCGCACCCGGAGCGGGCGCGAGGCCGCCAAGGCCCGAGGCGTGAAGTTTGGC | the 35S |
| | CCCCGCCCTACCCTCACCCCGGCACAGATCGCGCACGCCCGCGAGCTGATCGACCAGGAAGGCCGCA | promoter |
| | CCGTGAAAGAGGCGGCTGCACTGCTTGGCGTGCATCGCTCGACCCTGTACCGCGCACTTGAGCGCAG | And |
| | CGAGGAAGTGACGCCCACCGAGGCCAGGCGGCGCGGTGCCTTCCGTGAGGACGCATTGACCGAGGCC | construct for |
| | GACGCCCTGGCGGCCGCCGAGAATGAACGCCAAGAGGAACAAGCATGAAACCGCACCAGGACGGCCA | the TAL20 |
| | GGACGAACCGTTTTTCATTACCGAAGAGATCGAGGCGGAGATGATCGCGGCCGGGTACGTGTTCGAG | transcription |
| | CCGCCCGCGCACGGCTCAACCGTGCGGCTGCATGAAATCCTGGCCGGTTTGTCTGATGCCAAGCTGG | activator |
| | CGGCCTGGCCGGCCAGCTTGGCCGCTGAAGAAACCGAGCGCCGCCGTCTAAAAAGGTGATGTGTATT | under the |
| | TGAGTAAAACAGCTTGCGTCATGCGGTCGCTGCGTATATGATGCGATGAGTAAATAAACAAATACGC | control of |
| | AAGGGGAACGCATGAAGGTTATCGCTGTACTTAACCAGAAAGGCGGGTCAGGCAAGACGACCATCGC | the 35S |
| | AACCCATCTAGCCCGCGCCCTGCAACTCGCCGGGGCCGATGTTCTGTTAGTCGATTCCGATCCCCAG | promoter |
| | GGCAGTGCCCGCGATTGGGCGGCCGTGCGGGAAGATCAACCGCTAACCGTTGTCGGCATCGACCGCC | |
| | CGACGATTGACCGCGACGTGAAGGCCATCGGCCGGCGCGACTTCGTAGTGATCGACGGAGCGCCCCA | |
| | GGCGGCGGACTTGGCTGTGTCCGCGATCAAGGCAGCCGACTTCGTGCTGATTCCGGTGCAGCCAAGC | |
| | CCTTACGACATATGGGCCACCGCCGACCTGGTGGAGCTGGTTAAGCAGCGCATTGAGGTCACGGATG | |
| | GAAGGCTACAAGCGGCCTTTGTCGTGTCGCGGGCGATCAAAGGCACGCGCATCGGCGGTGAGGTTGC | |
| | CGAGGCGCTGGCCGGGTACGAGCTGCCCATTCTTGAGTCCCGTATCACGCAGCGCGTGAGCTACCCA | |
| | GGCACTGCCGCCGCCGGCACAACCGTTCTTGAATCAGAACCCGAGGGCGACGCTGCCCGCGAGGTCC | |
| | AGGCGCTGGCCGCTGAAATTAAATCAAAACTCATTTGAGTTAATGAGGTAAAGAGAAAATGAGCAAA | |
| | AGCACAAACACGCTAAGTGCCGGCCGTCCGAGCGCACGCAGCAGCAAGGCTGCAACGTTGGCCAGCC | |
| | TGGCAGACACGCCAGCCATGAAGCGGGTCAACTTTCAGTTGCCGGCGGAGGATCACACCAAGCTGAA | |
| | GATGTACGCGGTACGCCAAGGCAAGACCATTACCGAGCTGCTATCTGAATACATCGCGCAGCTACCA | |
| | GAGTAAATGAGCAAATGAATAAATGAGTAGATGAATTTTAGCGGCTAAAGGAGGCGGCATGGAAAAT | |
| | CAAGAACAACCAGGCACCGACGCCGTGGAATGCCCCATGTGTGGAGGAACGGGCGGTTGGCCAGGCG | |
| | TAAGCGGCTGGGTTGTCTGCCGGCCCTGCAATGGCACTGGAACCCCCAAGCCCGAGGAATCGGCGTG | |
| | ACGGTCGCAAACCATCCGGCCCGGTACAAATCGGCGCGGCGCTGGGTGATGACCTGGTGGAGAAGTT | |
| | GAAGGCCGCGCAGGCCGCCCAGCGGCAACGCATCGAGGCAGAAGCACGCCCCGGTGAATCGTGGCAA | |
| | GCGGCCGCTGATCGAATCCGCAAAGAATCCCGGCAACCGCCGGCAGCCGGTGCGCCGTCGATTAGGA | |
| | AGCCGCCCAAGGGCGACGAGCAACCAGATTTTTTCGTTCCGATGCTCTATGACGTGGGCACCCGCGA | |
| | TAGTCGCAGCATCATGGACGTGGCCGTTTTCCGTCTGTCGAAGCGTGACCGACGAGCTGGCGAGGTG | |
| | ATCCGCTACGAGCTTCCAGACGGGCACGTAGAGGTTTCCGCAGGGCCGGCCGGCATGGCCAGTGTGT | |
| | GGGATTACGACCTGGTACTGATGGCGGTTTCCCATCTAACCGAATCCATGAACCGATACCGGGAAGG | |
| | GAAGGGAGACAAGCCCGGCCGCGTGTTCCGTCCACACGTTGCGGACGTACTCAAGTTCTGCCGGCGA | |
| | GCCGATGGCGGAAAGCAGAAAGACGACCTGGTAGAAACCTGCATTCGGTTAAACACCACGCACGTTG | |
| | CCATGCAGCGTACGAAGAAGGCCAAGAACGGCCGCCTGGTGACGGTATCCGAGGGTGAAGCCTTGAT | |
| | TAGCCGCTACAAGATCGTAAAGAGCGAAACCGGGCGGCCGGAGTACATCGAGATCGAGCTAGCTGAT | |
| | TGGATGTACCGCGAGATCACAGAAGGCAAGAACCCGGACGTGCTGACGGTTCACCCCGATTACTTTT | |
| | TGATCGATCCCGGCATCGGCCGTTTTCTCTACCGCCTGGCACGCCGCGCCGCAGGCAAGGCAGAAGC | |
| | CAGATGGTTGTTCAAGACGATCTACGAACGCAGTGGCAGCGCCGGAGAGTTCAAGAAGTTCTGTTTC | |
| | ACCGTGCGCAAGCTGATCGGGTCAAATGACCTGCCGGAGTACGATTTGAAGGAGGAGGCGGGGCAGG | |
| | CTGGCCCGATCCTAGTCATGCGCTACCGCAACCTGATCGAGGGCGAAGCATCCGCCGGTTCCTAATG | |
| | TACGGAGCAGATGCTAGGGCAAATTGCCCTAGCAGGGGAAAAAGGTCGAAAAGGCCTCTTTCCTGTG | |
| | GATAGCACGTACATTGGGAACCCAAAGCCGTACATTGGGAACCGGAACCCGTACATTGGGAACCCAA | |
| | AGCCGTACATTGGGAACCGGTCACACATGTAAGTGACTGATATAAAAGAGAAAAAAGGCGATTTTTC | |
| | CGCCTAAAACTCTTTAAAACTTATTAAAACTCTTAAAACCCGCCTGGCCTGTGCATAACTGTCTGGC | |
| | CAGCGCACAGCCCAAGAGCTGCAAAAAGCGCCTACCCTTCGGTCGCTGCGCTCCCTACGCCCCGCCG | |
| | CTTCGCGTCGGCCTATCGCGGCCGCTGGCCGCTCAAAAATGGCTGGCCTACGGCCAGGCAATCTACC | |
| | AGGGCGCGGACAAGCCGCGCCGTCGCCACTCGACCGCCGGCGCCCACATCAAGGCACCCTGCCTCGC | |
| | GCGTTTCGGTGATGACGGTGAAAACCTCTGACACATGCAGCTCCCGGAAACGGTCACAGCTTGTCTG | |
| | TAAGCGGATGCCGGGAGCAGACAAGCCCGTCAGGGCGCGTCAGCGGGTGTTGGCGGGTGTCGGGGCG | |
| | CAGCCATGACCCAGTCACGTAGCGATAGCGGAGTGTATACTGGCTTAACTATGCGGCATCAGAGCAG | |
| | ATTGTACTGAGAGTGCACCATATGCGGTGTGAAATACCGCACAGATGCGTAAGGAGAAAATACCGCA | |
| | TCAGGCCCTCTTCCGCTTCCTCGCTCACTGACTCGCTGCGCTCGGTCGTTCGGCTGCGGCGAGCGGT | |
| | ATCAGCTCACTCAAAGGCGGTAATACGGTTATCCACAGAATCAGGGGATAACGCAGGAAAGAACATG | |
| | TGAGCAAAAGGCCAGCAAAAGGCCAGGAACCGTAAAAAGGCCGCGTTGCTGGCGTTTTTCCATAGGC | |
| | TCCGCCCCCCTGACGAGCATCACAAAAATCGACGCTCAAGTCAGAGGTGGCGAAACCCGACAGGACT | |
| | ATAAAGATACCAGGCGTTTCCCCCTGGAAGCTCCCTCGTGCGCTCTCCTGTTCCGACCCTGCCGCTT | |
| | ACCGGATACCTGTCCGCCTTTCTCCCTTCGGGAAGCGTGGCGCTTTCTCATAGCTCACGCTGTAGGT | |
| | ATCTCAGTTCGGTGTAGGTCGTTCGCTCCAAGCTGGGCTGTGTGCACGAACCCCCCGTTCAGCCCGA | |
| | CCGCTGCGCCTTATCCGGTAACTATCGTCTTGAGTCCAACCCGGTAAGACACGACTTATCGCCACTG | |
| | GCAGCAGCCACTGGTAACAGGATTAGCAGAGCGAGGTATGTAGGCGGTGCTACAGAGTTCTTGAAGT | |
| | GGTGGCCTAACTACGGCTACACTAGAAGGACAGTATTTGGTATCTGCGCTCTGCTGAAGCCAGTTAC | |
| | CTTCGGAAAAAGAGTTGGTAGCTCTTGATCCGGCAAACAAACCACCGCTGGTAGCGGTGGTTTTTTT | |
| | GTTTGCAAGCAGCAGATTACGCGCAGAAAAAAAGGATCTCAAGAAGATCCTTTGATCTTTTCTACGG | |
| | GGTCTGACGCTCAGTGGAACGAAAACTCACGTTAAGGGATTTTGGTCATGCATTCTAGGTACTAAAA | |
| | CAATTCATCCAGTAAAATATAATATTTTATTTTCTCCCAATCAGGCTTGATCCCCAGTAAGTCAAAA | |
| | AATAGCTCGACATACTGTTCTTCCCCGATATCCTCCCTGATCGACCGGACGCAGAAGGCAATGTCAT | |
| | ACCACTTGTCCGCCCTGCCGCTTCTCCCAAGATCAATAAAGCCACTTACTTTGCCATCTTTCACAAA | |
| | GATGTTGCTGTCTCCCAGGTCGCCGTGGGAAAAGACAAGTTCCTCTTCGGGCTTTTCCGTCTTTAAA | |
| | AAATCATACAGCTCGCGCGGATCTTTAAATGGAGTGTCTTCTTCCCAGTTTTCGCAATCCACATCGG | |
| | CCAGATCGTTATTCAGTAAGTAATCCAATTCGGCTAAGCGGCTGTCTAAGCTATTCGTATAGGGACA | |
| | ATCCGATATGTCGATGGAGTGAAAGAGCCTGATGCACTCCGCATACAGCTCGATAATCTTTTCAGGG | |
| | CTTTGTTCATCTTCATACTCTTCCGAGCAAAGGACGCCATCGGCCTCACTCATGAGCAGATTGCTCC | |
| | AGCCATCATGCCGTTCAAAGTGCAGGACCTTTGGAACAGGCAGCTTTCCTTCCAGCCATAGCATCAT | |
| | GTCCTTTTCCCGTTCCACATCATAGGTGGTCCCTTTATACCGGCTGTCCGTCATTTTTAAATATAGG | |
| | TTTTCATTTTCTCCCACCAGCTTATATACCTTAGCAGGAGACATTCCTTCCGTATCTTTTACGCAGC | |
| | GGTATTTTTCGATCAGTTTTTTCAATTCCGGTGATATTCTCATTTTAGCCATTTATTATTTCCTTCC | |
| | TCTTTTCTACAGTATTTAAAGATACCCCAAGAAGCTAATTATAACAAGACGAACTCCAATTCACTGT | |
| | TCCTTGCATTCTAAAACCTTAAATACCAGAAAACAGCTTTTTCAAAGTTGTTTTCAAAGTTGGCGTA | |
| | TAACATAGTATCGACGGAGCCGATTTTGAAACCGCGGTGATCACAGGCAGCAACGCTCTGTCATCGT | |
| | TACAATCAACATGCTACCCTCCGCGAGATCATCCGTGTTTCAAACCCGGCAGCTTAGTTGCCGTTCT | |
| | TCCGAATAGCATCGGTAACATGAGCAAAGTCTGCCGCCTTACAACGGCTCTCCCGCTGACGCCGTCC | |
| | CGGACTGATGGGCTGCCTGTATCGAGTGGTGATTTTGTGCCGAGCTGCCGGTCGGGGAGCTGTTGGC | |
| | TGGCTGGTGGCAGGATATATTGTGGTGTAAACAAATTGACGCTTAGACAACTTAATAACACATTGCG | |
| | GACGTTTTTAATGTACTGAATTAACGCCGAATTAATTCGGGGGATCTGGATTTTAGTACTGGATTTT | |
| | GGTTTTAGGAATTAGAAATTTTATTGATAGAAGTATTTTACAAATACAAATACATACTAAGGGTTTC | |
| | TTATATGCTCAACACATGAGCGAAACCCTATAGGAACCCTAATTCCCTTATCTGGGAACTACTCACA | |
| | CATTATTATGGAGAAACTCGAGCTTGTCGATCGACTCTAGCTAGAGGATCGATCCGAACCCCAGAGT | |
| | CCCGCTCAGAAGAACTCGTCAAGAAGGCGATAGAAGGCGATGCGCTGCGAATCGGGAGCGGCGATAC | |
| | CGTAAAGCACGAGGAAGCGGTCAGCCCATTCGCCGCCAAGTTCTTCAGCAATATCACGGGTAGCCAA | |
| | CGCTATGTCCTGATAGCGGTCCGCCACACCCAGCCGGCCACAGTCGATGAATCCAGAAAAGCGGCCA | |
| | TTTTCCACCATGATATTCGGCAAGCAGGCATCGCCATGTGTCACGACGAGATCCTCGCCGTCGGGCA | |
| | TGCGCGCCTTGAGCCTGGCGAACAGTTCGGCTGGCGCGAGCCCCTGATGTTCTTCGTCCAGATCATC | |
| | CTGATCGACAAGACCGGCTTCCATCCGAGTACGTGCTCGCTCGATGCGATGTTTCGCTTGGTGGTCG | |
| | AATGGGCAGGTAGCCGGATCAAGCGTATGCAGCCGCCGCATTGCATCAGCCATGATGGATACTTTCT | |
| | CGGCAGGAGCAAGGTGAGATGACAGGAGATCCTGCCCCGGCACTTCGCCCAATAGCAGCCAGTCCCT | |
| | TCCCGCTTCAGTGACAACGTCGAGCACAGCTGCGCAAGGAACGCCCGTCGTGGCCAGCCACGATAGC | |
| | CGCGCTGCCTCGTCCTGGAGTTCATTCAGGGCACCGGACAGGTCGGTCTTGACAAAAAGAACCGGGC | |
| | GCCCCTGCGCTGACAGCCGAAACACGGCGGCATCAGAGCAGCCGATTGTCTGTTGTGCCCAGTCATA | |
| | GCCGAATAGCCTCTCCACCCAAGCGGCCGGAGAACCTGCGTGCAATCCATCTTGTTCAATCCCCATG | |
| | GTCGATCGACAGATCTGCGAAAGCTCGAGAGAGATAGATTTGTAGAGAGAGACTGGTGATTTCAGCG | |
| | TGTCCTCTCCAAATGAAATGAACTTCCTTATATAGAGGAAGGGTCTTGCGAAGGATAGTGGGATTGT | |
| | GCGTCATCCCTTACGTCAGTGGAGATATCACATCAATCCACTTGCTTTGAAGACGTGGTTGGAACGT | |
| | CTTCTTTTTCCACGATGCTCCTCGTGGGTGGGGGTCCATCTTTGGGACCACTGTCGGCAGAGGCATC | |
| | TTGAACGATAGCCTTTCCTTTATCGCAATGATGGCATTTGTAGGTGCCACCTTCCTTTTCTACTGTC | |
| | CTTTTGATGAAGTGACAGATAGCTGGGCAATGGAATCCGAGGAGGTTTCCCGATATTACCCTTTGTT | |
| | GAAAAGTCTCAATAGCCCTTTGGTCTTCTGAGACTGTATCTTTGATATTCTTGGAGTAGACGAGAGT | |
| | GTCGTGCTCCACCATGTTCACATCAATCCACTTGCTTTGAAGACGTGGTTGGAACGTCTTCTTTTTC | |
| | CACGATGCTCCTCGTGGGTGGGGGTCCATCTTTGGGACCACTGTCGGCAGAGGCATCTTGAACGATA | |
| | GCCTTTCCTTTATCGCAATGATGGCATTTGTAGGTGCCACCTTCCTTTTCTACTGTCCTTTTGATGA | |
| | AGTGACAGATAGCTGGGCAATGGAATCCGAGGAGGTTTCCCGATATTACCCTTTGTTGAAAAGTCTC | |
| | AATAGCCCTTTGGTCTTCTGAGACTGTATCTTTGATATTCTTGGAGTAGACGAGAGTGTCGTGCTCC | |
| | ACCATGTTGGCAAGCTGCTCTAGCCAATACGCAAACCGCCTCTCCCCGCGCGTTGGCCGATTCATTA | |
| | ATGCAGCTGGCACGACAGGTTTCCCGACTGGAAAGCGGGCAGTGAGCGCAACGCAATTAATGTGAGT | |
| | TAGCTCACTCATTAGGCACCCCAGGCTTTACACTTTATGCTTCCGGCTCGTATGTTGTGTGGAATTG | |
| | TGAGCGGATAACAATTTCACACAGGAAACAGCTATGACATGATTACGAATTCGAGCTCGGTACCCAG | |
| | ATTTGCCTTTTCAATTTCAGAAAGAATGCTAACCCACAGATGGTTAGAGAGGCTTACGCAGCAGGTA | |
| | TCATCAAGACGATCTACCCGAGCAATAATCTCCAGGAAATCAAATACCTTCCCAAGAAGGTTAAAGA | |
| | TGCAGTCAAAAGATTCAGGACTAACTGCATCAAGAACACAGAGAAAGATATATTTCTCAAGATCAGA | |
| | AGTACTATTCCAGTATGGACGATTCAAGGCTTGCTTCACAAACCAAGGCAAGTAATAGAGATTGGAG | |
| | TCTCTAAAAAGGTAGTTCCCACTGAATCAAAGGCCATGGAGTCAAAGATTCAAATAGAGGACCTAAC | |
| | AGAACTCGCCGTAAAGACTGGCGAACAGTTCATACAGAGTCTCTTACGACTCAATGACAAGAAGAAA | |
| | ATCTTCGTCAACATGGTGGAGCACGACACACTTGTCTACTCCAAAAATATCAAAGATACAGTCTCAG | |
| | AAGACCAAAGGGCAATTGAGACTTTTCAACAAAGGGTAATATCCGGAAACCTCCTCGGATTCCATTG | |
| | CCCAGCTATCTGTCACTTTATTGTGAAGATAGTGGAAAAGGAAGGTGGCTCCTACAAATGCCATCAT | |
| | TGCGATAAAGGAAAGGCCATCGTTGAAGATGCCTCTGCCGACAGTGGTCCCAAAGATGGACCCCCAC | |
| | CCACGAGGAGCATCGTGGAAAAAGAAGACGTTCCAACCACGTCTTCAAAGCAAGTGGATTGATGTGA | |
| | TATCTCCACTGACGTAAGGGATGACGCACAATCCCACTATCCTTCGCAAGACCCTTCCTCTATATAA | |
| | GGAAGTTCATTTCATTTGGAGAGAACACGGGGGACTATGGATCCCATTCGTCCGCGCACGCCAAGTC | |
| | CTGCCCACGAACTTCTGGCCGGACCCCAGCCGGATAGGGTTCAGCCGCAGCCGACTGCAGATCGTGG | |
| | GGGGGCTCCGCCTGCCGGCAGCCCCCTGGATGGCTTGCCCGCTCGACGGACGATGTCCCGAACCCGT | |
| | CTCCCGTCTCCCCCTGCACCCTTGCCTGCGTTCTCAGCGGGCAGTTTCAGCGATCTGCTCCGTCAGT | |
| | TCGATCCGTCGCTTCTTGATACATCGCTTTTTAATTCGATGTCTGCCTTCGGCGCTCCTCATACAGA | |
| | GGCTGCCTCAGGAGAGGGGGATGAGGTGCAATCGGGTCTGCGTGCAGCCGATGACCCGCAAGCCACC | |
| | GTGCAGGTCGCTGTGACGGCCGCGCGACCGCCGCGCGCCAAGCCGGCGCCGCGACGGCGTGCTGCGC | |
| | ACACCTCTGACGCTTCGCCGGCCGGGCAGGTCGATCTATGCACGCTCGGCTACAGCCAGCAGCAGCA | |
| | AGAGAAGATCAAACTGAAGGCTCGTTCGACAGTAGCACAGCACCACGAGGCACTGATCGGCCATGGG | |
| | TTTACACGTGCGCACATCGTTGCGCTCAGCCAACACCCGGCAGCCTTAGGGACCGTCGCTGTCAAGT | |
| | ACCAGGCCATGATCGCGGCGTTGCCGGAGGCGACACACGAAGACATCGTTGGCGGCGGCAAACAGTG | |
| | GTCCGGCGCACGCGCCCTGGAAGCATTGCTCACGGTGTCGGGAGAGTTGAGAGGTCCACCGTTACAG | |
| | TTGGACACAGGTCAACTTCTCAAGATTGCAAAACGTGGCGGCGTGACCGCGGTGGAGGCAGTGCATG | |
| | CATGGCGCAATGCACTGACGGGCGCTCCCCTGAACCTGACCCCGGACCAGGTGGTGGCCATCGCCAG | |
| | CAATATTGGCGGCAAGCAGGCGCTGGAGACGGTGCAGCGGCTGTTGCCGGTGCTGTGCGAGCAACAT | |
| | GGCCTGACCCTGGACCAGGTGGTGGCCATCGCCAGCAATGGCGGCGGCAAGCAGGCGCTGGAGACGG | |
| | TGCAGCGGCTGTTGCCGGTGCTGTGCGAGCAACATGGTCTGACCCCGGACCAGGTGGTGGCTATCGC | |
| | CAGCAATATTGGCGGCAAGCAGGCGCTGGAGACGGTGCAGCGGCTGTTGCCGGTGCTGTGCGAGCAA | |
| | CATGGTCTGACCCCGGACCAGGTGGTGGCCATCGCCAGCAATAACGGCGGCAAGCAGGCGCTGGAGA | |
| | CGGTGCAGCGGCTGTTGCCGGTGCTGTGCGAGCAACATGGCCTGACCCCGGACCAGGTGGTGGCTAT | |
| | CGCCAGCAATATTGGCGGCAAGCAGGCGCTGGAGACGGTGCAGCGGCTGTTGCCGGTGCTGCGCCAG | |
| | GCCCATGGCCTGACCCCGGCGCAGGTGGTGGCCATCGCCAGCCACGATGGCGGCAAGCAGGCGCTGG | |
| | AGACGGTGCAGCAGCTGTTGCCGGTGCTGTGCGAGCAACATGGCCTGACCCCGGCGCAGGTGGTGGC | |
| | CATCGCCAGCAATAGCGGCGGCAAGCAGGCGCTGGAGACGGTGCAGCGGCTGTTGCCGGTGCTGCGC | |
| | CAGGCCCATGGCCTGACCCCGGACCAGGTGGTGGCCATCGCCAGCAATAGCGGCGGCAAGCCGGCGC | |
| | TGGAGACGGTGCAGCGGCTGTTGCCGGTGCTGTGCGAGCAACATGGTCTGACCCCGGACCAGGTGGT | |
| | GGCCATCGCCAGCAATAACGGCGGCAAGCCGGCGCTGGAGACGGTGCAGCGGCTGTTGCCGGTGCTG | |
| | TGCGAGCAACATGGCCTGACCCGGGCGCAGGTGGTGGCCATCGCCAGCAATGGCGGCGGCAAGCAGG | |
| | CGCTGGAGACGGTGCAGCGGCTGTTGCCGGTGCTGCGCCAGGCCCATGGCCTGACCCCGGCGCAGGT | |
| | GGTGGCCATCGCCAGCCACGATGGCGGCAAGCAGGCGCTGGAGACGGTGCAGCAGCTGTTGCCGGTG | |
| | CTGTGCGAGCAACATGGCCTGACCCCGGCGCAGGTGGTGGCCATCGCCAGCAATAGCGGCGGCAAGC | |
| | AGGCGCTGGAGACGGTGCAGCGGCTGTTGCCGGTGCTGCGCCAGGCCCATGGCCTGACCCCGGACCA | |
| | GGTGGTGGCCATCGCCAGCCACGATGGCGGCAAGCAGGCGCTGGAGACGGTGCAGCGGCTGTTGCCG | |
| | GTGCTGTGCGAGCAACATGGTCTGACCCCGGACCAGGTGGTGGCCATCGCCAGCAATAACGGCGGCA | |
| | AGCAGGCGCTGGAGACGGTGCAGCGGCTGTTGCCGGTGCTGTGCGAGCAACATGGCCTGACCCCGGA | |
| | CCAGGTGGTGGCCATCGCCAGCCACGATGGCGGCAAGCAGGCGCTGGAGACGGTGCAGCGGCTGTTG | |
| | CCGGTGCTGTGCGAGCAACATGGCCTGACCCCGGACCAGGTGGTGGCCATCGCCAGCCACGATGGCG | |
| | GCAAGCAGGCGCTGGAGACGGTGCAGCGGCTGTTGCCGGTGCTGCGCCAGGCCCATGGCCTGACCCC | |
| | GGCGCAGGTGGTGGCCATCGCCAGCCACGATGGCGGCAAGCCGGCGCTGGAGACGGTGCAGCGGCTG | |
| | TTGCCGGTGCTGTGCGAGCAACATGGCCTGACCCCGGACCAGGTGGTGGCTATCGCCAGCAATATTG | |
| | GCGGCAAGCAGGCGCTGGAGACGGTGCAGCGGCTGTTGCCGGTGCTGTGCGAGCAACATGGCCTGAC | |
| | CCCGGACCAGGTGGTGGCCATCGCCAGCAATGGCGGCGGCAAGCAGGCGCTGGAGACGGTGCAGCGG | |
| | CTGTTGCCGGTGCTGTGCGAGCAACATGGTCTGACCCCGGCGCAGGTGGTGGCCATCGCCAGCAATG | |
| | GCGGCGGCAGGCCGGCGCTGGAGAGCATTTTTGCCCAGTTATCTCGCCCTGATCAGGCGTTGGCCGC | |
| | GTTGACCAACGACCACCTCGTCGCCTTGGCCTGCCTCGGCGGGCGTCCTGCGCTGGAGGCAGTGAAA | |
| | AAGGGATTGCCGCACGCGCCGACCTTGATCAAAAGAACCAATCGCCGTCTTCCCGAACGCACGTCCC | |
| | ATCGCGTTGCCGACCACGCGCAAGTGGCTCGCGTGTTGGGTTTTTTCCAGTGCCACTCCCACCCAGC | |
| | GCAAGCATTTGATGAAGCCATGACGCAGTTCGGGATGAGCAGGCACGGGTTGTTACAGCTATTTCGC | |
| | AGAGCCGGCGTCACCGAACTCGAGGCCCACAGTGGAACGCTCCCCCCAGCCTCGCAGCGTTGGCACC | |
| | GTATCCTCCAGGCATCAGGGATGAAAAGGGCCGAACCGTCCGGTGCTTCCGCTCAAACGCCGGACCA | |
| | GGCGTCTTTGCATGCATTCGCCGATGCGCTGGAGCGTGAGCTGGATGCGCCCAGCCCAATAGACCGG | |
| | GCGGGCCAGGCGCTGGCAAGCAGCAGCCGTAAACGGTCCCGATCGGAGAGTTCTGTCACCGGCTCCT | |
| | TCGCACAGCAAGCTGTCGAGGTGCGCGTTCCCGAACAGCGCGATGCGCTGCATTTCCTCCCCCTCAG | |
| | CTGGGGTGTAAAACGCCCGCGTACCAGGATCGGGGGCGGCCTCCCGGATCCTGGTACGCCCATGGAC | |
| | GCCGACCTGGCACCGTCCAGCACCGTGATGTGGGAACAAGATGCTGACCCCTTCGCAGGGGCAGCGG | |
| | ATGATTTTCCGGCATTCAACGAAGAGGAGATGGCATGGTTGATGGAGCTATTTCCTCAGTGAGGGGA | |
| | TCGGCGCGCCAGATTTGCCTTTTCAATTTCAGAAAGAATGCTAACCCACAGATGGTTAGAGAGGCTT | |
| | ACGCAGCAGGTATCATCAAGACGATCTACCCGAGCAATAATCTCCAGGAAATCAAATACTTCCCAAG | |
| | AAGGTTAAAGATGCAGTCAAAAGATTCAGGACTAACTGCATCAAGAACACAGAGAAAGATATATTTC | |
| | TCAAGATCAGAAGTACTATTCCAGTATGGACGATTCAAGGCTTGCTTCACAAACCAAGGCAAGTAAT | |
| | AGAGATTGGAGTCTCTAAAAAGGTAGTTCCCACTGAATCAAAGGCCATGGAGTCAAAGATTCAAATA | |
| | GAGGACCTAACAGAACTCGCCGTAAAGACTGGCGAACAGTTCATACAGAGTCTCTTACGACTCAATG | |
| | ACAAGAAGAAAATCTTCGTCAACATGGTGGAGCACGACACACTTGTCTACTCCAAAAATATCAAAGA | |
| | TACAGTCTCAGAAGACCAAAGGGCAATTGAGACTTTTCAACAAAGGGTAATATCCGGAAACCTCCTC | |
| | GGATTCCATTGCCCAGCTATCTGTCACTTTATTGTGAAGATAGTGGAAAAGGAAGGTGGCTCCTACA | |
| | AATGCCATCATTGCGATAAAGGAAAGGCCATCGTTGAAGATGCCTCTGCCGACAGTGGTCCCAAAGA | |
| | TGGACCCCCACCCACGAGGAGCATCGTGGAAAAAGAAGACGTTCCAACCACGTCTTCAAAGCAAGTG | |
| | GATTGATGTGATATCTCCACTGACGTAAGGGATGACGCACAATCCCACTATCCTTCGCAAGACCCTT | |
| | CCTCTATATAAGGAAGTTCATTTCATTTGGAGAGAACACGGGGGACTCCTGCAGGAAAAATGGATCA | |
| | TTATCTTGATATTAGACTTAGACCTGATCCAGAATTTCCACCAGCTCAACTTATGTCTGTTCTTTTT | |
| | GGAAAACTTCATCAAGCTCTTGTTGCTCAAGGAGGAGATAGAATTGGAGTTTCTTTTCCTGATCTTG | |
| | ATGAATCAAGATCAAGACTTGGAGAAAGACTTAGAATTCATGCTTCTGCTGATGATCTTAGAGCTTT | |
| | GCTTGCTAGACCTTGGCTTGAAGGACTTAGAGATCATCTTCAATTTGGAGAACCAGCTGTTGTTCCA | |
| | CATCCAACTCCTTATAGACAAGTTTCAAGAGTTCAAGCTAAATCTAATCCAGAAAGACTTAGAAGAA | |
| | GACTTATGAGAAGACATGATCTTTCTGAAGAAGAAGCTAGAAAAAGAATTCCTGATACTGTTGCTAG | |
| | AGCTTTGGATTTGCCTTTTGTTACACTTAGATCACAATCTACTGGACAACATTTTAGACTTTTTATT | |
| | AGACATGGACCACTTCAAGTTACTGCTGAAGAAGGAGGATTTACTTGTTATGGACTTTCTAAGGGAG | |
| | GTTTTGTTCCTTGGTTTGGATCTGGAGCTACTAATTTTTCTCTTCTTAAGCAAGCTGGAGATGTTGA | |
| | AGAAAATCCTGGACCCATGGATAAGAAGTACTCTATCGGACTCGATATCGGAACTAACTCTGTGGGA | |
| | TGGGCTGTGATCACCGATGAGTACAAGGTGCCATCTAAGAAGTTCAAGGTTCTCGGAAACACCGATA | |
| | GGCACTCTATCAAGAAAAACCTTATCGGTGCTCTCCTCTTCGATTCTGGTGAAACTGCTGAGGCTAC | |
| | CAGACTCAAGAGAACCGCTAGAAGAAGGTACACCAGAAGAAAGAACAGGATCTGCTACCTCCAAGAG | |
| | ATCTTCTCTAACGAGATGGCTAAAGTGGATGATTCATTCTTCCACAGGCTCGAAGAGTCATTCCTCG | |
| | TGGAAGAAGATAAGAAGCACGAGAGGCACCCTATCTTCGGAAACATCGTTGATGAGGTGGCATACCA | |
| | CGAGAAGTACCCTACTATCTACCACCTCAGAAAGAAGCTCGTTGATTCTACTGATAAGGCTGATCTC | |
| | AGGCTCATCTACCTCGCTCTCGCTCACATGATCAAGTTCAGAGGACACTTCCTCATCGAGGGTGATC | |
| | TCAACCCTGATAACTCTGATGTGGATAAGTTGTTCATCCAGCTCGTGCAGACCTACAACCAGCTTTT | |
| | CGAAGAGAACCCTATCAACGCTTCAGGTGTGGATGCTAAGGCTATCCTCTCTGCTAGGCTCTCTAAG | |
| | TCAAGAAGGCTTGAGAACCTCATTGCTCAGCTCCCTGGTGAGAAGAAGAACGGACTTTTCGGAAACT | |
| | TGATCGCTCTCTCTCTCGGACTCACCCCTAACTTCAAGTCTAACTTCGATCTCGCTGAGGATGCAAA | |
| | GCTCCAGCTCTCAAAGGATACCTACGATGATGATCTCGATAACCTCCTCGCTCAGATCGGAGATCAG | |
| | TACGCTGATTTGTTCCTCGCTGCTAAGAACCTCTCTGATGCTATCCTCCTCAGTGATATCCTCAGAG | |
| | TGAACACCGAGATCACCAAGGCTCCACTCTCAGCTTCTATGATCAAGAGATACGATGAGCACCACCA | |
| | GGATCTCACACTTCTCAAGGCTCTTGTTAGACAGCAGCTCCCAGAGAAGTACAAAGAGATTTTCTTC | |
| | GATCAGTCTAAGAACGGATACGCTGGTTACATCGATGGTGGTGCATCTCAAGAAGAGTTCTACAAGT | |
| | TCATCAAGCCTATCCTCGAGAAGATGGATGGAACCGAGGAACTCCTCGTGAAGCTCAATAGAGAGGA | |
| | TCTTCTCAGAAAGCAGAGGACCTTCGATAACGGATCTATCCCTCATCAGATCCACCTCGGAGAGTTG | |
| | CACGCTATCCTTAGAAGGCAAGAGGATTTCTACCCATTCCTCAAGGATAACAGGGAAAAGATTGAGA | |
| | AGATTCTCACCTTCAGAATCCCTTACTACGTGGGACCTCTCGCTAGAGGAAACTCAAGATTCGCTTG | |
| | GATGACCAGAAAGTCTGAGGAAACCATCACCCCTTGGAACTTCGAAGAGGTGGTGGATAAGGGTGCT | |
| | AGTGCTCAGTCTTTCATCGAGAGGATGACCAACTTCGATAAGAACCTTCCAAACGAGAAGGTGCTCC | |
| | CTAAGCACTCTTTGCTCTACGAGTACTTCACCGTGTACAACGAGTTGACCAAGGTTAAGTACGTGAC | |
| | CGAGGGAATGAGGAAGCCTGCTTTTTTGTCAGGTGAGCAAAAGAAGGCTATCGTTGATCTCTTGTTC | |
| | AAGACCAACAGAAAGGTGACCGTGAAGCAGCTCAAAGAGGATTACTTCAAGAAAATCGAGTGCTTCG | |
| | ATTCAGTTGAGATTTCTGGTGTTGAGGATAGGTTCAACGCATCTCTCGGAACCTACCACGATCTCCT | |
| | CAAGATCATTAAGGATAAGGATTTCTTGGATAACGAGGAAAACGAGGATATCTTGGAGGATATCGTT | |
| | CTTACCCTCACCCTCTTTGAAGATAGAGAGATGATTGAAGAAAGGCTCAAGACCTACGCTCATCTCT | |
| | TCGATGATAAGGTGATGAAGCAGTTGAAGAGAAGAAGATACACTGGTTGGGGAAGGCTCTCAAGAAA | |
| | GCTCATTAACGGAATCAGGGATAAGCAGTCTGGAAAGACAATCCTTGATTTCCTCAAGTCTGATGGA | |
| | TTCGCTAACAGAAACTTCATGCAGCTCATCCACGATGATTCTCTCACCTTTAAAGAGGATATCCAGA | |
| | AGGCTCAGGTTTCAGGACAGGGTGATAGTCTCCATGAGCATATCGCTAACCTCGCTGGATCTCCTGC | |
| | AATCAAGAAGGGAATCCTCCAGACTGTGAAGGTTGTGGATGAGTTGGTGAAGGTGATGGGAAGGCAT | |
| | AAGCCTGAGAACATCGTGATCGAAATGGCTAGAGAGAACCAGACCACTCAGAAGGGACAGAAGAACT | |
| | CTAGGGAAAGGATGAAGAGGATCGAGGAAGGTATCAAAGAGCTTGGATCTCAGATCCTCAAAGAGCA | |
| | CCCTGTTGAGAACACTCAGCTCCAGAATGAGAAGCTCTACCTCTACTACCTCCAGAACGGAAGGGAT | |
| | ATGTATGTGGATCAAGAGTTGGATATCAACAGGCTCTCTGATTACGATGTTGATCATATCGTGCCAC | |
| | AGTCATTCTTGAAGGATGATTCTATCGATAACAAGGTGCTCACCAGGTCTGATAAGAACAGGGGTAA | |
| | GAGTGATAACGTGCCAAGTGAAGAGGTTGTGAAGAAAATGAAGAACTATTGGAGGCAGCTCCTCAAC | |
| | GCTAAGCTCATCACTCAGAGAAAGTTCGATAACTTGACTAAGGCTGAGAGGGGAGGACTCTCTGAAT | |
| | TGGATAAGGCAGGATTCATCAAGAGGCAGCTTGTGGAAACCAGGCAGATCACTAAGCACGTTGCACA | |
| | GATCCTCGATTCTAGGATGAACACCAAGTACGATGAGAACGATAAGTTGATCAGGGAAGTGAAGGTT | |
| | ATCACCCTCAAGTCAAAGCTCGTGTCTGATTTCAGAAAGGATTTCCAATTCTACAAGGTGAGGGAAA | |
| | TCAACAACTACCACCACGCTCACGATGCTTACCTTAACGCTGTTGTTGGAACCGCTCTCATCAAGAA | |
| | GTATCCTAAGCTCGAGTCAGAGTTCGTGTACGGTGATTACAAGGTGTACGATGTGAGGAAGATGATC | |
| | GCTAAGTCTGAGCAAGAGATCGGAAAGGCTACCGCTAAGTATTTCTTCTACTCTAACATCATGAATT | |
| | TCTTCAAGACCGAGATTACCCTCGCTAACGGTGAGATCAGAAAGAGGCCACTCATCGAGACAAACGG | |
| | TGAAACAGGTGAGATCGTGTGGGATAAGGGAAGGGATTTCGCTACCGTTAGAAAGGTGCTCTCTATG | |
| | CCACAGGTGAACATCGTTAAGAAAACCGAGGTGCAGACCGGTGGATTCTCTAAAGAGTCTATCCTCC | |
| | CTAAGAGGAACTCTGATAAGCTCATTGCTAGGAAGAAGGATTGGGACCCTAAGAAATACGGTGGTTT | |
| | CGATTCTCCTACCGTGGCTTACTCTGTTCTCGTTGTGGCTAAGGTTGAGAAGGGAAAGAGTAAGAAG | |
| | CTCAAGTCTGTTAAGGAACTTCTCGGAATCACTATCATGGAAAGGTCATCTTTCGAGAAGAACCCAA | |
| | TCGATTTCCTCGAGGCTAAGGGATACAAAGAGGTTAAGAAGGATCTCATCATCAAGCTCCCAAAGTA | |
| | CTCACTCTTCGAACTCGAGAACGGTAGAAAGAGGATGCTCGCTTCTGCTGGTGAGCTTCAAAAGGGA | |
| | AACGAGCTTGCTCTCCCATCTAAGTACGTTAACTTTCTTTACCTCGCTTCTCACTACGAGAAGTTGA | |
| | AGGGATCTCCAGAAGATAACGAGCAGAAGCAACTTTTCGTTGAGCAGCACAAGCACTACTTGGATGA | |
| | GATCATCGAGCAGATCTCTGAGTTCTCTAAAAGGGTGATCCTCGCTGATGCAAACCTCGATAAGGTG | |
| | TTGTCTGCTTACAACAAGCACAGAGATAAGCCTATCAGGGAACAGGCAGAGAACATCATCCATCTCT | |
| | TCACCCTTACCAACCTCGGTGCTCCTGCTGCTTTCAAGTACTTCGATACAACCATCGATAGGAAGAG | |
| | ATACACCTCTACCAAAGAAGTGCTCGATGCTACCCTCATCCATCAGTCTATCACTGGACTCTACGAG | |
| | ACTAGGATCGATCTCTCACAGCTCGGTGGTGATTCAAGGGCTGATCCTAAGAAGAAGAGGAAGGTTT | |
| | GACGTCGACGATATGAAGATGAAGATGAAATATTTGGTGTGTCAAATAAAAAGCTTGTGTGCTTAAG | |
| | TTTGTGTTTTTTTCTTGGCTTGTTGTGTTATGAATTTGTGGCTTTTTCTAATATTAAATGAATGTAA | |
| | GATCACATTATAATGAATAAACAAATGTTTCTATAATCCATTGTGAATGTTTTGTTGGATCTCTTCT | |
| | GCAGCATATAACTACTGTATGTGCTATGGTATGGACTATGGAATATGATTAAAGATAAGCCAGAGCT | |
| | CTGGTGACGGACGGCGCGCTGGCAGACATACTGTCCCACAAATGAAGATGGAATCTGTAAAAGAAAA | |
| | CGCGTGAAATAATGCGTCTGACAAAGGTTAGGTCGGCTGCCTTTAATCAATACCAAAGTGGTCCCTA | |
| | CCACGATGGAAAAACTGTGCAGTCGGTTTGGCTTTTTCTGACGAACAAATAAGATTCGTGGCCGACA | |
| | GGTGGGGGTCCACCATGTGAAGGCATCTTCAGACTCCAATAATGGAGCAATGACGTAAGGGCTTACG | |
| | AAATAAGTAAGGGTAGTTTGGGAAATGTCCACTCACCCGTCAGTCTATAAATACTTAGCCCCTCCCT | |
| | CATTGTTAAGGGAGCAAAATCTCAGAGAGATAGTCCTAGAGAGAGAAAGAGAGCAAGTAGCCTAGAA | |
| | GTAGTCAAGGCGGCGAAGTATTCAGGCACGTGGCCAGGAAGAAGAAAAGCCAAGACGACGAAAACAG | |
| | GTAAGAGCTAAGCTTCCTGCAGGTTCACTGCCGTATAGGCAGCATTAACATTACCATTAACGGTTTT | |
| | AGAGCTAGAAATAGCAAGTTAAAATAAGGCTAGTCCGTTATCAACTTGAAAAAGTGGCACCGAGTCG | |
| | GTGCGTTCACTGCCGTATAGGCAGAGGGACACCAATGTCCTGCTGTTTTAGAGCTAGAAATAGCAAG | |
| | TTAAAATAAGGCTAGTCCGTTATCAACTTGAAAAAGTGGCACCGAGTCGGTGCGTTCACTGCCGTAT | |
| | AGGCAGGTCGATCGACAAGCTCGAGTTTCTCCATAATAATGTGTGAGTAGTTCCCAGATAAGGGAAT | |
| | TAGGGTTCCTATAGGGTTTCGCTCATGTGTTGAGCATATAAGAAACCCTTAGTATGTATTTGTATTT | |
| | GTAAAATACTTCTATCAATAAAATTTCTAATTCCTAAAACCAAAATCCAGTACTAAAATCCAGATCC | |
| | CCCGAATTAGAGCTCTACCGGCGAGCTTTGGGTACGTCACGTGGCTCGAGCGCGTAGTCCTCGGTAG | |
| | GCAAGCTTATTTAATTCATACAGAAGCAATCTTTGTTTCAGATGTTCACTACAAAACTCATCCTCTT | |
| | CTTCAATATTTTTGGTTTCGGAATGATCGCTATCTTAACTCTTTTCCTTACACATGGCCGCAAACGC | |
| | GTTGATGTTCTTGGATGGATTTGCATGATCTTTGCTTTATGCGTGTTTGTTGCCCCCATGGGTATCA | |
| | TGGTGAGAATGCGAGTCGCAAATTTCAACACTTGCTTCTTTCTGTCTCTGACAGTTTTTTTTTTTTC | |
| | CCCTATAATTATATTGATTGATTTTTGTTTTCTCTCTTCTTTACTCTATTTTCCAGAGAAAAGTGAT | |
| | AAAAACGAAGAGTGTCGAGTTCATGCCATTTTCTTTATCATTCTTCCTCACCTTGACTGCGGTGATG | |
| | TGGTTCTTCTATGGTTTTCTAAAGAAAGACCTTTATGTTGCCGTAAGTTAACTATCACGCATGCATC | |
| | ATTATCACGTACATCTTTCTTTACATTCCACCAACTTTATCTTTCCCATTAATCATCAACCCAGCAA | |
| | CTATTTCTTATTCCCTTTTGATTAACTTCCACTTACAATTTCCTTTTTCTTGTCATGAACAGATTCC | |
| | AAACACATTGGGCTTTCTTTTTGGGATTGTCCAGATGGTGCTTTATTTAATCTACAGAAACCCCAAG | |
| | AAATTACCTGTAGAGGATCCTAAACTTCGCGAATTGTCCGAGCACATCGTCGACGTTGCAAAGCTGA | |
| | GTGCAACCCTCTGTTCCGAGATAACCACAGTAGTGGTTCCACAGCCCATAGACAATGGAAATGATGT | |
| | TGAAGGTCAAAAAATTAAGGAAGAAAACGAGCAGGACATTGGTGTCCCTGCAGACAAAGTTAAGACT | |
| | AATCTTTTTCTCTTTCTCATCTTTTCACTTCTCCAATCATTATCCTCGGCCGAATTCAGTAAAGGAG | |
| | AAGAACTTTTCACTGGAGTTGTCCCAATTCTTGTTGAATTAGATGGTGATGTTAATGGGCACAAATT | |
| | TTCTGTCAGTGGAGAGGGTGAAGGTGATGCAACATACGGAAAACTTACCCTTAAATTTATTTGCACT | |
| | ACTGGAAAACTACCTGTTCCATGGCCAACACTTGTCACTACTTTCACTTATGGTGTTCAATGCTTTT | |
| | CAAGATACCCAGATCATATGAAGCGGCACGACTTCTTCAAGAGCGCCATGCCTGAGGGATACGTGCA | |
| | GGAGAGGACCATCTCTTTCAAGGACGACGGGAACTACAAGACACGTGCTGAAGTCAAGTTTGAGGGA | |
| | GACACCCTCGTCAACAGGATCGAGCTTAAGGGAATCGATTTCAAGGAGGACGGAAACATCCTCGGCC | |
| | ACAAGTTGGAATACAACTACAACTCCCACAACGTATACATCACGGCAGACAAACAAAAGAATGGAAT | |
| | CAAAGCTAACTTCAAAATTAGACACAACATTGAAGATGGAAGCGTTCAACTAGCAGACCATTATCAA | |
| | CAAAATACTCCAATTGGCGATGGCCCTGTCCTTTTACCAGACAACCATTACCTGTCCACACAATCTG | |
| | CCCTTTCGAAAGATCCCAACGAAAAGAGAGACCACATGGTCCTTCTTGAGTTTGTAACAGCTGCTGG | |
| | GATTACACATGGCATGGATGAACTATACAAACATGATGAGCTTTGATAACATTAACATTACCATTAA | |
| | CGTGATCTTGGTTATGTTTTTTCTTTTTAATTTTGCATGTAATCGTTCAAAGTGGTGGTGCCATGTC | |
| | TACTTGTAAGGCTGCAATGCAGCCATGTTGTCTATTATGTCAAATCTAGTTCCATTTAATGTCAATC | |
| | TTTATTCTCAACCTAAAAGAAGAATATCAATCTTTATGTAATACGTTTTTTCGAGTAAATAAAATGT | |
| | CCAGTGAATTTACAGTTAATGTTAAATCAGCATTATATTTTAGGAAAATAGTATTCAACTTATAGTT | |
| | TAATGGTTGAAATTAAATATTAATTTTTATTTTATGATGTAATAATTTTAAATTTAAATTATAGCTC | |
| | CTGGCAAGAGTTATTAATAAAATAATACTGCCAATATTTTTTTCTAAATTTTATTTGAATTTGTTAT | |
| | TTATTTTATGGAAAATATTTTTAAAAAATAATTTTCATATTTTTTTATATAAGAAGAGCTCAAAAAA | |
| | ATTTTAAATCCATGTTATTTTACACTAAAAAACAGAAGTTTAAATAGGGGAGAAATTTTTACATTCG | |
| | CCAACAAAACTATATAAATTTTTGTTTTGAATTATAAAATAATAATTATTTTTCCTAAAAAGAATTC | |
| | TTCATGATTGTGCCAAATAAGTCTCAATGCAATTTTAAAAAAAATCCAGACAAAATTTGTCTTATTT | |
| | CTCACTGTGCTATTTTTCTAATAAGCATTTTCATTGTGCAATTAAATCTATTGGACTCTAATCAATA | |
| | ATAAAGAAAAGGGATACCTTTAATCTTTTATCGAAGATATCAACTAATTCTAGAGCGCGGTAATATC | |
| | GCAGAACAAAAGTACCTGATATCGAGTGTACTTCAAGTCACACCGGCG | |
| 10 | CCAGGCTTCATCCTAACCATTACAGGCAAGATGTTGTATGAAGAAGGGCGAACATGCAGATTGTTAA | Rice callus |
| | ACTGACACGTGATGGACAAGAATGACCGATTGGTGACCGGTCTGACAATGGTCATGTCGTCAGCAGA | specific |
| | CAGCCATCTCCCACGTCGCGCCTGCTTCCGGTGAAAGTGGAGGTAGGTATGGGCCGTCCCGTCAGAA | promoter |
| | GGTGATTCGGATGGCAGCGATACAAATCTCCGTCCATTAATGAAGAGAAGTCAAGTTGAAAGAAAGG | |
| | GAGGGAGAGATGGTGCATGTGGGATCCCCTTGGGATATAAAAGGAGGACCTTGCCCACTTAGAAAGG | |
| | AGAGGAGAAAGCAATCCCAGAAGAATCGGGGGCTGACTGGCACTTTGTAGCTTCTTCATACGCGAAT | |
| | CCACCAAAACACAGGAGTAGGGTATTACGCTTCTCAGCGGCCCGAACCTGTATACATCGCCCGTGTC | |
| | TTGTGTGTTTCCGCTCTTGCGAACCTTCCACAGATTGGGAGCTTAGAACCTCACCCAGGGCCCCCGG | |
| | CCGAACTGGCAAAGGGGGGCCTGCGCGGTCTCCCGGTGAGGAGCCCCACGCTCCGTCAGTTCTAAAT | |
| | TACCCGATGAGAAAGGGAGGGGGGGGGGAAAATCTGCCTTGTTTATTTACGATCCAACGGATTTGGT | |
| | CGACACCGATGAGGTGTCTTACCAGTTACCACGAGCTAGATTATAGTACTAATTACTTGAGGATTCG | |
| | GTTCCTAATTTTTTACCCGATCGACTTCGCCATGGAAAATTTTTTATTCGGGGGAGAATATCCACCC | |
| | TGTTTCGCTCCTAATTAAGATAGGAATTGTTACGATTAGCAACCTAATTCAGATCAGAATTGTTAGT | |
| | TAGCGGCGTTGGATCCCTCACCTCATCCCATCCCAATTCCCAAACCCAAACTCCTCTTCCAGTCGCC | |
| | GACCCAAACACGCATCCGCCGCCTATAAATCCCACCCGCATCGAGCCTATCAAGCCCAAAAAACCAC | |
| | AAACCCAACGAAGAAGGAAAAAAAAAGGAGGAAAAGAAAAGAGGAGGAAAGCGAAGAGGTTGGAGAG | |
| | AGACGCTCGTCTCCACGTCGCCGCC | |
| 11 | CCAGGCTTCATCCTAACCATTACAGGCAAGATGTTGTATGAAGAAGGGCGAACATGCAGATTGTTAA | Rice callus |
| | ACTGACACGTGATGGACAAGAATGACCGATTGGTGACCGGTCTGACAATGGTCATGTCGTCAGCAGA | specific |
| | CAGCCATCTCCCACGTCGCGCCTGCTTCCGGTGAAAGTGGAGGTAGGTATGGGCCGTCCCGTCAGAA | promoter |
| | GGTGATTCGGATGGCAGCGATACAAATCTCCGTCCATTAATGAAGAGAAGTCAAGTTGAAAGAAAGG | modified to |
| | GAGGGAGAGATGGTGCATGTGGGATCCCCTTGGGATATAAAAGGAGGACCTTGCCCACTTAGAAAGG | make the |
| | AGAGGAGAAAGCAATCCCAGAAGAATCGGGGGCTGACTGGCACTTTGTAGCTTCTTCATACGCGAAT | following |
| | CCACCAAAACACAGGAGTAGGGTATTACGCTTCTCAGCGGCCCGAACCTGTATACATCGCCCGTGTC | changes: |
| | TTGTGTGTTTCCGCTCTTGCGAACCTTCCACAGATTGGGAGCTTAGAACCTCACCCAGGGCCCCCGG | G563A, G626C, |
| | CCGAACTGGCAAAGGGGGGCCTGCGCAGTCTCCCGGTGAGGAGCCCCACGCTCCGTCAGTTCTAAAT | G1077A, and |
| | TACCCGATGAGAAAGGGAGGGGCGGGGGAAAATCTGCCTTGTTTATTTACGATCCAACGGATTTGGT | C1080A. The |
| | CGACACCGATGAGGTGTCTTACCAGTTACCACGAGCTAGATTATAGTACTAATTACTTGAGGATTCG | modifications |
| | GTTCCTAATTTTTTACCCGATCGACTTCGCCATGGAAAATTTTTTATTCGGGGGAGAATATCCACCC | remove three |
| | TGTTTCGCTCCTAATTAAGATAGGAATTGTTACGATTAGCAACCTAATTCAGATCAGAATTGTTAGT | internal |
| | TAGCGGCGTTGGATCCCTCACCTCATCCCATCCCAATTCCCAAACCCAAACTCCTCTTCCAGTCGCC | restriction |
| | GACCCAAACACGCATCCGCCGCCTATAAATCCCACCCGCATCGAGCCTATCAAGCCCAAAAAACCAC | enzyme |
| | AAACCCAACGAAGAAGGAAAAAAAAAGGAGGAAAAGAAAAGAGGAGGAAAGCGAAGAGGTTGGAGAG | recognition |
| | AGACACTAGTCTCCACGTCGCCGCC | sites that |
| | | could |
| | | interfere with |
| | | downstream |
| | | assembly and |
| | | to disrupt a |
| | | polyG(10) to |
| | | improve |
| | | nucleic acid |
| | | synthesis |
| 12 | GTAGGCGGTGCTACAGAGTTCTTGAAGTGGTGGCCTAACTACGGCTACACTAGAAGGACAGTATTTG | pMCS305 |
| | GTATCTGCGCTCTGCTGAAGCCAGTTACCTTCGGAAAAAGAGTTGGTAGCTCTTGATCCGGCAAACA | OsCSP::eGFP |
| | AACCACCGCTGGTAGCGGTGGTTTTTTTGTTTGCAAGCAGCAGATTACGCGCAGAAAAAAAGGATCT | (+Control) |
| | CAAGAAGATCCTTTGATCTTTTCTACGGGGTCTGACGCTCAGTGGAACGAAAACTCACGTTAAGGGA | |
| | TTTTGGTCATGCATTCTAGGTACTAAAACAATTCATCCAGTAAAATATAATATTTTATTTTCTCCCA | |
| | ATCAGGCTTGATCCCCAGTAAGTCAAAAAATAGCTCGACATACTGTTCTTCCCCGATATCCTCCCTG | |
| | ATCGACCGGACGCAGAAGGCAATGTCATACCACTTGTCCGCCCTGCCGCTTCTCCCAAGATCAATAA | |
| | AGCCACTTACTTTGCCATCTTTCACAAAGATGTTGCTGTCTCCCAGGTCGCCGTGGGAAAAGACAAG | |
| | TTCCTCTTCGGGCTTTTCCGTCTTTAAAAAATCATACAGCTCGCGCGGATCTTTAAATGGAGTGTCT | |
| | TCTTCCCAGTTTTCGCAATCCACATCGGCCAGATCGTTATTCAGTAAGTAATCCAATTCGGCTAAGC | |
| | GGCTGTCTAAGCTATTCGTATAGGGACAATCCGATATGTCGATGGAGTGAAAGAGCCTGATGCACTC | |
| | CGCATACAGCTCGATAATCTTTTCAGGGCTTTGTTCATCTTCATACTCTTCCGAGCAAAGGACGCCA | |
| | TCGGCCTCACTCATGAGCAGATTGCTCCAGCCATCATGCCGTTCAAAGTGCAGGACCTTTGGAACAG | |
| | GCAGCTTTCCTTCCAGCCATAGCATCATGTCCTTTTCCCGTTCCACATCATAGGTGGTCCCTTTATA | |
| | CCGGCTGTCCGTCATTTTTAAATATAGGTTTTCATTTTCTCCCACCAGCTTATATACCTTAGCAGGA | |
| | GACATTCCTTCCGTATCTTTTACGCAGCGGTATTTTTCGATCAGTTTTTTCAATTCCGGTGATATTC | |
| | TCATTTTAGCCATTTATTATTTCCTTCCTCTTTTCTACAGTATTTAAAGATACCCCAAGAAGCTAAT | |
| | TATAACAAGACGAACTCCAATTCACTGTTCCTTGCATTCTAAAACCTTAAATACCAGAAAACAGCTT | |
| | TTTCAAAGTTGTTTTCAAAGTTGGCGTATAACATAGTATCGACGGAGCCGATTTTGAAACCGCGGTG | |
| | ATCACAGGCAGCAACGCTCTGTCATCGTTACAATCAACATGCTACCCTCCGCGAGATCATCCGTGTT | |
| | TCAAACCCGGCAGCTTAGTTGCCGTTCTTCCGAATAGCATCGGTAACATGAGCAAAGTCTGCCGCCT | |
| | TACAACGGCTCTCCCGCTGACGCCGTCCCGGACTGATGGGCTGCCTGTATCGAGTGGTGATTTTGTG | |
| | CCGAGCTGCCGGTCGGGGAGCTGTTGGCTGGCTGGTGGCAGGATATATTGTGGTGTAAACAAATTGA | |
| | CGCTTAGACAACTTAATAACACATTGCGGACGTTTTTAATGTACTGAATTAACGCCGAATTAATTCG | |
| | GGGGATCTGGATTTTAGTACTGGATTTTGGTTTTAGGAATTAGAAATTTTATTGATAGAAGTATTTT | |
| | ACAAATACAAATACATACTAAGGGTTTCTTATATGCTCAACACATGAGCGAAACCCTATAGGAACCC | |
| | TAATTCCCTTATCTGGGAACTACTCACACATTATTATGGAGAAACTCGAGCTTGTCGATCGACAGAT | |
| | CCGGTCGGCATCTACTCTATTTCTTTGCCCTCGGACGAGTGCTGGGGCGTCGGTTTCCACTATCGGC | |
| | GAGTACTTCTACACAGCCATCGGTCCAGACGGCCGCGCTTCTGCGGGCGATTTGTGTACGCCCGACA | |
| | GTCCCGGCTCCGGATCGGACGATTGCGTCGCATCGACCCTGCGCCCAAGCTGCATCATCGAAATTGC | |
| | CGTCAACCAAGCTCTGATAGAGTTGGTCAAGACCAATGCGGAGCATATACGCCCGGAGTCGTGGCGA | |
| | TCCTGCAAGCTCCGGATGCCTCCGCTCGAAGTAGCGCGTCTGCTGCTCCATACAAGCCAACCACGGC | |
| | CTCCAGAAGAAGATGTTGGCGACCTCGTATTGGGAATCCCCGAACATCGCCTCGCTCCAGTCAATGA | |
| | CCGCTGTTATGCGGCCATTGTCCGTCAGGACATTGTTGGAGCCGAAATCCGCGTGCACGAGGTGCCG | |
| | GACTTCGGGGCAGTCCTCGGCCCAAAGCATCAGCTCATCGAGAGCCTGCGCGACGGACGCACTGACG | |
| | GTGTCGTCCATCACAGTTTGCCAGTGATACACATGGGGATCAGCAATCGCGCATATGAAATCACGCC | |
| | ATGTAGTGTATTGACCGATTCCTTGCGGTCCGAATGGGCCGAACCCGCTCGTCTGGCTAAGATCGGC | |
| | CGCAGCGATCGCATCCATAGCCTCCGCGACCGGTTGTAGAACAGCGGGCAGTTCGGTTTCAGGCAGG | |
| | TCTTGCAACGTGACACCCTGTGCACGGCGGGAGATGCAATAGGTCAGGCTCTCGCTAAACTCCCCAA | |
| | TGTCAAGCACTTCCGGAATCGGGAGCGCGGCCGATGCAAAGTGCCGATAAACATAACGATCTTTGTA | |
| | GAAACCATCGGCGCAGCTATTTACCCGCAGGACATATCCACGCCCTCCTACATCGAAGCTGAAAGCA | |
| | CGAGATTCTTCGCCCTCCGAGAGCTGCATCAGGTCGGACACGCTGTCGAACTTTTCGATCAGAAACT | |
| | TCTCGACAGACGTCGCGGTGAGTTCAGGCTTTTTCATAAGCTTCTGCAAAAGAGAACCAGACAACAG | |
| | GGTAAGTGCCTAGCAGTAAACAAACAGAACTCATCACAAGCAAACAGCAACATCATATTCATACCAA | |
| | CAGGTCATGTGTGTTCATCACATCATTAGTACTAAGCATGCCATCATCCAAGTATATCAAAGTAAGG | |
| | GCAAAGAGCATGCATGATCATCAGGTGCACAAAAGAATCATCAAATTGTAGCAGTACATATCTTCAT | |
| | CTATCATGCATATCTATCCATAACAGGACGATGCATGTTGACCAGGTAAAAGCTACAGGATCCTATA | |
| | GGAACAGCAGCGTATATATCTTCACCAATCTTAGCATCTTAATCATGTGGCACATGCAGTTTCAATT | |
| | TAAGCACATGAGCTAGTTGATTATGAGGTACCAGAGAATCATCAAACTGATGTAGCAGCATATATCC | |
| | TCATCTATCATGCAATCTAATCTAATCTAACTAAACAGGAAAGGTGTGCTATTCAGTTAAAAGCTAC | |
| | CGCATCATATACAAACGGCAGCATAAGAAAAAGCATAATCATCTTAATCATGCAACAAACGCAGATT | |
| | CATAATAAGCCCAAGAGCTAGCTTGTGATGATCTTATTCTACTCTGATCTACAGCAATCAGATAACG | |
| | ACCTAACCTTGCACATGGCAACAAAACAATCGATCGGACGAATCAGTTGTTTGTTCCTAGCTAGCAC | |
| | CATCGAACCAGATAATAGATGCACGTACAGATCCCGAAAACGAACCCAAAAACAGGGCAGACCTAGC | |
| | TGAACCTAGGCAGCGACCCAGCAGATCGTGAGAACGATCTCATCTACGAACAGCCTAGAAGCAACCC | |
| | CACGATTCCCGGACAAACGACCTAAAATCCCCACAAATCACATGAGCATGACAGGATAAACAGCGGA | |
| | ACCGATCAGATCTACACGAAAACCCCACCTCCCAGCCACCCACGATCAGGAAACACGCGGATCTAGC | |
| | ATGATTTCGTCAACGCCTCAGCCTAGTTCCTAGCCACAGACCAAGCAGAACCACCAAACCACGCCGA | |
| | GCGAGGAGATGGGGCAAGAGGACGGGGGAGACGATCGCCGTACCTTGAAGCGGGGGAAGGATCGCCG | |
| | AGGGTCGCGAGGAGAGCAATTTGGATTTGGAACCCGGGGGTTGTGCGCTCCGAACGATGAGACGATG | |
| | TGAGATTGTGGGAAGAGGCGCGGAGGGCCCTGTATTTATGGGCTGCGACGGGGGGAGGAGAGGTGGG | |
| | GAGGGTTGGGGAAGGAATCCCCCACCCGTGCCGTGACGGTTCCGGGCCGTGTGAGAGGAGCCCGCTC | |
| | GTCTCCGCCACGCAATTTCCGCGATCGGAGCGGAGCTTTCGAGAGGCGGCTGGATGGTTGGTGGCCG | |
| | TTAGATTTGTAGACGCCGTTAACGCCTCGCCTCCACCGGGAAGAGTTTTGAGCAGCCGCTTATGACA | |
| | ATGGCTTAACGACGTTAGACGGAGCGTTAGTGGCAGGCCATGATAGAATAGACGTATTGCAATGGGA | |
| | TTATAATTAAATAAATAAGAATATAATAAGATATGGCAAGTCGGCACTCATGACATGGTCTTCGAAA | |
| | TGATAGTGCTCACTTTCTTAGCCGAGAAAGTTGACGCGACTGATTTAGAAGTTAAGATATTATTTCT | |
| | CTCTTCTTTTCTTTCCTCGTCATATAAGGATGAAATAAACTTTAGAGATTGCCGGTAGTGATTTTGG | |
| | ATTTCGGCGATCAGGCTTGGTTTGCCGGTTTCGGACGGTGTGCCTTAGGCCACCCGCAGTGTATCTT | |
| | GTAATGTTCAACCGATAAGCAAGGGTGGGGCTCAAGCAAGTAGTAAACAACTATGTCAAATGTCACC | |
| | ATGGTTATGGTCTTGTTTAGTTGGCTTCTTGGCCGATTCATTAATGCAGCTGGCACGACAGGTTTCC | |
| | CGACTGGAAAGCGGGCAGTGAGCGCAACGCAATTAATGTGAGTTAGCTCACTCATTAGGCACCCCAG | |
| | GCTTTACACTTTATGCTTCCGGCTCGTATGTTGTGTGGAATTGTGAGCGGATAACAATTTCACACAG | |
| | GAAACAGCTATGACATGATTACGAATTCGAGCTCGGTACCCGGGGATCCCAGGCTTCATCCTAACCA | |
| | TTACAGGCAAGATGTTGTATGAAGAAGGGCGAACATGCAGATTGTTAAACTGACACGTGATGGACAA | |
| | GAATGACCGATTGGTGACCGGTCTGACAATGGTCATGTCGTCAGCAGACAGCCATCTCCCACGTCGC | |
| | GCCTGCTTCCGGTGAAAGTGGAGGTAGGTATGGGCCGTCCCGTCAGAAGGTGATTCGGATGGCAGCG | |
| | ATACAAATCTCCGTCCATTAATGAAGAGAAGTCAAGTTGAAAGAAAGGGAGGGAGAGATGGTGCATG | |
| | TGGGATCCCCTTGGGATATAAAAGGAGGACCTTGCCCACTTAGAAAGGAGAGGAGAAAGCAATCCCA | |
| | GAAGAATCGGGGGCTGACTGGCACTTTGTAGCTTCTTCATACGCGAATCCACCAAAACACAGGAGTA | |
| | GGGTATTACGCTTCTCAGCGGCCCGAACCTGTATACATCGCCCGTGTCTTGTGTGTTTCCGCTCTTG | |
| | CGAACCTTCCACAGATTGGGAGCTTAGAACCTCACCCAGGGCCCCCGGCCGAACTGGCAAAGGGGGG | |
| | CCTGCGCGGTCTCCCGGTGAGGAGCCCCACGCTCCGTCAGTTCTAAATTACCCGATGAGAAAGGGAG | |
| | GGGGGGGGGAAAATCTGCCTTGTTTATTTACGATCCAACGGATTTGGTCGACACCGATGAGGTGTCT | |
| | TACCAGTTACCACGAGCTAGATTATAGTACTAATTACTTGAGGATTCGGTTCCTAATTTTTTACCCG | |
| | ATCGACTTCGCCATGGAAAATTTTTTATTCGGGGGAGAATATCCACCCTGTTTCGCTCCTAATTAAG | |
| | ATAGGAATTGTTACGATTAGCAACCTAATTCAGATCAGAATTGTTAGTTAGCGGCGTTGGATCCCTC | |
| | ACCTCATCCCATCCCAATTCCCAAACCCAAACTCCTCTTCCAGTCGCCGACCCAAACACGCATCCGC | |
| | CGCCTATAAATCCCACCCGCATCGAGCCTATCAAGCCCAAAAAACCACAAACCCAACGAAGAAGGAA | |
| | AAAAAAAGGAGGAAAAGAAAAGAGGAGGAAAGCGAAGAGGTTGGAGAGAGACGCTCGTCTCCACGTC | |
| | GCCGCCATCAAAATCAATTCGATCCCATGGTGAGCAAGGGCGAGGAGCTGTTCACCGGGGTGGTGCC | |
| | CATCCTGGTCGAGCTGGACGGCGACGTAAACGGCCACAAGTTCAGCGTGTCCGGCGAGGGCGAGGGC | |
| | GATGCCACCTACGGCAAGCTGACCCTGAAGTTCATCTGCACCACCGGCAAGCTGCCCGTGCCCTGGC | |
| | CCACCCTCGTGACCACCTTCACCTACGGCGTGCAGTGCTTCAGCCGCTACCCCGACCACATGAAGCA | |
| | GCACGACTTCTTCAAGTCCGCCATGCCCGAAGGCTACGTCCAGGAGCGCACCATCTTCTTCAAGGAC | |
| | GACGGCAACTACAAGACCCGCGCCGAGGTGAAGTTCGAGGGCGACACCCTGGTGAACCGCATCGAGC | |
| | TGAAGGGCATCGACTTCAAGGAGGACGGCAACATCCTGGGGCACAAGCTGGAGTACAACTACAACAG | |
| | CCACAACGTCTATATCATGGCCGACAAGCAGAAGAACGGCATCAAGGTGAACTTCAAGATCCGCCAC | |
| | AACATCGAGGACGGCAGCGTGCAGCTCGCCGACCACTACCAGCAGAACACCCCCATCGGCGACGGCC | |
| | CCGTGCTGCTGCCCGACAACCACTACCTGAGCACCCAGTCCGCCCTGAGCAAAGACCCCAACGAGAA | |
| | GCGCGATCACATGGTCCTGCTGGAGTTCGTGACCGCCGCCGGGATCACTCACGGCATGGACGAGCTG | |
| | TACAAGTAAAGCGGCCGCCCGGCTGCAGGAATTGATCCGAGCTCGAATTTCCCCGATCGTTCAAACA | |
| | TTTGGCAATAAAGTTTCTTAAGATTGAATCCTGTTGCCGGTCTTGCGATGATTATCATATAATTTCT | |
| | GTTGAATTACGTTAAGCATGTAATAATTAACATGTAATGCATGACGTTATTTATGAGATGGGTTTTT | |
| | ATGATTAGAGTCCCGCAATTATACATTTAATACGCGATAGAAAACAAAATATAGCGCGCAAACTAGG | |
| | ATAAATTATCGCGCGCGGTGTCATCTATGTTACTAGATCGGGAATTCGTAATCATGTCATAGCTGTT | |
| | TCCTGTGTGAGTGTGGCACTGGCCGTCGTTTTACAACGTCGTGACTGGGAAAACCCTGGCGTTACCC | |
| | AACTTAATCGCCTTGCAGCACATCCCCCTTTCGCCAGCTGGCGTAATAGCGAAGAGGCCCGCACCGA | |
| | TCGCCCTTCCCAACAGTTGCGCAGCCTGAATGGCGAATGCTAGAGCAGCTTGAGCTTGGATCAGATT | |
| | GTCGTTTCCCGCCTTCAGTTTAAACTATCAGTGTTTGACAGGATATATTGGCGGGTAAACCTAAGAG | |
| | AAAAGAGCGTTTATTAGAATAATCGGATATTTAAAAGGGCGTGAAAAGGTTTATCCGTTCGTCCATT | |
| | TGTATGTGCATGCCAACCACAGGGTTCCCCTCGGGATCAAAGTACTTTGATCCAACCCCTCCGCTGC | |
| | TATAGTGCAGTCGGCTTCTGACGTTCAGTGCAGCCGTCTTCTGAAAACGACATGTCGCACAAGTCCT | |
| | AAGTTACGCGACAGGCTGCCGCCCTGCCCTTTTCCTGGCGTTTTCTTGTCGCGTGTTTTAGTCGCAT | |
| | AAAGTAGAATACTTGCGACTAGAACCGGAGACATTACGCCATGAACAAGAGCGCCGCCGCTGGCCTG | |
| | CTGGGCTATGCCCGCGTCAGCACCGACGACCAGGACTTGACCAACCAACGGGCCGAACTGCACGCGG | |
| | CCGGCTGCACCAAGCTGTTTTCCGAGAAGATCACCGGCACCAGGCGCGACCGCCCGGAGCTGGCCAG | |
| | GATGCTTGACCACCTACGCCCTGGCGACGTTGTGACAGTGACCAGGCTAGACCGCCTGGCCCGCAGC | |
| | ACCCGCGACCTACTGGACATTGCCGAGCGCATCCAGGAGGCCGGCGCGGGCCTGCGTAGCCTGGCAG | |
| | AGCCGTGGGCCGACACCACCACGCCGGCCGGCCGCATGGTGTTGACCGTGTTCGCCGGCATTGCCGA | |
| | GTTCGAGCGTTCCCTAATCATCGACCGCACCCGGAGCGGGCGCGAGGCCGCCAAGGCCCGAGGCGTG | |
| | AAGTTTGGCCCCCGCCCTACCCTCACCCCGGCACAGATCGCGCACGCCCGCGAGCTGATCGACCAGG | |
| | AAGGCCGCACCGTGAAAGAGGCGGCTGCACTGCTTGGCGTGCATCGCTCGACCCTGTACCGCGCACT | |
| | TGAGCGCAGCGAGGAAGTGACGCCCACCGAGGCCAGGCGGCGCGGTGCCTTCCGTGAGGACGCATTG | |
| | ACCGAGGCCGACGCCCTGGCGGCCGCCGAGAATGAACGCCAAGAGGAACAAGCATGAAACCGCACCA | |
| | GGACGGCCAGGACGAACCGTTTTTCATTACCGAAGAGATCGAGGCGGAGATGATCGCGGCCGGGTAC | |
| | GTGTTCGAGCCGCCCGCGCACGGCTCAACCGTGCGGCTGCATGAAATCCTGGCCGGTTTGTCTGATG | |
| | CCAAGCTGGCGGCCTGGCCGGCCAGCTTGGCCGCTGAAGAAACCGAGCGCCGCCGTCTAAAAAGGTG | |
| | ATGTGTATTTGAGTAAAACAGCTTGCGTCATGCGGTCGCTGCGTATATGATGCGATGAGTAAATAAA | |
| | CAAATACGCAAGGGGAACGCATGAAGGTTATCGCTGTACTTAACCAGAAAGGCGGGTCAGGCAAGAC | |
| | GACCATCGCAACCCATCTAGCCCGCGCCCTGCAACTCGCCGGGGCCGATGTTCTGTTAGTCGATTCC | |
| | GATCCCCAGGGCAGTGCCCGCGATTGGGCGGCCGTGCGGGAAGATCAACCGCTAACCGTTGTCGGCA | |
| | TCGACCGCCCGACGATTGACCGCGACGTGAAGGCCATCGGCCGGCGCGACTTCGTAGTGATCGACGG | |
| | AGCGCCCCAGGCGGCGGACTTGGCTGTGTCCGCGATCAAGGCAGCCGACTTCGTGCTGATTCCGGTG | |
| | CAGCCAAGCCCTTACGACATATGGGCCACCGCCGACCTGGTGGAGCTGGTTAAGCAGCGCATTGAGG | |
| | TCACGGATGGAAGGCTACAAGCGGCCTTTGTCGTGTCGCGGGCGATCAAAGGCACGCGCATCGGCGG | |
| | TGAGGTTGCCGAGGCGCTGGCCGGGTACGAGCTGCCCATTCTTGAGTCCCGTATCACGCAGCGCGTG | |
| | AGCTACCCAGGCACTGCCGCCGCCGGCACAACCGTTCTTGAATCAGAACCCGAGGGCGACGCTGCCC | |
| | GCGAGGTCCAGGCGCTGGCCGCTGAAATTAAATCAAAACTCATTTGAGTTAATGAGGTAAAGAGAAA | |
| | ATGAGCAAAAGCACAAACACGCTAAGTGCCGGCCGTCCGAGCGCACGCAGCAGCAAGGCTGCAACGT | |
| | TGGCCAGCCTGGCAGACACGCCAGCCATGAAGCGGGTCAACTTTCAGTTGCCGGCGGAGGATCACAC | |
| | CAAGCTGAAGATGTACGCGGTACGCCAAGGCAAGACCATTACCGAGCTGCTATCTGAATACATCGCG | |
| | CAGCTACCAGAGTAAATGAGCAAATGAATAAATGAGTAGATGAATTTTAGCGGCTAAAGGAGGCGGC | |
| | ATGGAAAATCAAGAACAACCAGGCACCGACGCCGTGGAATGCCCCATGTGTGGAGGAACGGGCGGTT | |
| | GGCCAGGCGTAAGCGGCTGGGTTGTCTGCCGGCCCTGCAATGGCACTGGAACCCCCAAGCCCGAGGA | |
| | ATCGGCGTGACGGTCGCAAACCATCCGGCCCGGTACAAATCGGCGCGGCGCTGGGTGATGACCTGGT | |
| | GGAGAAGTTGAAGGCCGCGCAGGCCGCCCAGCGGCAACGCATCGAGGCAGAAGCACGCCCCGGTGAA | |
| | TCGTGGCAAGCGGCCGCTGATCGAATCCGCAAAGAATCCCGGCAACCGCCGGCAGCCGGTGCGCCGT | |
| | CGATTAGGAAGCCGCCCAAGGGCGACGAGCAACCAGATTTTTTCGTTCCGATGCTCTATGACGTGGG | |
| | CACCCGCGATAGTCGCAGCATCATGGACGTGGCCGTTTTCCGTCTGTCGAAGCGTGACCGACGAGCT | |
| | GGCGAGGTGATCCGCTACGAGCTTCCAGACGGGCACGTAGAGGTTTCCGCAGGGCCGGCCGGCATGG | |
| | CCAGTGTGTGGGATTACGACCTGGTACTGATGGCGGTTTCCCATCTAACCGAATCCATGAACCGATA | |
| | CCGGGAAGGGAAGGGAGACAAGCCCGGCCGCGTGTTCCGTCCACACGTTGCGGACGTACTCAAGTTC | |
| | TGCCGGCGAGCCGATGGCGGAAAGCAGAAAGACGACCTGGTAGAAACCTGCATTCGGTTAAACACCA | |
| | CGCACGTTGCCATGCAGCGTACGAAGAAGGCCAAGAACGGCCGCCTGGTGACGGTATCCGAGGGTGA | |
| | AGCCTTGATTAGCCGCTACAAGATCGTAAAGAGCGAAACCGGGCGGCCGGAGTACATCGAGATCGAG | |
| | CTAGCTGATTGGATGTACCGCGAGATCACAGAAGGCAAGAACCCGGACGTGCTGACGGTTCACCCCG | |
| | ATTACTTTTTGATCGATCCCGGCATCGGCCGTTTTCTCTACCGCCTGGCACGCCGCGCCGCAGGCAA | |
| | GGCAGAAGCCAGATGGTTGTTCAAGACGATCTACGAACGCAGTGGCAGCGCCGGAGAGTTCAAGAAG | |
| | TTCTGTTTCACCGTGCGCAAGCTGATCGGGTCAAATGACCTGCCGGAGTACGATTTGAAGGAGGAGG | |
| | CGGGGCAGGCTGGCCCGATCCTAGTCATGCGCTACCGCAACCTGATCGAGGGCGAAGCATCCGCCGG | |
| | TTCCTAATGTACGGAGCAGATGCTAGGGCAAATTGCCCTAGCAGGGGAAAAAGGTCGAAAAGGCCTC | |
| | TTTCCTGTGGATAGCACGTACATTGGGAACCCAAAGCCGTACATTGGGAACCGGAACCCGTACATTG | |
| | GGAACCCAAAGCCGTACATTGGGAACCGGTCACACATGTAAGTGACTGATATAAAAGAGAAAAAAGG | |
| | CGATTTTTCCGCCTAAAACTCTTTAAAACTTATTAAAACTCTTAAAACCCGCCTGGCCTGTGCATAA | |
| | CTGTCTGGCCAGCGCACAGCCCAAGAGCTGCAAAAAGCGCCTACCCTTCGGTCGCTGCGCTCCCTAC | |
| | GCCCCGCCGCTTCGCGTCGGCCTATCGCGGCCGCTGGCCGCTCAAAAATGGCTGGCCTACGGCCAGG | |
| | CAATCTACCAGGGCGCGGACAAGCCGCGCCGTCGCCACTCGACCGCCGGCGCCCACATCAAGGCACC | |
| | CTGCCTCGCGCGTTTCGGTGATGACGGTGAAAACCTCTGACACATGCAGCTCCCGGAAACGGTCACA | |
| | GCTTGTCTGTAAGCGGATGCCGGGAGCAGACAAGCCCGTCAGGGCGCGTCAGCGGGTGTTGGCGGGT | |
| | GTCGGGGCGCAGCCATGACCCAGTCACGTAGCGATAGCGGAGTGTATACTGGCTTAACTATGCGGCA | |
| | TCAGAGCAGATTGTACTGAGAGTGCACCATATGCGGTGTGAAATACCGCACAGATGCGTAAGGAGAA | |
| | AATACCGCATCAGGCCCTCTTCCGCTTCCTCGCTCACTGACTCGCTGCGCTCGGTCGTTCGGCTGCG | |
| | GCGAGCGGTATCAGCTCACTCAAAGGCGGTAATACGGTTATCCACAGAATCAGGGGATAACGCAGGA | |
| | AAGAACATGTGAGCAAAAGGCCAGCAAAAGGCCAGGAACCGTAAAAAGGCCGCGTTGCTGGCGTTTT | |
| | TCCATAGGCTCCGCCCCCCTGACGAGCATCACAAAAATCGACGCTCAAGTCAGAGGTGGCGAAACCC | |
| | GACAGGACTATAAAGATACCAGGCGTTTCCCCCTGGAAGCTCCCTCGTGCGCTCTCCTGTTCCGACC | |
| | CTGCCGCTTACCGGATACCTGTCCGCCTTTCTCCCTTCGGGAAGCGTGGCGCTTTCTCATAGCTCAC | |
| | GCTGTAGGTATCTCAGTTCGGTGTAGGTCGTTCGCTCCAAGCTGGGCTGTGTGCACGAACCCCCCGT | |
| | TCAGCCCGACCGCTGCGCCTTATCCGGTAACTATCGTCTTGAGTCCAACCCGGTAAGACACGACTTA | |
| | TCGCCACTGGCAGCAGCCACTGGTAACAGGATTAGCAGAGCGAGGTAT | |
| 13 | ATGCCTGGCAGTTCCCTACTCTCGCGTTAACGCTTGCATGGATGTTTTCCCAGTCA | pMCS371 |
| | CGACGTTGTAAAACGACGGCCAGTCTTAAGCTCGGGCCCCAAATAATGATTTTATT | Active Cas9- |
| | TTGACTGATAGTGACCTGTTCGTTGCAACAAATTGATGAGCAATGCTTTTTTATAA | Act3.0 |
| | TGCCAACTTTGTACAAAAAAGCAGGCTCCGAATTCGCCCTTCACCATGGATTACAA | intermediate |
| | GGACCACGACGGGGATTACAAGGACCACGACATTGATTACAAGGATGATGATGACA | plasmid |
| | AGATGGCTCCGAAGAAGAAGAGGAAGGTTGGCATCCACGGGGTGCCAGCTGCTGAC | |
| | AAGAAGTACTCGATCGGCCTCGACATTGGGACTAACTCTGTTGGCTGGGCCGTGAT | |
| | CACCGACGAGTACAAGGTGCCCTCAAAGAAGTTCAAGGTCCTGGGCAACACCGATC | |
| | GGCATTCCATCAAGAAGAATCTCATTGGCGCTCTCCTGTTCGACAGCGGCGAGACG | |
| | GCTGAGGCTACGCGGCTCAAGCGCACCGCCCGCAGGCGGTACACGCGCAGGAAGAA | |
| | TCGCATCTGCTACCTGCAGGAGATTTTCTCCAACGAGATGGCGAAGGTTGACGATT | |
| | CTTTCTTCCACAGGCTGGAGGAGTCATTCCTCGTGGAGGAGGATAAGAAGCACGAG | |
| | CGGCATCCAATCTTCGGCAACATTGTCGACGAGGTTGCCTACCACGAGAAGTACCC | |
| | TACGATCTACCATCTGCGGAAGAAGCTCGTGGACTCCACAGATAAGGCGGACCTCC | |
| | GCCTGATCTACCTCGCTCTGGCCCACATGATTAAGTTCAGGGGCCATTTCCTGATC | |
| | GAGGGGGATCTCAACCCGGACAATAGCGATGTTGACAAGCTGTTCATCCAGCTCGT | |
| | GCAGACGTACAACCAGCTCTTCGAGGAGAACCCCATTAATGCGTCAGGCGTCGACG | |
| | CGAAGGCTATCCTGTCCGCTAGGCTCTCGAAGTCTCGGCGCCTCGAGAACCTGATC | |
| | GCCCAGCTGCCGGGCGAGAAGAAGAACGGCCTGTTCGGGAATCTCATTGCGCTCAG | |
| | CCTGGGGCTCACGCCCAACTTCAAGTCGAATTTCGATCTCGCTGAGGACGCCAAGC | |
| | TGCAGCTCTCCAAGGACACATACGACGATGACCTGGATAACCTCCTGGCCCAGATC | |
| | GGCGATCAGTACGCGGACCTGTTCCTCGCTGCCAAGAATCTGTCGGACGCCATCCT | |
| | CCTGTCTGATATTCTCAGGGTGAACACCGAGATTACGAAGGCTCCGCTCTCAGCCT | |
| | CCATGATCAAGCGCTACGACGAGCACCATCAGGATCTGACCCTCCTGAAGGCGCTG | |
| | GTCAGGCAGCAGCTCCCCGAGAAGTACAAGGAGATCTTCTTCGATCAGTCGAAGAA | |
| | CGGCTACGCTGGGTACATTGACGGCGGGGCCTCTCAGGAGGAGTTCTACAAGTTCA | |
| | TCAAGCCGATTCTGGAGAAGATGGACGGCACGGAGGAGCTGCTGGTGAAGCTCAAT | |
| | CGCGAGGACCTCCTGAGGAAGCAGCGGACATTCGATAACGGCAGCATCCCACACCA | |
| | GATTCATCTCGGGGAGCTGCACGCTATCCTGAGGAGGCAGGAGGACTTCTACCCTT | |
| | TCCTCAAGGATAACCGCGAGAAGATCGAGAAGATTCTGACTTTCAGGATCCCGTAC | |
| | TACGTCGGCCCACTCGCTAGGGGCAACTCCCGCTTCGCTTGGATGACCCGCAAGTC | |
| | AGAGGAGACGATCACGCCGTGGAACTTCGAGGAGGTGGTCGACAAGGGCGCTAGCG | |
| | CTCAGTCGTTCATCGAGAGGATGACGAATTTCGACAAGAACCTGCCAAATGAGAAG | |
| | GTGCTCCCTAAGCACTCGCTCCTGTACGAGTACTTCACAGTCTACAACGAGCTGAC | |
| | TAAGGTGAAGTATGTGACCGAGGGCATGAGGAAGCCGGCTTTCCTGTCTGGGGAGC | |
| | AGAAGAAGGCCATCGTGGACCTCCTGTTCAAGACCAACCGGAAGGTCACGGTTAAG | |
| | CAGCTCAAGGAGGACTACTTCAAGAAGATTGAGTGCTTCGATTCGGTCGAGATCTC | |
| | TGGCGTTGAGGACCGCTTCAACGCCTCCCTGGGGACCTACCACGATCTCCTGAAGA | |
| | TCATTAAGGATAAGGACTTCCTGGACAACGAGGAGAATGAGGATATCCTCGAGGAC | |
| | ATTGTGCTGACACTCACTCTGTTCGAGGACCGGGAGATGATCGAGGAGCGCCTGAA | |
| | GACTTACGCCCATCTCTTCGATGACAAGGTCATGAAGCAGCTCAAGAGGAGGAGGT | |
| | ACACCGGCTGGGGGAGGCTGAGCAGGAAGCTCATCAACGGCATTCGGGACAAGCAG | |
| | TCCGGGAAGACGATCCTCGACTTCCTGAAGAGCGATGGCTTCGCGAACCGCAATTT | |
| | CATGCAGCTGATTCACGATGACAGCCTCACATTCAAGGAGGATATCCAGAAGGCTC | |
| | AGGTGAGCGGCCAGGGGGACTCGCTGCACGAGCATATCGCGAACCTCGCTGGCTCG | |
| | CCAGCTATCAAGAAGGGGATTCTGCAGACCGTGAAGGTTGTGGACGAGCTGGTGAA | |
| | GGTCATGGGCAGGCACAAGCCTGAGAACATCGTCATTGAGATGGCCCGGGAGAATC | |
| | AGACCACGCAGAAGGGCCAGAAGAACTCACGCGAGAGGATGAAGAGGATCGAGGAG | |
| | GGCATTAAGGAGCTGGGGTCCCAGATCCTCAAGGAGCACCCGGTGGAGAACACGCA | |
| | GCTGCAGAATGAGAAGCTCTACCTGTACTACCTCCAGAATGGCCGCGATATGTATG | |
| | TGGACCAGGAGCTGGATATTAACAGGCTCAGCGATTACGACGTCGATCACATCGTT | |
| | CCACAGTCATTCCTGAAGGATGACTCCATTGACAACAAGGTCCTCACCAGGTCGGA | |
| | CAAGAACCGGGGCAAGTCTGATAATGTTCCTTCAGAGGAGGTCGTTAAGAAGATGA | |
| | AGAACTACTGGCGCCAGCTCCTGAATGCCAAGCTGATCACGCAGCGGAAGTTCGAT | |
| | AACCTCACAAAGGCTGAGAGGGGGGGGCTCTCTGAGCTGGACAAGGCGGGCTTCAT | |
| | CAAGAGGCAGCTGGTCGAGACACGGCAGATCACTAAGCACGTTGCGCAGATTCTCG | |
| | ACTCACGGATGAACACTAAGTACGATGAGAATGACAAGCTGATCCGCGAGGTGAAG | |
| | GTCATCACCCTGAAGTCAAAGCTCGTCTCCGACTTCAGGAAGGATTTCCAGTTCTA | |
| | CAAGGTTCGGGAGATCAACAATTACCACCATGCCCATGACGCGTACCTGAACGCGG | |
| | TGGTCGGCACAGCTCTGATCAAGAAGTACCCAAAGCTCGAGAGCGAGTTCGTGTAC | |
| | GGGGACTACAAGGTTTACGATGTGAGGAAGATGATCGCCAAGTCGGAGCAGGAGAT | |
| | TGGCAAGGCTACCGCCAAGTACTTCTTCTACTCTAACATTATGAATTTCTTCAAGA | |
| | CAGAGATCACTCTGGCCAATGGCGAGATCCGGAAGCGCCCCCTCATCGAGACGAAC | |
| | GGCGAGACGGGGGAGATCGTGTGGGACAAGGGCAGGGATTTCGCGACCGTCAGGAA | |
| | GGTTCTCTCCATGCCACAAGTGAATATCGTCAAGAAGACAGAGGTCCAGACTGGCG | |
| | GGTTCTCTAAGGAGTCAATTCTGCCTAAGCGGAACAGCGACAAGCTCATCGCCCGC | |
| | AAGAAGGACTGGGATCCGAAGAAGTACGGCGGGTTCGACAGCCCCACTGTGGCCTA | |
| | CTCGGTCCTGGTTGTGGCGAAGGTTGAGAAGGGCAAGTCCAAGAAGCTCAAGAGCG | |
| | TGAAGGAGCTGCTGGGGATCACGATTATGGAGCGCTCCAGCTTCGAGAAGAACCCG | |
| | ATCGATTTCCTGGAGGCGAAGGGCTACAAGGAGGTGAAGAAGGACCTGATCATTAA | |
| | GCTCCCCAAGTACTCACTCTTCGAGCTGGAGAACGGCAGGAAGCGGATGCTGGCTT | |
| | CCGCTGGCGAGCTGCAGAAGGGGAACGAGCTGGCTCTGCCGTCCAAGTATGTGAAC | |
| | TTCCTCTACCTGGCCTCCCACTACGAGAAGCTCAAGGGCAGCCCCGAGGACAACGA | |
| | GCAGAAGCAGCTGTTCGTCGAGCAGCACAAGCATTACCTCGACGAGATCATTGAGC | |
| | AGATTTCCGAGTTCTCCAAGCGCGTGATCCTGGCCGACGCGAATCTGGATAAGGTC | |
| | CTCTCCGCGTACAACAAGCACCGCGACAAGCCAATCAGGGAGCAGGCTGAGAATAT | |
| | CATTCATCTCTTCACCCTGACGAACCTCGGCGCCCCTGCTGCTTTCAAGTACTTCG | |
| | ACACAACTATCGATCGCAAGAGGTACACAAGCACTAAGGAGGTCCTGGACGCGACC | |
| | CTCATCCACCAGTCGATTACCGGCCTCTACGAGACGCGCATCGACCTGTCTCAGCT | |
| | CGGGGGCGACAAGCGGCCAGCGGCGACGAAGAAGGCGGGGCAGGCGAAGAAGAAGA | |
| | AGAAGGGAGACGGCTCTGGATCGGGGTCGGGTTCTGGCTCAGTCGACGATGCTCTT | |
| | GACGATTTTGACCTCGATATGCTCGACGCTCTTGATGATTTTGATCTCGACATGCT | |
| | CGATGCACTTGATGACTTTGACCTTGACATGCTCGACGCACTCGATGACTTCGACC | |
| | TCGACATGCTTGAGGGCAGAGGAAGTCTTCTAACATGCGGTGACGTGGAGGAGAAT | |
| | CCCGGCCCTATGGCGTCAAATTTCACGCAGTTTGTTTTGGTTGATAACGGCGGGAC | |
| | TGGCGACGTTACAGTAGCTCCATCAAATTTTGCGAACGGAGTCGCTGAGTGGATTA | |
| | GCTCAAATTCAAGGTCCCAGGCCTACAAGGTTACCTGTTCTGTTAGGCAGAGTTCT | |
| | GCGCAAAAAAGAAAATATACCATCAAGGTTGAAGTCCCTAAAGTTGCAACACAAAC | |
| | AGTCGGTGGTGTTGAGCTCCCTGTGGCAGCCTGGAGATCTTACTTAAACATGGAGC | |
| | TAACAATTCCAATATTCGCTACAAACTCTGATTGTGAACTGATTGTTAAGGCGATG | |
| | CAAGGTCTCTTGAAAGATGGAAACCCTATACCGTCCGCTATCGCAGCTAACAGCGG | |
| | TATCTATCCTAAGAAGAAGAGGAAGGTTGGCTCTGGATCGGGGTCGGGTTCTGGCT | |
| | CAGGATCCGGTACCTATCCCTATGACGTGCCCGATTATGCCAGCCTGGGCAGCGGC | |
| | TCCCCCAAGAAAAAACGCAAGGTGGAAGATCCTAAGAAAAAGCGGAAAGTGGACGG | |
| | CATTGGTAGTGGGAGCAACGGCAGCAGCGGATCCAACGGTCCGACTGACGCCGCGG | |
| | AAGAAGAACTTTTGAGCAAGAATTATCATCTTGAGAACGAAGTGGCTCGTCTTAAG | |
| | AAAGGTTCTGGCAGTGGAGAAGAACTGCTTTCAAAGAATTACCACCTGGAAAATGA | |
| | GGTAGCTAGACTGAAAAAGGGGAGCGGAAGTGGGGAGGAGTTGCTGAGCAAAAATT | |
| | ATCATTTGGAGAACGAAGTAGCACGACTAAAGAAAGGGTCCGGATCGGGTGAGGAG | |
| | TTACTCTCGAAAAATTATCATCTCGAAAACGAAGTGGCTCGGCTAAAAAAGGGCAG | |
| | TGGTTCTGGAGAAGAGCTATTATCTAAAAACTACCACCTCGAAAATGAGGTGGCAC | |
| | GCTTAAAAAAGGGAAGTGGCAGTGGTGAAGAGCTACTATCCAAGAATTATCATCTT | |
| | GAGAACGAGGTAGCGCGTTTGAAGAAGGGTTCCGGCTCAGGAGAGGAACTGCTCTC | |
| | GAAGAACTATCATCTTGAAAATGAGGTCGCTCGATTAAAAAAGGGATCGGGCAGTG | |
| | GTGAGGAACTACTTTCAAAGAATTACCACCTCGAAAACGAAGTAGCTCGATTAAAG | |
| | AAAGGTTCAGGGTCGGGTGAAGAATTACTGAGTAAAAATTATCATCTGGAAAATGA | |
| | GGTAGCGAGACTAAAAAAGGGGAGTGGTTCTGGCGAGGAATTGCTATCGAAAAATT | |
| | ATCATCTTGAGAACGAAGTTGCTAGGCTCAAAAAGGGCTCAGGCTCAGGCACCGCG | |
| | GTAAACATAGGTGGTGGAACCGGTCCGATGGATCTACAGCGGCCGCAAGGTGGAGG | |
| | TGGACCCAAGAAGAAGCGCAAGGTGTGAACTAGTTAAAGAGCTTTCGTTCGTATCA | |
| | TCGGTTTCGACAACGTTCGTCAAGTTCAATGCATCAGTTTCATTGCGCACACACCA | |
| | GAATCCTACTGAGTTTGAGTATTATGGCATTGGGAAAACTGTTTTTCTTGTACCAT | |
| | TTGTTGTGCTTGTAATTTACTGTGTTTTTTATTCGGTTTTCGCTATCGAACTGTGA | |
| | AATGGAAATGGATGGAGAAGAGTTAATGAATGATATGGTCCTTTTGTTCATTCTCA | |
| | AATTAATATTATTTGTTTTTTCTCTTATTTGTTGTGTGTTGAATTTGAAATTATAA | |
| | GAGATATGCAAACATTTTGTTTTGAGTAAAAATGTGTCAAATCGTGGCCTCTAATG | |
| | ACCGAAGTTAATATGAGGAGTAAAACACTTGTAGTTGTACCATTATGCTTATTCAC | |
| | TAGGCAACAAATATATTTTCAGACCTAGAAAAGCTGCAAATGTTACTGAATACAAG | |
| | TATGTCCTCTTGTGTTTTAGACATTTATGAACTTTCCTTTATGTAATTTTCCAGAA | |
| | TCCTTGTCAGATTCTAATCATTGCTTTATAATTATAGTTATACTCATGGATTTGTA | |
| | GTTGAGTATGAAAATATTTTTTAATGCATTTTATGACTTGCCCGTACGCTGCAGTG | |
| | CAGCGTGACCCGGTCGTGCCCCTCTCTAGAGATAATGAGCATTGCATGTCTAAGTT | |
| | ATAAAAAATTACCACATATTTTTTTTGTCACACTTGTTTGAAGTGCAGTTTATCTA | |
| | TCTTTATACATATATTTAAACTTTACTCTACGAATAATATAATCTATAGTACTACA | |
| | ATAATATCAGTGTTTTAGAGAATCATATAAATGAACAGTTAGACATGGTCTAAAGG | |
| | ACAATTGAGTATTTTGACAACAGGACTCTACAGTTTTATCTTTTTAGTGTGCATGT | |
| | GTTCTCCTTTTTTTTTGCAAATAGCTTCACCTATATAATACTTCATCCATTTTATT | |
| | AGTACATCCATTTAGGGTTTAGGGTTAATGGTTTTTATAGACTAATTTTTTTAGTA | |
| | CATCTATTTTATTCTATTTTAGCCTCTAAATTAAGAAAACTAAAACTCTATTTTAG | |
| | TTTTTTTATTTAATAATTTAGATATAAAATAGAATAAAATAAAGTGACTAAAAATT | |
| | AAACAAATACCCTTTAAGAAATTAAAAAAACTAAGGAAACATTTTTCTTGTTTCGA | |
| | GTAGATAATGCCAGCCTGTTAAACGCCGTCGACGAGTCTAACGGACACCAACCAGC | |
| | GAACCAGCAGCGTCGCGTCGGGCCAAGCGAAGCAGACGGCACGGCATCTCTGTCGC | |
| | TGCCTCTGGACCCCTCTCGAGAGTTCCGCTCCACCGTTGGACTTGCTCCGCTGTCG | |
| | GCATCCAGAAATTGCGTGGCGGAGCGGCAGACGTGAGCCGGCACGGCAGGCGGCCT | |
| | CCTCCTCCTCTCACGGCACCGGCAGCTACGGGGGATTCCTTTCCCACCGCTCCTTC | |
| | GCTTTCCCTTCCTCGCCCGCCGTAATAAATAGACACCCCCTCCACACCCTCTTTCC | |
| | CCAACCTCGTGTTGTTCGGAGCGCACACACACACAACCAGATCTCCCCCAAATCCA | |
| | CCCGTCGGCACCTCCGCTTCAAGGTACGCCGCTCGTCCTCCCCCCCCCCCCTCTCT | |
| | ACCTTCTCTAGATCGGCGTTCCGGTCCATGGTTAGGGCCCGGTAGTTCTACTTCTG | |
| | TTCATGTTTGTGTTAGATCCGTGTTTGTGTTAGATCCGTGCTGCTAGCGTTCGTAC | |
| | ACGGATGCGACCTGTACGTCAGACACGTTCTGATTGCTAACTTGCCAGTGTTTCTC | |
| | TTTGGGGAATCCTGGGATGGCTCTAGCCGTTCCGCAGACGGGATCGATTTCATGAT | |
| | TTTTTTTGTTTCGTTGCATAGGGTTTGGTTTGCCCTTTTCCTTTATTTCAATATAT | |
| | GCCGTGCACTTGTTTGTCGGGTCATCTTTTCATGCTTTTTTTTGTCTTGGTTGTGA | |
| | TGATGTGGTCTGGTTGGGCGGTCGTTCTAGATCGGAGTAGAATTAATTCTGTTTCA | |
| | AACTACCTGGTGGATTTATTAATTTTGGATCTGTATGTGTGTGCCATACATATTCA | |
| | TAGTTACGAATTGAAGATGATGGATGGAAATATCGATCTAGGATAGGTATACATGT | |
| | TGATGCGGGTTTTACTGATGCATATACAGAGATGCTTTTTGTTCGCTTGGTTGTGA | |
| | TGATGTGGTGTGGTTGGGCGGTCGTTCATTCGTTCTAGATCGGAGTAGAATACTGT | |
| | TTCAAACTACCTGGTGTATTTATTAATTTTGGAACTGTATGTGTGTGTCATACATC | |
| | TTCATAGTTACGAGTTTAAGATGGATGGAAATATCGATCTAGGATAGGTATACATG | |
| | TTGATGTGGGTTTTACTGATGCATATACATGATGGCATATGCAGCATCTATTCATA | |
| | TGCTCTAACCTTGAGTACCTATCTATTATAATAAACAAGTATGTTTTATAATTATT | |
| | TTGATCTTGATATACTTGGATGATGGCATATGCAGCAGCTATATGTGGATTTTTTT | |
| | AGCCCTGCCTTCATACGCTATTTATTTGCTTGGTACTGTTTCTTTTGTCGATGCTC | |
| | ACCCTGTTGTTTGGTGTTACTTCTGCAGGGCGCCATGGGGCCGGACATAGTCATGA | |
| | CACAAAGTCCGAGTTCGCTTTCTGCGAGTGTTGGGGATAGAGTTACGATAACCTGC | |
| | AGATCTAGTACGGGGGCCGTCACCACGTCGAATTACGCGTCATGGGTCCAAGAAAA | |
| | GCCAGGCAAACTGTTTAAGGGTCTTATAGGTGGCACGAACAACCGGGCTCCCGGAG | |
| | TCCCTTCCAGGTTTAGTGGATCACTTATTGGTGACAAGGCAACGCTGACGATATCT | |
| | TCCCTCCAGCCAGAAGATTTCGCGACCTACTTTTGTGCACTTTGGTATTCTAACCA | |
| | CTGGGTCTTTGGCCAAGGGACGAAGGTCGAGCTCAAACGGGGTGGCGGTGGGAGCG | |
| | GCGGGGGAGGATCAGGGGGCGGAGGTTCGAGTGGCGGGGGGAGCGAAGTCAAACTC | |
| | CTCGAGTCCGGTGGAGGATTGGTGCAACCAGGTGGTTCATTGAAGCTCTCGTGTGC | |
| | AGTTTCTGGTTTCTCTTTGACAGACTACGGTGTCAATTGGGTCCGGCAGGCACCGG | |
| | GGCGCGGTCTTGAGTGGATCGGCGTTATCTGGGGCGACGGAATAACAGATTATAAC | |
| | TCTGCTTTGAAGGACAGATTCATCATTAGCAAGGACAACGGGAAGAACACGGTGTA | |
| | CCTCCAGATGAGCAAAGTGCGCAGTGATGATACTGCCCTGTATTACTGCGTCACAG | |
| | GTTTGTTTGATTACTGGGGTCAAGGCACTCTTGTGACAGTCTCTTCGTATCCTTAT | |
| | GACGTGCCCGACTACGCTGGCGGAGGGGGCGGCAGCGGAGGCGGTGGATCCGGAGG | |
| | CGGTGGCTCAGGGGGCGGCGGGTCGCTTGATCCTGGTGGCGGAGGTTCTGGGAGCA | |
| | AAGGAGAGGAGCTCTTTACAGGTGTCGTCCCTATTTTGGTGGAGCTTGATGGAGAT | |
| | GTCAATGGCCATAAATTTTCCGTCAGGGGCGAAGGGGAGGGGGACGCGACTAATGG | |
| | AAAATTGACCCTCAAGTTTATCTGCACGACAGGAAAGTTGCCGGTCCCCTGGCCCA | |
| | CTCTGGTTACCACCCTCACTTATGGGGTCCAGTGCTTTAGCAGATACCCTGATCAT | |
| | ATGAAACGCCATGACTTCTTCAAATCAGCCATGCCAGAGGGTTATGTGCAGGAAAG | |
| | AACTATAAGCTTTAAGGACGATGGGACGTACAAAACGCGGGCAGAGGTTAAGTTTG | |
| | AGGGGGATACTCTTGTGAATAGGATCGAACTTAAGGGCATCGATTTTAAAGAAGAC | |
| | GGTAATATTCTTGGCCACAAGTTGGAGTATAATTTCAATTCTCACAACGTGTACAT | |
| | AACAGCAGATAAACAGAAAAATGGGATCAAGGCCAATTTTAAAATTAGGCATAATG | |
| | TTGAAGATGGGTCTGTGCAACTGGCAGATCATTATCAGCAGAATACTCCAATTGGC | |
| | GATGGCCCTGTCTTGTTGCCTGACAATCATTACCTTTCCACGCAGTCAGTCCTGTC | |
| | CAAAGATCCGAATGAGAAGCGGGACCACATGGTCTTGCTCGAATTCGTTACTGCCG | |
| | CCGGAATCACTCATGGCATGGACGAGCTTTACAAGGGAGGTGGAAGGACTGGAGGT | |
| | GGCGGAGGGGGGCTGCTTGACCCCGGAACCCCTATGGATGCCGACTTGGTGGCCTC | |
| | CTCCACGGTTGTTTGGGAACAGGATGCCGACCCTTTTGCGGGCACTGCTGACGATT | |
| | TTCCCGCTTTCAATGAGGAGGAGTTGGCTTGGCTGATGGAACTTCTCCCACAGGGT | |
| | GGTAGTGGTGGCCTCCTGGATCCGGGGACACCGATGGACGCAGACCTGGTGGCGTC | |
| | GTCAACGGTTGTGTGGGAACAGGATGCTGATCCGTTCGCAGGAACTGCCGACGATT | |
| | TTCCTGCATTCAACGAAGAGGAACTCGCATGGCTCATGGAGCTTCTGCCTCAGGGA | |
| | TCGGGTGGCGGGTCTCGAACGGAAGAATACAAGCTTATATTGAACGGTAAGACACT | |
| | GAAAGGTGAAACTACGACAGAAGCCGTTGACGCGGCAACTGCGGAGAAAGTCTTTA | |
| | AGCAGTATGCTAACGATAATGGCGTGGACGGGGAGTGGACGTATGACGACGCAACC | |
| | AAAACATTCACGGTCACCGAGGGGGGCGGCAGCGGAGGCGGTACCTCGCCCAAAAC | |
| | CCGGAGACGCCCTAGGCGCTCGCAGCGCAAAAGACCTCCCACGTAAAGAGCTTTCG | |
| | TTCGTATCATCGGTTTCGACAACGTTCGTCAAGTTCAATGCATCAGTTTCATTGCG | |
| | CACACACCAGAATCCTACTGAGTTTGAGTATTATGGCATTGGGAAAACTGTTTTTC | |
| | TTGTACCATTTGTTGTGCTTGTAATTTACTGTGTTTTTTATTCGGTTTTCGCTATC | |
| | GAACTGTGAAATGGAAATGGATGGAGAAGAGTTAATGAATGATATGGTCCTTTTGT | |
| | TCATTCTCAAATTAATATTATTTGTTTTTTCTCTTATTTGTTGTGTGTTGAATTTG | |
| | AAATTATAAGAGATATGCAAACATTTTGTTTTGAGTAAAAATGTGTCAAATCGTGG | |
| | CCTCTAATGACCGAAGTTAATATGAGGAGTAAAACACTTGTAGTTGTACCATTATG | |
| | CTTATTCACTAGGCAACAAATATATTTTCAGACCTAGAAAAGCTGCAAATGTTACT | |
| | GAATACAAGTATGTCCTCTTGTGTTTTAGACATTTATGAACTTTCCTTTATGTAAT | |
| | TTTCCAGAATCCTTGTCAGATTCTAATCATTGCTTTATAATTATAGTTATACTCAT | |
| | GGATTTGTAGTTGAGTATGAAAATATTTTTTAATGCATTTTATGACTTGCCAATTG | |
| | TTCCGGAACTCTAGATAAGCTTACCGGAAAGGGCGAATTCGCAACTTTGTATACAA | |
| | AAGTTGAACGAGAAACGTAAAATGATATAAATATCAATATATTAAATTAGATTTTG | |
| | CATAAAAAACAGACTACATAATACTGTAAAACACAACATATCCAGTCACTATGCCA | |
| | TCCAGCTGATATCCCCTATAGTGAGTCGTATTACATGGTCATAGCTGTTTCCTGGC | |
| | AGCTCTGGCCCGTGTCTCAAAATCTCTGATGTTACATTGCACAAGATAAAAATATA | |
| | TCATCATGCCTCCTCTGGACCAGCCAGGACAGAAATGCCTCGACTTCGCTGCTACC | |
| | CAAGGTTGCCGGGTGACGCACACCGTGGAAACGGATGAAGGCACGAACCCAGTGGA | |
| | CATAAGCCTGTTCGGTTCGTAAGCTGTAATGCAAGTAGCGTATGCGCTCACGCAAC | |
| | TGGTCCAGAACCTTGACCGAACGCAGCGGTGGTAACGGCGCAGTGGCGGTTTTCAT | |
| | GGCTTGTTATGACTGTTTTTTTGGGGTACAGTCTATGCCTCGGGCATCCAAGCAGC | |
| | AAGCGCGTTACGCCGTGGGTCGATGTTTGATGTTATGGAGCAGCAACGATGTTACG | |
| | CAGCAGGGCAGTCGCCCTAAAACAAAGTTAAACATTATGAGGGAAGCGGTGATCGC | |
| | CGAAGTATCGACTCAACTATCAGAGGTAGTTGGCGTCATCGAGCGCCATCTCGAAC | |
| | CGACGTTGCTGGCCGTACATTTGTACGGCTCCGCAGTGGATGGCGGCCTGAAGCCA | |
| | CACAGTGATATTGATTTGCTGGTTACGGTGACCGTAAGGCTTGATGAAACAACGCG | |
| | GCGAGCTTTGATCAACGACCTTTTGGAAACTTCGGCTTCCCCTGGAGAGAGCGAGA | |
| | TTCTCCGCGCTGTAGAAGTCACCATTGTTGTGCACGACGACATCATTCCGTGGCGT | |
| | TATCCAGCTAAGCGCGAACTGCAATTTGGAGAATGGCAGCGCAATGACATTCTTGC | |
| | AGGTATCTTCGAGCCAGCCACGATCGACATTGATCTGGCTATCTTGCTGACAAAAG | |
| | CAAGAGAACATAGCGTTGCCTTGGTAGGTCCAGCGGCGGAGGAACTCTTTGATCCG | |
| | GTTCCTGAACAGGATCTATTTGAGGCGCTAAATGAAACCTTAACGCTATGGAACTC | |
| | GCCGCCCGACTGGGCTGGCGATGAGCGAAATGTAGTGCTTACGTTGTCCCGCATTT | |
| | GGTACAGCGCAGTAACCGGCAAAATCGCGCCGAAGGATGTCGCTGCCGACTGGGCA | |
| | ATGGAGCGCCTGCCGGCCCAGTATCAGCCCGTCATACTTGAAGCTAGACAGGCTTA | |
| | TCTTGGACAAGAAGAAGATCGCTTGGCCTCGCGCGCAGATCAGTTGGAAGAATTTG | |
| | TCCACTACGTGAAAGGCGAGATCACCAAGGTAGTCGGCAAATAACCCTCGAGCCAC | |
| | CCATGACCAAAATCCCTTAACGTGAGTTACGCGTCGTTCCACTGAGCGTCAGACCC | |
| | CGTAGAAAAGATCAAAGGATCTTCTTGAGATCCTTTTTTTCTGCGCGTAATCTGCT | |
| | GCTTGCAAACAAAAAAACCACCGCTACCAGCGGTGGTTTGTTTGCCGGATCAAGAG | |
| | CTACCAACTCTTTTTCCGAAGGTAACTGGCTTCAGCAGAGCGCAGATACCAAATAC | |
| | TGTTCTTCTAGTGTAGCCGTAGTTAGGCCACCACTTCAAGAACTCTGTAGCACCGC | |
| | CTACATACCTCGCTCTGCTAATCCTGTTACCAGTGGCTGCTGCCAGTGGCGATAAG | |
| | TCGTGTCTTACCGGGTTGGACTCAAGACGATAGTTACCGGATAAGGCGCAGCGGTC | |
| | GGGCTGAACGGGGGGTTCGTGCACACAGCCCAGCTTGGAGCGAACGACCTACACCG | |
| | AACTGAGATACCTACAGCGTGAGCTATGAGAAAGCGCCACGCTTCCCGAAGGGAGA | |
| | AAGGCGGACAGGTATCCGGTAAGCGGCAGGGTCGGAACAGGAGAGCGCACGAGGGA | |
| | GCTTCCAGGGGGAAACGCCTGGTATCTTTATAGTCCTGTCGGGTTTCGCCACCTCT | |
| | GACTTGAGCGTCGATTTTTGTGATGCTCGTCAGGGGGGCGGAGCCTATGGAAAAAC | |
| | GCCAGCAACGCGGCCTTTTTACGGTTCCTGGCCTTTTGCTGGCCTTTTGCTCACAT | |
| | GTTCTTTCCTGCGTTATCCCCTGATTCTGTGGATAACCGTATTACCGCCTTTGAGT | |
| | GAGCTGATACCGCTCGCCGCAGCCGAACGACCGAGCGCAGCGAGTCAGTGAGCGAG | |
| | GAAGCGGAAGAGCGCCCAATACGCAAACCGCCTCTCCCCGCGCGTTGGCCGATTCA | |
| | TTAATGCAGCTGGCACGACAGGTTTCCCGACTGGAAAGCGGGCAGTGAGCGCAACG | |
| | CAATTAATACGCGTACCGCGAGCCAGGAAGAGTTTGTAGAAACGCAAAAAGGCCAT | |
| | CCGTCAGGATGGCCTTCTGCTTAGTTTGATGCCTGGCAGTTTATGGCGGGCGTCCT | |
| | GCCCGCCACCCTCCGGGCCGTTGCTTCACAACGTTCAAATCCGCTCCCGGCGGATT | |
| | TGTCCTACTCAGGAGAGCGTTCACCGACAAACAACAGATAAAACGAAAGGCCCAGT | |
| | CTTCCGACTGAGCCTTTCGTTTTATTTG | |
| 14 | CTGCATTAATGAATCGGCCAACGCGCGGGGAGAGGCGGTTTGCGTATTGGGCGCTC | pMCS408 |
| | TTCCGCTTCCTCGCTCACTGACTCGCTGCGCTCGGTCGTTCGGCTGCGGCGAGCGG | rice dRNA #1 |
| | TATCAGCTCACTCAAAGGCGGTAATACGGTTATCCACAGAATCAGGGGATAACGCA | entry plasmid |
| | GGAAAGAACATGAATTAATTCTCATGTTTGACAGCTTATCATCGATTAGCTTTAAT | |
| | GCGGTAGTTTATCACAGTTAAATTGCTAACGCAGTCAGGCACCGTGTATGAAATCT | |
| | AACAATGCGCTCATCGTCATCCTCGGCACCGTCACCCTGGATGCTGTAGGCATAGG | |
| | CTTGGTTATGCCGGTACTGCCGGGCCTCTTGCGGGATATCGTCCATTCCGACAGCA | |
| | TCGCCAGTCACTATGGCGTGCTGCTAGCGCTATATGCGTTGATGCAATTTCTATGC | |
| | GCACCCGTTCTCGGAGCACTGTCCGACCGCTTTGGCCGCCGCCCAGTCCTGCTCGC | |
| | TTCGCTACTTGGAGCCACTATCGACTACGCGATCATGGCGACCACACCCGTCCTGT | |
| | GGATTCTCTACGCCGGACGCATCGTGGCCGGCATCACCGGCGCCACAGGTGCGGTT | |
| | GCTGGCGCCTATATCGCCGACATCACCGATGGGGAAGATCGGGCTCGCCACTTCGG | |
| | GCTCATGAGCGCTTGTTTCGGCGTGGGTATGGTGGCAGGCCCCGTGGCCGGGGGAC | |
| | TGTTGGGCGCCATCTCCTTACATGCACCATTCCTTGCGGCGGCGGTGCTCAACGGC | |
| | CTCAACCTACTACTGGGCTGCTTCCTAATGCAGGAGTCGCATAAGGGAGAGCGCCG | |
| | ACCCATGCCCTTGAGAGCCTTCAACCCAGTCAGCTCCTTCCGGTGGGCGCGGGGCA | |
| | TGACTATCGTCGCCGCACTTATGACTGTCTTCTTTATCATGCAACTCGTAGGACAG | |
| | GTGCCGGCAGCGCTCTGGGTCATTTTCGGCGAGGACCGCTTTCGCTGGAGCGCGAC | |
| | GATGATCGGCCTGTCGCTTGCGGTATTCGGAATCTTGCACGCCCTCGCTCAAGCCT | |
| | TCGTCACTGGTCCCGCCACCAAACGTTTCGGCGAGAAGCAGGCCATTATCGCCGGC | |
| | ATGGCGGCCGACGCGCTGGGCTACGTCTTGCTGGCGTTCGCGACGCGAGGCTGGAT | |
| | GGCCTTCCCCATTATGATTCTTCTCGCTTCCGGCGGCATCGGGATGCCCGCGTTGC | |
| | AGGCCATGCTGTCCAGGCAGGTAGATGACGACCATCAGGGACAGCTTCAAGGATCG | |
| | CTCGCGGCTCTTACCAGCCTAACTTCGATCATTGGACCGCTGATCGTCACGGCGAT | |
| | TTATGCCGCCTCGGCGAGCACATGGAACGGGTTGGCATGGATTGTAGGCGCCGCCC | |
| | TATACCTTGTCTGCCTCCCCGCGTTGCGTCGCGGTGCATGGAGCCGGGCCACCTCG | |
| | ACCTGAATGGAAGCCGGCGGCACCTCGCTAACGGATTCACCACTCCAAGAATTGGA | |
| | GCCAATCAATTCTTGCGGAGAACTGTGAATGCGCAAACCAACCCTTGATCGGGGAA | |
| | GAACAGTATGTCGAGCTATTTTTTGACTTACTGGGGATCAAGCCTGATTGGGAGAA | |
| | AATAAAATATCCCCTATAGTGAGTCGTATTACATGGTCATAGCTGTTTCCTGGCAG | |
| | CTCTGGCCCGTGTCTCAAAATCTCTGATGTTACATTGCACAAGATAAAAATATATC | |
| | ATCATGCCTCCTCTAGAGGTCTCGCTATCCAGGCTTCATCCTAACCATTACAGGCA | |
| | AGATGTTGTATGAAGAAGGGCGAACATGCAGATTGTTAAACTGACACGTGATGGAC | |
| | AAGAATGACCGATTGGTGACCGGTCTGACAATGGTCATGTCGTCAGCAGACAGCCA | |
| | TCTCCCACGTCGCGCCTGCTTCCGGTGAAAGTGGAGGTAGGTATGGGCCGTCCCGT | |
| | CAGAAGGTGATTCGGATGGCAGCGATACAAATCTCCGTCCATTAATGAAGAGAAGT | |
| | CAAGTTGAAAGAAAGGGAGGGAGAGATGGTGCATGTGGGATCCCCTTGGGATATAA | |
| | AAGGAGGACCTTGCCCACTTAGAAAGGAGAGGAGAAAGCAATCCCAGAAGAATCGG | |
| | GGGCTGACTGGCACTTTGTAGCTTCTTCATACGCGAATCCACCAAAACACAGGAGT | |
| | AGGGTATTACGCTTCTCAGCGGCCCGAACCTGTATACATCGCCCGTGTCTTGTGTG | |
| | TTTCCGCTCTTGCGAACCTTCCACAGATTGGGAGCTTAGAACCTCACCCAGGGCCC | |
| | CCGGCCGAACTGGCAAAGGGGGGCCTGCGCAGTCTCCCGGTGAGGAGCCCCACGCT | |
| | CCGTCAGTTCTAAATTACCCGATGAGAAAGGGAGGGGCGGGGGAAAATCTGCCTTG | |
| | TTTATTTACGATCCAACGGATTTGGTCGACACCGATGAGGTGTCTTACCAGTTACC | |
| | ACGAGCTAGATTATAGTACTAATTACTTGAGGATTCGGTTCCTAATTTTTTACCCG | |
| | ATCGACTTCGCCATGGAAAATTTTTTATTCGGGGGAGAATATCCACCCTGTTTCGC | |
| | TCCTAATTAAGATAGGAATTGTTACGATTAGCAACCTAATTCAGATCAGAATTGTT | |
| | AGTTAGCGGCGTTGGATCCCTCACCTCATCCCATCCCAATTCCCAAACCCAAACTC | |
| | CTCTTCCAGTCGCCGACCCAAACACGCATCCGCCGCCTATAAATCCCACCCGCATC | |
| | GAGCCTATCAAGCCCAAAAAACCACAAACCCAACGAAGAAGGAAAAAAAAAGGAGG | |
| | AAAAGAAAAGAGGAGGAAAGCGAAGAGGTTGGAGAGAGACACTAGTCTCCACGTCG | |
| | CCGCCAACAAAGCACCAGTGGTCTAGTGGTAGAATAGTACCCTGCCACGGTACAGA | |
| | CCCGGGTTCGATTCCCGGCTGGTGCAAGAGACGAGATCTAGTCTGAGTCGACCGTC | |
| | TCTGTTTTAGAGCTAGGCCAACATGAGGATCACCCATGTCTGCAGGGCCTAGCAAG | |
| | TTAAAATAAGGCTAGTCCGTTATCAACTTGGCCAACATGAGGATCACCCATGTCTG | |
| | CAGGGCCAAGTGGCACCGAGTCGGTGCAACAAAGCACCAGTGGTCTAGTGGTAGAA | |
| | TAGTACCCTGCCACGGTACAGACCCGGGTTCGATTCCCGGCTGGTGCAATATGAAG | |
| | ATGAAGATGAAATATTTGGTGTGTCAAATAAAAAGCTTGTGTGCTTAAGTTTGTGT | |
| | TTTTTTCTTGGCTTGTTGTGTTATGAATTTGTGGCTTTTTCTAATATTAAATGAAT | |
| | GTAAGATCACATTATAATGAATAAACAAATGTTTCTATAATCCATTGTGAATGTTT | |
| | TGTTGGATCTCTTCTGCAGCATATAACTACTGTATGTGCTATGGTATGGACTATGG | |
| | AATATGATTAAAGATAAGCATGGGAGACCCTCGAGCCACCCATGACCAAAATCCCT | |
| | TAACGTGAGTTACGCGTCGTTCCACTGAGCGTCAGACCCCGTAGAAAAGATCAAAG | |
| | GATCTTCTTGAGATCCTTTTTTTCTGCGCGTAATCTGCTGCTTGCAAACAAAAAAA | |
| | CCACCGCTACCAGCGGTGGTTTGTTTGCCGGATCAAGAGCTACCAACTCTTTTTCC | |
| | GAAGGTAACTGGCTTCAGCAGAGCGCAGATACCAAATACTGTTCTTCTAGTGTAGC | |
| | CGTAGTTAGGCCACCACTTCAAGAACTCTGTAGCACCGCCTACATACCTCGCTCTG | |
| | CTAATCCTGTTACCAGTGGCTGCTGCCAGTGGCGATAAGTCGTGTCTTACCGGGTT | |
| | GGACTCAAGACGATAGTTACCGGATAAGGCGCAGCGGTCGGGCTGAACGGGGGGTT | |
| | CGTGCACACAGCCCAGCTTGGAGCGAACGACCTACACCGAACTGAGATACCTACAG | |
| | CGTGAGCTATGAGAAAGCGCCACGCTTCCCGAAGGGAGAAAGGCGGACAGGTATCC | |
| | GGTAAGCGGCAGGGTCGGAACAGGAGAGCGCACGAGGGAGCTTCCAGGGGGAAACG | |
| | CCTGGTATCTTTATAGTCCTGTCGGGTTTCGCCACCTCTGACTTGAGCGTCGATTT | |
| | TTGTGATGCTCGTCAGGGGGGCGGAGCCTATGGAAAAACGCCAGCAACGCGGCCTT | |
| | TTTACGGTTCCTGGCCTTTTGCTGGCCTTTTGCTCACATGTTCTTTCCTGCGTTAT | |
| | CCCCTGATTCTGTGGATAACCGTATTACCGCCTTTGAGTGAGCTGATACCGCTCGC | |
| | CGCAGCCGAACGACCGAGCGCAGCGAGTCAGTGAGCGAGGAAGCGGAAGAGCGCCC | |
| | AATACGCAAACCGCCTCTCCCCGCGCGTTGGCCGATTCATTAATGCAGCTGGCACG | |
| | ACAGGTTTCCCGACTGGAAAGCGGGCAGTGAGCGCAACGCAATTAATACGCGTACC | |
| | GCTAGCCAGGAAGAGTTTGTAGAAACGCAAAAAGGCCATCCGTCAGGATGGCCTTC | |
| | TGCTTAGTTTGATGCCTGGCAGTTTATGGCGGGCGTCCTGCCCGCCACCCTCCGGG | |
| | CCGTTGCTTCACAACGTTCAAATCCGCTCCCGGCGGATTTGTCCTACTCAGGAGAG | |
| | CGTTCACCGACAAACAACAGATAAAACGAAAGGCCCAGTCTTCCGACTGAGCCTTT | |
| | CGTTTTATTTGATGCCTGGCAGTTCCCTACTCTCGCGTT | |
| 15 | CTGCATTAATGAATCGGCCAACGCGCGGGGAGAGGCGGTTTGCGTATTGGGCGCTCTTCCGCTTCCT | pMCS409 |
| | CGCTCACTGACTCGCTGCGCTCGGTCGTTCGGCTGCGGCGAGCGGTATCAGCTCACTCAAAGGCGGT | rice dRNA #2 |
| | AATACGGTTATCCACAGAATCAGGGGATAACGCAGGAAAGAACATGAATTAATTCTCATGTTTGACA | entry plasmid |
| | GCTTATCATCGATTAGCTTTAATGCGGTAGTTTATCACAGTTAAATTGCTAACGCAGTCAGGCACCG | |
| | TGTATGAAATCTAACAATGCGCTCATCGTCATCCTCGGCACCGTCACCCTGGATGCTGTAGGCATAG | |
| | GCTTGGTTATGCCGGTACTGCCGGGCCTCTTGCGGGATATCGTCCATTCCGACAGCATCGCCAGTCA | |
| | CTATGGCGTGCTGCTAGCGCTATATGCGTTGATGCAATTTCTATGCGCACCCGTTCTCGGAGCACTG | |
| | TCCGACCGCTTTGGCCGCCGCCCAGTCCTGCTCGCTTCGCTACTTGGAGCCACTATCGACTACGCGA | |
| | TCATGGCGACCACACCCGTCCTGTGGATTCTCTACGCCGGACGCATCGTGGCCGGCATCACCGGCGC | |
| | CACAGGTGCGGTTGCTGGCGCCTATATCGCCGACATCACCGATGGGGAAGATCGGGCTCGCCACTTC | |
| | GGGCTCATGAGCGCTTGTTTCGGCGTGGGTATGGTGGCAGGCCCCGTGGCCGGGGGACTGTTGGGCG | |
| | CCATCTCCTTACATGCACCATTCCTTGCGGCGGCGGTGCTCAACGGCCTCAACCTACTACTGGGCTG | |
| | CTTCCTAATGCAGGAGTCGCATAAGGGAGAGCGCCGACCCATGCCCTTGAGAGCCTTCAACCCAGTC | |
| | AGCTCCTTCCGGTGGGCGCGGGGCATGACTATCGTCGCCGCACTTATGACTGTCTTCTTTATCATGC | |
| | AACTCGTAGGACAGGTGCCGGCAGCGCTCTGGGTCATTTTCGGCGAGGACCGCTTTCGCTGGAGCGC | |
| | GACGATGATCGGCCTGTCGCTTGCGGTATTCGGAATCTTGCACGCCCTCGCTCAAGCCTTCGTCACT | |
| | GGTCCCGCCACCAAACGTTTCGGCGAGAAGCAGGCCATTATCGCCGGCATGGCGGCCGACGCGCTGG | |
| | GCTACGTCTTGCTGGCGTTCGCGACGCGAGGCTGGATGGCCTTCCCCATTATGATTCTTCTCGCTTC | |
| | CGGCGGCATCGGGATGCCCGCGTTGCAGGCCATGCTGTCCAGGCAGGTAGATGACGACCATCAGGGA | |
| | CAGCTTCAAGGATCGCTCGCGGCTCTTACCAGCCTAACTTCGATCATTGGACCGCTGATCGTCACGG | |
| | CGATTTATGCCGCCTCGGCGAGCACATGGAACGGGTTGGCATGGATTGTAGGCGCCGCCCTATACCT | |
| | TGTCTGCCTCCCCGCGTTGCGTCGCGGTGCATGGAGCCGGGCCACCTCGACCTGAATGGAAGCCGGC | |
| | GGCACCTCGCTAACGGATTCACCACTCCAAGAATTGGAGCCAATCAATTCTTGCGGAGAACTGTGAA | |
| | TGCGCAAACCAACCCTTGATCGGGGAAGAACAGTATGTCGAGCTATTTTTTGACTTACTGGGGATCA | |
| | AGCCTGATTGGGAGAAAATAAAATATCCCCTATAGTGAGTCGTATTACATGGTCATAGCTGTTTCCT | |
| | GGCAGCTCTGGCCCGTGTCTCAAAATCTCTGATGTTACATTGCACAAGATAAAAATATATCATCATG | |
| | CCTCCTCTAGAGGTCTCCCATGCCAGGCTTCATCCTAACCATTACAGGCAAGATGTTGTATGAAGAA | |
| | GGGCGAACATGCAGATTGTTAAACTGACACGTGATGGACAAGAATGACCGATTGGTGACCGGTCTGA | |
| | CAATGGTCATGTCGTCAGCAGACAGCCATCTCCCACGTCGCGCCTGCTTCCGGTGAAAGTGGAGGTA | |
| | GGTATGGGCCGTCCCGTCAGAAGGTGATTCGGATGGCAGCGATACAAATCTCCGTCCATTAATGAAG | |
| | AGAAGTCAAGTTGAAAGAAAGGGAGGGAGAGATGGTGCATGTGGGATCCCCTTGGGATATAAAAGGA | |
| | GGACCTTGCCCACTTAGAAAGGAGAGGAGAAAGCAATCCCAGAAGAATCGGGGGCTGACTGGCACTT | |
| | TGTAGCTTCTTCATACGCGAATCCACCAAAACACAGGAGTAGGGTATTACGCTTCTCAGCGGCCCGA | |
| | ACCTGTATACATCGCCCGTGTCTTGTGTGTTTCCGCTCTTGCGAACCTTCCACAGATTGGGAGCTTA | |
| | GAACCTCACCCAGGGCCCCCGGCCGAACTGGCAAAGGGGGGCCTGCGCAGTCTCCCGGTGAGGAGCC | |
| | CCACGCTCCGTCAGTTCTAAATTACCCGATGAGAAAGGGAGGGGCGGGGGAAAATCTGCCTTGTTTA | |
| | TTTACGATCCAACGGATTTGGTCGACACCGATGAGGTGTCTTACCAGTTACCACGAGCTAGATTATA | |
| | GTACTAATTACTTGAGGATTCGGTTCCTAATTTTTTACCCGATCGACTTCGCCATGGAAAATTTTTT | |
| | ATTCGGGGGAGAATATCCACCCTGTTTCGCTCCTAATTAAGATAGGAATTGTTACGATTAGCAACCT | |
| | AATTCAGATCAGAATTGTTAGTTAGCGGCGTTGGATCCCTCACCTCATCCCATCCCAATTCCCAAAC | |
| | CCAAACTCCTCTTCCAGTCGCCGACCCAAACACGCATCCGCCGCCTATAAATCCCACCCGCATCGAG | |
| | CCTATCAAGCCCAAAAAACCACAAACCCAACGAAGAAGGAAAAAAAAAGGAGGAAAAGAAAAGAGGA | |
| | GGAAAGCGAAGAGGTTGGAGAGAGACACTAGTCTCCACGTCGCCGCCAACAAAGCACCAGTGGTCTA | |
| | GTGGTAGAATAGTACCCTGCCACGGTACAGACCCGGGTTCGATTCCCGGCTGGTGCAAGAGACGAGA | |
| | TCTAGTCTGAGTCGACCGTCTCTGTTTTAGAGCTAGGCCAACATGAGGATCACCCATGTCTGCAGGG | |
| | CCTAGCAAGTTAAAATAAGGCTAGTCCGTTATCAACTTGGCCAACATGAGGATCACCCATGTCTGCA | |
| | GGGCCAAGTGGCACCGAGTCGGTGCAACAAAGCACCAGTGGTCTAGTGGTAGAATAGTACCCTGCCA | |
| | CGGTACAGACCCGGGTTCGATTCCCGGCTGGTGCAATATGAAGATGAAGATGAAATATTTGGTGTGT | |
| | CAAATAAAAAGCTTGTGTGCTTAAGTTTGTGTTTTTTTCTTGGCTTGTTGTGTTATGAATTTGTGGC | |
| | TTTTTCTAATATTAAATGAATGTAAGATCACATTATAATGAATAAACAAATGTTTCTATAATCCATT | |
| | GTGAATGTTTTGTTGGATCTCTTCTGCAGCATATAACTACTGTATGTGCTATGGTATGGACTATGGA | |
| | ATATGATTAAAGATAAGGGACGGAGACCCTCGAGCCACCCATGACCAAAATCCCTTAACGTGAGTTA | |
| | CGCGTCGTTCCACTGAGCGTCAGACCCCGTAGAAAAGATCAAAGGATCTTCTTGAGATCCTTTTTTT | |
| | CTGCGCGTAATCTGCTGCTTGCAAACAAAAAAACCACCGCTACCAGCGGTGGTTTGTTTGCCGGATC | |
| | AAGAGCTACCAACTCTTTTTCCGAAGGTAACTGGCTTCAGCAGAGCGCAGATACCAAATACTGTTCT | |
| | TCTAGTGTAGCCGTAGTTAGGCCACCACTTCAAGAACTCTGTAGCACCGCCTACATACCTCGCTCTG | |
| | CTAATCCTGTTACCAGTGGCTGCTGCCAGTGGCGATAAGTCGTGTCTTACCGGGTTGGACTCAAGAC | |
| | GATAGTTACCGGATAAGGCGCAGCGGTCGGGCTGAACGGGGGGTTCGTGCACACAGCCCAGCTTGGA | |
| | GCGAACGACCTACACCGAACTGAGATACCTACAGCGTGAGCTATGAGAAAGCGCCACGCTTCCCGAA | |
| | GGGAGAAAGGCGGACAGGTATCCGGTAAGCGGCAGGGTCGGAACAGGAGAGCGCACGAGGGAGCTTC | |
| | CAGGGGGAAACGCCTGGTATCTTTATAGTCCTGTCGGGTTTCGCCACCTCTGACTTGAGCGTCGATT | |
| | TTTGTGATGCTCGTCAGGGGGGCGGAGCCTATGGAAAAACGCCAGCAACGCGGCCTTTTTACGGTTC | |
| | CTGGCCTTTTGCTGGCCTTTTGCTCACATGTTCTTTCCTGCGTTATCCCCTGATTCTGTGGATAACC | |
| | GTATTACCGCCTTTGAGTGAGCTGATACCGCTCGCCGCAGCCGAACGACCGAGCGCAGCGAGTCAGT | |
| | GAGCGAGGAAGCGGAAGAGCGCCCAATACGCAAACCGCCTCTCCCCGCGCGTTGGCCGATTCATTAA | |
| | TGCAGCTGGCACGACAGGTTTCCCGACTGGAAAGCGGGCAGTGAGCGCAACGCAATTAATACGCGTA | |
| | CCGCTAGCCAGGAAGAGTTTGTAGAAACGCAAAAAGGCCATCCGTCAGGATGGCCTTCTGCTTAGTT | |
| | TGATGCCTGGCAGTTTATGGCGGGCGTCCTGCCCGCCACCCTCCGGGCCGTTGCTTCACAACGTTCA | |
| | AATCCGCTCCCGGCGGATTTGTCCTACTCAGGAGAGCGTTCACCGACAAACAACAGATAAAACGAAA | |
| | GGCCCAGTCTTCCGACTGAGCCTTTCGTTTTATTTGATGCCTGGCAGTTCCCTACTCTCGCGTT | |
| 16 | CTGCATTAATGAATCGGCCAACGCGCGGGGAGAGGCGGTTTGCGTATTGGGCGCTC | pMCS410 |
| | TTCCGCTTCCTCGCTCACTGACTCGCTGCGCTCGGTCGTTCGGCTGCGGCGAGCGG | Arabidopsis |
| | TATCAGCTCACTCAAAGGCGGTAATACGGTTATCCACAGAATCAGGGGATAACGCA | dRNA #1 entry |
| | GGAAAGAACATGAATTAATTCTCATGTTTGACAGCTTATCATCGATTAGCTTTAAT | plasmid |
| | GCGGTAGTTTATCACAGTTAAATTGCTAACGCAGTCAGGCACCGTGTATGAAATCT | |
| | AACAATGCGCTCATCGTCATCCTCGGCACCGTCACCCTGGATGCTGTAGGCATAGG | |
| | CTTGGTTATGCCGGTACTGCCGGGCCTCTTGCGGGATATCGTCCATTCCGACAGCA | |
| | TCGCCAGTCACTATGGCGTGCTGCTAGCGCTATATGCGTTGATGCAATTTCTATGC | |
| | GCACCCGTTCTCGGAGCACTGTCCGACCGCTTTGGCCGCCGCCCAGTCCTGCTCGC | |
| | TTCGCTACTTGGAGCCACTATCGACTACGCGATCATGGCGACCACACCCGTCCTGT | |
| | GGATTCTCTACGCCGGACGCATCGTGGCCGGCATCACCGGCGCCACAGGTGCGGTT | |
| | GCTGGCGCCTATATCGCCGACATCACCGATGGGGAAGATCGGGCTCGCCACTTCGG | |
| | GCTCATGAGCGCTTGTTTCGGCGTGGGTATGGTGGCAGGCCCCGTGGCCGGGGGAC | |
| | TGTTGGGCGCCATCTCCTTACATGCACCATTCCTTGCGGCGGCGGTGCTCAACGGC | |
| | CTCAACCTACTACTGGGCTGCTTCCTAATGCAGGAGTCGCATAAGGGAGAGCGCCG | |
| | ACCCATGCCCTTGAGAGCCTTCAACCCAGTCAGCTCCTTCCGGTGGGCGCGGGGCA | |
| | TGACTATCGTCGCCGCACTTATGACTGTCTTCTTTATCATGCAACTCGTAGGACAG | |
| | GTGCCGGCAGCGCTCTGGGTCATTTTCGGCGAGGACCGCTTTCGCTGGAGCGCGAC | |
| | GATGATCGGCCTGTCGCTTGCGGTATTCGGAATCTTGCACGCCCTCGCTCAAGCCT | |
| | TCGTCACTGGTCCCGCCACCAAACGTTTCGGCGAGAAGCAGGCCATTATCGCCGGC | |
| | ATGGCGGCCGACGCGCTGGGCTACGTCTTGCTGGCGTTCGCGACGCGAGGCTGGAT | |
| | GGCCTTCCCCATTATGATTCTTCTCGCTTCCGGCGGCATCGGGATGCCCGCGTTGC | |
| | AGGCCATGCTGTCCAGGCAGGTAGATGACGACCATCAGGGACAGCTTCAAGGATCG | |
| | CTCGCGGCTCTTACCAGCCTAACTTCGATCATTGGACCGCTGATCGTCACGGCGAT | |
| | TTATGCCGCCTCGGCGAGCACATGGAACGGGTTGGCATGGATTGTAGGCGCCGCCC | |
| | TATACCTTGTCTGCCTCCCCGCGTTGCGTCGCGGTGCATGGAGCCGGGCCACCTCG | |
| | ACCTGAATGGAAGCCGGCGGCACCTCGCTAACGGATTCACCACTCCAAGAATTGGA | |
| | GCCAATCAATTCTTGCGGAGAACTGTGAATGCGCAAACCAACCCTTGATCGGGGAA | |
| | GAACAGTATGTCGAGCTATTTTTTGACTTACTGGGGATCAAGCCTGATTGGGAGAA | |
| | AATAAAATATCCCCTATAGTGAGTCGTATTACATGGTCATAGCTGTTTCCTGGCAG | |
| | CTCTGGCCCGTGTCTCAAAATCTCTGATGTTACATTGCACAAGATAAAAATATATC | |
| | ATCATGCCTCCTCTAGAGGTCTCGCTATGTTCTAGAATGTCGCGGAACAAATTTTA | |
| | AAACTAAATCCTAAATTTTTCTAATTTTGTTGCCAATAGTGGATATGTGGGCCGTA | |
| | TAGAAGGAATCTATTGAAGGCCCAAACCCATACTGACGAGCCCAAAGGTTCGTTTT | |
| | GCGTTTTATGTTTCGGTTCGATGCCAACGCCACATTCTGAGCTAGGCAAAAAACAA | |
| | ACGTGTCTTTGAATAGACTCCTCTCGTTAACACATGCAGCGGCTGCATGGTGACGC | |
| | CATTAACACGTGGCCTACAATTGCATGATGTCTCCATTGACACGTGACTTCTAGTC | |
| | TCCTTTCTTAATATATCTAACAAACACTCCTACCTCTTCCAAAATAACAAAGCACC | |
| | AGTGGTCTAGTGGTAGAATAGTACCCTGCCACGGTACAGACCCGGGTTCGATTCCC | |
| | GGCTGGTGCAAGAGACGAGATCTAGTCTGAGTCGACCGTCTCTGTTTTAGAGCTAG | |
| | GCCAACATGAGGATCACCCATGTCTGCAGGGCCTAGCAAGTTAAAATAAGGCTAGT | |
| | CCGTTATCAACTTGGCCAACATGAGGATCACCCATGTCTGCAGGGCCAAGTGGCAC | |
| | CGAGTCGGTGCAACAAAGCACCAGTGGTCTAGTGGTAGAATAGTACCCTGCCACGG | |
| | TACAGACCCGGGTTCGATTCCCGGCTGGTGCAACCCCACTGATGTCATCGTCATAG | |
| | TCCAATAACTCCAATGTCGGGGAGTTAGTTTATGAGGAATAAAGTGTTTAGAATTT | |
| | GATCAGGGGGAGATAATAAAAGCCGAGTTTGAATCTTTTTGTTATAAGTAATGTTT | |
| | ATGTGTGTTTCTATATGTTGTCAAATGGTACCATGTTTTTTTTCCTCTCTTTTTGT | |
| | AACTTGCAAGTGTTGTGTTGTACTTTATTTGGCTTCTTTGTAAGTTGGTAACGGTG | |
| | GTCTATATATGGAAAAGGTCTTGTTTTGTTAAACTTATGTTAGTTAACTGGATTCG | |
| | TCTTTAACCACAAAAAGTTTTCAATAAGCTACAAATTTAGACACGCAAGCCGATGC | |
| | AGTCATTAGTACATATATTTATTGCAAGTGATTACATGGCAACCCAAACTTCAAAA | |
| | ACAGTAGGTTGCTCCATTTAGTCATGGGAGACCCTCGAGCCACCCATGACCAAAAT | |
| | CCCTTAACGTGAGTTACGCGTCGTTCCACTGAGCGTCAGACCCCGTAGAAAAGATC | |
| | AAAGGATCTTCTTGAGATCCTTTTTTTCTGCGCGTAATCTGCTGCTTGCAAACAAA | |
| | AAAACCACCGCTACCAGCGGTGGTTTGTTTGCCGGATCAAGAGCTACCAACTCTTT | |
| | TTCCGAAGGTAACTGGCTTCAGCAGAGCGCAGATACCAAATACTGTTCTTCTAGTG | |
| | TAGCCGTAGTTAGGCCACCACTTCAAGAACTCTGTAGCACCGCCTACATACCTCGC | |
| | TCTGCTAATCCTGTTACCAGTGGCTGCTGCCAGTGGCGATAAGTCGTGTCTTACCG | |
| | GGTTGGACTCAAGACGATAGTTACCGGATAAGGCGCAGCGGTCGGGCTGAACGGGG | |
| | GGTTCGTGCACACAGCCCAGCTTGGAGCGAACGACCTACACCGAACTGAGATACCT | |
| | ACAGCGTGAGCTATGAGAAAGCGCCACGCTTCCCGAAGGGAGAAAGGCGGACAGGT | |
| | ATCCGGTAAGCGGCAGGGTCGGAACAGGAGAGCGCACGAGGGAGCTTCCAGGGGGA | |
| | AACGCCTGGTATCTTTATAGTCCTGTCGGGTTTCGCCACCTCTGACTTGAGCGTCG | |
| | ATTTTTGTGATGCTCGTCAGGGGGGCGGAGCCTATGGAAAAACGCCAGCAACGCGG | |
| | CCTTTTTACGGTTCCTGGCCTTTTGCTGGCCTTTTGCTCACATGTTCTTTCCTGCG | |
| | TTATCCCCTGATTCTGTGGATAACCGTATTACCGCCTTTGAGTGAGCTGATACCGC | |
| | TCGCCGCAGCCGAACGACCGAGCGCAGCGAGTCAGTGAGCGAGGAAGCGGAAGAGC | |
| | GCCCAATACGCAAACCGCCTCTCCCCGCGCGTTGGCCGATTCATTAATGCAGCTGG | |
| | CACGACAGGTTTCCCGACTGGAAAGCGGGCAGTGAGCGCAACGCAATTAATACGCG | |
| | TACCGCTAGCCAGGAAGAGTTTGTAGAAACGCAAAAAGGCCATCCGTCAGGATGGC | |
| | CTTCTGCTTAGTTTGATGCCTGGCAGTTTATGGCGGGCGTCCTGCCCGCCACCCTC | |
| | CGGGCCGTTGCTTCACAACGTTCAAATCCGCTCCCGGCGGATTTGTCCTACTCAGG | |
| | AGAGCGTTCACCGACAAACAACAGATAAAACGAAAGGCCCAGTCTTCCGACTGAGC | |
| | CTTTCGTTTTATTTGATGCCTGGCAGTTCCCTACTCTCGCGTT | |
| 17 | CTGCATTAATGAATCGGCCAACGCGCGGGGAGAGGCGGTTTGCGTATTGGGCGCTCTTCCGCTTCCT | pMCS411 |
| | CGCTCACTGACTCGCTGCGCTCGGTCGTTCGGCTGCGGCGAGCGGTATCAGCTCACTCAAAGGCGGT | Arabidopsis |
| | AATACGGTTATCCACAGAATCAGGGGATAACGCAGGAAAGAACATGAATTAATTCTCATGTTTGACA | dRNA #2 entry |
| | GCTTATCATCGATTAGCTTTAATGCGGTAGTTTATCACAGTTAAATTGCTAACGCAGTCAGGCACCG | plasmid |
| | TGTATGAAATCTAACAATGCGCTCATCGTCATCCTCGGCACCGTCACCCTGGATGCTGTAGGCATAG | |
| | GCTTGGTTATGCCGGTACTGCCGGGCCTCTTGCGGGATATCGTCCATTCCGACAGCATCGCCAGTCA | |
| | CTATGGCGTGCTGCTAGCGCTATATGCGTTGATGCAATTTCTATGCGCACCCGTTCTCGGAGCACTG | |
| | TCCGACCGCTTTGGCCGCCGCCCAGTCCTGCTCGCTTCGCTACTTGGAGCCACTATCGACTACGCGA | |
| | TCATGGCGACCACACCCGTCCTGTGGATTCTCTACGCCGGACGCATCGTGGCCGGCATCACCGGCGC | |
| | CACAGGTGCGGTTGCTGGCGCCTATATCGCCGACATCACCGATGGGGAAGATCGGGCTCGCCACTTC | |
| | GGGCTCATGAGCGCTTGTTTCGGCGTGGGTATGGTGGCAGGCCCCGTGGCCGGGGGACTGTTGGGCG | |
| | CCATCTCCTTACATGCACCATTCCTTGCGGCGGCGGTGCTCAACGGCCTCAACCTACTACTGGGCTG | |
| | CTTCCTAATGCAGGAGTCGCATAAGGGAGAGCGCCGACCCATGCCCTTGAGAGCCTTCAACCCAGTC | |
| | AGCTCCTTCCGGTGGGCGCGGGGCATGACTATCGTCGCCGCACTTATGACTGTCTTCTTTATCATGC | |
| | AACTCGTAGGACAGGTGCCGGCAGCGCTCTGGGTCATTTTCGGCGAGGACCGCTTTCGCTGGAGCGC | |
| | GACGATGATCGGCCTGTCGCTTGCGGTATTCGGAATCTTGCACGCCCTCGCTCAAGCCTTCGTCACT | |
| | GGTCCCGCCACCAAACGTTTCGGCGAGAAGCAGGCCATTATCGCCGGCATGGCGGCCGACGCGCTGG | |
| | GCTACGTCTTGCTGGCGTTCGCGACGCGAGGCTGGATGGCCTTCCCCATTATGATTCTTCTCGCTTC | |
| | CGGCGGCATCGGGATGCCCGCGTTGCAGGCCATGCTGTCCAGGCAGGTAGATGACGACCATCAGGGA | |
| | CAGCTTCAAGGATCGCTCGCGGCTCTTACCAGCCTAACTTCGATCATTGGACCGCTGATCGTCACGG | |
| | CGATTTATGCCGCCTCGGCGAGCACATGGAACGGGTTGGCATGGATTGTAGGCGCCGCCCTATACCT | |
| | TGTCTGCCTCCCCGCGTTGCGTCGCGGTGCATGGAGCCGGGCCACCTCGACCTGAATGGAAGCCGGC | |
| | GGCACCTCGCTAACGGATTCACCACTCCAAGAATTGGAGCCAATCAATTCTTGCGGAGAACTGTGAA | |
| | TGCGCAAACCAACCCTTGATCGGGGAAGAACAGTATGTCGAGCTATTTTTTGACTTACTGGGGATCA | |
| | AGCCTGATTGGGAGAAAATAAAATATCCCCTATAGTGAGTCGTATTACATGGTCATAGCTGTTTCCT | |
| | GGCAGCTCTGGCCCGTGTCTCAAAATCTCTGATGTTACATTGCACAAGATAAAAATATATCATCATG | |
| | CCTCCTCTAGAGGTCTCGCATGGTTCTAGAATGTCGCGGAACAAATTTTAAAACTAAATCCTAAATT | |
| | TTTCTAATTTTGTTGCCAATAGTGGATATGTGGGCCGTATAGAAGGAATCTATTGAAGGCCCAAACC | |
| | CATACTGACGAGCCCAAAGGTTCGTTTTGCGTTTTATGTTTCGGTTCGATGCCAACGCCACATTCTG | |
| | AGCTAGGCAAAAAACAAACGTGTCTTTGAATAGACTCCTCTCGTTAACACATGCAGCGGCTGCATGG | |
| | TGACGCCATTAACACGTGGCCTACAATTGCATGATGTCTCCATTGACACGTGACTTCTAGTCTCCTT | |
| | TCTTAATATATCTAACAAACACTCCTACCTCTTCCAAAATAACAAAGCACCAGTGGTCTAGTGGTAG | |
| | AATAGTACCCTGCCACGGTACAGACCCGGGTTCGATTCCCGGCTGGTGCAAGAGACGAGATCTAGTC | |
| | TGAGTCGACCGTCTCTGTTTTAGAGCTAGGCCAACATGAGGATCACCCATGTCTGCAGGGCCTAGCA | |
| | AGTTAAAATAAGGCTAGTCCGTTATCAACTTGGCCAACATGAGGATCACCCATGTCTGCAGGGCCAA | |
| | GTGGCACCGAGTCGGTGCAACAAAGCACCAGTGGTCTAGTGGTAGAATAGTACCCTGCCACGGTACA | |
| | GACCCGGGTTCGATTCCCGGCTGGTGCAACCCCACTGATGTCATCGTCATAGTCCAATAACTCCAAT | |
| | GTCGGGGAGTTAGTTTATGAGGAATAAAGTGTTTAGAATTTGATCAGGGGGAGATAATAAAAGCCGA | |
| | GTTTGAATCTTTTTGTTATAAGTAATGTTTATGTGTGTTTCTATATGTTGTCAAATGGTACCATGTT | |
| | TTTTTTCCTCTCTTTTTGTAACTTGCAAGTGTTGTGTTGTACTTTATTTGGCTTCTTTGTAAGTTGG | |
| | TAACGGTGGTCTATATATGGAAAAGGTCTTGTTTTGTTAAACTTATGTTAGTTAACTGGATTCGTCT | |
| | TTAACCACAAAAAGTTTTCAATAAGCTACAAATTTAGACACGCAAGCCGATGCAGTCATTAGTACAT | |
| | ATATTTATTGCAAGTGATTACATGGCAACCCAAACTTCAAAAACAGTAGGTTGCTCCATTTAGTGGA | |
| | CGGAGACCCTCGAGCCACCCATGACCAAAATCCCTTAACGTGAGTTACGCGTCGTTCCACTGAGCGT | |
| | CAGACCCCGTAGAAAAGATCAAAGGATCTTCTTGAGATCCTTTTTTTCTGCGCGTAATCTGCTGCTT | |
| | GCAAACAAAAAAACCACCGCTACCAGCGGTGGTTTGTTTGCCGGATCAAGAGCTACCAACTCTTTTT | |
| | CCGAAGGTAACTGGCTTCAGCAGAGCGCAGATACCAAATACTGTTCTTCTAGTGTAGCCGTAGTTAG | |
| | GCCACCACTTCAAGAACTCTGTAGCACCGCCTACATACCTCGCTCTGCTAATCCTGTTACCAGTGGC | |
| | TGCTGCCAGTGGCGATAAGTCGTGTCTTACCGGGTTGGACTCAAGACGATAGTTACCGGATAAGGCG | |
| | CAGCGGTCGGGCTGAACGGGGGGTTCGTGCACACAGCCCAGCTTGGAGCGAACGACCTACACCGAAC | |
| | TGAGATACCTACAGCGTGAGCTATGAGAAAGCGCCACGCTTCCCGAAGGGAGAAAGGCGGACAGGTA | |
| | TCCGGTAAGCGGCAGGGTCGGAACAGGAGAGCGCACGAGGGAGCTTCCAGGGGGAAACGCCTGGTAT | |
| | CTTTATAGTCCTGTCGGGTTTCGCCACCTCTGACTTGAGCGTCGATTTTTGTGATGCTCGTCAGGGG | |
| | GGCGGAGCCTATGGAAAAACGCCAGCAACGCGGCCTTTTTACGGTTCCTGGCCTTTTGCTGGCCTTT | |
| | TGCTCACATGTTCTTTCCTGCGTTATCCCCTGATTCTGTGGATAACCGTATTACCGCCTTTGAGTGA | |
| | GCTGATACCGCTCGCCGCAGCCGAACGACCGAGCGCAGCGAGTCAGTGAGCGAGGAAGCGGAAGAGC | |
| | GCCCAATACGCAAACCGCCTCTCCCCGCGCGTTGGCCGATTCATTAATGCAGCTGGCACGACAGGTT | |
| | TCCCGACTGGAAAGCGGGCAGTGAGCGCAACGCAATTAATACGCGTACCGCTAGCCAGGAAGAGTTT | |
| | GTAGAAACGCAAAAAGGCCATCCGTCAGGATGGCCTTCTGCTTAGTTTGATGCCTGGCAGTTTATGG | |
| | CGGGCGTCCTGCCCGCCACCCTCCGGGCCGTTGCTTCACAACGTTCAAATCCGCTCCCGGCGGATTT | |
| | GTCCTACTCAGGAGAGCGTTCACCGACAAACAACAGATAAAACGAAAGGCCCAGTCTTCCGACTGAG | |
| | CCTTTCGTTTTATTTGATGCCTGGCAGTTCCCTACTCTCGCGTT | |
| 18 | GCCGATTCCTCGGGCTTGGGGGTTCCAGTGCCATTGCAGGGCCGGCAGGCAACCCA | pMCS415 |
| | GCCGCTTACGCCTGGCCAACCGCCCGTTCCTCCACACATGGGGCATTCCACGGCGT | Arabidopsis |
| | CGGTGCCTGGTTGTTCTTGATTTTCCATGCCGCCTCCTTTAGCCGCTAAAATTCAT | egg cell- |
| | CTACTCATTTATTCATTTGCTCATTTACTCTGGTAGCTGCGCGATGTATTCAGATA | specific |
| | GCAGCTCGGTAATGGTCTTGCCTTGGCGTACCGCGTACATCTTCAGCTTGGTGTGA | destination |
| | TCCTCCGCCGGCAACTGAAAGTTGACCCGCTTCATGGCTGGCGTGTCTGCCAGGCT | vector |
| | GGCCAACGTTGCAGCCTTGCTGCTGCGTGCGCTCGGACGGCCGGCACTTAGCGTGT | |
| | TTGTGCTTTTGCTCATTTTCTCTTTACCTCATTAACTCAAATGAGTTTTGATTTAA | |
| | TTTCAGCGGCCAGCGCCTGGACCTCGCGGGCAGCGTCGCCCTCGGGTTCTGATTCA | |
| | AGAACGGTTGTGCCGGCGGCGGCAGTGCCTGGGTAGCTCACGCGCTGCGTGATACG | |
| | GGACTCAAGAATGGGCAGCTCGTACCCGGCCAGCGCCTCGGCAACCTCACCGCCGA | |
| | TGCGCGTGCCTTTGATCGCCCGCGACACGACAAAGGCCGCTTGTAGCCTTCCATCC | |
| | GTGACCTCAATGCGCTGCTTAACCAGCTCCACCAGGTCGGCGGTGGCCCATATGTC | |
| | GTAAGGGCTTGGCTGCACCGGAATCAGCACGAAGTCGGCTGCCTTGATCGCGGACA | |
| | CAGCCAAGTCCGCCGCCTGGGGCGCTCCGTCGATCACTACGAAGTCGCGCCGGCCG | |
| | ATGGCCTTCACGTCGCGGTCAATCGTCGGGCGGTCGATGCCGACAACGGTTAGCGG | |
| | TTGATCTTCCCGCACGGCCGCCCAATCGCGGGCACTGCCCTGGGGATCGGAATCGA | |
| | CTAACAGAACATCGGCCCCGGCGAGTTGCAGGGCGCGGGCTAGATGGGTTGCGATG | |
| | GTCGTCTTGCCTGACCCGCCTTTCTGGTTAAGTACAGCGATAACCTTCATGCGTTC | |
| | CCCTTGCGTATTTGTTTATTTACTCATCGCATCATATACGCAGCGACCGCATGACG | |
| | CAAGCTGTTTTACTCAAATACACATCACCTTTTTAGACGGCGGCGCTCGGTTTCTT | |
| | CAGCGGCCAAGCTGGCCGGCCAGGCCGCCAGCTTGGCATCAGACAAACCGGCCAGG | |
| | ATTTCATGCAGCCGCACGGTTGAGACGTGCGCGGGCGGCTCGAACACGTACCCGGC | |
| | CGCGATCATCTCCGCCTCGATCTCTTCGGTAATGAAAAACGGTTCGTCCTGGCCGT | |
| | CCTGGTGCGGTTTCATGCTTGTTCCTCTTGGCGTTCATTCTCGGCGGCCGCCAGGG | |
| | CGTCGGCCTCGGTCAATGCGTCCTCACGGAAGGCACCGCGCCGCCTGGCCTCGGTG | |
| | GGCGTCACTTCCTCGCTGCGCTCAAGTGCGCGGTACAGGGTCGAGCGATGCACGCC | |
| | AAGCAGTGCAGCCGCCTCTTTCACGGTGCGGCCTTCCTGGTCGATCAGCTCGCGGG | |
| | CGTGCGCGATCTGTGCCGGGGTGAGGGTAGGGCGGGGGCCAAACTTCACGCCTCGG | |
| | GCCTTGGCGGCCTCGCGCCCGCTCCGGGTGCGGTCGATGATTAGGGAACGCTCGAA | |
| | CTCGGCAATGCCGGCGAACACGGTCAACACCATGCGGCCGGCCGGCGTGGTGGTGT | |
| | CGGCCCACGGCTCTGCCAGGCTACGCAGGCCCGCGCCGGCCTCCTGGATGCGCTCG | |
| | GCAATGTCCAGTAGGTCGCGGGTGCTGCGGGCCAGGCGGTCTAGCCTGGTCACTGT | |
| | CACAACGTCGCCAGGGCGTAGGTGGTCAAGCATCCTGGCCAGCTCCGGGCGGTCGC | |
| | GCCTGGTGCCGGTGATCTTCTCGGAAAACAGCTTGGTGCAGCCGGCCGCGTGCAGT | |
| | TCGGCCCGTTGGTTGGTCAAGTCCTGGTCGTCGGTGCTGACGCGGGCATAGCCCAG | |
| | CAGGCCAGCGGCGGCGCTCTTGTTCATGGCGTAATGTCTCCGGTTCTAGTCGCAAG | |
| | TATTCTACTTTATGCGACTAAAACACGCGACAAGAAAACGCCAGGAAAAGGGCAGG | |
| | GCGGCAGCCTGTCGCGTAACTTAGGACTTGTGCGACATGTCGTTTTCAGAAGACGG | |
| | CTGCACTGAACGTCAGAAGCCGACTGCACTATAGCAGCGGAGGGGTTGGATCAAAG | |
| | TACTTTGATCCCGAGGGGAACCCTGTGGTTGGCATGCACATACAAATGGACGAACG | |
| | GATAAACCTTTTCACGCCCTTTTAAATATCCGATTATTCTAATAAACGCTCTTTTC | |
| | TCTTAGGTTTACCCGCCAATATATCCTGTCAAACACTGATAGTTTAAACTGAAGGC | |
| | GGGAAACGACAATCTGATCCAAGCTCAAGCTGCTCTAGCATTCGCCATTCAGGCTG | |
| | CGCAACTGTTGGGAAGGGCGATCGGTGCGGGCCTCTTCGCTATTACGCCAGCTGGC | |
| | GAAAGGGGGATGTGCTGCAAGGCGATTAAGTTGGGTAACGCCAGGGTTTTCCCAGT | |
| | CACGACGTTGTAAAACGACGGCCAGTGCCAAGCTGAATAAAAGCATTTGCGTTTGG | |
| | TTTATCATTGCGTTTATACAAGGACAGAGATCCACTGAGCTGGAATAGCTTAAAAC | |
| | CATTATCAGAACAAAATAAACCATTTTTTGTTAAGAATCAGAGCATAGTAAACAAC | |
| | AGAAACAACCTAAGAGAGGTAACTTGTCCAAGAAGATAGCTAATTATATCTATTTT | |
| | ATAAAAGTTATCATAGTTTGTAAGTCACAAAAGATGCAAATAACAGAGAAACTAGG | |
| | AGACTTGAGAATATACATTCTTGTATATTTGTATTCGAGATTGTGAAAATTTGACC | |
| | ATAAGTTTAAATTCTTAAAAAGATATATCTGATCTAGGTGATGGTTATAGACTGTA | |
| | ATTTTACCACATGTTTAATGATGGATAGTGACACACATGACACATCGACAACACTA | |
| | TAGCATCTTATTTAGATTACAACATGAAATTTTTCTGTAATACATGTCTTTGTACA | |
| | TAATTTAAAAGTAATTCCTAAGAAATATATTTATACAAGGAGTTTAAAGAAAACAT | |
| | AGCATAAAGTTCAATGAGTAGTAAAAACCATATACAGTATATAGCATAAAGTTCAA | |
| | TGAGTTTATTACAAAAGCATTGGTTCACTTTCTGTAACACGACGTTAAACCTTCGT | |
| | CTCCAATAGGAGCGCTACTGATTCAACATGCCAATATATACTAAATACGTTTCTAC | |
| | AGTCAAATGCTTTAACGTTTCATGATTAAGTGACTATTTACCGTCAATCCTTTCCC | |
| | ATTCCTCCCACTAATCCAACTTTTTAATTACTCTTAAATCACCACTAAGCTAGTAA | |
| | CGCCTATCATGAATTAGCTCTACTAAATCTAGCAACCTTTCAAATTTGCAGTATTG | |
| | CAGGTGTCTCTGTGTCTTTAAAATAGTTGCCTTATGATTTCTTCGGTTTCAAGATG | |
| | ATCAAATAGTTATAGATTTCATGCTCACACATGCTCATTAGATGTGTACATACTTT | |
| | ACTTACCCAAATCTATTTTCTCGCAAAGATTTTGATGGTAAAGCTGATTTGGTTCT | |
| | ATTGAACTAAATCAAACGAGTTTCAGACTGAGTGATTCTAATCCGGCCCATTAGCC | |
| | CCTAAACAGACCCACTAATTACGCAGCTTTTAATAGAGTAATTACACCTAGTTTAC | |
| | CCACTAAACCACTAAGCACTAATTATCTCACAATCTAATGAGCTTCCCTCGTAATT | |
| | ACTTGGGCTTTCACTCTACCATTTATTTGTAACAGTCAAGTCTCTACTGTCTCTAT | |
| | ATAAACTCTCTAAAGTTAACACACAATTCTCATCACAAACAAATCAACCAAAGCAA | |
| | CTTCTACTCTTTCTTCTTTCGACCTTATCAATCTGTTGAGAACGCGCCAAGCTATC | |
| | AAACAAGTTTGTACAAAAAAGCTGAACGAGAAACGTAAAATGATATAAATATCAAT | |
| | ATATTAAATTAGATTTTGCATAAAAAACAGACTACATAATACTGTAAAACACAACA | |
| | TATCCAGTCACTATGGCGGCCGCATTAGGCACCCCAGGCTTTACACTTTATGCTTC | |
| | CGGCTCGTATAATGTGTGGATTTTGAGTTAGGATCCGTCGAGATTTTCAGGAGCTA | |
| | AGGAAGCTAAAATGGAGAAAAAAATCACTGGATATACCACCGTTGATATATCCCAA | |
| | TGGCATCGTAAAGAACATTTTGAGGCATTTCAGTCAGTTGCTCAATGTACCTATAA | |
| | CCAGACCGTTCAGCTGGATATTACGGCCTTTTTAAAGACCGTAAAGAAAAATAAGC | |
| | ACAAGTTTTATCCGGCCTTTATTCACATTCTTGCCCGCCTGATGAATGCTCATCCG | |
| | GAATTCCGTATGGCAATGAAAGACGGTGAGCTGGTGATATGGGATAGTGTTCACCC | |
| | TTGTTACACCGTTTTCCATGAGCAAACTGAAACGTTTTCATCGCTCTGGAGTGAAT | |
| | ACCACGACGATTTCCGGCAGTTTCTACACATATATTCGCAAGATGTGGCGTGTTAC | |
| | GGTGAAAACCTGGCCTATTTCCCTAAAGGGTTTATTGAGAATATGTTTTTCGTCTC | |
| | AGCCAATCCCTGGGTGAGTTTCACCAGTTTTGATTTAAACGTGGCCAATATGGACA | |
| | ACTTCTTCGCCCCCGTTTTCACCATGGGCAAATATTATACGCAAGGCGACAAGGTG | |
| | CTGATGCCGCTGGCGATTCAGGTTCATCATGCCGTTTGTGATGGCTTCCATGTCGG | |
| | CAGAATGCTTAATGAATTACAACAGTACTGCGATGAGTGGCAGGCGGGGCGTAATC | |
| | TAGAGGATCCGGCTTACTAAAAGCCAGATAACAGTATGCGTATTTGCGCGCTGATT | |
| | TTTGCGGTATAAGAATATATACTGATATGTATACCCGAAGTATGTCAAAAAGAGGT | |
| | ATGCTATGAAGCAGCGTATTACAGTGACAGTTGACAGCGACAGCTATCAGTTGCTC | |
| | AAGGCATATATGATGTCAATATCTCCGGTCTGGTAAGCACAACCATGCAGAATGAA | |
| | GCCCGTCGTCTGCGTGCCGAACGCTGGAAAGCGGAAAATCAGGAAGGGATGGCTGA | |
| | GGTCGCCCGGTTTATTGAAATGAACGGCTCTTTTGCTGACGAGAACAGGGGCTGGT | |
| | GAAATGCAGTTTAAGGTTTACACCTATAAAAGAGAGAGCCGTTATCGTCTGTTTGT | |
| | GGATGTACAGAGTGATATTATTGACACGCCCGGGCGACGGATGGTGATCCCCCTGG | |
| | CCAGTGCACGTCTGCTGTCAGATAAAGTCCCCCGTGAACTTTACCCGGTGGTGCAT | |
| | ATCGGGGATGAAAGCTGGCGCATGATGACCACCGATATGGCCAGTGTGCCGGTCTC | |
| | CGTTATCGGGGAAGAAGTGGCTGATCTCAGCCACCGCGAAAATGACATCAAAAACG | |
| | CCATTAACCTGATGTTCTGGGGAATATAAATGTCAGGCTCCCTTATACACAGCCAG | |
| | TCTGCAGGTCGACCATAGTGACTGGATATGTTGTGTTTTACAGTATTATGTAGTCT | |
| | GTTTTTTATGCAAAATCTAATTTAATATATTGATATTTATATCATTTTACGTTTCT | |
| | CGTTCAGCTTTCTTGTACAAAGTGGTTCGATAATTCTTAATTAACTAGTTCTAGAG | |
| | CGGCCGCCACCGCGGTGGAGCTCGAATTTCCCCGATCGTTCAAACATTTGGCAATA | |
| | AAGTTTCTTAAGATTGAATCCTGTTGCCGGTCTTGCGATGATTATCATATAATTTC | |
| | TGTTGAATTACGTTAAGCATGTAATAATTAACATGTAATGCATGACGTTATTTATG | |
| | AGATGGGTTTTTATGATTAGAGTCCCGCAATTATACATTTAATACGCGATAGAAAA | |
| | CAAAATATAGCGCGCAAACTAGGATAAATTATCGCGCGCGGTGTCATCTATGTTAC | |
| | TAGATCGGGAATTCGTAATCATGTCATAGCTGTTTCCTGTGTGAAATTGTTATCCG | |
| | CTCACAATTCCACACAACATACGAGCCGGAAGCATAAAGTGTAAAGCCTGGGGTGC | |
| | CTAATGAGTGAGCTAACTCACATTAATTGCGTTGCGCTCACTGCCCGCTTTCCAGT | |
| | CGGGAAACCTGTCGTGCCAGCTGCATTAATGAATCGGCCAACGCGCGGGGAGAGGC | |
| | GGTTTGCGTATTGGCTAGAGCAGCTTGCCAACATGGTGGAGCACGACACTCTCGTC | |
| | TACTCCAAGAATATCAAAGATACAGTCTCAGAAGACCAAAGGGCTATTGAGACTTT | |
| | TCAACAAAGGGTAATATCGGGAAACCTCCTCGGATTCCATTGCCCAGCTATCTGTC | |
| | ACTTCATCAAAAGGACAGTAGAAAAGGAAGGTGGCACCTACAAATGCCATCATTGC | |
| | GATAAAGGAAAGGCTATCGTTCAAGATGCCTCTGCCGACAGTGGTCCCAAAGATGG | |
| | ACCCCCACCCACGAGGAGCATCGTGGAAAAAGAAGACGTTCCAACCACGTCTTCAA | |
| | AGCAAGTGGATTGATGTGAACATGGTGGAGCACGACACTCTCGTCTACTCCAAGAA | |
| | TATCAAAGATACAGTCTCAGAAGACCAAAGGGCTATTGAGACTTTTCAACAAAGGG | |
| | TAATATCGGGAAACCTCCTCGGATTCCATTGCCCAGCTATCTGTCACTTCATCAAA | |
| | AGGACAGTAGAAAAGGAAGGTGGCACCTACAAATGCCATCATTGCGATAAAGGAAA | |
| | GGCTATCGTTCAAGATGCCTCTGCCGACAGTGGTCCCAAAGATGGACCCCCACCCA | |
| | CGAGGAGCATCGTGGAAAAAGAAGACGTTCCAACCACGTCTTCAAAGCAAGTGGAT | |
| | TGATGTGATATCTCCACTGACGTAAGGGATGACGCACAATCCCACTATCCTTCGCA | |
| | AGACCCTTCCTCTATATAAGGAAGTTCATTTCATTTGGAGAGGACACGCTGAAATC | |
| | ACCAGTCTCTCTCTACAAATCTATCTCTCTCGAGCTTTCGCAGATCCGGGGGGCAA | |
| | TGAGATATGAAAAAGCCTGAACTCACCGCGACGTCTGTCGAGAAGTTTCTGATCGA | |
| | AAAGTTCGACAGCGTCTCCGACCTGATGCAGCTCTCGGAGGGCGAAGAATCTCGTG | |
| | CTTTCAGCTTCGATGTAGGAGGGCGTGGATATGTCCTGCGGGTAAATAGCTGCGCC | |
| | GATGGTTTCTACAAAGATCGTTATGTTTATCGGCACTTTGCATCGGCCGCGCTCCC | |
| | GATTCCGGAAGTGCTTGACATTGGGGAGTTTAGCGAGAGCCTGACCTATTGCATCT | |
| | CCCGCCGTTCACAGGGTGTCACGTTGCAAGACCTGCCTGAAACCGAACTGCCCGCT | |
| | GTTCTACAACCGGTCGCGGAGGCTATGGATGCGATCGCTGCGGCCGATCTTAGCCA | |
| | GACGAGCGGGTTCGGCCCATTCGGACCGCAAGGAATCGGTCAATACACTACATGGC | |
| | GTGATTTCATATGCGCGATTGCTGATCCCCATGTGTATCACTGGCAAACTGTGATG | |
| | GACGACACCGTCAGTGCGTCCGTCGCGCAGGCTCTCGATGAGCTGATGCTTTGGGC | |
| | CGAGGACTGCCCCGAAGTCCGGCACCTCGTGCACGCGGATTTCGGCTCCAACAATG | |
| | TCCTGACGGACAATGGCCGCATAACAGCGGTCATTGACTGGAGCGAGGCGATGTTC | |
| | GGGGATTCCCAATACGAGGTCGCCAACATCTTCTTCTGGAGGCCGTGGTTGGCTTG | |
| | TATGGAGCAGCAGACGCGCTACTTCGAGCGGAGGCATCCGGAGCTTGCAGGATCGC | |
| | CACGACTCCGGGCGTATATGCTCCGCATTGGTCTTGACCAACTCTATCAGAGCTTG | |
| | GTTGACGGCAATTTCGATGATGCAGCTTGGGCGCAGGGTCGATGCGACGCAATCGT | |
| | CCGATCCGGAGCCGGGACTGTCGGGCGTACACAAATCGCCCGCAGAAGCGCGGCCG | |
| | TCTGGACCGATGGCTGTGTAGAAGTACTCGCCGATAGTGGAAACCGACGCCCCAGC | |
| | ACTCGTCCGAGGGCAAAGAAATAGAGTAGATGCCGACCGGGATCTGTCGATCGACA | |
| | AGCTCGAGTTTCTCCATAATAATGTGTGAGTAGTTCCCAGATAAGGGAATTAGGGT | |
| | TCCTATAGGGTTTCGCTCATGTGTTGAGCATATAAGAAACCCTTAGTATGTATTTG | |
| | TATTTGTAAAATACTTCTATCAATAAAATTTCTAATTCCTAAAACCAAAATCCAGT | |
| | ACTAAAATCCAGATCCCCCGAATTAATTCGGCGTTAATTCAGTACATTAAAAACGT | |
| | CCGCAATGTGTTATTAAGTTGTCTAAGCGTCAATTTGTTTACACCACAATATATCC | |
| | TGCCACCAGCCAGCCAACAGCTCCCCGACCGGCAGCTCGGCACAAAATCACCACTC | |
| | GATACAGGCAGCCCATCAGTCCGGGACGGCGTCAGCGGGAGAGCCGTTGTAAGGCG | |
| | GCAGACTTTGCTCATGTTACCGATGCTATTCGGAAGAACGGCAACTAAGCTGCCGG | |
| | GTTTGAAACACGGATGATCTCGCGGAGGGTAGCATGTTGATTGTAACGATGACAGA | |
| | GCGTTGCTGCCTGTGATCACCGCGGTTTCAAAATCGGCTCCGTCGATACTATGTTA | |
| | TACGCCAACTTTGAAAACAACTTTGAAAAAGCTGTTTTCTGGTATTTAAGGTTTTA | |
| | GAATGCAAGGAACAGTGAATTGGAGTTCGTCTTGTTATAATTAGGGAAGGTGCGAA | |
| | CAAGTCCCTGATATGAGATCATGTTTGTCATCTGGAGCCATAGAACAGGGTTCATC | |
| | ATGAGTCATCAACTTACCTTCGCCGACAGTGAATTCAGCAGTAAGCGCCGTCAGAC | |
| | CAGAAAAGAGATTTTCTTGTCCCGCATGGAGCAGATTCTGCCATGGCAAAACATGG | |
| | TGGAAGTCATCGAGCCGTTTTACCCCAAGGCTGGTAATGGCCGGCGACCTTATCCG | |
| | CTGGAAACCATGCTACGCATTCACTGCATGCAGCATTGGTACAACCTGAGCGATGG | |
| | CGCGATGGAAGATGCTCTGTACGAAATCGCCTCCATGCGTCTGTTTGCCCGGTTAT | |
| | CCCTGGATAGCGCCTTGCCGGACCGCACCACCATCATGAATTTCCGCCACCTGCTG | |
| | GAGCAGCATCAACTGGCCCGCCAATTGTTCAAGACCATCAATCGCTGGCTGGCCGA | |
| | AGCAGGCGTCATGATGACTCAAGGCACCTTGGTCGATGCCACCATCATTGAGGCAC | |
| | CCAGCTCGACCAAGAACAAAGAGCAGCAACGCGATCCGGAGATGCATCAGACCAAG | |
| | AAAGGCAATCAGTGGCACTTTGGCATGAAGGCCCACATTGGTGTCGATGCCAAGAG | |
| | TGGCCTGACCCACAGCCTGGTCACCACCGCGGCCAACGAGCATGACCTCAATCAGC | |
| | TGGGTAATCTGCTGCATGGAGAGGAGCAATTTGTCTCAGCCGATGCCGGCTACCAA | |
| | GGGGCGCCACAGCGCGAGGAGCTGGCCGAGGTGGATGTGGACTGGCTGATCGCCGA | |
| | GCGCCCCGGCAAGGTAAGAACCTTGAAACAGCATCCACGCAAGAACAAAACGGCCA | |
| | TCAACATCGAATACATGAAAGCCAGCATCCGGGCCAGGGTGGAGCACCCATTTCGC | |
| | ATCATCAAGCGACAGTTCGGCTTCGTGAAAGCCAGATACAAGGGGTTGCTGAAAAA | |
| | CGATAACCAACTGGCGATGTTATTCACGCTGGCCAACCTGTTTCGGGCGGACCAAA | |
| | TGATACGTCAGTGGGAGAGATCTCACTAAAAACTGGGGATAACGCCTTAAATGGCG | |
| | AAGAAACGGTCTAAATAGGCTGATTCAAGGCATTTACGGGAGAAAAAATCGGCTCA | |
| | AACATGAAGAAATGAAATGACTGAGTCAGCCGAGAAGAATTTCCCCGCTTATTCGC | |
| | ACCTTCCTTAGCTTCTTGGGGTATCTTTAAATACTGTAGAAAAGAGGAAGGAAATA | |
| | ATAAATGGCTAAAATGAGAATATCACCGGAATTGAAAAAACTGATCGAAAAATACC | |
| | GCTGCGTAAAAGATACGGAAGGAATGTCTCCTGCTAAGGTATATAAGCTGGTGGGA | |
| | GAAAATGAAAACCTATATTTAAAAATGACGGACAGCCGGTATAAAGGGACCACCTA | |
| | TGATGTGGAACGGGAAAAGGACATGATGCTATGGCTGGAAGGAAAGCTGCCTGTTC | |
| | CAAAGGTCCTGCACTTTGAACGGCATGATGGCTGGAGCAATCTGCTCATGAGTGAG | |
| | GCCGATGGCGTCCTTTGCTCGGAAGAGTATGAAGATGAACAAAGCCCTGAAAAGAT | |
| | TATCGAGCTGTATGCGGAGTGCATCAGGCTCTTTCACTCCATCGACATATCGGATT | |
| | GTCCCTATACGAATAGCTTAGACAGCCGCTTAGCCGAATTGGATTACTTACTGAAT | |
| | AACGATCTGGCCGATGTGGATTGCGAAAACTGGGAAGAAGACACTCCATTTAAAGA | |
| | TCCGCGCGAGCTGTATGATTTTTTAAAGACGGAAAAGCCCGAAGAGGAACTTGTCT | |
| | TTTCCCACGGCGACCTGGGAGACAGCAACATCTTTGTGAAAGATGGCAAAGTAAGT | |
| | GGCTTTATTGATCTTGGGAGAAGCGGCAGGGCGGACAAGTGGTATGACATTGCCTT | |
| | CTGCGTCCGGTCGATCAGGGAGGATATCGGGGAAGAACAGTATGTCGAGCTATTTT | |
| | TTGACTTACTGGGGATCAAGCCTGATTGGGAGAAAATAAAATATTATATTTTACTG | |
| | GATGAATTGTTTTAGTACCTAGAATGCATGACCAAAATCCCTTAACGTGAGTTTTC | |
| | GTTCCACTGAGCGTCAGACCCCGTAGAAAAGATCAAAGGATCTTCTTGAGATCCTT | |
| | TTTTTCTGCGCGTAATCTGCTGCTTGCAAACAAAAAAACCACCGCTACCAGCGGTG | |
| | GTTTGTTTGCCGGATCAAGAGCTACCAACTCTTTTTCCGAAGGTAACTGGCTTCAG | |
| | CAGAGCGCAGATACCAAATACTGTCCTTCTAGTGTAGCCGTAGTTAGGCCACCACT | |
| | TCAAGAACTCTGTAGCACCGCCTACATACCTCGCTCTGCTAATCCTGTTACCAGTG | |
| | GCTGCTGCCAGTGGCGATAAGTCGTGTCTTACCGGGTTGGACTCAAGACGATAGTT | |
| | ACCGGATAAGGCGCAGCGGTCGGGCTGAACGGGGGGTTCGTGCACACAGCCCAGCT | |
| | TGGAGCGAACGACCTACACCGAACTGAGATACCTACAGCGTGAGCTATGAGAAAGC | |
| | GCCACGCTTCCCGAAGGGAGAAAGGCGGACAGGTATCCGGTAAGCGGCAGGGTCGG | |
| | AACAGGAGAGCGCACGAGGGAGCTTCCAGGGGGAAACGCCTGGTATCTTTATAGTC | |
| | CTGTCGGGTTTCGCCACCTCTGACTTGAGCGTCGATTTTTGTGATGCTCGTCAGGG | |
| | GGGCGGAGCCTATGGAAAAACGCCAGCAACGCGGCCTTTTTACGGTTCCTGGCCTT | |
| | TTGCTGGCCTTTTGCTCACATGTTCTTTCCTGCGTTATCCCCTGATTCTGTGGATA | |
| | ACCGTATTACCGCCTTTGAGTGAGCTGATACCGCTCGCCGCAGCCGAACGACCGAG | |
| | CGCAGCGAGTCAGTGAGCGAGGAAGCGGAAGAGCGCCTGATGCGGTATTTTCTCCT | |
| | TACGCATCTGTGCGGTATTTCACACCGCATATGGTGCACTCTCAGTACAATCTGCT | |
| | CTGATGCCGCATAGTTAAGCCAGTATACACTCCGCTATCGCTACGTGACTGGGTCA | |
| | TGGCTGCGCCCCGACACCCGCCAACACCCGCTGACGCGCCCTGACGGGCTTGTCTG | |
| | CTCCCGGCATCCGCTTACAGACAAGCTGTGACCGTCTCCGGGAGCTGCATGTGTCA | |
| | GAGGTTTTCACCGTCATCACCGAAACGCGCGAGGCAGGGTGCCTTGATGTGGGCGC | |
| | CGGCGGTCGAGTGGCGACGGCGCGGCTTGTCCGCGCCCTGGTAGATTGCCTGGCCG | |
| | TAGGCCAGCCATTTTTGAGCGGCCAGCGGCCGCGATAGGCCGACGCGAAGCGGCGG | |
| | GGCGTAGGGAGCGCAGCGACCGAAGGGTAGGCGCTTTTTGCAGCTCTTCGGCTGTG | |
| | CGCTGGCCAGACAGTTATGCACAGGCCAGGCGGGTTTTAAGAGTTTTAATAAGTTT | |
| | TAAAGAGTTTTAGGCGGAAAAATCGCCTTTTTTCTCTTTTATATCAGTCACTTACA | |
| | TGTGTGACCGGTTCCCAATGTACGGCTTTGGGTTCCCAATGTACGGGTTCCGGTTC | |
| | CCAATGTACGGCTTTGGGTTCCCAATGTACGTGCTATCCACAGGAAAGAGACCTTT | |
| | TCGACCTTTTTCCCCTGCTAGGGCAATTTGCCCTAGCATCTGCTCCGTACATTAGG | |
| | AACCGGCGGATGCTTCGCCCTCGATCAGGTTGCGGTAGCGCATGACTAGGATCGGG | |
| | CCAGCCTGCCCCGCCTCCTCCTTCAAATCGTACTCCGGCAGGTCATTTGACCCGAT | |
| | CAGCTTGCGCACGGTGAAACAGAACTTCTTGAACTCTCCGGCGCTGCCACTGCGTT | |
| | CGTAGATCGTCTTGAACAACCATCTGGCTTCTGCCTTGCCTGCGGCGCGGCGTGCC | |
| | AGGCGGTAGAGAAAACGGCCGATGCCGGGATCGATCAAAAAGTAATCGGGGTGAAC | |
| | CGTCAGCACGTCCGGGTTCTTGCCTTCTGTGATCTCGCGGTACATCCAATCAGCTA | |
| | GCTCGATCTCGATGTACTCCGGCCGCCCGGTTTCGCTCTTTACGATCTTGTAGCGG | |
| | CTAATCAAGGCTTCACCCTCGGATACCGTCACCAGGCGGCCGTTCTTGGCCTTCTT | |
| | CGTACGCTGCATGGCAACGTGCGTGGTGTTTAACCGAATGCAGGTTTCTACCAGGT | |
| | CGTCTTTCTGCTTTCCGCCATCGGCTCGCCGGCAGAACTTGAGTACGTCCGCAACG | |
| | TGTGGACGGAACACGCGGCCGGGCTTGTCTCCCTTCCCTTCCCGGTATCGGTTCAT | |
| | GGATTCGGTTAGATGGGAAACCGCCATCAGTACCAGGTCGTAATCCCACACACTGG | |
| | CCATGCCGGCCGGCCCTGCGGAAACCTCTACGTGCCCGTCTGGAAGCTCGTAGCGG | |
| | ATCACCTCGCCAGCTCGTCGGTCACGCTTCGACAGACGGAAAACGGCCACGTCCAT | |
| | GATGCTGCGACTATCGCGGGTGCCCACGTCATAGAGCATCGGAACGAAAAAATCTG | |
| | GTTGCTCGTCGCCCTTGGGCGGCTTCCTAATCGACGGCGCACCGGCTGCCGGCGGT | |
| | TGCCGGGATTCTTTGCGGATTCGATCAGCGGCCGCTTGCCACGATTCACCGGGGCG | |
| | TGCTTCTGCCTCGATGCGTTGCCGCTGGGCGGCCTGCGCGGCCTTCAACTTCTCCA | |
| | CCAGGTCATCACCCAGCGCCGCGCCGATTTGTACCGGGCCGGATGGTTTGCGACCG | |
| | CTCAC | |
| 19 | TTTGCGACCGCTCACGCCGATTCCTCGGGCTTGGGGGTTCCAGTGCCATTGCAGGGCCGGCAGGCAA | pMCS416 |
| | CCCAGCCGCTTACGCCTGGCCAACCGCCCGTTCCTCCACACATGGGGCATTCCACGGCGTCGGTGCC | Arabidopsis |
| | TGGTTGTTCTTGATTTTCCATGCCGCCTCCTTTAGCCGCTAAAATTCATCTACTCATTTATTCATTT | embryo- |
| | GCTCATTTACTCTGGTAGCTGCGCGATGTATTCAGATAGCAGCTCGGTAATGGTCTTGCCTTGGCGT | specific |
| | ACCGCGTACATCTTCAGCTTGGTGTGATCCTCCGCCGGCAACTGAAAGTTGACCCGCTTCATGGCTG | destination |
| | GCGTGTCTGCCAGGCTGGCCAACGTTGCAGCCTTGCTGCTGCGTGCGCTCGGACGGCCGGCACTTAG | vector |
| | CGTGTTTGTGCTTTTGCTCATTTTCTCTTTACCTCATTAACTCAAATGAGTTTTGATTTAATTTCAG | |
| | CGGCCAGCGCCTGGACCTCGCGGGCAGCGTCGCCCTCGGGTTCTGATTCAAGAACGGTTGTGCCGGC | |
| | GGCGGCAGTGCCTGGGTAGCTCACGCGCTGCGTGATACGGGACTCAAGAATGGGCAGCTCGTACCCG | |
| | GCCAGCGCCTCGGCAACCTCACCGCCGATGCGCGTGCCTTTGATCGCCCGCGACACGACAAAGGCCG | |
| | CTTGTAGCCTTCCATCCGTGACCTCAATGCGCTGCTTAACCAGCTCCACCAGGTCGGCGGTGGCCCA | |
| | TATGTCGTAAGGGCTTGGCTGCACCGGAATCAGCACGAAGTCGGCTGCCTTGATCGCGGACACAGCC | |
| | AAGTCCGCCGCCTGGGGCGCTCCGTCGATCACTACGAAGTCGCGCCGGCCGATGGCCTTCACGTCGC | |
| | GGTCAATCGTCGGGCGGTCGATGCCGACAACGGTTAGCGGTTGATCTTCCCGCACGGCCGCCCAATC | |
| | GCGGGCACTGCCCTGGGGATCGGAATCGACTAACAGAACATCGGCCCCGGCGAGTTGCAGGGCGCGG | |
| | GCTAGATGGGTTGCGATGGTCGTCTTGCCTGACCCGCCTTTCTGGTTAAGTACAGCGATAACCTTCA | |
| | TGCGTTCCCCTTGCGTATTTGTTTATTTACTCATCGCATCATATACGCAGCGACCGCATGACGCAAG | |
| | CTGTTTTACTCAAATACACATCACCTTTTTAGACGGCGGCGCTCGGTTTCTTCAGCGGCCAAGCTGG | |
| | CCGGCCAGGCCGCCAGCTTGGCATCAGACAAACCGGCCAGGATTTCATGCAGCCGCACGGTTGAGAC | |
| | GTGCGCGGGCGGCTCGAACACGTACCCGGCCGCGATCATCTCCGCCTCGATCTCTTCGGTAATGAAA | |
| | AACGGTTCGTCCTGGCCGTCCTGGTGCGGTTTCATGCTTGTTCCTCTTGGCGTTCATTCTCGGCGGC | |
| | CGCCAGGGCGTCGGCCTCGGTCAATGCGTCCTCACGGAAGGCACCGCGCCGCCTGGCCTCGGTGGGC | |
| | GTCACTTCCTCGCTGCGCTCAAGTGCGCGGTACAGGGTCGAGCGATGCACGCCAAGCAGTGCAGCCG | |
| | CCTCTTTCACGGTGCGGCCTTCCTGGTCGATCAGCTCGCGGGCGTGCGCGATCTGTGCCGGGGTGAG | |
| | GGTAGGGCGGGGGCCAAACTTCACGCCTCGGGCCTTGGCGGCCTCGCGCCCGCTCCGGGTGCGGTCG | |
| | ATGATTAGGGAACGCTCGAACTCGGCAATGCCGGCGAACACGGTCAACACCATGCGGCCGGCCGGCG | |
| | TGGTGGTGTCGGCCCACGGCTCTGCCAGGCTACGCAGGCCCGCGCCGGCCTCCTGGATGCGCTCGGC | |
| | AATGTCCAGTAGGTCGCGGGTGCTGCGGGCCAGGCGGTCTAGCCTGGTCACTGTCACAACGTCGCCA | |
| | GGGCGTAGGTGGTCAAGCATCCTGGCCAGCTCCGGGCGGTCGCGCCTGGTGCCGGTGATCTTCTCGG | |
| | AAAACAGCTTGGTGCAGCCGGCCGCGTGCAGTTCGGCCCGTTGGTTGGTCAAGTCCTGGTCGTCGGT | |
| | GCTGACGCGGGCATAGCCCAGCAGGCCAGCGGCGGCGCTCTTGTTCATGGCGTAATGTCTCCGGTTC | |
| | TAGTCGCAAGTATTCTACTTTATGCGACTAAAACACGCGACAAGAAAACGCCAGGAAAAGGGCAGGG | |
| | CGGCAGCCTGTCGCGTAACTTAGGACTTGTGCGACATGTCGTTTTCAGAAGACGGCTGCACTGAACG | |
| | TCAGAAGCCGACTGCACTATAGCAGCGGAGGGGTTGGATCAAAGTACTTTGATCCCGAGGGGAACCC | |
| | TGTGGTTGGCATGCACATACAAATGGACGAACGGATAAACCTTTTCACGCCCTTTTAAATATCCGAT | |
| | TATTCTAATAAACGCTCTTTTCTCTTAGGTTTACCCGCCAATATATCCTGTCAAACACTGATAGTTT | |
| | AAACTGAAGGCGGGAAACGACAATCTGATCCAAGCTCAAGCTGCTCTAGCATTCGCCATTCAGGCTG | |
| | CGCAACTGTTGGGAAGGGCGATCGGTGCGGGCCTCTTCGCTATTACGCCAGCTGGCGAAAGGGGGAT | |
| | GTGCTGCAAGGCGATTAAGTTGGGTAACGCCAGGGTTTTCCCAGTCACGACGTTGTAAAACGACGGC | |
| | CAGTGCCAAGCTTTCGACAAAGACTGGTCGGTCGGTTTTGGTAGACAATTGAAATTAGATGGATGGT | |
| | CCGGTTCGGTATACTATAAGATTAAAAACAGTTTTAAATTCAGCTAAACCGAACTCATTTGATTTTA | |
| | TTAAACCGGAATCATCCGATTCGAGTTTGTAAAAAATACCGAAATTGAAAACACTAAACAAAAACTG | |
| | TATTAAACTGTTACTGAAATAAGAGAATCTCCCAATTCGGTTTACGTACTACTCTTCAGAAATCAGA | |
| | ACCAAAAATTCAGAAATCGGATTGAACCAAACTTAAATTGACGGTCCGGTTAGTTTTCGGCTCTACA | |
| | AATTAAAGGCCCAAGTTTCTGCTTTAAAAGAACGAAATAGTTAATGGGCTCAAACCATAGACCAGGT | |
| | AAGTCATGGGCTTGGTTAGTCCGGGTCAACCCGGTAGACCCGATTCCTGAAGAAAACCTAGTGGAAG | |
| | GTTTAAAGTTGTAAACTTTCCGACCAAATAAACAAAATCGTTTTCCAGCTTCTTCCGTCGCCACTAA | |
| | ACCCTGAGGCTAAACCTAGACGAGTCAAAGTGTAAAATCGTTAAACCCTAAGAGGGAGTGAGAGAGA | |
| | GAAGAATCTAGATAGGAGATCAACATGAGGTACATGGCGCGCCAAGCTATCAAACAAGTTTGTACAA | |
| | AAAAGCTGAACGAGAAACGTAAAATGATATAAATATCAATATATTAAATTAGATTTTGCATAAAAAA | |
| | CAGACTACATAATACTGTAAAACACAACATATCCAGTCACTATGGCGGCCGCATTAGGCACCCCAGG | |
| | CTTTACACTTTATGCTTCCGGCTCGTATAATGTGTGGATTTTGAGTTAGGATCCGTCGAGATTTTCA | |
| | GGAGCTAAGGAAGCTAAAATGGAGAAAAAAATCACTGGATATACCACCGTTGATATATCCCAATGGC | |
| | ATCGTAAAGAACATTTTGAGGCATTTCAGTCAGTTGCTCAATGTACCTATAACCAGACCGTTCAGCT | |
| | GGATATTACGGCCTTTTTAAAGACCGTAAAGAAAAATAAGCACAAGTTTTATCCGGCCTTTATTCAC | |
| | ATTCTTGCCCGCCTGATGAATGCTCATCCGGAATTCCGTATGGCAATGAAAGACGGTGAGCTGGTGA | |
| | TATGGGATAGTGTTCACCCTTGTTACACCGTTTTCCATGAGCAAACTGAAACGTTTTCATCGCTCTG | |
| | GAGTGAATACCACGACGATTTCCGGCAGTTTCTACACATATATTCGCAAGATGTGGCGTGTTACGGT | |
| | GAAAACCTGGCCTATTTCCCTAAAGGGTTTATTGAGAATATGTTTTTCGTCTCAGCCAATCCCTGGG | |
| | TGAGTTTCACCAGTTTTGATTTAAACGTGGCCAATATGGACAACTTCTTCGCCCCCGTTTTCACCAT | |
| | GGGCAAATATTATACGCAAGGCGACAAGGTGCTGATGCCGCTGGCGATTCAGGTTCATCATGCCGTT | |
| | TGTGATGGCTTCCATGTCGGCAGAATGCTTAATGAATTACAACAGTACTGCGATGAGTGGCAGGCGG | |
| | GGCGTAATCTAGAGGATCCGGCTTACTAAAAGCCAGATAACAGTATGCGTATTTGCGCGCTGATTTT | |
| | TGCGGTATAAGAATATATACTGATATGTATACCCGAAGTATGTCAAAAAGAGGTATGCTATGAAGCA | |
| | GCGTATTACAGTGACAGTTGACAGCGACAGCTATCAGTTGCTCAAGGCATATATGATGTCAATATCT | |
| | CCGGTCTGGTAAGCACAACCATGCAGAATGAAGCCCGTCGTCTGCGTGCCGAACGCTGGAAAGCGGA | |
| | AAATCAGGAAGGGATGGCTGAGGTCGCCCGGTTTATTGAAATGAACGGCTCTTTTGCTGACGAGAAC | |
| | AGGGGCTGGTGAAATGCAGTTTAAGGTTTACACCTATAAAAGAGAGAGCCGTTATCGTCTGTTTGTG | |
| | GATGTACAGAGTGATATTATTGACACGCCCGGGCGACGGATGGTGATCCCCCTGGCCAGTGCACGTC | |
| | TGCTGTCAGATAAAGTCCCCCGTGAACTTTACCCGGTGGTGCATATCGGGGATGAAAGCTGGCGCAT | |
| | GATGACCACCGATATGGCCAGTGTGCCGGTCTCCGTTATCGGGGAAGAAGTGGCTGATCTCAGCCAC | |
| | CGCGAAAATGACATCAAAAACGCCATTAACCTGATGTTCTGGGGAATATAAATGTCAGGCTCCCTTA | |
| | TACACAGCCAGTCTGCAGGTCGACCATAGTGACTGGATATGTTGTGTTTTACAGTATTATGTAGTCT | |
| | GTTTTTTATGCAAAATCTAATTTAATATATTGATATTTATATCATTTTACGTTTCTCGTTCAGCTTT | |
| | CTTGTACAAAGTGGTTCGATAATTCTTAATTAACTAGTTCTAGAGCGGCCGCCACCGCGGTGGAGCT | |
| | CGAATTTCCCCGATCGTTCAAACATTTGGCAATAAAGTTTCTTAAGATTGAATCCTGTTGCCGGTCT | |
| | TGCGATGATTATCATATAATTTCTGTTGAATTACGTTAAGCATGTAATAATTAACATGTAATGCATG | |
| | ACGTTATTTATGAGATGGGTTTTTATGATTAGAGTCCCGCAATTATACATTTAATACGCGATAGAAA | |
| | ACAAAATATAGCGCGCAAACTAGGATAAATTATCGCGCGCGGTGTCATCTATGTTACTAGATCGGGA | |
| | ATTCGTAATCATGTCATAGCTGTTTCCTGTGTGAAATTGTTATCCGCTCACAATTCCACACAACATA | |
| | CGAGCCGGAAGCATAAAGTGTAAAGCCTGGGGTGCCTAATGAGTGAGCTAACTCACATTAATTGCGT | |
| | TGCGCTCACTGCCCGCTTTCCAGTCGGGAAACCTGTCGTGCCAGCTGCATTAATGAATCGGCCAACG | |
| | CGCGGGGAGAGGCGGTTTGCGTATTGGCTAGAGCAGCTTGCCAACATGGTGGAGCACGACACTCTCG | |
| | TCTACTCCAAGAATATCAAAGATACAGTCTCAGAAGACCAAAGGGCTATTGAGACTTTTCAACAAAG | |
| | GGTAATATCGGGAAACCTCCTCGGATTCCATTGCCCAGCTATCTGTCACTTCATCAAAAGGACAGTA | |
| | GAAAAGGAAGGTGGCACCTACAAATGCCATCATTGCGATAAAGGAAAGGCTATCGTTCAAGATGCCT | |
| | CTGCCGACAGTGGTCCCAAAGATGGACCCCCACCCACGAGGAGCATCGTGGAAAAAGAAGACGTTCC | |
| | AACCACGTCTTCAAAGCAAGTGGATTGATGTGAACATGGTGGAGCACGACACTCTCGTCTACTCCAA | |
| | GAATATCAAAGATACAGTCTCAGAAGACCAAAGGGCTATTGAGACTTTTCAACAAAGGGTAATATCG | |
| | GGAAACCTCCTCGGATTCCATTGCCCAGCTATCTGTCACTTCATCAAAAGGACAGTAGAAAAGGAAG | |
| | GTGGCACCTACAAATGCCATCATTGCGATAAAGGAAAGGCTATCGTTCAAGATGCCTCTGCCGACAG | |
| | TGGTCCCAAAGATGGACCCCCACCCACGAGGAGCATCGTGGAAAAAGAAGACGTTCCAACCACGTCT | |
| | TCAAAGCAAGTGGATTGATGTGATATCTCCACTGACGTAAGGGATGACGCACAATCCCACTATCCTT | |
| | CGCAAGACCCTTCCTCTATATAAGGAAGTTCATTTCATTTGGAGAGGACACGCTGAAATCACCAGTC | |
| | TCTCTCTACAAATCTATCTCTCTCGAGCTTTCGCAGATCCGGGGGGCAATGAGATATGAAAAAGCCT | |
| | GAACTCACCGCGACGTCTGTCGAGAAGTTTCTGATCGAAAAGTTCGACAGCGTCTCCGACCTGATGC | |
| | AGCTCTCGGAGGGCGAAGAATCTCGTGCTTTCAGCTTCGATGTAGGAGGGCGTGGATATGTCCTGCG | |
| | GGTAAATAGCTGCGCCGATGGTTTCTACAAAGATCGTTATGTTTATCGGCACTTTGCATCGGCCGCG | |
| | CTCCCGATTCCGGAAGTGCTTGACATTGGGGAGTTTAGCGAGAGCCTGACCTATTGCATCTCCCGCC | |
| | GTTCACAGGGTGTCACGTTGCAAGACCTGCCTGAAACCGAACTGCCCGCTGTTCTACAACCGGTCGC | |
| | GGAGGCTATGGATGCGATCGCTGCGGCCGATCTTAGCCAGACGAGCGGGTTCGGCCCATTCGGACCG | |
| | CAAGGAATCGGTCAATACACTACATGGCGTGATTTCATATGCGCGATTGCTGATCCCCATGTGTATC | |
| | ACTGGCAAACTGTGATGGACGACACCGTCAGTGCGTCCGTCGCGCAGGCTCTCGATGAGCTGATGCT | |
| | TTGGGCCGAGGACTGCCCCGAAGTCCGGCACCTCGTGCACGCGGATTTCGGCTCCAACAATGTCCTG | |
| | ACGGACAATGGCCGCATAACAGCGGTCATTGACTGGAGCGAGGCGATGTTCGGGGATTCCCAATACG | |
| | AGGTCGCCAACATCTTCTTCTGGAGGCCGTGGTTGGCTTGTATGGAGCAGCAGACGCGCTACTTCGA | |
| | GCGGAGGCATCCGGAGCTTGCAGGATCGCCACGACTCCGGGCGTATATGCTCCGCATTGGTCTTGAC | |
| | CAACTCTATCAGAGCTTGGTTGACGGCAATTTCGATGATGCAGCTTGGGCGCAGGGTCGATGCGACG | |
| | CAATCGTCCGATCCGGAGCCGGGACTGTCGGGCGTACACAAATCGCCCGCAGAAGCGCGGCCGTCTG | |
| | GACCGATGGCTGTGTAGAAGTACTCGCCGATAGTGGAAACCGACGCCCCAGCACTCGTCCGAGGGCA | |
| | AAGAAATAGAGTAGATGCCGACCGGGATCTGTCGATCGACAAGCTCGAGTTTCTCCATAATAATGTG | |
| | TGAGTAGTTCCCAGATAAGGGAATTAGGGTTCCTATAGGGTTTCGCTCATGTGTTGAGCATATAAGA | |
| | AACCCTTAGTATGTATTTGTATTTGTAAAATACTTCTATCAATAAAATTTCTAATTCCTAAAACCAA | |
| | AATCCAGTACTAAAATCCAGATCCCCCGAATTAATTCGGCGTTAATTCAGTACATTAAAAACGTCCG | |
| | CAATGTGTTATTAAGTTGTCTAAGCGTCAATTTGTTTACACCACAATATATCCTGCCACCAGCCAGC | |
| | CAACAGCTCCCCGACCGGCAGCTCGGCACAAAATCACCACTCGATACAGGCAGCCCATCAGTCCGGG | |
| | ACGGCGTCAGCGGGAGAGCCGTTGTAAGGCGGCAGACTTTGCTCATGTTACCGATGCTATTCGGAAG | |
| | AACGGCAACTAAGCTGCCGGGTTTGAAACACGGATGATCTCGCGGAGGGTAGCATGTTGATTGTAAC | |
| | GATGACAGAGCGTTGCTGCCTGTGATCACCGCGGTTTCAAAATCGGCTCCGTCGATACTATGTTATA | |
| | CGCCAACTTTGAAAACAACTTTGAAAAAGCTGTTTTCTGGTATTTAAGGTTTTAGAATGCAAGGAAC | |
| | AGTGAATTGGAGTTCGTCTTGTTATAATTAGGGAAGGTGCGAACAAGTCCCTGATATGAGATCATGT | |
| | TTGTCATCTGGAGCCATAGAACAGGGTTCATCATGAGTCATCAACTTACCTTCGCCGACAGTGAATT | |
| | CAGCAGTAAGCGCCGTCAGACCAGAAAAGAGATTTTCTTGTCCCGCATGGAGCAGATTCTGCCATGG | |
| | CAAAACATGGTGGAAGTCATCGAGCCGTTTTACCCCAAGGCTGGTAATGGCCGGCGACCTTATCCGC | |
| | TGGAAACCATGCTACGCATTCACTGCATGCAGCATTGGTACAACCTGAGCGATGGCGCGATGGAAGA | |
| | TGCTCTGTACGAAATCGCCTCCATGCGTCTGTTTGCCCGGTTATCCCTGGATAGCGCCTTGCCGGAC | |
| | CGCACCACCATCATGAATTTCCGCCACCTGCTGGAGCAGCATCAACTGGCCCGCCAATTGTTCAAGA | |
| | CCATCAATCGCTGGCTGGCCGAAGCAGGCGTCATGATGACTCAAGGCACCTTGGTCGATGCCACCAT | |
| | CATTGAGGCACCCAGCTCGACCAAGAACAAAGAGCAGCAACGCGATCCGGAGATGCATCAGACCAAG | |
| | AAAGGCAATCAGTGGCACTTTGGCATGAAGGCCCACATTGGTGTCGATGCCAAGAGTGGCCTGACCC | |
| | ACAGCCTGGTCACCACCGCGGCCAACGAGCATGACCTCAATCAGCTGGGTAATCTGCTGCATGGAGA | |
| | GGAGCAATTTGTCTCAGCCGATGCCGGCTACCAAGGGGCGCCACAGCGCGAGGAGCTGGCCGAGGTG | |
| | GATGTGGACTGGCTGATCGCCGAGCGCCCCGGCAAGGTAAGAACCTTGAAACAGCATCCACGCAAGA | |
| | ACAAAACGGCCATCAACATCGAATACATGAAAGCCAGCATCCGGGCCAGGGTGGAGCACCCATTTCG | |
| | CATCATCAAGCGACAGTTCGGCTTCGTGAAAGCCAGATACAAGGGGTTGCTGAAAAACGATAACCAA | |
| | CTGGCGATGTTATTCACGCTGGCCAACCTGTTTCGGGCGGACCAAATGATACGTCAGTGGGAGAGAT | |
| | CTCACTAAAAACTGGGGATAACGCCTTAAATGGCGAAGAAACGGTCTAAATAGGCTGATTCAAGGCA | |
| | TTTACGGGAGAAAAAATCGGCTCAAACATGAAGAAATGAAATGACTGAGTCAGCCGAGAAGAATTTC | |
| | CCCGCTTATTCGCACCTTCCTTAGCTTCTTGGGGTATCTTTAAATACTGTAGAAAAGAGGAAGGAAA | |
| | TAATAAATGGCTAAAATGAGAATATCACCGGAATTGAAAAAACTGATCGAAAAATACCGCTGCGTAA | |
| | AAGATACGGAAGGAATGTCTCCTGCTAAGGTATATAAGCTGGTGGGAGAAAATGAAAACCTATATTT | |
| | AAAAATGACGGACAGCCGGTATAAAGGGACCACCTATGATGTGGAACGGGAAAAGGACATGATGCTA | |
| | TGGCTGGAAGGAAAGCTGCCTGTTCCAAAGGTCCTGCACTTTGAACGGCATGATGGCTGGAGCAATC | |
| | TGCTCATGAGTGAGGCCGATGGCGTCCTTTGCTCGGAAGAGTATGAAGATGAACAAAGCCCTGAAAA | |
| | GATTATCGAGCTGTATGCGGAGTGCATCAGGCTCTTTCACTCCATCGACATATCGGATTGTCCCTAT | |
| | ACGAATAGCTTAGACAGCCGCTTAGCCGAATTGGATTACTTACTGAATAACGATCTGGCCGATGTGG | |
| | ATTGCGAAAACTGGGAAGAAGACACTCCATTTAAAGATCCGCGCGAGCTGTATGATTTTTTAAAGAC | |
| | GGAAAAGCCCGAAGAGGAACTTGTCTTTTCCCACGGCGACCTGGGAGACAGCAACATCTTTGTGAAA | |
| | GATGGCAAAGTAAGTGGCTTTATTGATCTTGGGAGAAGCGGCAGGGCGGACAAGTGGTATGACATTG | |
| | CCTTCTGCGTCCGGTCGATCAGGGAGGATATCGGGGAAGAACAGTATGTCGAGCTATTTTTTGACTT | |
| | ACTGGGGATCAAGCCTGATTGGGAGAAAATAAAATATTATATTTTACTGGATGAATTGTTTTAGTAC | |
| | CTAGAATGCATGACCAAAATCCCTTAACGTGAGTTTTCGTTCCACTGAGCGTCAGACCCCGTAGAAA | |
| | AGATCAAAGGATCTTCTTGAGATCCTTTTTTTCTGCGCGTAATCTGCTGCTTGCAAACAAAAAAACC | |
| | ACCGCTACCAGCGGTGGTTTGTTTGCCGGATCAAGAGCTACCAACTCTTTTTCCGAAGGTAACTGGC | |
| | TTCAGCAGAGCGCAGATACCAAATACTGTCCTTCTAGTGTAGCCGTAGTTAGGCCACCACTTCAAGA | |
| | ACTCTGTAGCACCGCCTACATACCTCGCTCTGCTAATCCTGTTACCAGTGGCTGCTGCCAGTGGCGA | |
| | TAAGTCGTGTCTTACCGGGTTGGACTCAAGACGATAGTTACCGGATAAGGCGCAGCGGTCGGGCTGA | |
| | ACGGGGGGTTCGTGCACACAGCCCAGCTTGGAGCGAACGACCTACACCGAACTGAGATACCTACAGC | |
| | GTGAGCTATGAGAAAGCGCCACGCTTCCCGAAGGGAGAAAGGCGGACAGGTATCCGGTAAGCGGCAG | |
| | GGTCGGAACAGGAGAGCGCACGAGGGAGCTTCCAGGGGGAAACGCCTGGTATCTTTATAGTCCTGTC | |
| | GGGTTTCGCCACCTCTGACTTGAGCGTCGATTTTTGTGATGCTCGTCAGGGGGGCGGAGCCTATGGA | |
| | AAAACGCCAGCAACGCGGCCTTTTTACGGTTCCTGGCCTTTTGCTGGCCTTTTGCTCACATGTTCTT | |
| | TCCTGCGTTATCCCCTGATTCTGTGGATAACCGTATTACCGCCTTTGAGTGAGCTGATACCGCTCGC | |
| | CGCAGCCGAACGACCGAGCGCAGCGAGTCAGTGAGCGAGGAAGCGGAAGAGCGCCTGATGCGGTATT | |
| | TTCTCCTTACGCATCTGTGCGGTATTTCACACCGCATATGGTGCACTCTCAGTACAATCTGCTCTGA | |
| | TGCCGCATAGTTAAGCCAGTATACACTCCGCTATCGCTACGTGACTGGGTCATGGCTGCGCCCCGAC | |
| | ACCCGCCAACACCCGCTGACGCGCCCTGACGGGCTTGTCTGCTCCCGGCATCCGCTTACAGACAAGC | |
| | TGTGACCGTCTCCGGGAGCTGCATGTGTCAGAGGTTTTCACCGTCATCACCGAAACGCGCGAGGCAG | |
| | GGTGCCTTGATGTGGGCGCCGGCGGTCGAGTGGCGACGGCGCGGCTTGTCCGCGCCCTGGTAGATTG | |
| | CCTGGCCGTAGGCCAGCCATTTTTGAGCGGCCAGCGGCCGCGATAGGCCGACGCGAAGCGGCGGGGC | |
| | GTAGGGAGCGCAGCGACCGAAGGGTAGGCGCTTTTTGCAGCTCTTCGGCTGTGCGCTGGCCAGACAG | |
| | TTATGCACAGGCCAGGCGGGTTTTAAGAGTTTTAATAAGTTTTAAAGAGTTTTAGGCGGAAAAATCG | |
| | CCTTTTTTCTCTTTTATATCAGTCACTTACATGTGTGACCGGTTCCCAATGTACGGCTTTGGGTTCC | |
| | CAATGTACGGGTTCCGGTTCCCAATGTACGGCTTTGGGTTCCCAATGTACGTGCTATCCACAGGAAA | |
| | GAGACCTTTTCGACCTTTTTCCCCTGCTAGGGCAATTTGCCCTAGCATCTGCTCCGTACATTAGGAA | |
| | CCGGCGGATGCTTCGCCCTCGATCAGGTTGCGGTAGCGCATGACTAGGATCGGGCCAGCCTGCCCCG | |
| | CCTCCTCCTTCAAATCGTACTCCGGCAGGTCATTTGACCCGATCAGCTTGCGCACGGTGAAACAGAA | |
| | CTTCTTGAACTCTCCGGCGCTGCCACTGCGTTCGTAGATCGTCTTGAACAACCATCTGGCTTCTGCC | |
| | TTGCCTGCGGCGCGGCGTGCCAGGCGGTAGAGAAAACGGCCGATGCCGGGATCGATCAAAAAGTAAT | |
| | CGGGGTGAACCGTCAGCACGTCCGGGTTCTTGCCTTCTGTGATCTCGCGGTACATCCAATCAGCTAG | |
| | CTCGATCTCGATGTACTCCGGCCGCCCGGTTTCGCTCTTTACGATCTTGTAGCGGCTAATCAAGGCT | |
| | TCACCCTCGGATACCGTCACCAGGCGGCCGTTCTTGGCCTTCTTCGTACGCTGCATGGCAACGTGCG | |
| | TGGTGTTTAACCGAATGCAGGTTTCTACCAGGTCGTCTTTCTGCTTTCCGCCATCGGCTCGCCGGCA | |
| | GAACTTGAGTACGTCCGCAACGTGTGGACGGAACACGCGGCCGGGCTTGTCTCCCTTCCCTTCCCGG | |
| | TATCGGTTCATGGATTCGGTTAGATGGGAAACCGCCATCAGTACCAGGTCGTAATCCCACACACTGG | |
| | CCATGCCGGCCGGCCCTGCGGAAACCTCTACGTGCCCGTCTGGAAGCTCGTAGCGGATCACCTCGCC | |
| | AGCTCGTCGGTCACGCTTCGACAGACGGAAAACGGCCACGTCCATGATGCTGCGACTATCGCGGGTG | |
| | CCCACGTCATAGAGCATCGGAACGAAAAAATCTGGTTGCTCGTCGCCCTTGGGCGGCTTCCTAATCG | |
| | ACGGCGCACCGGCTGCCGGCGGTTGCCGGGATTCTTTGCGGATTCGATCAGCGGCCGCTTGCCACGA | |
| | TTCACCGGGGCGTGCTTCTGCCTCGATGCGTTGCCGCTGGGCGGCCTGCGCGGCCTTCAACTTCTCC | |
| | ACCAGGTCATCACCCAGCGCCGCGCCGATTTGTACCGGGCCGGATGG | |

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

A “genetically modified” cell refers to a cell in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

The terms “genome modification” and “genome editing” refer to processes by which a specific nucleic acid sequence in a genome is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences may also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) may be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm may be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP may be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs may be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

As various changes could be made in the above-described cells and methods without departing from the scope of the invention, it is intended that all matter contained in the above description and in the examples given below, shall be interpreted as illustrative and not in a limiting sense.

### EXAMPLES

The publications discussed above are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes may be made in the disclosure and still obtain a like or similar result without departing from the spirit and scope of the disclosure, therefore all matter set forth is to be interpreted as illustrative and not in a limiting sense.

### Example 1. Visualization of Repair Template Integration

A homologous recombination composition was used to fuse a GFP reporter at the C-terminus of the MeSWEET10 protein in cassava callus tissue. As shown in FIG. 8, the strategy comprised providing combining the CRISPR-Cas9 binary vector (containing two gRNAs targeted to the C-terminus of MeSWEET10a), a repair template (GFP flanked by ˜850 bp homology arms) and the TAL20 transcriptional activator, driven by the tissue-specific (FEC) promoter.

Specifically, two plasmids were prepared. One plasmid (169; SEQ ID NO: 7) comprises a construct for expressing the AtCas9 protein in combination with the csy4 CRISPR RNA processing protein from Pseudomonas aeruginosa under the control of the 35S promoter. The sequence for expressing AtCas9 protein is codon optimized for expression in Arabidopsis. The plasm id further comprises a construct for expressing the two gRNAs of the system under the control of the CYMLV promoter. A first gRNA targets MeSWEET10a just before stop codon and a second gRNA targets region just after MeSWEET10a stop codon. The two gRNAs are separated by csy4 binding sites for processing the two gRNAs. The plasm id also comprises the donor nucleic acid sequence (repair template), an expression construct for expression of a selectable reporter (NPTII), and T-DNA borders for transformation into cassava cells. A construct for expressing the TAL20 transcription activator under the control of the 35S promoter was inserted into the 169 plasmid to generate plasmid 171 (SEQ ID NO: 9). As such, plasm id 171 provides all the components of the homologous recombination composition, whereas plasm id 169 may be used as a control wherein the transcription activator is not present.

The composition was introduced into cassava via Agrobacterium-mediated transformation using T-DNA, and callus cells (specifically Friable Embryonic Calli, or FEC cells) were screened for GFP signal using epifluorescence. Through this screening process, five GFP-positive sectors of FEC cells were identified out of many hundreds that harbored the T-DNA. PCR and sequencing confirmed integration of GFP in frame at the C-terminus of MeSWEET10a, just 5′ of the stop codon, exactly matching the repair template, in one of these FEC populations (FIG. 8, Panel 5A). This demonstrates that Cas9-facilitated sequence integration coupled with a TA step is a viable strategy for identifying edited cells. Further, correct recombination was confirmed in leaves of cassava generated using the identified GFP-positive sectors of FEC cells (FIG. 8, Panel 5B). This was particularly encouraging since this work was done both in tissue culture (a typical step for plant transformation), and in a species that is relatively far from being a model for plants.

### Example 2. Visualization of Repair Template Integration Using a Tissue Specific Promoter

A similar experiment as described in Example 1 may be performed, wherein the transcription activator is under the control of the Manes.17G095200 callus-specific promoter.

Two plasm ids are prepared. One plasm id comprises a construct for expressing the AtCas9 protein in combination with the csy4 CRISPR RNA processing protein, a construct for expressing the two gRNAs of the system under the control of the CYMLV promoter, and the donor nucleic acid sequence. The plasmid is plasmid 169 described in Example 1 above. A construct for expressing the TAL20 transcription activator under the control of the tissue-specific Manes.17G095200 is inserted into the 169 plasmid to generate plasmid 170 (SEQ ID NO: 8). Plasmid 170 provides all the components of the homologous recombination composition, whereas plasmid 169 is used as a control wherein the transcription activator is not present.

When used in cassava callus cells, the callus transformed with the 169 plasm id only shows background fluorescence. Conversely, cells transformed with the 170 plasm id shows some cells clearly expressing GFP over the background fluorescence, thereby identifying accurate homologous recombination events in these cells.

### Example 3. Knock-Outs by Knock-Ins: Using a Homologous Recombination Composition for Single-Gene Knockouts and Large Deletions

A powerful, but perhaps counterintuitive strategy is to use a homologous recombination composition to generate knockouts (“KOs”), reducing the time and cost associated with genotyping regenerated plants. Typically, when aiming to KO a gene with CRISPR, regenerants must be genotyped unless the phenotype is obvious. Using the homologous recombination composition of the disclosure, stop codons are introduced downstream of the fluorescent protein fusion (FIG. 3A). In the case of a single gene knockout, the reporter replaces the start codon, and/or 5′, 3′, or internal coding exons, or adds a new terminated exon, in each case disrupting the function of the GOI and leaving in its place a visual marker that is induced by the TA in the tissue in which screening is performed. This accelerates the process of genotyping, since the disruption results from successful HR.

A homologous recombination composition is also used to generate deletions or sequence replacements between two genes of interest. This is achieved by targeting a pair of genes that are located some distance from one another and using HR to simultaneously introduce two reporters into these genes, while also replacing the intervening sequence with a different nucleic acid sequence. Expression of both reporters after HR indicates replacement of the original nucleic acid sequence between the two genes of interest (FIG. 3B). Expression of both reporters after HR indicates replacement of the original nucleic acid sequence between the two genes of interest.

### Example 4. Tagging Specific Members of Highly Similar Multi-Gene Families

Specific members of highly similar multigene families are tagged using 5′ or 3′ UTR differences. In this application of the disclosed compositions, an RNA aptamer strategy as described below is used. This strategy is used to tag specific members of the PPR gene family in Arabidopsis, as it is comprised of hundreds of members with a wide range of similarities, and family members are targets of many small RNAs.

### Example 4. Tagging Using Fluorescence RNA Aptamers

When relying on fluorescent proteins, non-coding RNAs cannot be tagged (without introducing an ORF). Fluorescent RNA are non-coding, fluorophore-binding RNA sequences of ˜70 nt. The ability to tag an RNA transcript in situ and track these transcripts is a powerful technique for studying gene function.

Intended uses of fluorescent aptamers includes: (i) use to direct homologous recombination with small translational epitope fusions in the 5′ or 3′ UTRs adjacent to a gene of interest (FIG. 6), or (ii) insert aptamers in noncoding RNAs, including miRNAs (FIG. 7). Use to tag the 5′ or 3′ UTRs adjacent to a gene of interest may be as described in Example 1.

For noncoding RNAs, a composition is used to tag TAS3 lncRNA and miR390 in cassava, Setaria, and Arabidosis with RNA aptamers that may ultimately allow the localization and quantification of these molecules, using super resolution microscopy. An additional application includes RNA capture. For example, an additional RNA sequence is added for RNA capture, such as the BoxB sequence bound by lambda protein “N”.

For tagging lncRNA modifications, numerous possibilities are used, and are as schematically described in FIG. 7. Of particular interest is conversion of 22-nt miRNA to 21-nt miRNAs (FIG. 7, panel C), altering their ability to trigger secondary siRNAs at target transcripts. In cassava and many eudicots, miR482 that targets NB-LRR R-genes is of particular interest. Poorly understood reproductive phasiRNAs in grasses are also be targeted to insert a BoxB RNA binding site (for lambda N) into a Setaria phasiRNA precursor, into both the 3′ and 5′ precursor ends (FIG. 7, panels E and F).

### Example 5. High-Throughput Applications: The Potential to Target Every Gene in a Genome

Methods of the disclosure are upscaled to whole-genome applications. In other words, whole-genome methods have long been deployed in Arabidopsis. Disclosed compositions are used to create genome-wide knock-in libraries in diverse species in addition to Arabidopsis. Compositions are used to knock-out or epitope tag every gene in a species recalcitrant to homologous recombination. Constructs for a composition of the disclosure are prepared wherein gene-specific components are concentrated into a single cassette that can be synthesized and cloned in bulk (FIG. 5). Agilent oligo synthesis using inkjet printing-based methods are used to synthesize up to 100,000+custom oligos that are over 150 nt in length. Overlapping oligos are designed, and annealing plus a fill-in reaction are performed to generate thousands of unique fragments of 100's of nts that are cloned en masse, to create a complex library for bulk transformation in methods of the disclosure. Methods are used for either forward (screening of anonymous but targeted knockout libraries) or reverse (epitope tagging, or deconvoluted knockout lines) genetics approaches. For deconvolution, the gene-specific construct is amplified and sequenced using a multi-dimensional pooling strategy. This type of strategy has been implemented in numerous large-scale CRISPR library screens in cell lines but has not been implemented in species recalcitrant to homologous recombination such as plants. To enable this application, constructs are constructed to co-locate the gene-specific components, flanked by BsaI sites, for intermediate cloning to a vector with Gateway cloning sites, enabling highly efficient ligation. Colocating components specific to the GOI (see FIG. 5) enables production on one cassette of two guide RNAs (Cpf1 for HR, dCas9 for TA), and the insertion fragment) totaling ˜350 bp.

In an initial experiment, a set of 48 target genes are targeted, for which cassettes are synthesized using overlapping oligos in 96-well plates. This 48-plex library is transformed by dip transformation into Arabidopsis or flax, and is de-convoluted by sequencing the gene-specific cassette for each resulting line before assessing the target site modifications. For the 48 genes, components of a single pathway are selected, such as small RNA biogenesis (Dicers, AGOs, etc.).

### Example 6. The CRISPR-Act3.0 Transcriptional Activation (TA) System

A modified SureFire HR system (SureFire HR v2) was devised and constructed. In SureFire HR v2, a single CRISPR system was used for inducing homologous recombination and for transcriptional activation. In short, the CRISPR system comprised a CRISPR nuclease and a modified gRNA scaffold comprising MS2-binding RNA aptamers that recruit transcriptional activators modified to bind the RNA aptamers. In this study, the CRISPR nuclease was a Cas9 nuclease optimized for use in maize (zCas9) and the modified gRNA scaffold and transcriptional activators were the CRISPR-Act3.0 activator system described in Pan et al (Nature Plants volume 7, pages 942-953 (2021)) the disclosure of which is incorporated herein in its entirety (see FIG. 9). All components colored in green are modifications unique to Surefire HRv2). Plasm ids were constructed for use in Arabidopsis and rice comprising the system under the control of various promoters and are summarized in Table 1. The vector assembly methods described in Pan et al. (doi: 10.1038/s41477-021-00953-7.) was utilized, including: i) Esp3I (isomer of BsmBI) insertion of gRNA duplex into entry plasm ids; ii) BsaI golden gate assembly of sgRNAs into Gateway entry plasmid; and 3.) LR multi-fragment GW assembly of guides and Cas-TA into binary vector. This new vector building strategy enabled the future SureFire user a simple method to program SureFire HRv2 for gene specific targeting, repair, and tissue specific transcriptional activation for selection.

The constructs shown in Table 1 can express dRNAs, gRNAs, or both under the control of tissue specific promoters or constitutive promoters. Further, the promoters can be plant- and plant species-specific. The tissue might be different for every species that is targeted, and it depends on the transformation method and selection methods used. Here, a seed coat specific promoter for Arabidopsis (oleosin1 Promoter—atOLE1) was used to drive the dRNAs, because the selection of positive HR events is done in seeds. A callus specific promoter for rice was also used, because the selection of positive HR transformants is done during callus regeneration. Additionally, by using these tissue specific promoters, the system enabled the study of native expression of the gene of interest (or as close as possible).

### Example 7. One Single Active Cas9 Using Dead RNAs

Surefire version 1 used two CRISPR systems, one for double-stranded DNA cleavage and the other for transcriptional activation (TA). Surefire HR version 2

(Boxes labelled in green contain modifications unique to SureFire HRv2)

The constructs shown in Table 1 can express dRNAs, gRNAs, or both under the control of tissue specific promoters or constitutive promoters. Further, the promoters can be plant- and plant species-specific. The tissue might be different for every species that is targeted, and it depends on the transformation method and selection methods used. Here, a seed coat specific promoter for Arabidopsis (oleosin1 Promoter—atOLE1) was used to drive the dRNAs, because the selection of positive HR events is done in seeds. A callus specific promoter for rice was also used, because the selection of positive HR transformants is done during callus regeneration. Additionally, by using these tissue specific promoters, the system enabled the study of native expression of the gene of interest (or as close as possible).

### Example 7. One Single Active Cas9 Using Dead RNAs

Surefire version 1 used two CRISPR systems, one for double-stranded DNA cleavage and the other for transcriptional activation (TA). Surefire HR version 2 modifies the system to allow for the use of a single CRISPR system for achieving both homologous recombination and transcriptional activation. Version 2 can accomplish this by making use of truncated guide RNAs known as deadRNAs (dRNAs) which are about 11 to 15 nucleotides-long. dRNAs can guide catalytically active CRISPR nucleases to their target sequences but prevent induction of the nuclease function of the CRISPR nuclease. Accordingly, a single CRISPR system can be used in SureFire v2 for homologous recombination when guided by gRNAs, and for transcriptional regulation when guided by dRNAs.

In this study, twenty nucleotides (20 nt) gRNAs were used to guide CRISPR-Act3.0 to the desired homologous recombination target site and endogenously cleave the GOI at the desired site to induce DNA repair by homologous recombination with the repair/donor template. Additionally, 15 nt-dRNAs were used to target the CRISPR-Act3.0 upstream of a GOI's transcription start-site (TSS) for transcriptional regulation.

An expression construct expressing 15 nt dRNAs under the control of the At U3 ubiquitous promoter was introduced into pRD238 Arabidopsis lines comprising expression constructs for expressing zCas9-Act3.0 under the control of the At UBQ10 promoter. The dRNAs guide the CRISPR transcriptional activator to the FT locus to overexpress the FT locus. In five pRD238 T1 lines, the 15 nt deadRNAs triggered transcriptional activation of the FT locus as evidenced by the early flowering in 21-day old (FIG. 11) and 17-day-old (FIG. 12) plants. There was little to no presence of rosettes in 45-day-old plants (FIG. 13). The 15-nt dRNAs also triggered transcriptional activation of the FT locus in T2 lines as indicated by At.FT expression in 45-day old Arabidopsis bud/floral tissue (FIG. 14), and in T3 lines as indicated by At.FT expression in 18-day old Arabidopsis bud/floral tissue (FIG. 15).

### Example 9. Callus Specific dRNA Promoter for Rice

A reporter expression construct (SEQ ID NO: 12) was prepared by fusing a rice callus specific promoter (OsCSP; SEQ ID NO: 10) amplified from a rice genome to a GFP reporter for transformation into rice callus cells. The expression construct was capable of inducing GFP expression in callus (see FIG. 11). Additionally, it was observed that the GFP signal from the OsCSP:GFP construct was no longer visible in mature plants. This suggested that OsCSP is active only in callus, making it a great candidate for SureFire HR in rice and potentially across different monocot species.

For SureFire HRv2, a modified version of OsCSP (1097 bp; SEQ ID NO: 11) was designed and synthesized, including the removal of three internal restriction enzyme recognition sites that would interfere with downstream assembly. They were BsaI-(G563A), Esp3I-(G1077A); and Esp3I-(C1080A). Substitution G626C was also made to disrupt a polyG(10) to tolerate nucleic acid synthesis. DsDNA fragments containing OsCSP were synthesized by twist bioscience and then cloned into the dead RNA entry vectors.

### Example 10. Generation of Three Arabidopsis zCas9::Act3.0-Expressing Parental Lines (for Future Sequential Transformation)

Double stranded DNA breaks were generated in meiotic germ line cells to produce heritable gene insertions using SureFire HR. Homologous recombination occurs mainly during the G and G2 phases of the cell cycle. To generate the parental lines, one ubiquitous promoter and two tissue specific promoters were selected to drive Active zCas9::Act3.0 that have been shown to induce hereditable mutations in subsequent generations of Arabidopsis transformants. For that, the ubiquitous promoter UBQ10, the egg-cell specific promoter At.EC1.2e1.1p, and the embryo specific promoter At.YAO were fused to the nucleic acid sequence that expresses CRISPR-Act3.0. The parental lines and expression of zCas9::Act3.0 are as described in Table 2. Expression of zCas9-Act3.0 was detected in Arabidopsis leaf and bud tissue by RT-PCR (FIGS. 16 and 17).

| | | | zCas9- | | | |
|---|---|---|---|---|---|---|
| | | | Act3.0 | | # T1 | |
| # | Plasmid ID | Transgene | expression | Resistance | Hygro^{R} | T-DNA |
| 1 | pRD243 | At. UBQ10ₚᵣₒ::Cas9- | Ubiquitous | Hygro | 13 | 12 |
| | | Act3.0 | | | | |
| 2 | pRD265 | At. | Egg cell | Hygro | 3 | 3 |
| | | EC(1.2e/1.1p)ₚᵣₒ::Cas9- | | | | |
| | | Act3.0 | | | | |
| 3 | pRD266 | At. YAOₚᵣₒ::Cas9-Act3.0 | Embryo | Hygro | 18 | 7 |

What is claimed is:

1. A homologous recombination system for detecting an accurate homologous recombination event in a gene of interest in a cell, the recombination system comprising:
a. an expression construct for expressing a programmable RNA-guided nuclease transcription regulator, the expression construct comprising a promoter operably linked to a nucleic acid sequence encoding the programmable RNA-guided nuclease transcription regulator;
b. one or more expression constructs for expressing a guide RNA (gRNA) and a deadRNA (dRNA), the one or more expression constructs comprising a promoter operably linked to a nucleic acid sequence comprising the gRNA, a promoter operably linked to a nucleic acid sequence comprising the dRNA an expression construct comprising a promoter operably linked to a dRNA, or a promoter operably linked to a nucleic acid sequence comprising the gRNA and a nucleic acid sequence comprising the dRNA, wherein the gRNA targets the programmable RNA-guided nuclease transcription regulator to a first nucleic acid sequence at a homologous recombination site in a gene of interest and the dRNA targets the programmable RNA-guided nuclease transcription regulator to a second nucleic acid sequence in a regulatory sequence of the gene of interest; and
c. a donor polynucleotide comprising a nucleic acid sequence encoding a reporter flanked by regions homologous to nucleic acid sequences at the homologous recombination site;
wherein a programmable RNA-guided nuclease transcription regulator targeted by the gRNA induces a homologous recombination event at the homologous recombination site and a programmable RNA-guided nuclease transcription regulator targeted by the dRNA regulates expression of the gene of interest.

2. The homologous recombination system of claim 1, wherein the programmable RNA-guided nuclease transcription regulator comprises an RNA-guided clustered regularly interspersed short palindromic repeats (CRISPR)/CRISPR-associated (Cas) (CRISPR/Cas) nuclease, a single guide RNA (sgRNA) scaffold comprising one or more aptamers, and one or more transcriptional activator linked to the one or more aptamers.

3. The homologous recombination system of claim 1, wherein one or more of the expression constructs are under control of a ubiquitous promoter or a tissue-specific promoter.

4. The homologous recombination system of claim 3, wherein the ubiquitous promoter or tissue-specific promoter is At. UBQ10 ubiquitous promoter, Arabidopsis (oleosin1 Promoter—atOLE1) promoter, At U3 ubiquitous promoter, rice callus specific promoter (OsCSP; SEQ ID NO: 10), modified rice callus specific promoter (OsCSP; SEQ ID NO: 11), egg-cell specific promoter At.EC1.2e1.1p, or embryo specific promoter At.YAO.

5. The homologous recombination system of claim 1, wherein the programmable RNA-guided nuclease transcription regulator is zCas9-Act3.0 transcriptional activator.

6. The homologous recombination system of claim 5, wherein the zCas9-Act3.0 transcriptional activator is encoded by a nucleic acid sequence comprising at least about 75% or more, at least about 85% or more, at least about 95% or more, or 100% sequence identity with base 1 to base 6,250 of SEQ ID NO: 13.

7. The homologous recombination system of claim 6, wherein zCas9-Act3.0 transcriptional activator is under control of the At. UBQ10 ubiquitous promoter, egg-cell specific promoter At. EC1.2e1.1p, or embryo specific promoter At.YAO.

8. The homologous recombination system of claim 1, wherein an expression construct for expressing a dRNA comprises a modified OsCSP promoter operably linked to a nucleic acid sequence comprising a dRNA.

9. The homologous recombination system of claim 8, wherein the expression construct comprises a nucleic acid sequence comprising at least about 75% or more, at least about 85% or more, at least about 95% or more, or 100% sequence identity with base 1,750 to base 3,340 of SEQ ID NO: 14.

10. The homologous recombination system of claim 8, wherein the expression construct comprises a nucleic acid sequence comprising at least about 75% or more, at least about 85% or more, at least about 95% or more, or 100% sequence identity with base 1,750 to base 3,340 of SEQ ID NO: 15.

11. The homologous recombination system of claim 1, wherein an expression construct for expressing a dRNA comprises an atOLE1 promoter operably linked to a nucleic acid sequence comprising a dRNA.

12. The homologous recombination system of claim 11, wherein the expression construct comprises a nucleic acid sequence comprising at least about 75% or more, at least about 85% or more, at least about 95% or more, or 100% sequence identity with base 1,750 to base 3,340 of SEQ ID NO: 16.

13. The homologous recombination system of claim 11, wherein the expression construct comprises a nucleic acid sequence comprising at least about 75% or more, at least about 85% or more, at least about 95% or more, or 100% sequence identity with base 1,750 to base 2,950 of SEQ ID NO: 17.

14. The homologous recombination system of claim 1, wherein the cell is a plant or part thereof, plant cell, or seed.

15. A genetically modified cell for detecting an accurate homologous recombination event in a gene of interest of the cell, the cell comprising a stably integrated expression construct for expressing a programmable RNA-guided nuclease transcription regulator.

16. The genetically modified cell of claim 15, wherein the expression construct comprises a promoter operably linked to a nucleic acid sequence encoding the programmable RNA-guided nuclease transcription regulator.

17. The genetically modified cell of claim 15, wherein the programmable RNA-guided nuclease transcription regulator comprises an RNA-guided clustered regularly interspersed short palindromic repeats (CRISPR)/CRISPR-associated (Cas) (CRISPR/Cas) nuclease, a single guide RNA (sgRNA) scaffold comprising one or more aptamers, and one or more transcriptional activator linked to the one or more aptamers.

18. The genetically modified cell of claim 15, wherein the programmable RNA-guided nuclease transcription regulator is zCas9-Act3.0 transcriptional activator.

19. The genetically modified cell of claim 18, wherein the zCas9-Act3.0 transcriptional activator is encoded by a nucleic acid sequence comprising at least about 75% or more, at least about 85% or more, at least about 95% or more, or 100% sequence identity with base 1 to base 6,250 of SEQ ID NO: 13.

20. The genetically modified cell of claim 18, wherein the zCas9-Act3.0 transcriptional activator is encoded by a nucleic acid sequence comprising at least about 75% or more, at least about 85% or more, at least about 95% or more, or 100% sequence identity with base 1 to base 6,250 of SEQ ID NO: 14.

21. The genetically modified cell of claim 18, wherein zCas9-Act3.0 transcriptional activator is under control of the At. UBQ10 ubiquitous promoter, egg-cell specific promoter At. EC1.2e1.1p, or embryo specific promoter At.YAO.

22. A kit for detecting one or more accurate homologous recombination events in a cell, the kit comprising a homologous recombination system of claim 1.
